(19) United States
(12) Patent Application Publication
(10) Pub. No.: US 2024/0238445 A1
(43) Pub. Date: Jul. 18, 2024

(54) INHIBITORS AND USES THEREOF

(71) Applicants: NEURACLE SCIENCE CO., LTD., Seoul (KR); KOREA UNIVERSITY RESEARCH AND BUSINESS FOUNDATION, Seoul (KR); KOREA UNIVERSITY RESEARCH AND BUSINESS FOUNDATION, SEJONG CAMPUS, Seoul (KR)

(72) Inventors: Jae Young SEONG, Seoul (KR); Youngjoo BYUN, Daejon (KR); Hoyun KWAK, Hanam-si (KR); Sitaek OH, Seoul (KR); Min-Hyeok LEE, Seoul (KR); Yongwoo JEONG, Seoul (KR); Nui HA, Anyang-si (KR); Eun-Ho CHO, Seoul (KR); Suhyun LEE, Seoul (KR); Sang-Myeong LEE, Suwon-si (KR); Yerim LEE, Seoul (KR); Eun Bee CHO, Seoul (KR); Jae Keun LEE, Seoul (KR); Han-Byul KIM, Seoul (KR); Soon-gu KWON, Seoul (KR)

(73) Assignee: NEURACLE SCIENCE CO., LTD., Seoul (KR)

(21) Appl. No.: 18/552,290

(22) PCT Filed: Jul. 8, 2022
(86) PCT No.: PCT/IB2022/056358

§ 371 Date: Sep. 25, 2023

Related U.S. Application Data
(60) Provisional application No. 63/219,683, filed on Jul. 8, 2021.

(51) Int. Cl. A61K 48/00; A61K 31/055; A61K 31/085; A61K 31/136; A61K 31/137; A61K 38/08; A61K 38/10; A61K 38/17; A61K 45/06; A61K 47/64; A61P 25/02; A61P 25/28; C12N 5/0793

(52) U.S. Cl. CPC A61K 48/005; A61K 31/055; A61K 31/085; A61K 31/136; A61K 31/137; A61K 38/08; A61K 38/10; A61K 38/1709; A61K 45/06; A61K 47/64; A61P 25/02; A61P 25/28; C12N 5/0619

(57) ABSTRACT

The present disclosure provides mimic molecules that are capable of specifically targeting a FAM19A5 protein, and thereby, inhibiting, reducing, and/or dissociating the interaction between members of the LRRC4 protein family and the FAM19A5 protein. The present disclosure also provides methods for promoting neurite outgrowth, by administering a mimic molecule described herein.

### FIELD OF THE DISCLOSURE

This PCT application claims the priority benefit of U.S. Provisional Application No. 63/219,683, filed Jul. 8, 2021, which is herein incorporated by reference in its entirety.

### REFERENCE TO SEQUENCE LISTING SUBMITTED ELECTRONICALLY VIA EFS-WEB

The content of the electronically submitted sequence listing in .XML file (Name: 3763.019PC01_Seqlisting_ST26.xml, Size: 159,077 bytes; and Date of Creation: Jul. 8, 2022) submitted in this application is incorporated herein by reference in its entirety.

### FIELD OF THE DISCLOSURE

The present disclosure provides LRRC4 family mimic molecules that are capable of specifically inhibiting, reducing, and/or dissociating the interaction between a member of the LRRC4 protein family and a FAM19A5 protein.

### BACKGROUND OF THE DISCLOSURE

Mammalian neurons constantly protrude neurites, including axons and dendrites, to form synapses with other neurons, muscles, and blood vessels. At the same time, neurons retract neurites to disassemble unnecessary synapses (e.g., those that have not been used for an extended period of time). This balance of gain and loss of synapses is critical for healthy central and peripheral nervous systems.

However, various factors (e.g., aging, cytotoxic microenvironment, acute damages, genetic mutations) can result in abnormal loss of synapses. Such increased loss of synapses is associated with various neurological disorders, including mental retardation, schizophrenia, autism spectrum disorder, Alzheimer's disease, Parkinson's disease, amyotrophic lateral sclerosis, multiple sclerosis, Huntington's disease, prion diseases, neuropathic pain, spinal cord injury, and stroke. See, e.g., Hayashi-Takagi, Neurosci Res 114: 3-8 (January 2017); Wang et al., Prog Neuropsychopharmacol Biol Psychiatry 84(Pt B): 398-415 (June 2018); Jha et al., J Alzheimers Dis 57(4): 1017-1039 (2017); Mitoma et al., Int J Mol Sci 21(14): 4936 (July 2020); and Brose et al., Biochem Soc Trans 38(2): 443-4 (April 2010).

Because the underlying causes of neurological disorders are not always fully understood, many of the current treatment options merely focus on treating the symptoms associated with the disorders. And, where there are treatments available, they can be associated with adverse side effects and/or limited efficacy. Therefore, there is a current need for alternative treatment options that are more effective for neurological disorders, such as those associated with abnormal loss of synapses.

### BRIEF SUMMARY OF THE DISCLOSURE

Provided herein is Leucine Rich Repeat Containing 4 (“LRRC4”) family mimic molecule that is capable of inhibiting, reducing, and/or dissociating an interaction between a Family with Sequence Similarity 19, Member A5 (“FAM19A5”) protein and a member of a LRRC4 protein family. In some aspects, the LRRC4 family mimic molecule is not an antibody or an antigen-binding portion thereof. In some aspects, the LRRC4 family mimic molecule comprises a peptide. In some aspects, the LRRC4 family mimic molecule comprises a small molecule.

In some aspects, the LRRC4 family mimic molecule is a small molecule of formula (I):

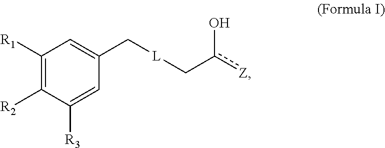

or a pharmaceutically acceptable salt thereof, wherein:
(i) R₁, R₂ and R₃ are independently selected from hydrogen, fluoro, chloro, bromo, methyl, ethyl, n-propyl, iso-propyl, n-butyl, iso-butyl, t-butyl, n-pentyl, iso-pentyl, fluoromethyl, difluoromethyl, trifluoromethyl, 2-fluoroethyl, 1-fluoroethyl, 2,2-difluoroethyl, 1,2-difluoroethyl, 1,1-difluoroethyl, 2,2,2-trifluoroethyl, methoxy, ethoxy, n-propyloxy, iso-propyloxy, n-butoxy, trifluoromethoxy, difluoromethoxy, fluoromethoxy, acetyl, propionyl, n-butanoyl, iso-butanoyl, n-pentanoyl, nitro, amino, N-methylamino, N-ethylamino, N-n-propylamino, N,N-dimethylamino, N-acetylamino, N-propionylamino, N-(trifluoroacetyl)amino, formyl, hydroxy, methylthio, ethylthio, n-propylthio, methylsulfonyl, ethylsulfonyl, n-propylsulfonyl, phenyl, hydroxymethyl, 1-hydroxyethyl and 2-hydroxyethyl;
(ii) [symbol] is a single or double bond;
(iii) Z is selected from a straight chain or branched (C₁-C₈)alkyl, a straight chain or branched (C₂-C₈)alkenyl, a straight chain or branched (C₂-C₈)alkynyl, (C₃-C₈)cycloalkyl, (C₅-C₈)cycloalkenyl, (3-8 membered) heterocycloalkyl, (C₇-C₁₄)bicycloalkyl, (C₇-C₁₄) bicycloalkenyl, (7-14 membered) heterobicycloalkyl, (C₆-C₁₀) aryl, (5-10-membered) heteroaryl, and —CH—C(O)—CH═CH-Q, wherein Q is (C₃-C₈)cycloalkyl, (C₅-C₈)cycloalkenyl, (3-8 membered)heterocycloalkyl, (C₆-C₁₀)aryl, and (5-6-membered)heteroaryl; wherein each cycloalkyl, cycloalkenyl, heterocyclylalkyl, aryl, and heteroaryl are optionally substituted with one, two, three, four, or five substituents independently selected from (C₁-C₆)alkoxy, (C₁-C₆)alkyl, halo, (C₁-C₆)haloalkoxy, nitro, amino, N-methylamino, N-ethylamino, N—N-propylamino, N,N-dimethylamino, formyl, and hydroxy, and
(iv) L is single, double or triple bond, and
   wherein the LRRC4 family mimic molecule is not a small molecule selected from:

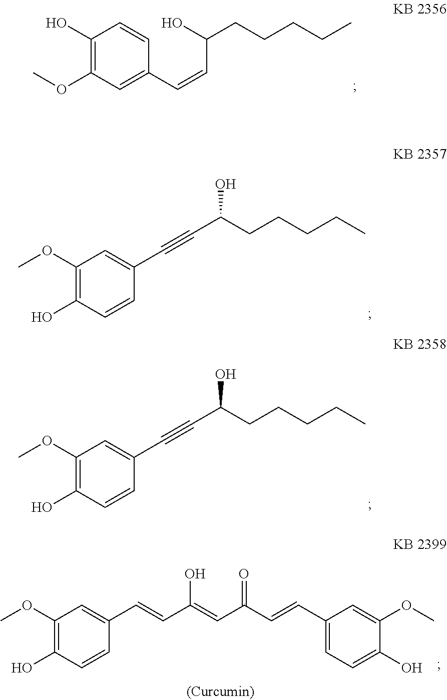

or a pharmaceutically acceptable salt thereof.

In some aspects, the LRRC4 family mimic molecule is a small molecule of formula (II):

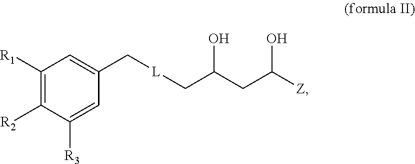

or a pharmaceutically acceptable salt thereof, wherein:
(i) R1, R2 and R3 are independently selected from hydrogen, fluoro, chloro, bromo, methyl, ethyl, n-propyl, iso-propyl, n-butyl, iso-butyl, t-butyl, n-pentyl, iso-pentyl, fluoromethyl, difluoromethyl, trifluoromethyl, 2-fluoroethyl, 1-fluoroethyl, 2,2-difluoroethyl, 1,2-difluoroethyl, 1,1-difluoroethyl, 2,2,2-trifluoroethyl, methoxy, ethoxy, n-propyloxy, iso-propyloxy, n-butoxy, trifluoromethoxy, difluoromethoxy, fluoromethoxy, acetyl, propionyl, n-butanoyl, iso-butanoyl, n-pentanoyl, nitro, amino, N-methylamino, N-ethylamino, N-n-propylamino, N,N-dimethylamino, N-acetylamino, N-propionylamino, N-(trifluoroacetyl)amino, formyl, hydroxy, methylthio, ethylthio, n-propylthio, methylsulfonyl, ethylsulfonyl, n-propylsulfonyl, phenyl, hydroxymethyl, 1-hydroxyethyl and 2-hydroxyethyl,
(ii) Z is selected from a straight chain or branched (C₁-C₈)alkyl, a straight chain or branched (C₂-C₈)alkenyl, a straight chain or branched (C₂-C₈)alkynyl, (C₃-C₈)cycloalkyl, (C₅-C₈)cycloalkenyl, (3-8 membered) heterocycloalkyl, (C₇-C₁₄)bicycloalkyl, (C₇-C₁₄) bicycloalkenyl, (7-14 membered) heterobicycloalkyl, (C₆-C₁₀) aryl, (5-10-membered) heteroaryl, and —CH═CH-Q, wherein Q is (C₃-C₈)cycloalkyl, (C₅-C₈)cycloalkenyl, (3-8 membered)heterocycloalkyl, (C₆-C₁₀)aryl, and (5-6-membered)heteroaryl; wherein each cycloalkyl, cycloalkenyl, heterocyclylalkyl, aryl, and heteroaryl are optionally substituted with one, two, three, four, or five substituents independently selected from (C₁-C₆)alkoxy, (C₁-C₆)alkyl, halo, (C₁-C₆)haloalkoxy, nitro, amino, N-methylamino, N-ethylamino, N—N-propylamino, N,N-dimethylamino, formyl, and hydroxy, and
(iii) L is single, double or triple bond.

In some aspects, the LRRC4 family mimic molecule is selected from:

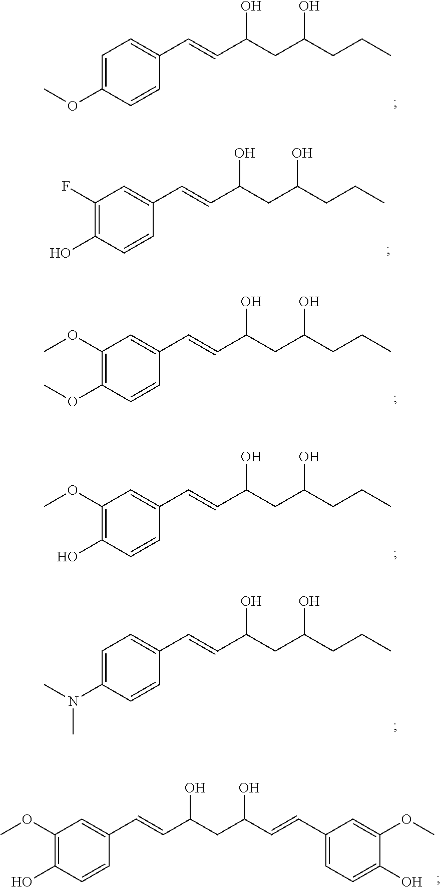

or a pharmaceutically acceptable salt thereof.

In some aspects, the LRRC4 family mimic molecule is a small molecule of formula (III):

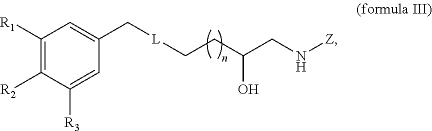

or a pharmaceutically acceptable salt thereof, wherein:
(i) R1, R2 and R3 are independently selected from hydrogen, fluoro, chloro, bromo, methyl, ethyl, n-propyl, iso-propyl, n-butyl, iso-butyl, t-butyl, n-pentyl, iso-pentyl, fluoromethyl, difluoromethyl, trifluoromethyl, 2-fluoroethyl, 1-fluoroethyl, 2,2-difluoroethyl, 1,2-difluoroethyl, 1,1-difluoroethyl, 2,2,2-trifluoroethyl, methoxy, ethoxy, n-propyloxy, iso-propyloxy, n-butoxy, trifluoromethoxy, difluoromethoxy, fluoromethoxy, acetyl, propionyl, n-butanoyl, iso-butanoyl, n-pentanoyl, nitro, amino, N-methylamino, N-ethylamino, N-n-propylamino, N,N-dimethylamino, N-acetylamino, N-propionylamino, N-(trifluoroacetyl)amino, formyl, hydroxy, methylthio, ethylthio, n-propylthio, methylsulfonyl, ethylsulfonyl, n-propylsulfonyl, phenyl, hydroxymethyl, 1-hydroxyethyl and 2-hydroxyethyl,
(ii) Z is selected from a straight chain or branched (C₁-C₈)alkyl, a straight chain or branched (C₂-C₈)alkenyl, a straight chain or branched (C₂-C₈)alkynyl, —Y—(C₃-C₈)cycloalkyl, —Y—(C₅-C₈)cycloalkenyl, —Y-(3-8 membered) heterocycloalkyl, —Y—(C₇-C₁₄)bicycloalkyl, —Y—(C₇-C₁₄) bicycloalkenyl, —Y-(7-14 membered) heterobicycloalkyl, —Y—(C₆-C₁₀)aryl, and —Y-(5-10-membered) heteroaryl, wherein Y is a bond or a C₁-C₃ straight or branched alkylene, and wherein the cycloalkyl, the cycloalkenyl, the heterocyclylalkyl, the aryl, and the heteroaryl are optionally substituted with one, two, three, four, or five substituents independently selected from C₁-C₆alkoxy, C₁-C₆alkyl, halo, C₁-C₆haloalkoxy, nitro, amino, N-methylamino, N-ethylamino, N—N-propylamino, N,N-dimethylamino, formyl, and hydroxy,
(iii) L is single, double or triple bond, and
(iv) n is 0 or 1.

In some aspects, the LRRC4 family mimic molecule is selected from:

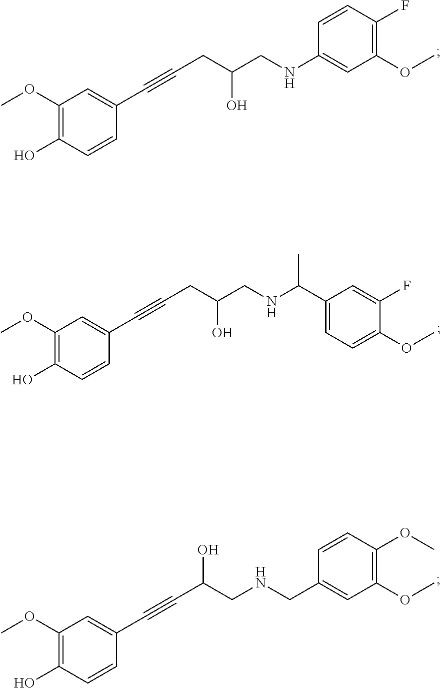

or a pharmaceutically acceptable salt thereof.

In some aspects, the LRRC4 family mimic molecule comprises a polypeptide comprising, consisting of, or consisting essentially of a domain of a LRRC4 protein family member, wherein the domain is capable of binding to a FAM19A5 protein (“FAM19A5 binding domain”), and wherein the polypeptide is shorter than the corresponding full-length LRRC4 protein family member (e.g., SEQ ID NO: 4; SEQ ID NO: 5; or SEQ ID NO: 6).

In some aspects, the FAM19A5 binding domain is about 10 to about 23 amino acids in length. In some aspects, the FAM19A5 binding domain is about 10, about 11, about 12, about 13, about 14, about 15, about 16, about 17, about 18, about 19, about 20, about 21, about 22, or about 23 amino acids in length. In some aspects, the FAM19A5 binding domain is about 10 amino acids in length.

In some aspects, the LRRC4 family mimic molecule comprises a polypeptide, which comprises an amino acid sequence having the following formula (from N-terminus to C-terminus):

(Formula IV)
A-(T/S)-B,

(i) A comprises X1-(T/S)-(Y/F)-F-X5; wherein:
   X1 is tyrosine (Y), phenylalanine (F), valine (V), leucine (L), or isoleucine (I);
   (T/S) is threonine (T) or serine (S);
   (Y/F) is tyrosine (Y) or Phenylalanine (F); and
   X5 is any amino acid; and
(ii) B comprises (V/I)-T-V-(E/V); wherein:
   (V/I) is valine (V) or isoleucine (I); and
   (E/V) is glutamic acid (E) or valine (V).

In some aspects, the LRRC4 family mimic molecule comprises a polypeptide, which comprises an amino acid sequence having the following formula (from N-terminus to C-terminus):

(Formula IV)
A-(T/S)-B,

(i) A comprises (Y/W/M)-(T/Y)-(Y/W)-(F/Y/W)-(T/Y); wherein:
   (Y/W/M) is tyrosine (Y), tryptophan (W), or methionine (M);
   (T/Y) is threonine (T) or tyrosine (Y);
   (Y/W) is tyrosine (Y) or tryptophan (W); and
   (F/Y/W) is phenylalanine (F), tyrosine (Y), or tryptophan (W); and
(ii) B comprises X7-(T/S/Y)-X9-X10; wherein:
   X7 is valine (V), tyrosine (Y), phenylalanine (F), leucine (L), tryptophan (W), or methionine (M);
   (T/S/Y) is threonine (T), serine (S), or tyrosine (Y);
   X9 is valine (V), isoleucine (I), tyrosine (Y), phenylalanine (F), leucine (L), tryptophan (W), or methionine (M); and
   X10 is glutamic acid (E), aspartic acid (D), isoleucine (I), tyrosine (Y), phenylalanine (F), methionine (M), or tryptophan (W).

Also provided herein is a LRRC4 family mimic molecule comprising a polypeptide, which comprises an amino sequence having the following formula (from N-terminus to C-terminus):

(Formula V)
X1-X2-X3-F-X5-T-X7-T-V-X10,

wherein:
X1 is Y, F, V, L, or I;
X2 is T or S;
X3 is Y or F;
X5 is any amino acid;
X7 is V or I; and/or
X10 is E or V, and
wherein the polypeptide is capable of binding to a FAM19A5 protein, and thereby, inhibiting, reducing, and/or dissociating an interaction between the FAM19A5 protein and a member of the LRRC4 protein family.

Also provided herein is a LRRC4 family mimic molecule comprising a polypeptide, which comprises an amino acid sequence having the following formula: (from N-terminus to C-terminus):

(Formula VI)
X1-X2-X3-X4-X5-X6-X7-X8-X9-X10,

wherein:
X1 is Y, F, V, L, I, W, or M;
X2 is T, S, or Y;
X3 is Y, F, or W;
X4 is F, Y, or W;
X5 is any amino acids, e.g., T, S, or Y;
X6 is T, S, or Y;
X7 is V, I, Y, F, L, W, or M;
X8 is T, S, or Y;
X9 is V, I, Y, F, L, W, or M; and/or
X10 is E, D, V, I, Y, F, M, or W, and
wherein the polypeptide is capable of binding to a FAM19A5 protein, and thereby, inhibiting, reducing, and/or dissociating an interaction between the FAM19A5 protein and a member of the LRRC4 protein family.

In some aspects, X1 is Y, F, V, L, or I. In some aspects, X2 is T or S. In some aspects, X3 is Y or F. In some aspects, X4 is F. In some aspects, X5 is T or S. In some aspects, X6 is T. In some aspects, X7 is V or I. In some aspects, X8 is T. In some aspects, X9 is V. In some aspects, X10 is E or V.

In some aspects, the LRRC4 family mimic molecule comprises a polypeptide which comprises the amino acid sequence set forth in SEQ ID NO: 29 (YTYFTTVTVE). In some aspects, the polypeptide consists of the amino acid sequence set forth in SEQ ID NO: 29. In some aspects, the LRRC4 family mimic molecule comprises a polypeptide which comprises the amino acid sequence set forth in SEQ ID NO: 20 (NYSFFTTVTVETTEISPEDTTRK). In some aspects, the polypeptide consists of the amino acid sequence set forth in SEQ ID NO: 20 (NYSFFTTVTVETTEISPEDTTRK). In some aspects, the LRRC4 family mimic molecule comprises the amino acid sequence set forth in SEQ ID NO: 30 (YSFFTTVTVE). In some aspects, the LRRC4 family mimic molecule consists of the amino acid sequence set forth in SEQ ID NO: 30 (YSFFTTVTVE). In some aspects, the LRRC4 family mimic molecule comprises a polypeptide which comprises the amino acid sequence set forth in SEQ ID NO: 21 (NFSYFSTVTVETMEPSQDERTTR). In some aspects, the polypeptide consists of the amino acid sequence set forth in SEQ ID NO: 21 (NFSYFSTVTVETMEPSQDERTTR). In some aspects, the LRRC4 family mimic molecule comprises the amino acid sequence set forth in SEQ ID NO: 31 (FSYFSTVTVE). In some aspects, the LRRC4 family mimic molecule consists of the amino acid sequence set forth in SEQ ID NO: 31 (FSYFSTVTVE).

In some aspects, the LRRC4 family mimic molecule comprises a polypeptide which comprises the amino acid sequence set forth in SEQ ID NO: 18 (GYTYFTTVTVETLETQPGEE). In some aspects, the polypeptide consists of the amino acid sequence set forth in SEQ ID NO: 18 (GYTYFTTVTVETLETQPGEE). In some aspects, amino acid residues T12 and L13 are modified (e.g., substituted) relative to the corresponding residues of SEQ ID NO: 18. In some aspects, the polypeptide comprises the amino acid sequence set forth in any one of SEQ ID NOs: 123-142. In some aspects, the polypeptide consists of the amino acid sequence set forth in any one of SEQ ID NOs: 123-142. In some aspects, one or more of the amino acid residues are in the form of a D-amino acid.

In some aspects, the LRRC4 family mimic molecule comprises the amino acid sequence set forth in SEQ ID NO: 17 (GYTYFTTVTVETLETQ). In some aspects, the LRRC4 family mimic molecule consists of the amino acid sequence set forth in SEQ ID NO: 17 (GYTYFTTVTVETLETQ). In some aspects, the LRRC4 family mimic molecule comprises the amino acid sequence set forth in SEQ ID NO: 19 (GYTYFTTVTVETLETQPGEKEPPGPTTD). In some aspects, the LRRC4 family mimic molecule consists of the amino acid sequence set forth in SEQ ID NO: 19 (GYTYFTTVTVETLETQPGEKEPPGPTTD).

In some aspects, the LRRC4 family mimic molecule comprises the amino acid sequence set forth in SEQ ID NO: 143 (GYTYFTTVTVETLETQPGEEA). In some aspects, the LRRC4 family mimic molecule consists of the amino acid sequence set forth in SEQ ID NO: 143 (GYTYFTTVTVETLETQPGEEA). In some aspects, amino acid residues T12 and L13 are modified (e.g., substituted) relative to the corresponding residues of SEQ ID NO: 143. In some aspects, the LRRC4 family mimic molecule comprises the amino acid sequence set forth in any one of SEQ ID NOs: 123-149. In some aspects, the LRRC4 family mimic molecule consists of the amino acid sequence set forth in any one of SEQ ID NOs: 123-149.

In some aspects, the amino acid at X2 is phosphorylated or O-glycosylated.

In some aspects, any of the LRRC4 family mimic molecule provided herein is conjugated to a moiety. In some aspects, the moiety is capable of increasing one or more of the following properties of the polypeptide: (1) binding affinity to a FAM19A5 protein, (2) solubility, (3) resistance to degradation from protease and/or peptidase, (4) suitability for in vivo administration, (5) ability to inhibit FAM19A5-LRRC4 protein family member interaction, or (6) any combination of (1) to (5). In some aspects, the moiety comprises a juxta-membrane sequence of the LRRC4 protein family members. In some aspects, the juxta-membrane comprises the sequence set forth in SEQ ID NO: 151 (LDEVMKTTK) or SEQ ID NO: 152 (IDEVMKTTK). In some aspects, the juxta-membrane consists of the sequence set forth in SEQ ID NO: 151 (LDEVMKTTK) or SEQ ID NO: 152 (IDEVMKTTK).

Present disclosure further provides a LRRC4 family mimic molecule comprising a polypeptide, which comprises an amino acid sequence having at least about 60%, at least about 70%, at least about 80%, at least about 90%, at least about 95%, or at least about 99% sequence identity to the amino acid sequence set forth in SEQ ID NO: 29 (YTYFTTVTVE), wherein the polypeptide is capable of binding to a FAM19A5 protein, and thereby, inhibiting, reducing, and/or dissociating an interaction between the FAM19A5 protein and a member of the LRRC4 protein family.

Also provided herein is a LRRC4 family mimic molecule comprising a polypeptide, which comprises an amino acid sequence that is at least about 70%, at least about 75%, at least about 80%, at least about 85%, at least about 90%, at least about 95%, at least about 96%, at least about 97%, at least about 98%, or about 99% identical to the amino acid sequence set forth in SEQ ID NO: 5, SEQ ID NO: 4, or SEQ ID NO: 6, and contains at least one amino acid modification relative to the amino acid sequence set forth in SEQ ID NO: 5, SEQ ID NO: 4, or SEQ ID NO: 6, respectively, and wherein the polypeptide is capable of binding to a FAM19A5 protein, and thereby, inhibiting, reducing, and/or dissociating an interaction between the FAM19A5 protein and a member of the LRRC4 protein family.

For such a LRRC4 family mimic molecule, in some aspects, the at least one amino acid modification increases the binding of the polypeptide to the FAM19A5 protein. In some aspects, the at least one amino acid modification increases the stability of the polypeptide. In some aspects, the increase in the binding and/or stability improves the ability of the polypeptide to inhibit, reduce, or dissociate the interaction between the FAM19A5 protein and the member of the LRRC4 protein family. In some aspects, the ability of the polypeptide to inhibit, reduce, or dissociate the interaction between a FAM19A5 protein and a member of the LRRC4 protein family is increased by at least about 0.5-fold, at least about 1-fold, at least about 2-fold, at least about 3-fold, at least about 4-fold, at least about 5-fold, at least about 10-fold, at least about 20-fold, at least about 30-fold, at least about 40-fold, or at least about 50-fold, compared to a corresponding polypeptide without the at least one amino acid modification.

In some aspects, the amino acid residue at position 453 of SEQ ID NO: 5 (e.g., position 5 of SEQ ID NO: 29) is T or modified to S or Y. In some aspects, the amino acid residue at position 454 of SEQ ID NO: 5 (e.g., position 6 of SEQ ID NO: 29) is T or modified to S or Y. In some aspects, the amino acid residue at position 449 of SEQ ID NO: 5 (e.g., position 1 of SEQ ID NO: 29) is Y or modified to F, V, L, I, W, or M. In some aspects, the amino acid residue at position 450 of SEQ ID NO: 5 (e.g., position 2 of SEQ ID NO: 29) is T or modified to S or Y. In some aspects, the amino acid residue at position 451 of SEQ ID NO: 5 (e.g., position 3 of SEQ ID NO: 29) is Y or modified to F or W. In some aspects, the amino acid residue at position 452 of SEQ ID NO: 5 (e.g., position 4 of SEQ ID NO: 29) is F or modified to Y or W. In some aspects, the amino acid residue at position 455 of SEQ ID NO: 5 (e.g., position 7 of SEQ ID NO: 29) is V or modified to I, Y, F, L, W, or M. In some aspects, the amino acid residue at position 456 of SEQ ID NO: 5 (e.g., position 8 of SEQ ID NO: 29) is T or modified to S or Y. In some aspects, the amino acid residue at position 457 of SEQ ID NO: 5 (e.g., position 9 of SEQ ID NO: 29) is V or modified to I, Y, F, L, W, or M. In some aspects, the amino acid residue at position 458 of SEQ ID NO: 5 (e.g., position 10 of SEQ ID NO: 29) is E or modified to D, V, I, Y, F, M, or W.

In some aspects, one or more of the amino acid residues of the above LRRC4 family mimic molecules are in a D-form. In some aspects, the D-form amino acid is at the N-terminus, C-terminus, or both.

In some aspects, a LRRC4 family mimic molecule described herein (such as those provided above) is conjugated to a moiety. In some aspects, the moiety is capable of increasing one or more of the following properties of the polypeptide: (1) binding affinity to a FAM19A5 protein, (2) solubility, (3) resistance to degradation from protease and/or peptidase, (4) suitability for in vivo administration, (5) ability to inhibit FAM19A5-LRRC4 protein family member interaction, or (6) any combination of (1) to (5). In some aspects, the moiety comprises a juxta-membrane sequence of the LRRC4 protein family members. In some aspects, the juxta-membrane comprises the sequence set forth in SEQ ID NO: 151 (LDEVMKTTK) or SEQ ID NO: 152 (IDEVMKTTK). In some aspects, the juxta-membrane consists of the sequence set forth in SEQ ID NO: 151 (LDEVMKTTK) or SEQ ID NO: 152 (IDEVMKTTK).

In some aspects, a LRRC4 family mimic molecule described herein does not comprise the transmembrane domain and/or the intracellular domain of a member of the LRRC4 protein family. In some aspects, a LRRC4 family mimic molecule described herein is capable of competing with the member of the LRRC4 protein family for binding to the FAM19A5 protein.

In some aspects, the member of the LRRC4 protein family comprises a LRRC4 protein, LRRC4B protein, LRRC4C protein, or combinations thereof.

In some aspects, a LRRC4 family mimic molecule described herein further comprises one or more additional amino acids at the N-terminus of the polypeptide, the C-terminus of the polypeptide, or both the N-terminus and the C-terminus of the polypeptide. In some aspects, the one or more additional amino acids are hydrophilic amino acids. In some aspects, the one or more additional amino acids are D-amino acids.

In some aspects, a LRRC4 family mimic molecule described herein comprises a polypeptide, wherein the N-terminus, C-terminus, or both the N-terminus and the C-terminus of the polypeptide comprise a modification which increases the stability of the polypeptide. In some aspects, the modification comprises a Fmoc, PEGylation, acetylation, methylation, cyclization, or combinations thereof.

In some aspects, a LRRC4 family mimic molecule described herein comprises a fusion protein. In some aspects, a LRRC4 family mimic molecule described herein further comprises a half-life extending moiety. In some aspects, the half-life extending moiety comprises a Fc, albumin, an albumin-binding polypeptide, a Pro/Ala/Ser (PAS), a C-terminal peptide (CTP) of the ß subunit of human chorionic gonadotropin, a polyethylene glycol (PEG), a long unstructured hydrophilic sequences of amino acids (XTEN), a hydroxyethyl starch (HES), an albumin-binding small molecule, or a combination thereof.

Provided herein is a nucleic acid molecule encoding a LRRC4 family mimic molecules described herein. In some aspects, the nucleic acid is a DNA or a RNA. In some aspects, the nucleic acid is a mRNA. In some aspects, the nucleic acid comprises a nucleic acid analog.

Also provided herein is a vector comprising any of the nucleic acids described herein. Provided herein is a cell comprising any of the vectors described herein. Provided herein is a protein conjugate comprising any of the LRRC4 family mimic molecules described herein, which is linked to an agent.

Also provided herein is a composition comprising any of the LRRC4 family mimic molecules, nucleic acids, vectors, cells, or protein conjugates described herein. In some aspects, the composition further comprises a pharmaceutically acceptable carrier.

Provided herein is a kit comprising the LRRC4 family mimic molecules, nucleic acids, vectors, cells, protein conjugates, or compositions described herein, and instructions for use.

Present disclosure also provides a method of producing molecule that is capable of inhibiting, reducing, and/or dissociating an interaction between a FAM19A5 protein and a member of a LRRC4 protein family, wherein the method comprises synthesizing any of the LRRC4 family small molecules described herein or culturing the cells described herein under suitable conditions such that the molecule is produced. In some aspects, the method further comprises isolating the molecules which have been produced.

Provided herein is a method of increasing a neurite outgrowth and/or synapse formation in neurons, comprising contacting a neuron with any of the LRRC4 family mimic molecules, nucleic acids, vectors, cell, protein conjugates, or compositions described herein. In some aspects, the contacting occurs in vivo in a subject in need thereof. In such aspects, the method can comprise administering the LRRC4 family mimic molecule, the nucleic acid, the vector, the cell, the protein conjugate, or the composition to the subject prior to the contacting. In some aspects, the contacting occurs ex vivo.

In some aspects, the contacting increases neurite outgrowth in the neuron by at least about 0.5-fold, at least about 1-fold, at least about 2-fold, at least about 3-fold, at least about 4-fold, at least about 5-fold, at least about 10-fold, at least about 20-fold, at least about 30-fold, at least about 40-fold, or at least about 50-fold, compared to a reference (e.g., neurite outgrowth in a corresponding neuron that was not contacted). In some aspects, the contacting increases synapse formation in the neuron by at least about 0.5-fold, at least about 1-fold, at least about 2-fold, at least about 3-fold, at least about 4-fold, at least about 5-fold, at least about 10-fold, at least about 20-fold, at least about 30-fold, at least about 40-fold, or at least about 50-fold, compared to a reference (e.g., synapse formation in a corresponding neuron that was not contacted).

In some aspects, the increase in neurite outgrowth and/or synapse formation reduces one or more symptoms associated with a disease or condition selected from an amyotrophic lateral sclerosis (ALS), Alzheimer's disease, glaucoma, diabetic retinopathy, neuropathic pain, spinal cord injury, traumatic brain injury, stroke, Parkinson's disease, or combinations thereof.

Present disclosure further provides a method of inhibiting or decreasing a formation of a complex between a FAM19A5 protein and a member of the LRRC4 protein family in a subject in need thereof, comprising administering to the subject any of the LRRC4 family mimic molecules, nucleic acids, vectors, cell, protein conjugates, or compositions described herein.

In some aspects, formation of a complex between a FAM19A5 protein and a LRRC4 protein family member is decreased by at least about 5%, at least about 10%, at least about 20%, at least about 30%, at least about 40%, at least about 50%, at least about 60%, at least about 70%, at least about 80%, at least about 90%, or about 100% after the administration. In some aspects, decrease in the formation of a complex between a FAM19A5 protein and the LRRC4 protein family member increases an activity of the LRRC4 protein family member in the subject.

Also provided herein is a method of treating a disease or condition in a subject in need thereof, comprising administering to the subject any of the LRRC4 family mimic molecules, nucleic acids, vectors, cell, protein conjugates, or compositions described herein, wherein the disease or condition is selected from an amyotrophic lateral sclerosis (ALS), Alzheimer's disease, glaucoma, diabetic retinopathy, neuropathic pain, spinal cord injury, traumatic brain injury, stroke, Parkinson's disease, or combinations thereof.

Provided herein is a method of increasing a neurite outgrowth and/or synapse formation in neurons, comprising contacting a neuron with a LRRC4 family mimic molecule, wherein the LRRC4 family mimic molecule is capable of inhibiting, reducing, and/or dissociating an interaction between a Family with Sequence Similarity 19, Member A5 (“FAM19A5”) protein and a member of a LRRC4 protein family, and wherein the LRRC4 family mimic molecule is a small molecule selected from:

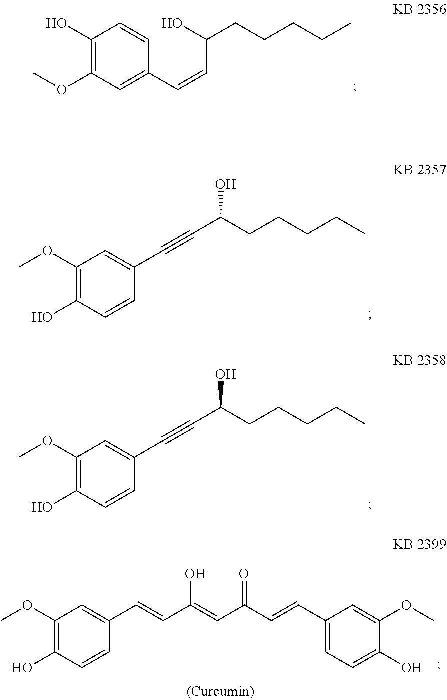

or a pharmaceutically acceptable salt thereof.

In some aspects, the contacting occurs in vivo in a subject in need thereof. In such aspects, the method can comprise administering the LRRC4 family mimic molecule to the subject prior to the contacting. In some aspects, the contacting occurs ex vivo.

In some aspects, the contacting increases neurite outgrowth in the neuron by at least about 0.5-fold, at least about 1-fold, at least about 2-fold, at least about 3-fold, at least about 4-fold, at least about 5-fold, at least about 10-fold, at least about 20-fold, at least about 30-fold, at least about 40-fold, or at least about 50-fold, compared to a reference (e.g., neurite outgrowth in a corresponding neuron that was not contacted). In some aspects, the contacting increases synapse formation in the neuron by at least about 0.5-fold, at least about 1-fold, at least about 2-fold, at least about 3-fold, at least about 4-fold, at least about 5-fold, at least about 10-fold, at least about 20-fold, at least about 30-fold, at least about 40-fold, or at least about 50-fold, compared to a reference (e.g., synapse formation in a corresponding neuron that was not contacted).

In some aspects, the increase in neurite outgrowth and/or synapse formation reduces one or more symptoms associated with a disease or condition selected from an amyotrophic lateral sclerosis (ALS), Alzheimer's disease, glaucoma, diabetic retinopathy, neuropathic pain, spinal cord injury, traumatic brain injury, stroke, Parkinson's disease, or combinations thereof.

Present disclosure also provides a method of inhibiting or decreasing a formation of a complex between a FAM19A5 protein and a member of the LRRC4 protein family in a subject in need thereof, comprising administering to the subject a LRRC4 family mimic molecule, wherein the LRRC4 family mimic molecule is capable of inhibiting, reducing, and/or dissociating an interaction between a Family with Sequence Similarity 19, Member A5 (“FAM19A5”) protein and a member of a LRRC4 protein family, and wherein the LRRC4 family mimic molecule is a small molecule selected from:

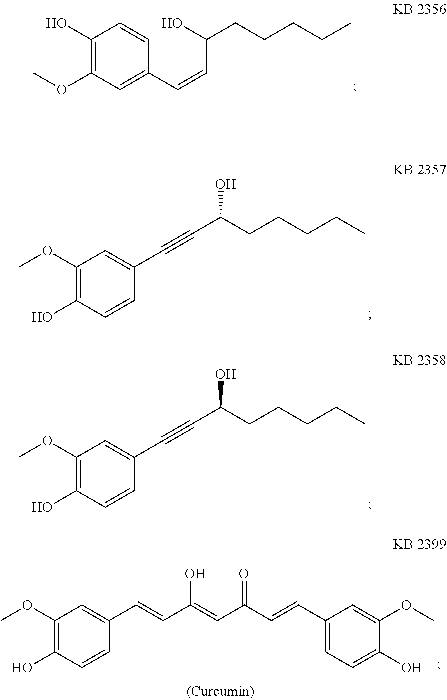

or a pharmaceutically acceptable salt thereof.

In some aspects, the formation of a complex between a FAM19A5 protein and a LRRC4 protein family member is decreased by at least about 5%, at least about 10%, at least about 20%, at least about 30%, at least about 40%, at least about 50%, at least about 60%, at least about 70%, at least about 80%, at least about 90%, or about 100% after the administration. In some aspects, the decrease in the formation of a complex between a FAM19A5 protein and a LRRC4 protein family member increases an activity of the LRRC4 protein family member in the subject.

Provided herein is a method of treating a disease or condition in a subject in need thereof, comprising administering to the subject a LRRC4 family mimic molecule, wherein the LRRC4 family mimic molecule is capable of inhibiting, reducing, and/or dissociating an interaction between a FAM19A5 protein and a member of a LRRC4 protein family, wherein the LRRC4 family mimic molecule is a small molecule selected from:

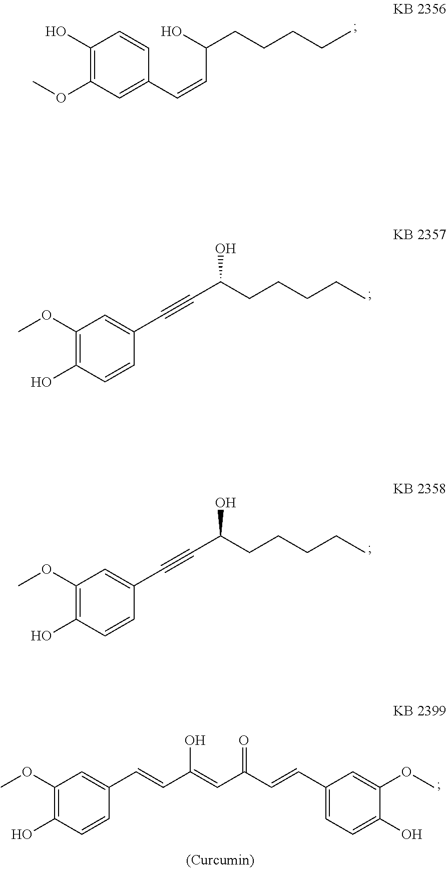

or a pharmaceutically acceptable salt thereof, and wherein the disease or condition is selected from an amyotrophic lateral sclerosis (ALS), Alzheimer's disease, glaucoma, diabetic retinopathy, neuropathic pain, spinal cord injury, traumatic brain injury, stroke, Parkinson's disease, or combinations thereof.

### BRIEF DESCRIPTION OF THE DRAWINGS

FIGS. 1A, 1B, 1C, and 1D show the ability of different members of the LRRC4 protein family (i.e., LRRC4C, LRRC4, and LRRC4B proteins) to bind to FAM19A5 protein, as measured using co-immunoprecipitation (FIGS. 1A and 1B) or immunofluorescence assay (FIGS. 1C and 1D). In FIG. 1A, cell lysates (from HEK293 cells expressing FLAG-tagged LRRC4C, LRRC4, or LRRC4B protein and treated with recombinant FAM19A5 protein) were immunoprecipitated with anti-FLAG antibody, and the immunoprecipitated proteins were immunoblotted with anti-FLAG (top row) and anti-FAM19A5 (3-2) (bottom row) antibodies. In FIG. 1B, cell lysates (from HEK293 cells expressing FLAG-tagged LRRC4B protein and treated with recombinant FAM19A5 protein) were immunoprecipitated with either human IgG (“IgG”) or anti-FAM19A5 (1-65) antibody (“1-65”). The immunoprecipitated proteins were immunoblotted with anti-FLAG (top row) and anti-FAM19A5 (3-2) (bottom row) antibodies. In FIG. 1C, HEK293 cells expressing FLAG-tagged LRRC4B protein were treated with recombinant FAM19A5 protein and immunostained with anti-FLAG and anti-FAM19A5 (3-2) antibodies. In FIG. 1D, primary cortical neurons were treated with recombinant FAM19A5 protein and immunostained with anti-FAM19A5 (3-2) and anti-LRRC4B antibodies. In FIGS. 1C and 1D, the nuclei were stained with Hoechst33342. Additionally, the images provided in the 2^{nd} (FAM19A5 protein staining alone), 3^{rd} (LRRC4B protein staining alone), and 4^{th} (overlay of FAM19A5 and LRRC4B staining) rows of FIGS. 1C and 1D are magnified views of the boxed region in the image provided in the first row. Colocalized signals are indicated by arrowheads. Scale bar=30 μm.

FIGS. 2A, 2B, 2C, and 2D show the binding of LRRC4B protein to isoform 1 and isoform 2 of the FAM19A5 protein as measured using either immunofluorescence (FIGS. 2A and 2B) or co-immunoprecipitation assay (FIGS. 2C and 2D). FIG. 2A provides the immunofluorescence data showing the interaction between LRRC4B protein and FAM19A5 isoform 1. FIG. 2B provides the immunofluorescence data showing the interaction between LRRC4B protein and FAM19A5 isoform 2. In FIG. 2C, cell lysates from the co-transfected HEK293 cells were immunoprecipitated with the anti-FLAG antibody, and then, immunoblotted with anti-FLAG (top row) and anti-FAM19A5 (3-2) (bottom row) antibodies. In FIG. 2D, cells lysates from the co-transfected HEK293 cells were immunoprecipitated with the following: (i) human IgG antibody (“IgG”); (ii) anti-FAM19A5 (1-65) antibody (“1-65”); or (iii) anti-FAM19A5 (3-2) antibody (“3-2”). The immunoprecipitated proteins were immunoblotted with anti-FLAG (top row) and anti-FAM19A5 (3-2) (bottom row) antibodies.

FIGS. 3A and 3B show the binding of different LRRC4B protein deletion constructs to the FAM19A5 protein. FIG. 3A provides a schematic of the different domains of the LRRC4B protein, and shows the domains included in the different deletion constructs. The LRRC4B domains shown include: “SP”=signal peptide; “LRR”=leucine-rich repeat; “IG”=immunoglobulin-like C2-type; “Thr”=threonine-rich; “TM”=transmembrane; and “PB”=PSD95-binding. The column under “Binding” shows whether the particular LRRC4B protein fragment was able to bind to the FAM19A5 protein: “O”=binding; “X”=no binding; and “N.D.”=not determined. FIG. 3B shows the binding of the different LRRC4B protein deletion constructs to FAM19A5 protein, as measured using a co-immunoprecipitation assay.

FIGS. 4A and 4B show the binding of FAM19A5 protein to the ectodomain of LRRC4 protein family members, as measured using ELISA. FIG. 4A provides data showing the binding of FAM19A5 protein to the full-length ectodomain of LRRC4 (amino acids 39-527 of SEQ ID NO: 1; i.e., SEQ ID NO: 4) (“1”), LRRC4B (amino acids 36-576 of SEQ ID NO: 2; i.e., SEQ ID NO: 5) (“2”), and LRRC4C (amino acids 45-527 of SEQ ID NO: 3; i.e., SEQ ID NO: 6) (“3”) proteins. FIG. 4B provides data showing the binding of FAM19A5 protein to different fragments of the LRRC4B protein: (a) amino acids 453-576 of SEQ ID NO: 2 (i.e., SEQ ID NO: 7); (b) amino acids 484-576 of SEQ ID NO: 2 (i.e., SEQ ID NO: 8); (c) amino acids 482-576 of SEQ ID NO: 2 (i.e., SEQ ID NO: 9); (d) amino acids 482-497 of SEQ ID NO: 2 (i.e., SEQ ID NO: 10); and (e) amino acids 498-576 of SEQ ID NO: 2 (i.e., SEQ ID NO: 11).

FIGS. 5A and 5B show the binding of FAM19A5 protein to the following protein fragments of the members of the LRRC4 protein family: (1) LRRC4 (amino acids 451-483 of SEQ ID NO: 1) (i.e., SEQ ID NO: 12); (2) LRRC4C (amino acids 451-484 of SEQ ID NO: 3) (i.e., SEQ ID NO: 13); and (3) LRRC4B (amino acids 484-522 of SEQ ID NO: 2) (i.e., SEQ ID NO: 14). FIG. 5A provides a schematic of the different domains present within members of the LRRC4 protein family, including the amino acid sequences of the protein fragments tested. The domains shown include: “SP”=signal peptide; “LRR”=leucine-rich repeat; “IG”=immunoglobulin-like C2-type; “Thr”=threonine-rich; “TM”=transmembrane; and “PB”=PSD95-binding. The column under “Binding” shows whether the particular LRRC4B protein fragment was able to bind to the FAM19A5 protein: “O”=binding; “X”=no binding; and “N.D.”=not determined. FIG. 5B shows the interaction between FAM19A5 protein and the different LRRC4 family protein fragments. Cell lysates were immunoprecipitated with anti-FLAG antibody, and immunoprecipitated proteins were immunoblotted with anti-FLAG (top gels) and anti-FAM19A5 (3-2) (bottom gels) antibodies.

FIG. 6 shows the ability of the following peptide fragments, comprising the YTYFTTVTVETLE (SEQ ID NO: 15) sequence of the LRRC4B protein, to bind to FAM19A5 protein: (1) “FB-16”=16 amino acids in length (SEQ ID NO: 17); (2) “FB-20”=20 amino acids in length (SEQ ID NO: 18); and (3) “FB-28”=28 amino acids in length (SEQ ID NO: 19).

FIG. 7 shows the ability of LRRC4B peptide fragment (amino acids 453-576 of SEQ ID NO: 2) (i.e., SEQ ID NO: 7) (bottom row) in inducing the dissociation of the interaction between FAM19A5 (isoform 2) and full-length LRRC4B proteins in HEK293 cells, as measured using immunofluorescence microscopy. HEK293 cells treated with a mutant form of the LRRC4B peptide fragment (contains alanine substitutions at positions 488 and 489 of SEQ ID NO: 2) (“MT”) were used as control. Image in the box (see bottom row, 4^{th} box from the left) was magnified to those stained with anti-hIgG alone (top) and with both anti-hIgG and anti-FLAG antibody (bottom). Closed arrowheads in magnified image indicate FAM19A5 signals dissociated from LRRC4B. Open arrowheads represent LRRC4B(453-576)-hFc, which remains where LRRC4B present. Scale bar=30 μm.

FIGS. 8A and 8B provide competitive inhibition assay data comparing the ability of different LRRC4B peptide fragments to inhibit the binding of FAM19A5 protein to the full-length ectodomain of the LRRC4B protein (i.e., amino acids 36-576 of SEQ ID NO: 2) (SEQ ID NO: 5). FIG. 8A provides data for the following LRRC4B peptide fragments: (1) LRRC4B (amino acids 453-576 of SEQ ID NO: 2) (SEQ ID NO: 7); (2) LRRC4B mutant (amino acids 453-576 of SEQ ID NO: 2 with AA mutation at positions 488 and 489) (SEQ ID NO: 16); (3) LRRC4B (amino acids 484-576 of SEQ ID NO: 2) (SEQ ID NO: 8); (4) LRRC4B (amino acids 482-576 of SEQ ID NO: 2)(SEQ ID NO: 9); (5) LRRC4B (amino acids 482-497 of SEQ ID NO: 2) (SEQ ID NO: 10); and (6) LRRC4B (amino acids 498-576 of SEQ ID NO: 1) (SEQ ID NO: 11). FIG. 8B provides competitive inhibition assay data showing the ability of (1) FB-28, (2) FB-20, and (3) FB-16 peptides (described in FIG. 6) to inhibit the binding of FAM19A5 protein to the full-length ectodomain of the LRRC4B protein.

FIGS. 9A, 9B, and 9C compare the ability of different LRRC4B peptide fragments to inhibit the binding of FAM19A5 protein to the full-length ectodomain of different members of the LRRC4 protein family: LRRC4 (amino acid residues 39-572 of SEQ ID NO: 1) (i.e., SEQ ID NO: 4), LRRC4B (amino acid residues 36-576 of SEQ ID NO: 2) (i.e., SEQ ID NO: 5), and LRRC4C (amino acid residues 45-527 of SEQ ID NO: 3) (i.e., SEQ ID NO: 6), respectively. The different LRRC4B peptide fragments shown include: (1) LRRC4B (amino acids 453-576 of SEQ ID NO: 2) (SEQ ID NO: 7); (2) LRRC4B mutant (amino acids 453-576 of SEQ ID NO: 2 with AA mutation at positions 488 and 489) (i.e., SEQ ID NO: 16); and (3) FB-20 (i.e., 20-amino acid long peptide fragment comprising the YTYFTTVTVETLE sequence of the LRRC4B protein; GYTYFTTVTVETLETQPGEE; SEQ ID NO: 18).

FIGS. 10A and 10B compare the ability of FBC4-23 and FBC4C-23 peptide fragments to inhibit binding of FAM19A5 protein to either the full-length ectodomain of the LRRC4B protein (FIG. 10A) or the threonine-rich domain of the LRRC4B protein (i.e., amino acids 453-576 of SEQ ID NO: 2; i.e., SEQ ID NO: 7) (FIG. 10B). The FBC4-23 peptide fragment comprised the FAM19A5 binding domain (bolded and italicized) of the LRRC4 protein and had the following sequence: NYSFFTTVTVETTEISPEDTTRK (SEQ ID NO: 20). The FBC4C-23 peptide fragment comprised the FAM19A5 binding domain (bolded and italicized) of the LRRC4C protein and had the following sequence: NFSYFSTVTVETMEPSQDERTTR (SEQ ID NO: 21). FB-20 peptide (see FIG. 6) was also used for comparison purposes.

FIGS. 11A and 11B show the ability of different FB-20 peptide fragment variants to inhibit the binding of FAM19A5 protein to either the full-length ectodomain of the LRRC4B protein (FIG. 11A) or the LRRC4B protein fragment comprising the FAM19A5 binding domain (i.e., amino acids 453-576 of SEQ ID NO: 2; SEQ ID NO: 7) (FIG. 11B). The different FB-20 variants shown are as follows: (1) FB-m11dC, (2) FB-m10dC, (3) FB-m9dC, (4) FB-m8dC, (5) FB-m7dC, (6) FB-m6dC, (7) FB-m10dN, (8) FB-m9dN, (9) FB-m8dN, and (10) FB-m7dN. As described in Example 6, each of the FB-20 variants included one or more amino acid deletions at either the C-terminal end or the N-terminal end of a LRRC4B protein domain capable of binding to FAM19A5 protein, YTYFTTVTVETLE (SEQ ID NO: 15). The specific amino acid sequences of the FB-20 variants are provided in Table 9.

FIGS. 12A and 12B show the ability of different FB-20 peptide fragment variants with an alanine (A) or asparagine (N) substitution to inhibit the binding of FAM19A5 protein to either the full-length ectodomain of the LRRC4B protein (FIG. 12A) or LRRC4B protein fragment comprising the FAM19A5 binding domain (i.e., amino acids 453-576 of SEQ ID NO: 2; SEQ ID NO: 7) (FIG. 12B). As described in Example 7, an alanine or asparagine substitutions were independently introduced into the FB-20 peptide fragment at one of the amino acid residues of a LRRC4B protein domain capable of binding to a FAM19A5 protein, i.e., YTYFTTVTVETLE (SEQ ID NO: 15). The specific amino acid sequences of the FB-20 variants are provided in Table 10. For each of the FB-20 peptide variants shown (except FB-20[12-L] and FB-20[13-E]), the first bar is for the alanine substitution and the second bar is for the asparagine substitution. For variants FB-20[12-L] and FB-20[13-E], only the alanine substitution is shown.

FIGS. 13A and 13B show the transcript level of members of the FAM19A5 family (FIG. 13A) or LRRC4B and PTPRF genes (FIG. 13B) in mouse hippocampal cultures. As described in Example 8, primary hippocampal neurons derived from the mouse brain at postnatal day 1 were cultured for 15 days in vitro. The transcript level of the different genes were measured at days 1, 3, 7, 10, and 15 post initial culture, and quantified using RNA-seq analysis. In FIG. 13A, for each of the days shown, the first, second, and third bars (from left to right) correspond to FAM19A1, FAM19A2, and FAM19A5, respectively. FAM19A3 and FAM19A4 transcripts were not detected. Data are mean±SEM of triplicates.

FIGS. 14A, 14B, 14C, and 14D show the ability of a LRRC4B peptide fragment (amino acid residues 453-576 of SEQ ID NO: 2; SEQ ID NO: 7) to promote neurite growth of mouse primary cortical neurons in vitro at various concentrations (x-axis) (0.006-60 nM). As described in Example 8, mouse primary cortical neurons (at postnatal day one) were treated with the LRRC4B protein fragment at days 1 and 2 post initial culture, and the following were quantified at day 3 by immunostaining with beta-tubulin III antibody: (i) average total neurite growth (FIG. 14A), (ii) number of primary dendrites (FIG. 14B), (iii) number of branching points (FIG. 14C), and (iv) number of secondary neurites (FIG. 14D). Data represent the mean±SEM. Statistical significance was evaluated using one-way ANOVA followed by Bonferroni post hoc tests; a, P<0.01 versus vehicle control.

FIGS. 15A, 15B, and 15C show the effect of LRRC4B peptide fragment (amino acid residues 453-576 of SEQ ID NO: 2; SEQ ID NO: 7) on the expression of synaptophysin (SYP; a presynaptic marker) and PSD95 (postsynaptic marker) in mouse hippocampal neurons. FIGS. 15A and 15B show the total fluorescence intensity for SYN and PSD-95, respectively, in dendrites/neurites of hippocampal neurons with the LRRC4B peptide fragment (6 or 60 nM), as measured using IMARIS software (IMARIS 9.0 Bitplane, Switzerland). FIG. 15C shows the number of colocalized voxels between SYP and PSD95 signals in the treated dendrites/neurites of hippocampal neurons. In each of FIGS. 15A-15C, mouse hippocampal neurons treated with vehicle (“Veh”) and the LRRC4B peptide fragment mutant (MT) (60 nM) (i.e., comprising alanine substitutions at positions 488 and 489 of SEQ ID NO: 2; SEQ ID NO: 16) were used as controls. As described elsewhere in the present disclosure, the LRRC4B MT were not able to bind to FAM19A5 protein. Data represent the mean±SEM. Number of neurons used in the quantification of fluorescent intensity were denoted in the parentheses of the bar graph. Statistical significance was evaluated using one-way ANOVA followed by Bonferroni post hoc tests; a, P<0.05 vs Veh; b, P<0.05 vs LRRC4B MT (60 nM).

FIGS. 16A, 16B, and 16C show the ability of LRRC4B peptide fragment (amino acid residues 453-576 of SEQ ID NO: 2; “WT”) (i.e., SEQ ID NO: 7) to promote synaptic formation in the hippocampal CA1 of APP/PS1 mice. As further described in Example 8, APP/PS1 mice were treated with the LRRC4B peptide fragment (30 mg/kg; intravenous administration) for four consecutive weeks, and then synaptic formation was assessed by fluorescence microscopy using antibodies against SYP and PSD95. Control animals received no treatment (“Cont”) or the mutant LRRC4B peptide fragment (60 nM) (i.e., comprising alanine substitutions at positions 488 and 489 of SEQ ID NO: 2; SEQ ID NO: 16). FIG. 16A provides representative fluorescent photomicrographs. FIGS. 16B and 16C show the SYP and PSD95 intensity, respectively.

FIGS. 17A, 17B, and 17C show the ability of LRRC4B peptide fragment (amino acid residues 453-576 of SEQ ID NO: 2; “WT”; SEQ ID NO: 7) to promote synaptic formation in the hippocampal CA3 of APP/PS1 mice. The animals were treated and analyzed as described in FIGS. 16A-16C. FIG. 17A provides representative fluorescent photomicrographs. FIGS. 17B and 17C show the SYP and PSD95 intensity, respectively.

FIGS. 18A, 18B, 18C, 18D, and 18E show neurite growth in mouse primary cortical neurons treated in vitro with the FB-16, FB-20, and FB-28 peptides (described in FIG. 6). The primary cortical neurons were treated for two days and then, at day 3 neurite growth was assessed by immunostaining with anti-beta-tubulin III antibody. FIG. 18A provides representative microscopy images from each of the treatment groups. FIGS. 18B, 18C, 18D, and 18E show the (i) average length of total neurite growth, (ii) number of primary dendrites, (iii) number of branching points, and (iv) number of secondary neurites, respectively. Data represent the mean±SEM. Statistical significance was evaluated using one-way ANOVA followed by Bonferroni post hoc tests; a, P<0.01 versus control (CTRL).

FIGS. 19A, 19B, and 19C show the increased expression of synaptophysin (SYP; a presynaptic marker) and PSD95 (postsynaptic marker) in mouse primary hippocampal neurons treated in vitro with the FB-16, FB-20, and FB-28 peptides (described in FIG. 6). FIGS. 19A and 19B show the total fluorescence intensity for SYN and PSD-95, respectively, in the dendrites/neurites of hippocampal neurons, as measured using IMARIS software (IMARIS 9.0 Bitplane, Switzerland). FIG. 19C show the number of colocalized voxels between SYP and PSD95 signals in the treated dendrites/neurites of hippocampal neurons. Data represent the mean±SEM. Number of neurons used in the quantification of fluorescent intensity were denoted in the parentheses of the bar graph. Statistical significance was evaluated using one-way ANOVA followed by Bonferroni post hoc tests; *, P<0.05 vs CTRL; **, P<0.05 vs CTRL.

FIG. 20 shows sequence alignment of domains of interest (i.e., capable of binding to FAM19A5 protein) in LRRC4 protein family members from different vertebrate species.

FIGS. 21A and 21B provide the effect of different amino acid modifications on the binding affinity of LRRC4B fragments as assessed via in silico residue scanning of FAM19A5-LRRC4 family complex using Schrodinger platform. FIG. 21A provides predictive value of Gibbs free energy change upon alanine substitution at each of amino acid residues of the FB-20 fragment (SEQ ID NO: 18). FIG. 21B provides predictive value of Gibbs free energy change for the top twenty FB-20 double mutants (comprising amino acid substitutions at residues T12 and L13 of SEQ ID NO: 18) with enhanced affinity for FAM19A5 protein. The sequences for the FB-20 double mutants shown are provided in Example 9 (Table 12).

FIGS. 22A, 22B, and 22C show the ability of different FB-21 peptide mutants to bind to FAM19A5 protein. FIG. 22A provides a comparison of the inhibitory effect of the following FB-21 peptide fragments on the interaction between hFc-fused hLRRC4B and rcFAM19A5 as determined by competitive inhibition assay: (1) wild-type FB-21 (SEQ ID NO: 143), (2) FB-21 (P12Y13) (SEQ ID NO: 144), (3) FB-21 (H12F13) (SEQ ID NO: 145), (4) FB-21 (Q12R13) (SEQ ID NO: 146), (5) FB-21 (W12Y13) (SEQ ID NO: 147), (5) FB-21 (M12R13) (SEQ ID NO: 148), and (6) FB-21 (I12F13) (SEQ ID NO: 149). FIG. 22B a comparison of the inhibitory effect of the following FB-21 peptide fragments on interaction between HIS0TEV LRRC4B and rcFAM19A5 protein as determined by competitive inhibition assay: (1) FB-21 (wild-type) (SEQ ID NO: 143), (2) FB-21 (W12Y13) (SEQ ID NO: 147), (3) FB-21 (D12Y13) (SEQ ID NO: 131), (4) FB-21 (F12F13) (SEQ ID NO: 132), (5) FB-21 (H12Y13) (SEQ ID NO: 133), (6) FB-21 (D12F13) (SEQ ID NO: 135), and (7) FB-21 (D12I13) (SEQ ID NO: 136). FIG. 22C provides the results for the following FB-21 peptide fragments which contained D-form amino acids at the amino and carboxyl terminus, with L-form amino acids at all other residues: (1) d-form FB-21 peptide with juxta-membrane (JM) sequence (“dFB-JM-31”), (2) d-form FB-21 peptide with BBB penetrating sequence at each end of the sequence (“dFB-BBB-39”), and (3) d-form FB-21 mutant peptide with DY replacement and additional JM sequence (“dFB-DY-JM31”).

FIG. 22D provides a sequence alignment of different members of the LRRC4 family (i.e., LRRC4, LRRC4B, and LRRC4C proteins). The following domains are boxed: (1) FAM19A5 binding domain (“FB”); (2) juxta-membrane domain (“JM”), and (3) transmembrane domain (“TM”).

FIGS. 23A, 23B, 23C, and 23D show the effect of different FB-21 peptide fragments described herein on amyloid beta-induced synapse loss in mouse primary neurons. FIG. 23A provides representative images for PSD95 (top row), SYP (middle row) and merge (bottom row) of hippocampal neurons treated with FB-21, FB-13-JM, or FB-BBB-39 (all 6.6 nM; see FIG. 22C for description of the different FB-21 peptide fragments tested). Nuclei of cells were stained with Hoechst (blue). Scale bar=50 μm. FIG. 23B provide a comparison of the number of colocalized voxels between SYP and PSD95 signals dendrites/neurites of hippocampal neurons treated with FB-21, FB-13-JM, or FB-BBB-39 (all 6.6 nM). The number of colocalized voxels was calculated via IMARIS software (left panel, IMARIS 9.0 Bitplane, Switzerland). FIGS. 23C and 23D provide comparison of the total fluorescence intensity for PSD95 and SYN, respectively, in dendrites/neurites of hippocampal neurons treated with FB-21, FB-13-JM, or FB-BBB-39 (all 6.6 nM) as measured using IMARIS. Data represent the mean±S.E.M. Statistical significance was evaluated using one-way ANOVA followed by Bonferroni post hoc tests; *, P<0.05 and *, P<0.01 versus NT.

FIGS. 24A and 24B show the effect of certain FB-21 peptide fragments described herein (i.e., dFB-dWY-JM31 and dFB-DY-JM31) on the promotion of neurite outgrowth of primary mouse spinal motor neurons. FIG. 24A provides representative merged images of non-treated (NT) or FB-21 peptide fragment treated spinal motor neurons which were immunostained with Tau-5 antibody. Neuronal soma was stained and detected by Hoechst (Blue). Scale bar=100 μm. FIG. 24B provides a quantitative comparison of the average total neurite length of primary spinal motor neurons from the different treatment groups. Data represents the mean #S.E.M. Statistical significance was evaluated using one-way ANOVA followed by Bonferroni post hoc tests; *, P<0.01 versus NT.

FIGS. 25A and 25B show the effect of a FB-21 peptide variant described herein (dFB-dWY-JM31) on 6-OHDA induced cell death in LUHMES cells. FIG. 25A provides a quantitative comparison of luminescence expression after treatment with the FB-21 peptide variant with or without 6-OHDA treatment. FIG. 25B provides a quantitative comparison of luminescence expression after treatment of the FB-21 peptide variant with 6-OHDA treatment. Data represents the mean±S.E.M. Statistical significance was evaluated using one-way ANOVA followed by Bonferroni post hoc tests; *, P<0.01 versus NT.

FIGS. 26A and 26B shows the effect of certain FB-21 peptide variant described herein (dFB-dDY-JM31) in a chronic constriction injury (CCI) rat model. FIG. 26A provides a comparison of paw withdrawal threshold in response to a mechanical allodynia at various timepoints after CCI induction in mice treated with a vehicle control (circle) or the FB-21 peptide variant (square). Data represents the mean±S.E.M. FIG. 26B provides a comparison of the overall Area Under Curve (AUC) for the data provided in FIG. 26A. Statistical analysis for the AUC was conducted with one-tailed unpaired t-test. *, p<0.05.

FIG. 27 show the effect of a FB-21 peptide variant described herein (dFB-dDY-JM31) on retinal dysfunction and modulation of neural oscillation. Electroretinogram (ERG) was recorded to measure electrical signals emitted by the retina in response to flashes of light using a diabetic retinopathic mouse model (db/db). ERG amplitudes of b-wave measured between the groups; heterogenous wild type (WT, db/+, black), DR control (db/db, red), and dFB-dDY-JM31 treated DR (blue). Data represents the mean #S.E.M. Statistical analysis was conducted with a one-way ANOVA followed by Bonferroni multiple comparison test was used. ***, p<0.001, **, p<0.01.

FIGS. 28A and 28B show the effect of a FB-21 peptide variant described herein (dFB-dWY-JM-31) in a mouse model of traumatic brain injury. FIG. 28A provides representative Hoechst staining of each group. FIG. 28B provides a quantitative comparison of the lesion volume based on the data provided in FIG. 28A. Data represents the mean±S.E.M. Statistical analysis was conducted with two-tailed unpaired t-test. ***, p<0.001.

FIGS. 29A and 29B provide competitive inhibition assay results showing the ability of various chemical compounds described herein to inhibit the interaction between Fc conjugated LRRC4B (amino acids 453-576 of SEQ ID NO: 2; SEQ ID NO: 7) protein fragment and a FAM19A5 protein. In FIG. 29A, the results for the following chemical compounds are provided: (1) KB734, (2) KB761, (3) KB763, (4) KB1157, (5) KB1161, (6) KB1542, (7) KB1543, (8) KB2256, (9) KB2258, (10) KB2310, (11) KB2357, (12) KB2718, (13) KB2719, (14) KB3111, (15) KB3112, (16) KB3220, (17) KB3201, (18) KB3250, and (19) KB3251. FIG. 29B shows the ability of KB734, KB2310, and KB2357 chemical compounds to inhibit LRRC4B-FAM19A5 interaction at various concentrations.

FIGS. 30A and 30B provide competitive inhibition assay results showing the ability of different KB2357 derivatives to inhibit the interaction between Fc conjugated LRRC4B (amino acids 453-576 of SEQ ID NO: 2; SEQ ID NO: 7) protein fragment and a FAM19A5 protein. FIG. 30A provides the results for the following derivatives: (1) KB2304, (2) KB2305, (3) KB2308, (4) KB2309, (5) KB2314, (6) KB2315, (7) KB2324, (8) KB2325, (9) KB2328, (10) KB2329, (11) KB2336, (12) KB2337, (13) KB2350, (14) KB2356, (15) KB2358, (16) KB2359, (17) KB2369, (18) KB2372, and (19) KB2399. FIG. 30B shows the ability of KB2356, KB2358, and KB2399 chemical compounds to inhibit LRRC4B-FAM19A5 interaction at various concentrations. In both FIGS. 30A and 30B, KB2357 is also included for comparison purposes.

### DETAILED DESCRIPTION OF THE DISCLOSURE

Disclosed herein is a mimic molecule that is capable of inhibiting, reducing, and/or dissociating the binding between a FAM19A5 protein and a LRRC4 protein family member. Specifically, the present application shows for the first time that a FAM19A5 protein can bind to LRRC4 protein family members and thereby, inhibit LRRC4 protein family member activity. The disclosed mimic molecules share certain properties (e.g., structural and/or functional) such that they can target a FAM19A5 protein. By inhibiting, reducing, and/or dissociating the interaction between FAM19A5 and members of the LRRC4 protein family, the mimic molecules described herein can restore the activity of the endogenous LRRC4 protein family members. Additional aspects of the present disclosure are provided throughout the present application.

To facilitate an understanding of the disclosure disclosed herein, a number of terms and phrases are defined. Additional definitions are set forth throughout the detailed description.

### I. Definitions

Throughout this disclosure, the term “a” or “an” entity refers to one or more of that entity; for example, “a molecule” is understood to represent one or more molecules. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, the term “alkenyl” refers to a group containing hydrogen and carbon and containing at least one carbon-carbon double bond.

As used herein, the term “alkoxy” refers to an alkyl group attached to the parent molecular moiety through an oxygen atom.

As used herein, the term “alkyl” refers to a group containing hydrogen and carbon and containing no double bonds.

As used herein, the term “alkynyl” refers to a group containing hydrogen and carbon and containing at least one carbon-carbon triple bond.

As used herein, the term “amino” refers to —NH₂.

As used herein, the term “bicycloalkenyl” refers to a fused, spirocyclic, or bridged bicyclic hydrocarbon ring system containing at least one double bond.

As used herein, the term “bicycloalkyl” refers to a fused, spirocyclic, or bridged bicyclic cycloalkyl ring.

As used herein, the term “cycloalkenyl” refers to an unsaturated non-aromatic monocyclic hydrocarbon ring system having zero heteroatoms. Representative examples of cycloalkenyl groups include, but are not limited to, cyclopentenyl, cyclohexenyl, cycloheptenyl, and cyclooctenyl.

As used herein, the term “cycloalkyl” refers to a saturated monocyclic hydrocarbon ring system having zero heteroatoms. Representative examples of cycloalkyl groups include, but are not limited to, cyclopropyl, cyclobutyl, cyclopentyl, and cyclohexyl.

As used herein, the term “formyl” refers to —CHO.

As used herein, the terms “halo” and “halogen” refer to Cl, Br, I, or F.

As used herein, the term “haloalkoxy” refers to a haloalkyl group attached to the parent molecular moiety through an oxygen atom.

As used herein, the term “haloalkyl” refers to an alkyl group substituted with one, two, three, or four halogen atoms.

As used herein, the term “heteroaryl” refers to an aromatic ring containing one, two, or three heteroatoms independently selected from nitrogen, oxygen, and sulfur. Representative examples of heteroaryl groups include, but are not limited to, furyl, imidazolyl, pyrazolyl, pyridinyl, pyrrolyl, thiazolyl, and thienyl.

As used herein, the term “heterobicycloalkyl” refers to a non-aromatic bicyclic ring system containing one, two, three, or four heteroatoms independently selected from nitrogen, oxygen, and sulfur and optionally containing one or more double bonds. The heterobicycloalkyl groups of the present disclosure can be attached to the parent molecular moiety through any carbon atom or nitrogen atom in the group.

As used herein, the term “heterocycloalkyl” refers to a non-aromatic ring containing one, two, three, or four heteroatoms independently selected from nitrogen, oxygen, and sulfur and optionally containing one or more double bonds. The heterocycloalkyl groups of the present disclosure can be attached to the parent molecular moiety through any carbon atom or nitrogen atom in the group. Representative examples of heterocycloalkyl groups include, but are not limited to, morpholinyl, piperazinyl, piperidinyl, tetrahydrofuranyl, tetrahydropyranyl, and thiomorpholinyl.

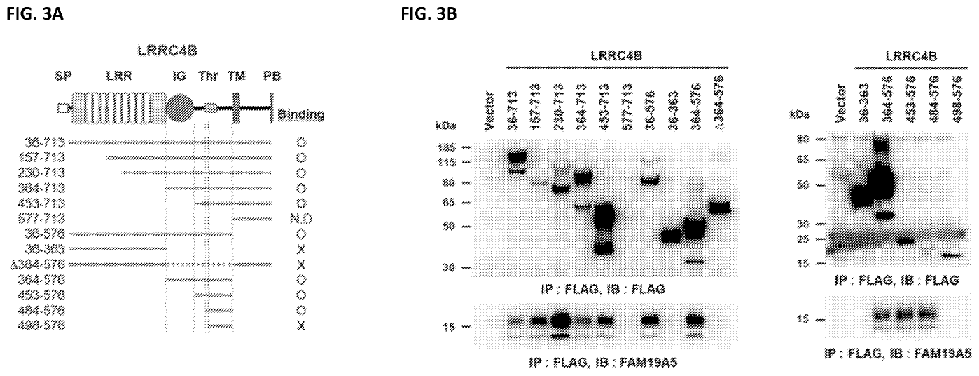

The term “leucine-rich repeat-containing 4 protein family” or “LRRC4 protein family” (including derivatives thereof) refers to a family of proteins that are key synaptic organizers and have been described to play a role in various steps of neural circuit formation, including neuronal migration, neurite outgrowth, and both the formation and functional assembly of synaptic contacts. See, e.g., Woo et al., Mol Cell Neurosci 42(1): 1-10 (September 2009). The LRRC4 protein family includes three members: (1) LRRC4, (2) LRRC4B, and (3) LRRC4C (collectively referred to herein as “LRRC4 protein family member” or “member of the LRRC4 protein family” (or derivatives thereof). The members of the LRRC4 protein family generally contains nine leucine-rich repeat (LRR) domains flanking LRR N-terminus and C-terminus (see FIG. 3A). These LRR domains are known to interact with fibronectin type III domains of presynaptic receptor protein tyrosine phosphatase (RPTP) proteins. See, e.g., Won et al., Mol Cells 41(7): 622-630 (July 2018). The LRR domains are followed by immunoglobulin-like C2-type (IG) and threonine (Thr)-rich domains, which together form the extracellular portion of members of the LRRC4 protein family. Unlike the other members, the LRRC4B protein has an extra glycine (Gly)-rich domain between the IG and Thr-rich domains. In addition to the extracellular portion, members of the LRRC4 protein family additionally include a transmembrane (TM) domain and a postsynaptic density-binding (PB) domain at the C-terminus of the protein.

In humans, gene encoding the LRRC4 protein is located on chromosome 7 (nucleotides 128,027,071-128,032,107 of GenBank Accession Number NC_000007.14; minus strand orientation). LRRC4 protein synonyms are known and examples include: “Nasopharyngeal Carcinoma-Associated Gene 14 Protein,” “Brain Tumor-Associated Protein BAG,” “Netrin-G2 Ligand,” “NAG14,” “NGL-2,” and “BAG.” The amino acid sequence for the LRRC4 protein is 653 amino acids in length and provided in Table 1 (below). The full-length ectodomain of the LRRC4 protein corresponds to amino acid residues 39-527 of SEQ ID NO: 1 (i.e., SEQ ID NO: 4). Unless indicated otherwise, the term “LRRC4 protein” (including its synonyms) includes any variants or isoforms of the LRRC4 protein which are naturally expressed by cells.

TABLE 1
LRRC4 Protein Sequence

| | |
|---|---|
| Human LRRC4 protein | MKLLWQVTVHHHTWNAILLPFVYLTAQVWILCAAIAAAASAGPQNCPS |
| (UniProt: Q9HBW1) | VCSCSNQFSKVVCTRRGLSEVPQGIPSNTRYLNLMENNIQMIQADTFR |
| (signal peptide | HLHHLEVLQLGRNSIRQIEVGAFNGLASLNTLELFDNWLTVIPSGAFE |
| is bolded) | YLSKLRELWLRNNPIESIPSYAFNRVPSLMRLDLGELKKLEYISEGAF |
| | EGLFNLKYLNLGMCNIKDMPNLTPLVGLEELEMSGNHFPEIRPGSFHG |
| | LSSLKKLWVMNSQVSLIERNAFDGLASLVELNLAHNNLSSLPHDLFTP |
| | LRYLVELHLHHNPWNCDCDILWLAWWLREYIPTNSTCCGRCHAPMHMR |
| | GRYLVEVDQASFQCSAPFIMDAPRDLNISEGRMAELKCRTPPMSSVKW |
| | LLPNGTVLSHASRHPRISVLNDGTLNFSHVLLSDTGVYTCMVTNVAGN |
| | SNASAYLNVSTAELNTSNYSFFTTVTVETTEISPEDTTRKYKPVPTTS |
| | TGYQPAYTTSTTVLIQTTRVPKQVAVPATDTTDKMQTSLDEVMKTTKI |
| | IIGCFVAVTLLAAAMLIVFYKLRKRHQQRSTVTAARTVEIIQVDEDIP |
| | AATSAAATAAPSGVSGEGAVVLPTIHDHINYNTYKPAHGAHWTENSLG |
| | NSLHPTVTTISEPYIIQTHTKDKVQETQI (SEQ ID NO: 1) |
| Human LRRC4 | ASAGPQNCPSVCSCSNQFSKVVCTRRGLSEVPQGIPSNTRYLNLMENN |
| ectodomain | IQMIQADTFRHLHHLEVLQLGRNSIRQIEVGAFNGLASLNTLELFDNW |
| protein (i.e., | LTVIPSGAFEYLSKLRELWLRNNPIESIPSYAFNRVPSLMRLDLGELK |
| amino acids | KLEYISEGAFEGLFNLKYLNLGMCNIKDMPNLTPLVGLEELEMSGNHF |
| 39-527 of | PEIRPGSFHGLSSLKKLWVMNSQVSLIERNAFDGLASLVELNLAHNNL |
| SEQ ID NO: 1) | SSLPHDLFTPLRYLVELHLHHNPWNCDCDILWLAWWLREYIPTNSTCC |
| | GRCHAPMHMRGRYLVEVDQASFQCSAPFIMDAPRDLNISEGRMAELKC |
| | RTPPMSSVKWLLPNGTVLSHASRHPRISVLNDGTLNFSHVLLSDTGVY |
| | TCMVTNVAGNSNASAYLNVSTAELNTSNYSFFTTVTVETTEISPEDTT |
| | RKYKPVPTTSTGYQPAYTTSTTVLIQTTRVPKQVAVPATDTTDKMQTS |
| | LDEVMKTTK (SEQ ID NO: 4) |

In humans, the gene encoding the LRRC4B protein is located on chromosome 19 (nucleotides 50,516,892-50,568,435 of GenBank Accession Number NC_000019.10; minus strand orientation). Synonyms of the LRRC4B protein are known and non-limiting examples include: “Netrin-G3 Ligand,” “LRIG4,” “NGL-3,” “HSM,” and “DKFZp761A179.” The amino acid sequence for the LRRC4B protein is 713 amino acids in length and provided in Table 2 (below). The full-length ectodomain of the LRRC4B protein corresponds to amino acid residues 36-576 of SEQ ID NO: 2 (i.e., SEQ ID NO: 5). Unless indicated otherwise, the term “LRRC4B protein” (including its synonyms) includes any variants or isoforms of the LRRC4B protein which are naturally expressed by cells.

TABLE 2
LRRC4B Protein Sequence

| | |
|---|---|
| Human LRRC4B protein | MARARGSPCPPLPPGRMSWPHGALLFLWLFSPPLGAGGGGVAVTSAAG |
| (UniProt: Q9NT99) | GGSPPATSCPVACSCSNQASRVICTRRDLAEVPASIPVNTRYLNLQEN |
| (signal peptide | GIQVIRTDTFKHLRHLEILQLSKNLVRKIEVGAFNGLPSLNTLELFDN |
| is bolded) | RLTTVPTQAFEYLSKLRELWLRNNPIESIPSYAFNRVPSLRRLDLGEL |
| | KRLEYISEAAFEGLVNLRYLNLGMCNLKDIPNLTALVRLEELELSGNR |
| | LDLIRPGSFQGLTSLRKLWLMHAQVATIERNAFDDLKSLEELNLSHNN |
| | LMSLPHDLFTPLHRLERVHLNHNPWHCNCDVLWLSWWLKETVPSNTTC |
| | CARCHAPAGLKGRYIGELDQSHFTCYAPVIVEPPTDLNVTEGMAAELK |
| | CRTGTSMTSVNWLTPNGTLMTHGSYRVRISVLHDGTLNFTNVTVQDTG |
| | QYTCMVTNSAGNTTASATLNVSAVDPVAAGGTGSGGGGPGGSGGVGGG |
| | SGGYTYFTTVTVETLETQPGEEALQPRGTEKEPPGPTTDGVWGGGRPG |
| | DAAGPASSSTTAPAPRSSRPTEKAFTVPITDVTENALKDLDDVMKTTK |
| | IIIGCFVAITFMAAVMLVAFYKLRKQHQLHKHHGPTRTVEIINVEDEL |
| | PAASAVSVAAAAAVASGGGVGGDSHLALPALERDHLNHHHYVAAAFKA |
| | HYSSNPSGGGCGGKGPPGLNSIHEPLLFKSGSKENVQETQI (SEQ |
| | ID NO: 2) |
| Human LRRC4B | AGGGGVAVTSAAGGGSPPATSCPVACSCSNQASRVICTRRDLAEVPAS |
| ectodomain | IPVNTRYLNLQENGIQVIRTDTFKHLRHLEILQLSKNLVRKIEVGAFN |
| protein (i.e., | GLPSLNTLELFDNRLTTVPTQAFEYLSKLRELWLRNNPIESIPSYAFN |
| amino acids | RVPSLRRLDLGELKRLEYISEAAFEGLVNLRYLNLGMCNLKDIPNLTA |
| 36-576 of | LVRLEELELSGNRLDLIRPGSFQGLTSLRKLWLMHAQVATIERNAFDD |
| SEQ ID NO: 2) | LKSLEELNLSHNNLMSLPHDLFTPLHRLERVHLNHNPWHCNCDVLWLS |
| | WWLKETVPSNTTCCARCHAPAGLKGRYIGELDQSHFTCYAPVIVEPPT |
| | DLNVTEGMAAELKCRTGTSMTSVNWLTPNGTLMTHGSYRVRISVLHDG |
| | TLNFTNVTVQDTGQYTCMVTNSAGNTTASATLNVSAVDPVAAGGTGSG |
| | GGGPGGSGGVGGGSGGYTYFTTVTVETLETQPGEEALQPRGTEKEPPG |
| | PTTDGVWGGGRPGDAAGPASSSTTAPAPRSSRPTEKAFTVPITDVTEN |
| | ALKDLDDVMKTTK (SEQ ID NO: 5) |

In humans, the gene encoding the LRRC4C protein is located on chromosome 11 (nucleotides 40,107,066-41,460,419 of GenBank Accession Number NC_000011.10; minus strand orientation). Synonyms of the LRRC4C protein are known and examples include: “NGL-1,” “Netrin-G1 Ligand,” and “KIAA1580.” The amino acid sequence for the LRRC4C protein is 640 amino acids in length and provided in Table 3 (below). The full-length ectodomain of the LRRC4C protein corresponds to amino acid residues 45-527 of SEQ ID NO: 3 (i.e., SEQ ID NO: 6). Unless indicated otherwise, the term “LRRC4C protein” (including its synonyms) includes any variants or isoforms of the LRRC4C protein which are naturally expressed by cells.

TABLE 3
LRRC4C Protein Sequence)

| | |
|---|---|
| Human LRRC4C protein | MLNKMTLHPQQIMIGPRFNRALFDPLLVVLLALQLLVVAGLVRAQTCP |
| (UniProt: Q9HCJ2) | SVCSCSNQFSKVICVRKNLREVPDGISTNTRLLNLHENQIQIIKVNSF |
| (signal peptide | KHLRHLEILQLSRNHIRTIEIGAFNGLANLNTLELFDNRLTTIPNGAF |
| is bolded) | VYLSKLKELWLRNNPIESIPSYAFNRIPSLRRLDLGELKRLSYISEGA |
| | FEGLSNLRYLNLAMCNLREIPNLTPLIKLDELDLSGNHLSAIRPGSFQ |
| | GLMHLQKLWMIQSQIQVIERNAFDNLQSLVEINLAHNNLTLLPHDLFT |
| | PLHHLERIHLHHNPWNCNCDILWLSWWIKDMAPSNTACCARCNTPPNL |
| | KGRYIGELDQNYFTCYAPVIVEPPADLNVTEGMAAELKCRASTSLTSV |
| | SWITPNGTVMTHGAYKVRIAVLSDGTLNFTNVTVQDTGMYTCMVSNSV |
| | GNTTASATLNVTAATTTPFSYFSTVTVETMEPSQDEARTTDNNVGPTP |
| | VVDWETTNVTTSLTPQSTRSTEKTFTIPVTDINSGIPGIDEVMKTTKI |
| | IIGCFVAITLMAAVMLVIFYKMRKQHHRQNHHAPTRTVEIINVDDEIT |
| | GDTPMESHLPMPAIEHEHLNHYNSYKSPFNHTTTVNTINSIHSSVHEP |
| | LLIRMNSKDNVQETQI (SEQ ID NO: 3) |
| Human LRRC4C | QTCPSVCSCSNQFSKVICVRKNLREVPDGISTNTRLLNLHENQIQIIK |
| ectodomain | VNSFKHLRHLEILQLSRNHIRTIEIGAFNGLANLNTLELFDNRLTTIP |
| protein (i.e., | NGAFVYLSKLKELWLRNNPIESIPSYAFNRIPSLRRLDLGELKRLSYI |
| amino acids | SEGAFEGLSNLRYLNLAMCNLREIPNLTPLIKLDELDLSGNHLSAIRP |
| 45-527 of | GSFQGLMHLQKLWMIQSQIQVIERNAFDNLQSLVEINLAHNNLTLLPH |
| SEQ ID NO: 3) | DLFTPLHHLERIHLHHNPWNCNCDILWLSWWIKDMAPSNTACCARCNT |
| | PPNLKGRYIGELDQNYFTCYAPVIVEPPADLNVTEGMAAELKCRASTS |
| | LTSVSWITPNGTVMTHGAYKVRIAVLSDGTLNFTNVTVQDTGMYTCMV |
| | SNSVGNTTASATLNVTAATTTPFSYFSTVTVETMEPSQDEARTTDNNV |
| | GPTPVVDWETTNVTTSLTPQSTRSTEKTFTIPVTDINSGIPGIDEVMK |
| | TTK (SEQ ID NO: 6) |

As used herein, the term “FAM19A5 binding domain” refers to a segment/fragment of a member of the LRRC4 protein family that is capable of binding to a FAM19A5 protein.

The term “family with sequence similarity 19, member A5” or “FAM19A5” refers to a protein that belongs to the TAFA family (also known as FAM19 family) of five highly homologous proteins and is predominantly expressed in brain and the spinal cord. FAM19A5 is also known as “TAFA5” or “Chemokine-like protein TAFA-5.”

In humans, the gene encoding FAM19A5 is located on chromosome 22. There are multiple human FAM19A5 (UniProt: Q7Z5A7) isoforms, which are believed to be produced by alternative splicing: isoform 1 (UniProt: Q7Z5A7-1), which consists of 132 amino acids, isoform 2 (UniProt: Q7Z5A7-2), which consists of 125 amino acids, and isoform 3 (UniProt: Q7Z5A7-3), which consists of 53 amino acids. Human FAM19A5 protein is believed to exist as both membrane bound and soluble (secreted) forms. Isoform 1 is believed to be a membrane protein with one transmembrane region. Isoform 2, which was reported in Tang T. Y. et al., Genomics 83(4):727-34 (2004) as a secreted protein (soluble), contains a signal peptide at amino acid positions 1-25. Isoform 1 is believed to be a membrane protein and predicted based on EST data. Table 4 (below) provides the amino acid sequences of the three known human FAM19A5 isoforms. Unless indicated otherwise, the term “FAM19A5” includes any variants or isoforms of the FAM19A5 protein which are naturally expressed by cells. Accordingly, in some aspects, a polypeptide described herein (e.g., comprising a FAM19A5 binding domain of a member of the LRRC4 protein family) can inhibit the binding of FAM19A5 isoform 1, isoform 2, and/or isoform 3 to the LRRC4 protein family members.

TABLE 4
FAM19A5 Protein Sequences

| | |
|---|---|
| Protein (Isoform 1) | TLDRDSSQPRRTIARQTARCA |
| (UniProt: Q7Z5A7-1, | CRKGQIAGTTRARPACVDARI |
| transmembrane protein): | IKTKQWCDMLPCLEGEGCDLL |
| this isoform has been | INRSGWTCTQPGGRIKTTTVS |
| chosen as the canonical | (SEQ ID NO: 22) |
| sequence. | |
| Protein (Isoform 2) | QPRRTIARQTARCACRKGQIA |
| (UniProt: Q7Z5A7-2, | GTTRARPACVDARIIKTKQWC |
| soluble protein) | DMLPCLEGEGCDLLINRSGWT |
| | CTQPGGRIKTTTVS |
| | (SEQ ID NO: 23) |
| Human FAM19A5 | MYHHREWPARIIKTKQWCDML |
| Protein (Isoform 3) | PCLEGEGCDLLINRSGWTCTQ |
| (UniProt: Q7Z5A7-3) | PGGRIK |
| | TTTVS (SEQ ID NO: 24) |

The term “endogenous,” when used to describe members of the LRRC4 protein family, refers to LRRC4 family proteins that naturally exist in a subject. As described herein, the mimic molecules of the present disclosure differ (structurally and/or functionally) from endogenous LRRC4 protein family members.

“Binding affinity” generally refers to the strength of the sum total of non-covalent interactions between a single binding site of a molecule (e.g., a LRRC4 mimic molecule) and its binding partner (e.g., a FAM19A5 protein). Unless indicated otherwise, as used herein, “binding affinity” refers to intrinsic binding affinity which reflects a 1:1 interaction between members of a binding pair. The affinity of a molecule X (e.g., mimic molecules described herein, which comprise a FAM19A5 binding domain of LRRC4 protein family members) for its partner Y (e.g., FAM19A5) can generally be represented by the dissociation constant (K_{D}). Affinity can be measured and/or expressed in a number of ways known in the art, including, but not limited to, equilibrium dissociation constant (K_{D}), and equilibrium association constant (K_{A}). The K_{D} is calculated from the quotient of k_{off}/kₒₙ and is expressed as a molar concentration (M), whereas K_{A} is calculated from the quotient of kₒₙ/k_{off}. kₒₙ refers to the association rate constant of, e.g., an antibody to an antigen, and k_{off} refers to the dissociation of, e.g., an antibody to an antigen. The kₒₙ and k_{off} can be determined by techniques known to one of ordinary skill in the art, such as immunoassays (e.g., enzyme-linked immunosorbent assay (ELISA)), BIACORE® or kinetic exclusion assay (KinExA).

As used herein, the terms “specifically binds,” “specifically recognizes,” “specific binding,” “selective binding,” and “selectively binds,” are analogous terms and refer to molecules (e.g., LRRC4 family mimic molecules) that bind to an antigen (e.g., FAM19A5 protein) as such binding is understood by one skilled in the art. For example, a molecule that specifically binds to an antigen can bind to other peptides or polypeptides, generally with lower affinity as determined by, e.g., immunoassays, BIACORE®, KinExA 3000 instrument (Sapidyne Instruments, Boise, ID), or other assays known in the art. In some aspects, molecules that specifically bind to an antigen bind to the antigen with a K_{A} that is at least about 2 logs, at least about 2.5 logs, at least about 3 logs, at least about 4 logs or greater than the K_{A} when the molecules bind to another antigen.

As used herein, the term “antigen” refers to any natural or synthetic immunogenic substance, such as a protein, peptide, or hapten. As is apparent from the present disclosure, an antigen can be a FAM19A5 protein or a fragment thereof.

Molecules (e.g., LRRC4 family mimic molecules) that “compete with another protein for binding to a target” refer to molecules that inhibit (partially or completely) the binding of the other protein (e.g., naturally existing members of the LRRC4 protein family) to the target. Whether two compounds compete with each other for binding to a target, i.e., whether and to what extent a LRRC4 family mimic molecule described herein inhibits the binding of the naturally existing members of the LRRC4 protein family to a FAM19A5 protein, can be determined using known competition experiments. In some aspects, a LRRC4 family mimic molecule described herein competes with, and inhibits the binding of the naturally existing members of the LRRC4 protein family to the FAM19A5 protein by at least about 10%, at least about 20%, at least about 30%, at least about 40%, at least about 50%, at least about 60%, at least about 70%, at least about 80%, at least about 90% or at least about 100%. Competition assays can be conducted as described herein or, for example, in Ed Harlow and David Lane, Cold Spring Harb Protoc; 2006; doi: 10.1101/pdb.prot4277 or in Chapter 11 of “Using Antibodies” by Ed Harlow and David Lane, Cold Spring Harbor Laboratory Press, Cold Spring Harbor, NY, USA 1999.

Other competitive binding assays that can be used with the present disclosure include: solid phase direct or indirect radioimmunoassay (RIA), solid phase direct or indirect enzyme immunoassay (EIA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using 1-125 label (see Morel et al., Mol. Immunol. 25(1):7 (1988)); solid phase direct biotin-avidin EIA (Cheung et al., Virology 176:546 (1990)); and direct labeled RIA. (Moldenhauer et al., Scand. J. Immunol. 32:77 (1990)).

The term “naturally-occurring” or “naturally-existing,” as used herein, refers to the fact that an object (e.g., protein) can be found in nature. For example, a polypeptide or polynucleotide sequence that is present in an organism (including viruses) that can be isolated from a source in nature and which has not been intentionally modified by man in the laboratory is naturally-occurring. As further described elsewhere in the present disclosure, LRRC4 family mimic molecules useful for the present disclosure are not naturally-occurring.

A “mimic molecule” refers to a molecule that resembles another molecule (“reference molecule”) in structure and/or function. For instance, in some aspects, a mimic molecule can share a partial structure or sequence with the reference molecule, such that mimic molecule can exhibit one or more properties of the reference molecule. However, as is apparent from the present disclosure, a structural or sequence similarity is not always required. In some aspects, a mimic molecule can differ in structure but behave similarly to the reference molecule. As described herein, in some aspects, a mimic molecule comprises a small molecule. In some aspects, a mimic molecule comprises a peptide. In some aspects, a mimic molecule is not an antibody or an antigen-binding portion thereof.

A “polypeptide” refers to a chain comprising at least two consecutively linked amino acid residues, with no upper limit on the length of the chain. One or more amino acid residues in the protein can contain a modification such as, but not limited to, glycosylation, phosphorylation, or disulfide bond formation. A “protein” can comprise one or more polypeptides.

The term “nucleic acids” or “nucleic acid molecule,” as used herein, is intended to include DNA molecules and RNA molecules. A nucleic acid molecule can be single-stranded or double-stranded, and can be cDNA.

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”) In general, expression vectors of utility in recombinant DNA techniques are often in the form of plasmids. In the present specification, “plasmid” and “vector” can be used interchangeably as the plasmid is the most commonly used form of vector. However, also included are other forms of expression vectors, such as viral vectors (e.g., replication defective retroviruses, adenoviruses and adeno-associated viruses), which serve equivalent functions.

The term “recombinant host cell” (or simply “host cell”), as used herein, is intended to refer to a cell that comprises a nucleic acid that is not naturally present in the cell, and can be a cell into which a recombinant expression vector has been introduced. It should be understood that such terms are intended to refer not only to the particular subject cell but to the progeny of such a cell. Because certain modifications can occur in succeeding generations due to either mutation or environmental influences, such progeny cannot, in fact, be identical to the parent cell, but are still included within the scope of the term “host cell” as used herein.

As used herein, “administering” refers to the physical introduction of a molecule (e.g., LRRC4 mimic molecule) or a composition comprising the molecule to a subject, using any of the various methods and delivery systems known to those skilled in the art. Non-limiting examples of routes of administration that can be used include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, a molecule described herein (e.g., LRRC4 family mimic molecules described herein) can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

As used herein, the term “subject” includes any human or non-human animal. The term “non-human animal” includes all vertebrates, e.g., mammals and non-mammals, such as non-human primates, sheep, dog, cow, chickens, amphibians, reptiles, etc.

The term “neuron” includes electrically excitable cells that process and transmit information through electrical and chemical signals. Neurons are the major components of the brain and spinal cord of the CNS, and of the ganglia of the peripheral nervous system (PNS), and can connect to each other to form neural networks. A typical neuron is composed of a cell body (soma), dendrites, and an axon. The soma (the cell body) of a neuron contains the nucleus. The dendrites of a neuron are cellular extensions with many branches, where the majority of input to the neuron occurs. The axon is a finer, cable-like projection extending from the soma and carries nerve signals away from the soma and certain types of information back to the soma.

The term “therapeutically effective amount” as used herein refers to an amount of a substance (e.g., LRRC4 mimic molecules described herein), alone or in combination with another therapeutic agent, effective to “treat” a disease or disorder in a subject or reduce the risk, potential, possibility or occurrence of a disease or disorder (e.g., a neurological disease described herein). A “therapeutically effective amount” includes an amount of a substance or a therapeutic agent that provides some improvement or benefit to a subject having or at risk of having a disease or disorder (e.g., a neurological disease described herein). Thus, a “therapeutically effective” amount is an amount that reduces the risk, potential, possibility or occurrence of a disease or provides disorder or some alleviation, mitigation, and/or reduces at least one indicator, and/or decrease in at least one clinical symptom of a disease or disorder.

### II. Mimic Molecules

Disclosed herein are mimic molecules of members of the LRRC4 protein family (referred to herein as “LRRC4 family mimic molecules”). As demonstrated herein for the first time (see, e.g., Example 1), FAM19A5 protein exhibits high binding affinity to all members of the LRRC4 protein family. The mimic molecules described herein resemble members of the LRRC4 family in that they can compete for binding to a FAM19A5 protein. In some aspects, the LRRC4 family mimic molecules described herein exhibit one or more properties (e.g., increased binding affinity and/or stability), such that they can out compete the naturally existing members of the LRRC4 protein family for binding to a FAM19A5 protein. As demonstrated herein, this can result in the inhibition, reduction, and/or dissociation of the interaction between members of the LRRC4 protein family and FAM19A5 protein.

As is apparent from the present disclosure, while the LRRC4 family mimic molecules described herein share certain properties with the members of the LRRC4 protein family, the LRRC4 family mimic molecules differ (structurally and/or functionally) from naturally existing members of the LRRC4 protein family. For instance, in some aspects, a LRRC4 family mimic molecule useful for the present disclosure comprises a small molecule. As further described elsewhere in the present disclosure, in some aspects, a LRRC4 family mimic molecule comprises a polypeptide, wherein the polypeptide comprises, consists of, or consists essentially of a domain of a member of the LRRC4 protein family, wherein the domain is capable of binding to the FAM19A5 protein (also referred to herein as the “FAM19A5 binding domain”). For such aspects, the polypeptides can comprise one or more amino acid substitutions within the FAM19A5 binding domain. As described elsewhere in the present disclosure, in some aspects, such amino acid substitutions can improve one or more properties of the polypeptides, e.g., increase the stability and/or binding affinity of the polypeptides to the FAM19A5 protein. In some aspects, the polypeptides can comprise the FAM19A5 binding domain but lack one or more other domains of the members of the LRRC4 protein family. For instance, in some aspects, a polypeptide comprises the FAM19A5 binding domain, but does not comprise the transmembrane domain. In some aspects, a polypeptide comprises the FAM19A5 binding domain, but does not comprise the intracellular domain of members of the LRRC4 protein family (e.g., postsynaptic density-binding (PB) domain). In some aspects, a polypeptide comprises the FAM19A5 binding domain, but does not comprise both the transmembrane domain and the intracellular domain. Accordingly, in some aspects, polypeptides described herein are shorter than the naturally existing LRRC4 protein family members. Additionally, in carrying out their biological activity (e.g., neural circuit formation), members of the LRRC4 protein family (LRRC4, LRRC4B, and LRRC4C) interact with their ligand (netrin-G2, receptor tyrosine phosphatase LAR, and netrin-G1, respectively). See, e.g., Li et al., Mol Cancer 13: 266 (December 2014). Because the polypeptides of the present disclosure do not comprise all the domains of LRRC4 protein family members, in some aspects, the polypeptides do not bind to the LRRC4 protein family ligands and instead, specifically target the FAM19A5 protein. Accordingly, in some aspects, the polypeptides described herein do not replace the endogenous LRRC4 protein family members. Instead, in some aspects, by inhibiting, reducing, and/or dissociating the interaction between FAM19A5 and members of the LRRC4 protein family, the polypeptides of the present disclosure can free up the endogenous LRRC4 family proteins and allow them to carry out their natural biological activity.

Where the LRRC4 family mimic molecules comprise small molecule compounds, in some aspects, the mimic molecule is:

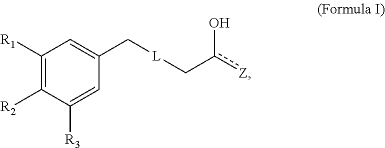

or a pharmaceutically acceptable salt thereof, wherein:
(i) R₁, R₂ and R₃ are independently selected from hydrogen, fluoro, chloro, bromo, methyl, ethyl, n-propyl, iso-propyl, n-butyl, iso-butyl, t-butyl, n-pentyl, iso-pentyl, fluoromethyl, difluoromethyl, trifluoromethyl, 2-fluoroethyl, 1-fluoroethyl, 2,2-difluoroethyl, 1,2-difluoroethyl, 1,1-difluoroethyl, 2,2,2-trifluoroethyl, methoxy, ethoxy, n-propyloxy, iso-propyloxy, n-butoxy, trifluoromethoxy, difluoromethoxy, fluoromethoxy, acetyl, propionyl, n-butanoyl, iso-butanoyl, n-pentanoyl, nitro, amino, N-methylamino, N-ethylamino, N-n-propylamino, N,N-dimethylamino, N-acetylamino, N-propionylamino, N-(trifluoroacetyl)amino, formyl, hydroxy, methylthio, ethylthio, n-propylthio, methylsulfonyl, ethylsulfonyl, n-propylsulfonyl, phenyl, hydroxymethyl, 1-hydroxyethyl and 2-hydroxyethyl;
(ii) [symbol] is a single or double bond;
(iii) Z is selected from a straight chain or branched (C₁-C₈)alkyl, a straight chain or branched (C₂-C₈)alkenyl, a straight chain or branched (C₂-C₈)alkynyl, (C₃-C₈)cycloalkyl, (C₅-C₈)cycloalkenyl, (3-8 membered) heterocycloalkyl, (C₇-C₁₄)bicycloalkyl, (C₇-C₁₄) bicycloalkenyl, (7-14 membered) heterobicycloalkyl, (C₆-C₁₀) aryl, (5-10-membered) heteroaryl, and —CH—C(O)—CH═CH-Q, wherein is Q (C₃-C₈)cycloalkyl, (C₅-C₈)cycloalkenyl, (3-8 membered)heterocycloalkyl, (C₆-C₁₀)aryl, and (5-6-membered)heteroaryl; wherein each cycloalkyl, cycloalkenyl, heterocyclylalkyl, aryl, and heteroaryl are optionally substituted with one, two, three, four, or five substituents independently selected from (C₁-C₆)alkoxy, (C₁-C₆)alkyl, halo, (C₁-C₆)haloalkoxy, nitro, amino, N-methylamino, N-ethylamino, N—N-propylamino, N,N-dimethylamino, formyl, and hydroxy, and
(iv) L is single, double or triple bond.

In some aspects, R₁, R₂ and R₃ are selected from hydrogen, hydroxy, methoxy, ethoxy, n-propyloxy, iso-propyloxy, n-butoxy, trifluoromethoxy, difluoromethoxy, and fluoromethoxy. In some aspects, R₁, R₂ and R₃ are selected from hydrogen, hydroxy, methoxy, ethoxy, n-propyloxy, iso-propyloxy, n-butoxy. In some aspects, R₁, R₂ and R₃ are selected from hydrogen, hydroxy, and methoxy. In some aspects, R₁ and R₂ are selected from hydroxy and methoxy and R₃ is hydrogen.

In some aspects, Z is selected from a straight chain or branched (C₁-C₈)alkyl, a straight chain or branched (C₂-C₈)alkenyl, a straight chain or branched (C₂-C₈)alkynyl, and —CH—C(O)—CH═CH-Q, wherein Q is selected from (C₆-C₁₀)aryl, and (5-6-membered)heteroaryl; wherein the aryl, and the heteroaryl are optionally substituted with one, two, or three substituents independently selected from (C₁-C₆)alkoxy, (C₁-C₆)alkyl, halo, (C₁-C₆)haloalkoxy, and hydroxy. In some aspects, Z is selected from a straight chain or branched (C₁-C₈)alkyl, a straight chain or branched (C₂-C₈)alkenyl, and —CH—C(O)—CH═CH-Q, wherein Q is (C₆-C₁₀)aryl optionally substituted with one, two, or three substituents independently selected from (C₁-C₆)alkoxy, (C₁-C₆)alkyl, halo, (C₁-C₆)haloalkoxy, and hydroxyl.

In some aspects, the LRRC4 family mimic molecule is a small molecule of formula (I) wherein:
(i) R₁, R₂ and R₃ are selected from hydrogen, hydroxy, methoxy, ethoxy, n-propyloxy, iso-propyloxy, n-butoxy, trifluoromethoxy, difluoromethoxy, and fluoromethoxy;
(ii) [symbol] is a single or double bond;
(iii) Z is selected from a straight chain or branched (C₁-C₈)alkyl, a straight chain or branched (C₂-C₈)alkenyl, a straight chain or branched (C₂-C₈)alkynyl, and CH—C(O)—CH═CH-Q, wherein Q is selected from (C₆-C₁₀)aryl, and (5-6-membered)heteroaryl; wherein the aryl, and the heteroaryl are optionally substituted with one, two, or three substituents independently selected from (C₁-C₆)alkoxy, (C₁-C₆)alkyl, halo, (C₁-C₆)haloalkoxy, and hydroxy; and
(iv) L is a double or triple bond.

In some aspects, the LRRC4 family mimic molecule is a small molecule of formula (I) wherein:
(i) R₁, R₂ and R₃ are selected from hydrogen, hydroxy, methoxy, ethoxy, n-propyloxy, iso-propyloxy, and n-butoxy;
(ii) [symbol] is a single or double bond;
(iii) Z is selected from a straight chain or branched (C₁-C₈)alkyl, a straight chain or branched (C₂-C₈)alkenyl, and —CH—C(O)—CH═CH-Q, wherein Q is (C₆-C₁₀)aryl optionally substituted with one, two, or three substituents independently selected from (C₁-C₆)alkoxy, (C₁-C₆)alkyl, halo, (C₁-C₆)haloalkoxy, and hydroxy; and
(iv) L is a double or triple bond.

In some aspects, the LRRC4 family mimic molecule is selected from:

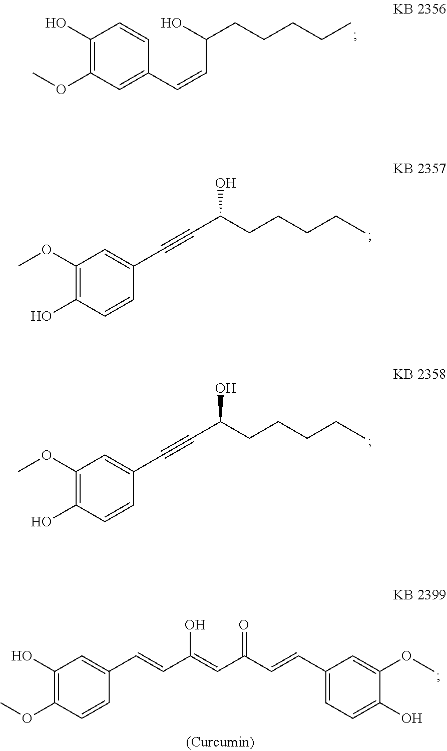

or a pharmaceutically acceptable salt thereof.

In some aspects, the LRRC4 family mimic molecule is:

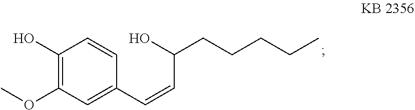 [SMILES: CCCCCC(O)/C=C\c1ccc(O)c(OC)c1]

or a pharmaceutically acceptable salt thereof.

In some aspects, the LRRC4 family mimic molecule is:

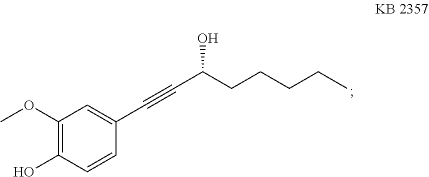 [SMILES: CCCCC[C@@H](O)C#Cc1ccc(O)c(OC)c1]

or a pharmaceutically acceptable salt thereof.

In some aspects, the LRRC4 family mimic molecule is:

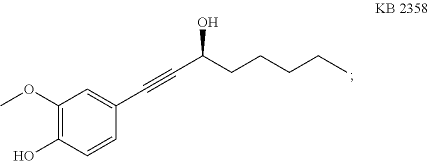 [SMILES: CCCCC[C@H](O)C#Cc1ccc(O)c(OC)c1]

or a pharmaceutically acceptable salt thereof.

In some aspects, the LRRC4 family mimic molecule is:

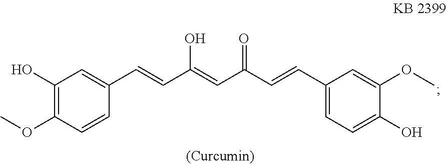

or a pharmaceutically acceptable salt thereof.

In some aspects, the LRRC4 family mimic molecule is:

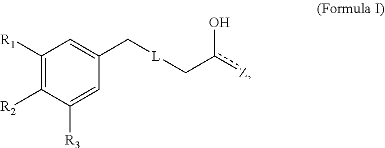

or a pharmaceutically acceptable salt thereof, wherein:
(i) R₁, R₂ and R₃ are independently selected from hydrogen, fluoro, chloro, bromo, methyl, ethyl, n-propyl, iso-propyl, n-butyl, iso-butyl, t-butyl, n-pentyl, iso-pentyl, fluoromethyl, difluoromethyl, trifluoromethyl, 2-fluoroethyl, 1-fluoroethyl, 2,2-difluoroethyl, 1,2-difluoroethyl, 1,1-difluoroethyl, 2,2,2-trifluoroethyl, methoxy, ethoxy, n-propyloxy, iso-propyloxy, n-butoxy, trifluoromethoxy, difluoromethoxy, fluoromethoxy, acetyl, propionyl, n-butanoyl, iso-butanoyl, n-pentanoyl, nitro, amino, N-methylamino, N-ethylamino, N-n-propylamino, N,N-dimethylamino, N-acetylamino, N-propionylamino, N-(trifluoroacetyl)amino, formyl, hydroxy, methylthio, ethylthio, n-propylthio, methylsulfonyl, ethylsulfonyl, n-propylsulfonyl, phenyl, hydroxymethyl, 1-hydroxyethyl and 2-hydroxyethyl;
(ii) [symbol] is a single or double bond;
(iii) Z is selected from a straight chain or branched (C₁-C₈)alkyl, a straight chain or branched (C₂-C₈)alkenyl, a straight chain or branched (C₂-C₈)alkynyl, (C₃-C₈)cycloalkyl, (C₅-C₈)cycloalkenyl, (3-8 membered) heterocycloalkyl, (C₇-C₁₄)bicycloalkyl, (C₇-C₁₄) bicycloalkenyl, (7-14 membered) heterobicycloalkyl, (C₆-C₁₀) aryl, (5-10-membered) heteroaryl, and —CH—C(O)—CH═CH-Q, wherein Q is (C₃-C₈)cycloalkyl, (C₅-C₈)cycloalkenyl, (3-8 membered)heterocycloalkyl, (C₆-C₁₀)aryl, and (5-6-membered)heteroaryl; wherein each cycloalkyl, cycloalkenyl, heterocyclylalkyl, aryl, and heteroaryl are optionally substituted with one, two, three, four, or five substituents independently selected from (C₁-C₆)alkoxy, (C₁-C₆)alkyl, halo, (C₁-C₆)haloalkoxy, nitro, amino, N-methylamino, N-ethylamino, N—N-propylamino, N,N-dimethylamino, formyl, and hydroxy, and
(iv) L is single, double or triple bond, and wherein the LRRC4 family mimic molecule is not selected from:

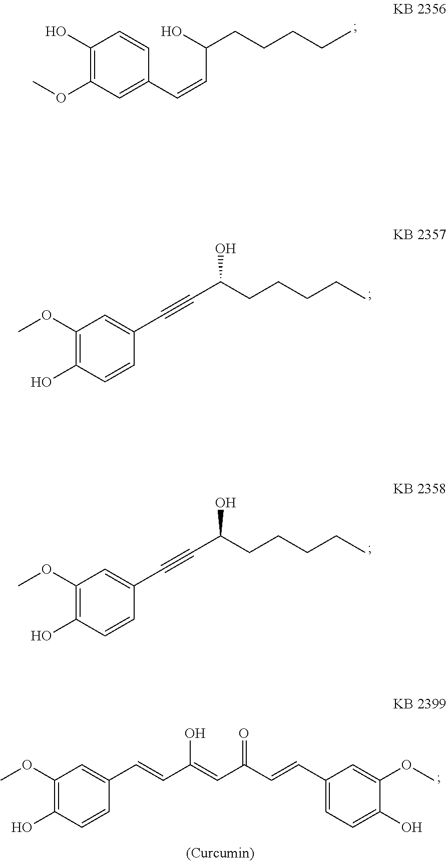

or a pharmaceutically acceptable salt thereof.

In some aspects, the LRRC4 family mimic molecule is:

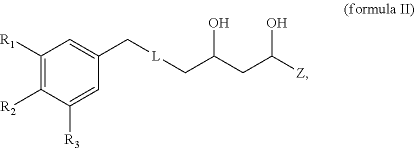

or a pharmaceutically acceptable salt thereof, wherein:
(i) R1, R2 and R3 are independently selected from hydrogen, fluoro, chloro, bromo, methyl, ethyl, n-propyl, iso-propyl, n-butyl, iso-butyl, t-butyl, n-pentyl, iso-pentyl, fluoromethyl, difluoromethyl, trifluoromethyl, 2-fluoroethyl, 1-fluoroethyl, 2,2-difluoroethyl, 1,2-difluoroethyl, 1,1-difluoroethyl, 2,2,2-trifluoroethyl, methoxy, ethoxy, n-propyloxy, iso-propyloxy, n-butoxy, trifluoromethoxy, difluoromethoxy, fluoromethoxy, acetyl, propionyl, n-butanoyl, iso-butanoyl, n-pentanoyl, nitro, amino, N-methylamino, N-ethylamino, N-n-propylamino, N,N-dimethylamino, N-acetylamino, N-propionylamino, N-(trifluoroacetyl)amino, formyl, hydroxy, methylthio, ethylthio, n-propylthio, methylsulfonyl, ethylsulfonyl, n-propylsulfonyl, phenyl, hydroxymethyl, 1-hydroxyethyl and 2-hydroxyethyl,
(ii) Z is selected from a straight chain or branched (C₁-C₈)alkyl, a straight chain or branched (C₂-C₈)alkenyl, a straight chain or branched (C₂-C₈)alkynyl, (C₃-C₈)cycloalkyl, (C₅-C₈)cycloalkenyl, (3-8 membered) heterocycloalkyl, (C₇-C₁₄)bicycloalkyl, (C₇-C₁₄) bicycloalkenyl, (7-14 membered) heterobicycloalkyl, (C₆-C₁₀) aryl, (5-10-membered) heteroaryl, and —CH═CH-Q, wherein Q is (C₃-C₈)cycloalkyl, (C₅-C₈)cycloalkenyl, (3-8 membered)heterocycloalkyl, (C₆-C₁₀)aryl, and (5-6-membered)heteroaryl; wherein each cycloalkyl, cycloalkenyl, heterocyclylalkyl, aryl, and heteroaryl are optionally substituted with one, two, three, four, or five substituents independently selected from (C₁-C₆)alkoxy, (C₁-C₆)alkyl, halo, (C₁-C₆)haloalkoxy, nitro, amino, N-methylamino, N-ethylamino, N—N-propylamino, N,N-dimethylamino, formyl, and hydroxy, and
(iii) L is single, double or triple bond.

In some aspects, R₁, R₂ and R₃ are selected from hydrogen, fluoro, chloro, bromo, hydroxy, methoxy, ethoxy, n-propyloxy, iso-propyloxy, n-butoxy, trifluoromethoxy, difluoromethoxy, fluoromethoxy, amino, N-methylamino, N-ethylamino, N—N-propylamino, and N,N-dimethylamino. In some aspects, R₁, R₂ and R₃ are selected from hydrogen, fluoro, chloro, bromo, hydroxy, methoxy, ethoxy, n-propyloxy, amino, N-methylamino, N-ethylamino, N-N-propylamino, and N,N-dimethylamino. In some aspects, R₁, R₂ and R₃ are selected from hydrogen, fluoro, hydroxy, methoxy, and N,N-dimethylamino. In some aspects, R₁ and R₂ are selected from fluoro, hydroxy, methoxy, and N,N-dimethylamino, and R₃ is hydrogen.

In some aspects, Z is selected from a straight chain or branched (C₁-C₈)alkyl, a straight chain or branched (C₂-C₈)alkenyl, a straight chain or branched (C₂-C₈)alkynyl, and —CH═CH-Q, wherein Q is selected from (C₆-C₁₀)aryl, and (5-6-membered)heteroaryl; wherein the aryl, and the heteroaryl are optionally substituted with one, two, or three substituents independently selected from (C₁-C₆)alkoxy, (C₁-C₆)alkyl, halo, (C₁-C₆)haloalkoxy, and hydroxy. In some aspects, Z is selected from a straight chain or branched (C₁-C₆)alkyl, a straight chain or branched (C₂-C₈)alkenyl, and —CH═CH-Q, wherein Q is (C₆-C₁₀)aryl optionally substituted with one, two, or three substituents independently selected from (C₁-C₆)alkoxy, (C₁-C₆)alkyl, halo, (C₁-C₆)haloalkoxy, and hydroxyl.

In some aspects, the LRRC4 family mimic molecule is a small molecule of formula (II) wherein:
(i) R₁, R₂ and R₃ are selected from hydrogen, fluoro, chloro, bromo, hydroxy, methoxy, ethoxy, n-propyloxy, iso-propyloxy, n-butoxy, trifluoromethoxy, difluoromethoxy, fluoromethoxy, amino, N-methylamino, N-ethylamino, N—N-propylamino, and N,N-dimethylamino. In some aspects, R₁, R₂ and R₅ are selected from hydrogen, fluoro, chloro, bromo, hydroxy, methoxy, ethoxy, n-propyloxy, amino, N-methylamino, N-ethylamino, N—N-propylamino, and N,N-dimethylamino;
(ii) Z is selected from a straight chain or branched (C₁-C₈)alkyl, a straight chain or branched (C₂-C₈)alkenyl, a straight chain or branched (C₂-C₈)alkynyl, and —CH═CH-Q, wherein Q is selected from (C₆-C₁₀)aryl, and (5-6-membered)heteroaryl; wherein the aryl, and the heteroaryl are optionally substituted with one, two, or three substituents independently selected from (C₁-C₆)alkoxy, (C₁-C₆)alkyl, halo, (C₁-C₆)haloalkoxy, and hydroxy; and
(iii) L is a double bond.

In some aspects, the LRRC4 family mimic molecule is a small molecule of formula (ii) wherein:
(i) R₁, R₂ and R₅ are selected from hydrogen, fluoro, chloro, bromo, hydroxy, methoxy, ethoxy, n-propyloxy, amino, N-methylamino, N-ethylamino, N—N-propylamino, and N,N-dimethylamino;
(ii) Z is selected from a straight chain or branched (C₁-C₈)alkyl, a straight chain or branched (C₂-C₈)alkenyl, and —CH═CH-Q, wherein Q is (C₆-C₁₀)aryl optionally substituted with one, two, or three substituents independently selected from (C₁-C₆)alkoxy, (C₁-C₆)alkyl, halo, (C₁-C₆)haloalkoxy, and hydroxy; and
(iii) L is a double bond.

In some aspects, the LRRC4 family molecule is selected from:

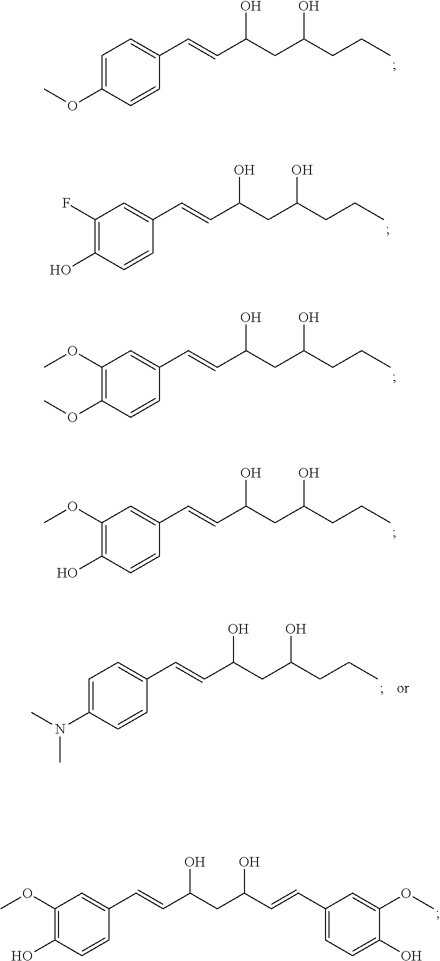

or a pharmaceutically acceptable salt thereof.

In some aspects, the LRRC4 family mimic molecule is:

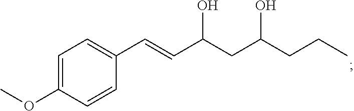 [SMILES: CCCC(O)CC(O)/C=C/c1ccc(OC)cc1]

or a pharmaceutically acceptable salt thereof.

In some aspects, the LRRC4 family mimic molecule is:

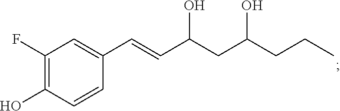 [SMILES: CCCC(O)CC(O)/C=C/c1ccc(O)c(F)c1]

or a pharmaceutically acceptable salt thereof.

In some aspects, the LRRC4 family mimic molecule is:

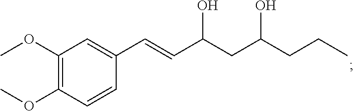 [SMILES: CCCC(O)CC(O)/C=C/c1ccc(OC)c(OC)c1]

or a pharmaceutically acceptable salt thereof.

In some aspects, the LRRC4 family mimic molecule is:

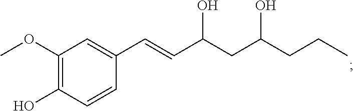 [SMILES: CCCC(O)CC(O)/C=C/c1ccc(O)c(OC)c1]

or a pharmaceutically acceptable salt thereof.

In some aspects, the LRRC4 family mimic molecule is:

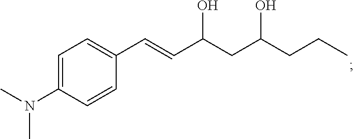 [SMILES: CCCC(O)CC(O)/C=C/c1ccc(N(C)C)cc1]

or a pharmaceutically acceptable salt thereof.

In some aspects, the LRRC4 family mimic molecule is:

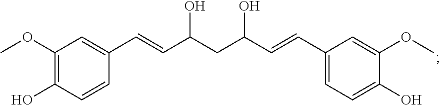

or a pharmaceutically acceptable salt thereof.

In some aspects, the LRRC4 family mimic molecule is:

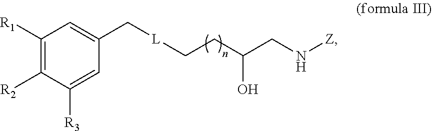

or a pharmaceutically acceptable salt thereof, wherein:
(i) R1, R2 and R3 are independently selected from hydrogen, fluoro, chloro, bromo, methyl, ethyl, n-propyl, iso-propyl, n-butyl, iso-butyl, t-butyl, n-pentyl, iso-pentyl, fluoromethyl, difluoromethyl, trifluoromethyl, 2-fluoroethyl, 1-fluoroethyl, 2,2-difluoroethyl, 1,2-difluoroethyl, 1,1-difluoroethyl, 2,2,2-trifluoroethyl, methoxy, ethoxy, n-propyloxy, iso-propyloxy, n-butoxy, trifluoromethoxy, difluoromethoxy, fluoromethoxy, acetyl, propionyl, n-butanoyl, iso-butanoyl, n-pentanoyl, nitro, amino, N-methylamino, N-ethylamino, N-n-propylamino, N,N-dimethylamino, N-acetylamino, N-propionylamino, N-(trifluoroacetyl)amino, formyl, hydroxy, methylthio, ethylthio, n-propylthio, methylsulfonyl, ethylsulfonyl, n-propylsulfonyl, phenyl, hydroxymethyl, 1-hydroxyethyl and 2-hydroxyethyl,
(ii) Z is selected from a straight chain or branched (C₁-C₈)alkyl, a straight chain or branched (C₂-C₈)alkenyl, a straight chain or branched (C₂-C₈)alkynyl, —Y—(C₃-C₈)cycloalkyl, —Y—(C₅-C₈)cycloalkenyl, —Y-(3-8 membered) heterocycloalkyl, —Y—(C₇-C₁₄)bicycloalkyl, —Y—(C₇-C₁₄) bicycloalkenyl, —Y-(7-14 membered) heterobicycloalkyl, —Y—(C₆-C₁₀)aryl, and —Y-(5-10-membered) heteroaryl, wherein Y is a bond or a C₁-C₃ straight or branched alkylene, and wherein the cycloalkyl, the cycloalkenyl, the heterocyclylalkyl, the aryl, and the heteroaryl are optionally substituted with one, two, three, four, or five substituents independently selected from C₁-C₆alkoxy, C₁-C₆alkyl, halo, C₁-C₆haloalkoxy, nitro, amino, N-methylamino, N-ethylamino, N—N-propylamino, N,N-dimethylamino, formyl, and hydroxy,
(iii) L is single, double or triple bond, and
(iv) n is 0 or 1.

In some aspects, R₁, R₂ and R₃ are selected from hydrogen, hydroxy, methoxy, ethoxy, n-propyloxy, iso-propyloxy, n-butoxy, trifluoromethoxy, difluoromethoxy, and fluoromethoxy. In some aspects, R₁, R₂ and R₃ are selected from hydrogen, hydroxy, methoxy, ethoxy, n-propyloxy, iso-propyloxy, n-butoxy. In some aspects, R₁, R₂ and R₃ are selected from hydrogen, hydroxy, and methoxy. In some aspects, R₁ and R₂ are selected from hydroxy and methoxy and R₃ is hydrogen.

In some aspects, Z is selected from —Y—(C₃-C₈)cycloalkyl, —Y—(C₅-C₈)cycloalkenyl, —Y-(3-8 membered) heterocycloalkyl, —Y—(C₇-C₁₄)bicycloalkyl, —Y—(C₇-C₁₄) bicycloalkenyl, —Y-(7-14 membered) heterobicycloalkyl, —Y—(C₆-C₁₀)aryl, and —Y-(5-10-membered) heteroaryl, wherein Y is a bond or a C₁-C₃ straight or branched alkylene, and wherein the cycloalkyl, the cycloalkenyl, the heterocyclylalkyl, the aryl, and the heteroaryl are optionally substituted with one, two, three, four, or five substituents independently selected from C₁-C₆alkoxy, C₁-C₆alkyl, halo, C₁-C₆haloalkoxy, nitro, amino, N-methylamino, N-ethylamino, N—N-propylamino, N,N-dimethylamino, formyl, and hydroxy. In some aspects, Z is selected from —Y—(C₆-C₁₀)aryl, and —Y-(5-10-membered) heteroaryl wherein Y is a bond or a C₁-C₃ straight or branched alkylene, and wherein the aryl and the heteroaryl are optionally substituted with one, two, or three substituents independently selected from C₁-C₆alkoxy, and halo.

In some aspects, the LRRC4 family mimic molecule is a small molecule of formula (III) wherein:
(i) R₁, R₂ and R₃ are selected from hydrogen, hydroxy, methoxy, ethoxy, n-propyloxy, iso-propyloxy, n-butoxy, trifluoromethoxy, difluoromethoxy, and fluoromethoxy;
(ii) Z is selected from —Y—(C₃-C₈)cycloalkyl, —Y—(C₅-C₈)cycloalkenyl, —Y-(3-8 membered) heterocycloalkyl, —Y—(C₇-C₁₄)bicycloalkyl, —Y—(C₇-C₁₄) bicycloalkenyl, —Y-(7-14 membered) heterobicycloalkyl, —Y—(C₆-C₁₀)aryl, and —Y-(5-10-membered) heteroaryl, wherein Y is a bond or a C₁-C₃ straight or branched alkylene, and wherein the cycloalkyl, the cycloalkenyl, the heterocyclylalkyl, the aryl, and the heteroaryl are optionally substituted with one, two, three, four, or five substituents independently selected from C₁-C₆alkoxy, C₁-C₆alkyl, halo, C₁-C₆haloalkoxy, nitro, amino, N-methylamino, N-ethylamino, N—N-propylamino, N,N-dimethylamino, formyl, and hydroxy; and
(iii) L is a triple bond.

In some aspects, the LRRC4 family mimic molecule is a small molecule of formula (III) wherein:
(i) R₁, R₂ and R₃ are selected from hydrogen, hydroxy, methoxy, ethoxy, n-propyloxy, iso-propyloxy, and n-butoxy;
(ii) Z is selected from —Y—(C₆-C₁₀)aryl, and —Y-(5-10-membered) heteroaryl wherein Y is a bond or a C₁-C₃ straight or branched alkylene, and wherein the aryl and the heteroaryl are optionally substituted with one, two, or three substituents independently selected from C₁-C₆alkoxy, and halo; and
(iii) L is a triple bond.

In some aspects, the LRRC4 family mimic molecule is selected from:

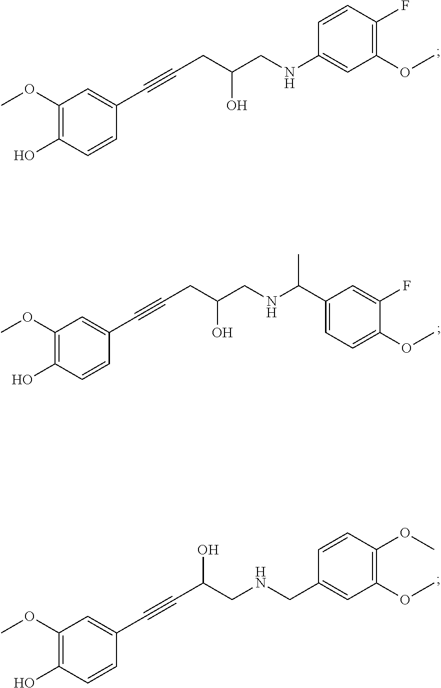

or a pharmaceutically acceptable salt thereof.

In some aspects, the LRRC4 family mimic molecule is:

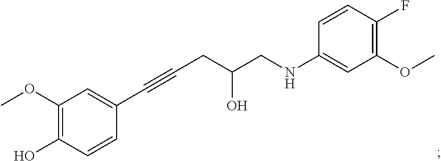

or a pharmaceutically acceptable salt thereof.

In some aspects, the LRRC4 family mimic molecule is:

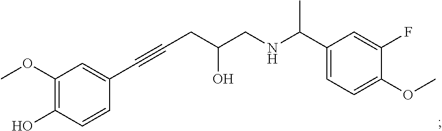

or a pharmaceutically acceptable salt thereof.

In some aspects, the LRRC4 family mimic molecule is:

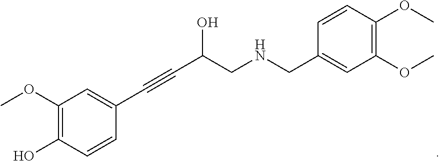

As described herein, where the LRRC4 family mimic molecule useful for the present disclosure comprises a polypeptide, in some aspects, the polypeptide comprises at least the FAM19A5 binding domain of members of the LRRC4 protein family. Unless indicated otherwise, the overall length of the FAM19A5 binding domain is not particularly limited, as long as the domain is capable of binding to the FAM19A5 protein. In some aspects, the FAM19A5 binding domain is at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, or at least about 30 amino acids in length. In some aspects, the FAM19A5 binding domain is about 10 to about 23 amino acids in length. In some aspects, the FAM19A5 binding domain is about 10, about 11, about 12, about 13, about 14, about 15, about 16, about 17, about 18, about 19, about 20, about 21, about 22, or about 23 amino acids in length. In some aspects, the FAM19A5 binding domain of a LRRC4 family mimic molecule is about 10 amino acids in length.

In some aspects, the polypeptide of a LRRC4 family mimic molecule comprises an amino acid sequence having the following formula (from N-terminus to C-terminus):

(Formula IV)
(SEQ ID NO: 25)
A-(T/S)-B,

wherein:
(i) “A” comprises X1-(T/S)-(Y/F)-F-X5, and (ii) “B” comprises (V/I)-T-V-(E/V), and
wherein:
X1 is tyrosine (Y), phenylalanine (F), valine (V), leucine (L), or isoleucine (I);
(T/S) is threonine (T) or serine (S);
(Y/F) is tyrosine (Y) or Phenylalanine (F);
X5 is any amino acids;
(V/I) is valine (V) or isoleucine (I); and
(E/V) is glutamic acid (E) or valine (V).

In some aspects, the polypeptide of a LRRC4 family mimic molecule described herein comprises an amino acid sequence having the formula (from N-terminus to C-terminus):

(Formula IV)
(SEQ ID NO: 26)
A-(T/S)-B,

wherein:
(i) “A” comprises (Y/W/M)-(T/Y)-(Y/W)-(F/Y/W)-(T/Y), and (ii) “B” comprises X7-(T/S/Y)-X9-X10, and wherein
(Y/W/M) is tyrosine (Y), tryptophan (W), or methionine (M);
(T/Y) is threonine (T) or tyrosine (Y);
(Y/W) is tyrosine (Y) or tryptophan (W);
(F/Y/W) is phenylalanine (F), tyrosine (Y), or tryptophan (W);
X7 is valine (V), tyrosine (Y), phenylalanine (F), leucine (L), tryptophan (W), or methionine (M);
(T/S/Y) is threonine (T), serine (S), or tyrosine (Y);
X9 is valine (V), isoleucine (I), tyrosine (Y), phenylalanine (F), leucine (L), tryptophan (W), or methionine (M); and
X10 is glutamic acid (E), aspartic acid (D), isoleucine (I), tyrosine (Y), phenylalanine (F), methionine (M), or tryptophan (W).

In some aspects, a LRRC4 family mimic molecule useful for the present disclosure comprises a polypeptide that comprises an amino acid sequence having the following formula (from N-terminus to C-terminus):

(Formula V)
(SEQ ID NO: 27)
X1-X2-X3-F-X5-T-X7-T-V-X10,

wherein:
X1 is Y, F, V, L, or I;
X2 is T or S;
X3 is Y or F;
X5 is any amino acid;
X7 is V or I; and/or
X10 is E or V,
   and wherein the polypeptide is capable of binding to a FAM19A5 protein, and thereby inhibiting, reducing, and/or dissociating the interaction between the FAM19A5 protein and members of the LRRC4 protein family.

In some aspects, a LRRC4 family mimic molecule described herein comprises a polypeptide that comprises an amino acid sequence having the following formula (from N-terminus to C-terminus):

(Formula VI)
(SEQ ID NO: 28)
X1-X2-X3-X4-X5-X6-X7-X8-X9-X10,

wherein:
X1 is Y, F, V, L, I, W, or M;
X2 is T, S, or Y;
X3 is Y, F, or W;
X4 is F, Y, or W;
X5 is any amino acids, e.g., T, S, or Y;
X6 is T, S, or Y;
X7 is V, I, Y, F, L, W, or M;
X8 is T, S, or Y;
X9 is V, I, Y, F, L, W, or M; and/or
X10 is E, D, V, I, Y, F, M, or W,
   and wherein the polypeptide is capable of binding to a FAM19A5 protein, and thereby inhibiting, reducing, and/or dissociating the interaction between the FAM19A5 protein and members of the LRRC4 protein family.

For any of the above LRRC4 family mimic molecules, in some aspects, (i) X1 is Y, F, V, L, or I; (ii) X2 is T or S; (iii) X3 is Y or F; (iv) X4 is F; (v) X5 is T or S; (vi) X6 is T; (vii) X7 is V or I; (viii) X8 is T; (ix) X9 is V; (x) X10 is E or V; and (xi) any combinations of (i)-(x). In some aspects, X1 is Y, F, V, L, or I. In some aspects, X2 is T or S. In some aspects, X3 is Y or F. In some aspects, X4 is F. In some aspects, X5 is T or S. In some aspects, X6 is T. In some aspects, X7 is V or I. In some aspects, X8 is T. In some aspects, X9 is V. In some aspects, X10 is E or V. In some aspects, the amino acid at position X2 is phosphorylated. In some aspects, the amino acid at position X2 is O-glycosylated.

In some aspects, a polypeptide of a LRRC4 family mimic molecule described herein comprises the amino acid sequence set forth in SEQ ID NO: 29 (YTYFTTVTVE), with one, two, three, four, five, or six amino acids different from the amino acid sequence (e.g., substitutions). In some aspects, a polypeptide of a LRRC4 family mimic molecule disclosed herein consists of the amino acid sequence set forth in SEQ ID NO: 29 (YTYFTTVTVE), with one, two, three, four, five, or six amino acids different from the amino acid sequence (e.g., substitutions). In some aspects, a polypeptide of a LRRC4 family mimic molecule disclosed herein consists essentially of the amino acid sequence set forth in SEQ ID NO: 29 (YTYFTTVTVE), with one, two, three, four, five, or six amino acids different from the amino acid sequence (e.g., substitutions).

In some aspects, a polypeptide of a LRRC4 family mimic molecule comprises the amino acid sequence set forth in SEQ ID NO: 29 (YTYFTTVTVE). In some aspects, the polypeptide of a LRRC4 family mimic molecule consists of the amino acid sequence set forth in SEQ ID NO: 29 (YTYFTTVTVE). In some aspects, the polypeptide of a LRRC4 family mimic molecule consists essentially of the amino acid sequence set forth in SEQ ID NO: 29 (YTYFTTVTVE). As demonstrated herein, the amino acid sequence set forth in SEQ ID NO: 29 (YTYFTTVTVE) corresponds to the FAM19A5 binding domain of the LRRC4B protein.

In some aspects, a polypeptide of a LRRC4 family mimic molecule comprises the amino acid sequence set forth in SEQ ID NO: 30 (YSFFTTVTVE). In some aspects, the polypeptide of a LRRC4 family mimic molecule consists of the amino acid sequence set forth in SEQ ID NO: 30 (YSFFTTVTVE). In some aspects, the polypeptide of a LRRC4 family mimic molecule consists essentially of the amino acid sequence set forth in SEQ ID NO: 30 (YSFFTTVTVE). As demonstrated herein, the amino acid sequence set forth in SEQ ID NO: 30 (YSFFTTVTVE) corresponds to the FAM19A5 binding domain of the LRRC4 protein.

In some aspects, a polypeptide of a LRRC4 family mimic molecule comprises the amino acid sequence set forth in SEQ ID NO: 31 (FSYFSTVTVE). In some aspects, the polypeptide of a LRRC4 family mimic molecule consists of the amino acid sequence set forth in SEQ ID NO: 31 (FSYFSTVTVE). In some aspects, the polypeptide of a LRRC4 family mimic molecule consists essentially of the amino acid sequence set forth in SEQ ID NO: 31 (FSYFSTVTVE). As demonstrated herein, the amino acid sequence set forth in SEQ ID NO: 31 (FSYFSTVTVE) corresponds to the FAM19A5 binding domain of the LRRC4C protein.

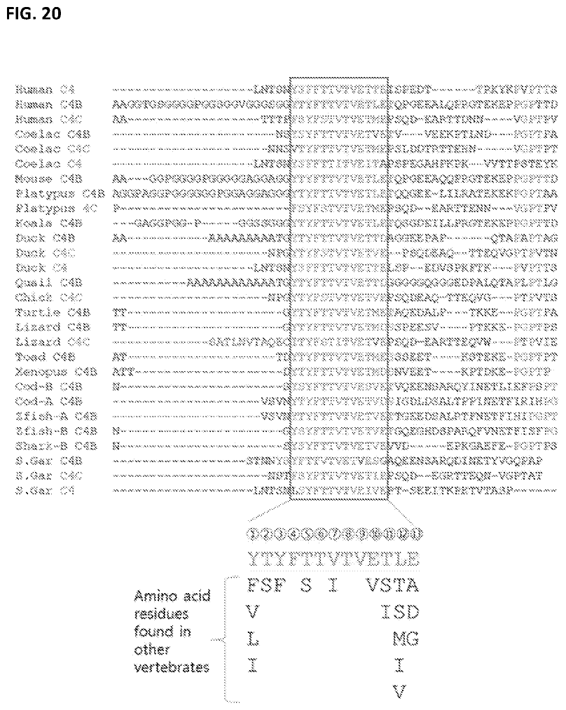

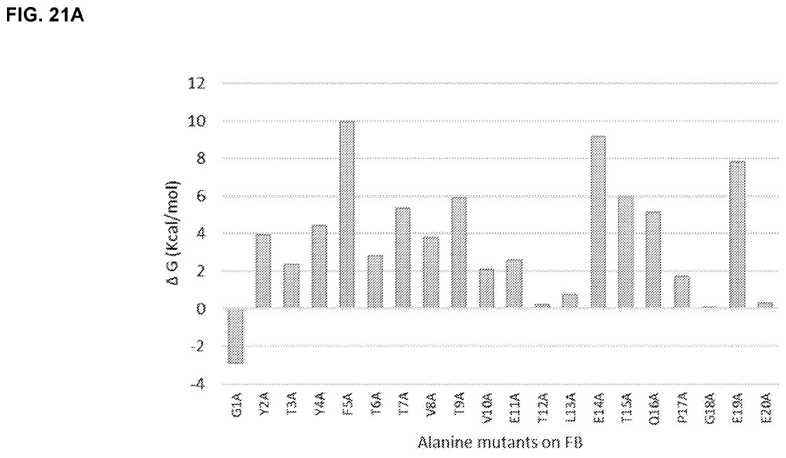

As described herein, the FAM19A5 binding domains of members of the LRRC4 protein family are largely conserved among vertebrates (see, e.g., FIG. 20). Accordingly, not to be bound by any one theory, one or more amino acid residues of the amino acid sequence set forth in any one of SEQ ID NOs: 29 (YTYFTTVTVE), 30 (YSFFTTVTVE), and 31 (FSYFSTVTVE) can be substituted with an amino acid present in the corresponding residue in other vertebrates. Examples of such substitutions are provided herein (see, e.g., FIG. 20).

In some aspects, one or more amino acid residues of the amino acid sequence set forth in any one of SEQ ID NOs: 29 (YTYFTTVTVE), 30 (YSFFTTVTVE), and 31 (FSYFSTVTVE) can be substituted with an amino acid sharing similar biochemical properties. For instance, in the amino acid sequence set forth in SEQ ID NO: 29 (YTYFTTVTVE), in some aspects, the Y at position I can be substituted with other hydrophobic amino acids (e.g., F, V, L, I, W, or M). In some aspects, the T at position 2 can be substituted with other amino acids having a similar hydroxyl (OH) group in its side chain (e.g., S or Y). In some aspects, the Y at position 3 can be substituted with other amino acids having common aromatic ring in its side chain that can participate in Van der Waals interaction (e.g., F or W). In some aspects, the F at position 4 can be substituted with amino acids, such as Y or W. In some aspects, the T at position 5 can be substituted with amino acids, such as S or Y. In some aspects, the T at position 6 can be substituted with amino acids, such as S or Y. In some aspects, the V at position 7 can be substituted with other amino acids having hydrophobic bulky side chains (e.g., I, Y, F, L, W, or M). In some aspects, the T at position 8 can be substituted with other amino acids, such as S or Y. In some aspects, the V at position 9 can be substituted with other amino acids, such as I, Y, F, L, W, or M. In some aspects, the E at position 10 can be substituted with other amino acids that have an acidic side chain (e.g., I, Y, F, M, or W).

In some aspects, a polypeptide of a LRRC4 family mimic molecule described herein comprises an amino acid sequence that is at least about 40%, at least about 50%, at least about 60%, at least about 70%, at least about 80%, or at least about 90% identical to the amino acid sequence set forth in SEQ ID NO: 29 (YTYFTTVTVE), wherein the polypeptide is capable of binding to a FAM19A5 protein, and thereby inhibiting, reducing, and/or dissociating the interaction between the FAM19A5 protein and members of the LRRC4 protein family. In some aspects, a polypeptide of a LRRC4 family mimic molecule comprises an amino acid sequence that is at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to the amino acid sequence set forth in SEQ ID NO: 5, wherein the polypeptide is capable of binding to a FAM19A5 protein, and thereby inhibiting, reducing, and/or dissociating the interaction between the FAM19A5 protein and members of the LRRC4 protein family. In some aspects, a polypeptide of a LRRC4 family mimic molecule comprises an amino acid sequence that is at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to the amino acid sequence set forth in SEQ ID NO: 4, wherein the polypeptide is capable of binding to a FAM19A5 protein, and thereby inhibiting, reducing, and/or dissociating the interaction between the FAM19A5 protein and members of the LRRC4 protein family. In some aspects, a polypeptide of a LRRC4 family mimic molecule comprises an amino acid sequence that is at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to the amino acid sequence set forth in SEQ ID NO: 6, wherein the polypeptide is capable of binding to a FAM19A5 protein, and thereby inhibiting, reducing, and/or dissociating the interaction between the FAM19A5 protein and members of the LRRC4 protein family.

As is apparent from the present disclosure, in some aspects, polypeptides of the LRRC4 family mimic molecules described herein (e.g., comprising a FAM19A5 binding domain of members of the LRRC4 protein family) comprise one or more amino acid modifications. In some aspects, the one or more amino acid modifications can increase the binding affinity of the LRRC4 family mimic molecule to the FAM19A5 protein. Accordingly, in some aspects, the binding affinity of a LRRC4 family mimic molecule described herein to a FAM19A5 protein is increased by at least about 0.5-fold, at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 35-fold, at least about 40-fold, at least about 45-fold, or at least about 50-fold, compared to a reference (e.g., corresponding LRRC4 family mimic molecule without the amino acid modification(s) or naturally-existing members of the LRRC4 protein family). In some aspects, the one or more amino acid modifications can improve the stability of the LRRC4 family mimic molecule. Accordingly, in some aspects, the stability of a LRRC4 family mimic molecule described herein is increased by at least about 0.5-fold, at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 35-fold, at least about 40-fold, at least about 45-fold, or at least about 50-fold, compared to a reference (e.g., corresponding LRRC4 family mimic molecule without the amino acid modification(s) or naturally-existing members of the LRRC4 protein family).

In some aspects, the one or more amino acid modifications can improve the ability of the LRRC4 family mimic molecules described herein to inhibit the interaction between a FAM19A5 protein and members of the LRRC4 protein family (e.g., by increasing the binding affinity and/or stability). Accordingly, in some aspects, the ability of the LRRC4 family mimic molecule to inhibit the interaction between a FAM19A5 protein and members of the LRRC4 protein family is increased by at least about 0.5-fold, at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 35-fold, at least about 40-fold, at least about 45-fold, or at least about 50-fold, compared to a reference (e.g., corresponding LRRC4 family mimic molecule without the amino acid modification(s) or naturally-existing members of the LRRC4 protein family).

Non-limiting examples of amino acid modifications that are useful for the present disclosure are provided throughout the present disclosure. For instance, in some aspects, a polypeptide described herein comprises one of the FAM19A5 binding domains of a members of the LRRC4 protein family—i.e., YTYFTTVTVE (SEQ ID NO: 29), YSFFTTVTVE (SEQ ID NO: 30), or FSYFSTVTVE (SEQ ID NO: 31)—and one or more amino acids at the N-terminus, C-terminus, or both at the N-terminus and C-terminus of the polypeptide. In some aspects, a polypeptide useful for the present disclosure comprises at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, or at least about 20 additional amino acids at the N-terminus of the polypeptide. In some aspects, the polypeptide comprises at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, or at least about additional 20 amino acids at the C-terminus of the polypeptide. In some aspects, the polypeptide comprises: (i) at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, or at least about 20 additional amino acids at the N-terminus of the polypeptide; and (ii) at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, or at least about 20 additional amino acids at the C-terminus of the polypeptide. As demonstrated herein (see, e.g., Example 9), in some aspects, the one or more amino acids differ from the amino acids present at the particular residues in a naturally existing LRRC4 protein family member.

For instance, in some aspects, a polypeptide described herein comprises the amino acid sequence set forth in SEQ ID NO: 18 (GYTYFTTVTVETLETQPGEE) with one or more amino acid modifications (e.g., substitutions). In some aspects, a polypeptide described herein comprises the amino acid sequence set forth in SEQ ID NO: 18 (GYTYFTTVTVETLETQPGEE) with two amino acid modifications (e.g., substitutions). In some aspects, the amino acid modifications are at residues T12 and L13 of SEQ ID NO: 18. In some aspects, a polypeptide described herein consists of the amino acid sequence set forth in SEQ ID NO: 18 (GYTYFTTVTVETLETQPGEE) with one or more amino acid modifications (e.g., substitutions). In some aspects, a polypeptide described herein consists of the amino acid sequence set forth in SEQ ID NO: 18 (GYTYFTTVTVETLETQPGEE) with two amino acid modifications (e.g., substitutions). In some aspects, the amino acid modifications are at residues T12 and L13 of SEQ ID NO: 18. In some aspects, a polypeptide described herein consists essentially of the amino acid sequence set forth in SEQ ID NO: 18 (GYTYFTTVTVETLETQPGEE) with one or more amino acid modifications (e.g., substitutions). In some aspects, a polypeptide described herein consists essentially of the amino acid sequence set forth in SEQ ID NO: 18 (GYTYFTTVTVETLETQPGEE) with two amino acid modifications (e.g., substitutions). In some aspects, the amino acid modifications are at residues T12 and L13 of SEQ ID NO: 18.

In some aspects, a polypeptide described herein comprises the amino acid sequence set forth in SEQ ID NO: 17 (GYTYFTTVTVETLETQ) with one or more amino acid modifications (e.g., substitutions). In some aspects, a polypeptide described herein comprises the amino acid sequence set forth in SEQ ID NO: 17 (GYTYFTTVTVETLETQ) with two amino acid modifications (e.g., substitutions). In some aspects, the amino acid modifications are at residues T12 and L13 of SEQ ID NO: 17. In some aspects, a polypeptide described herein consists of the amino acid sequence set forth in SEQ ID NO: 17 (GYTYFTTVTVETLETQ) with one or more amino acid modifications (e.g., substitutions). In some aspects, a polypeptide described herein consists of the amino acid sequence set forth in SEQ ID NO: 17 (GYTYFTTVTVETLETQ) with two amino acid modifications (e.g., substitutions). In some aspects, the amino acid modifications are at residues T12 and L13 of SEQ ID NO: 17. In some aspects, a polypeptide described herein consists essentially of the amino acid sequence set forth in SEQ ID NO: 17 (GYTYFTTVTVETLETQ) with one or more amino acid modifications (e.g., substitutions). In some aspects, a polypeptide described herein consists essentially of the amino acid sequence set forth in SEQ ID NO: 17 (GYTYFTTVTVETLETQ) with two amino acid modifications (e.g., substitutions). In some aspects, the amino acid modifications are at residues T12 and L13 of SEQ ID NO: 17.

In some aspects, a polypeptide described herein comprises the amino acid sequence set forth in SEQ ID NO: 19 (GYTYFTTVTVETLETQPGEKEPPGPTTD) with one or more amino acid modifications (e.g., substitutions). In some aspects, a polypeptide described herein comprises the amino acid sequence set forth in SEQ ID NO: 19 (GYTYFTTVTVETLETQPGEKEPPGPTTD) with two amino acid modifications (e.g., substitutions). In some aspects, the amino acid modifications are at residues T12 and L13 of SEQ ID NO: 19. In some aspects, a polypeptide described herein consists of the amino acid sequence set forth in SEQ ID NO: 19 (GYTYFTTVTVETLETQPGEKEPPGPTTD) with one or more amino acid modifications (e.g., substitutions). In some aspects, a polypeptide described herein consists of the amino acid sequence set forth in SEQ ID NO: 19 (GYTYFTTVTVETLETQPGEKEPPGPTTD) with two amino acid modifications (e.g., substitutions). In some aspects, the amino acid modifications are at residues T12 and L13 of SEQ ID NO: 19. In some aspects, a polypeptide described herein consists essentially of the amino acid sequence set forth in SEQ ID NO: 19 (GYTYFTTVTVETLETQPGEKEPPGPTTD) with one or more amino acid modifications (e.g., substitutions). In some aspects, a polypeptide described herein consists essentially of the amino acid sequence set forth in SEQ ID NO: 19 (GYTYFTTVTVETLETQPGEKEPPGPTTD) with two amino acid modifications (e.g., substitutions). In some aspects, the amino acid modifications are at residues T12 and L13 of SEQ ID NO: 19.

In some aspects, a polypeptide described herein comprises the amino acid sequence set forth in SEQ ID NO: 143 (GYTYFTTVTVETLETQPGEEA) with one or more amino acid modifications (e.g., substitutions). In some aspects, a polypeptide described herein comprises the amino acid sequence set forth in SEQ ID NO: 143 (GYTYFTTVTVETLETQPGEEA) with two amino acid modifications (e.g., substitutions). In some aspects, the amino acid modifications are at residues T12 and L13 of SEQ ID NO: 143. In some aspects, a polypeptide described herein consists of amino the acid sequence set forth in SEQ ID NO: 143 (GYTYFTTVTVETLETQPGEEA) with one or more amino acid modifications (e.g., substitutions). In some aspects, a polypeptide described herein consists of the amino acid sequence set forth in SEQ ID NO: 143 (GYTYFTTVTVETLETQPGEEA) with two amino acid modifications (e.g., substitutions). In some aspects, the amino acid modifications are at residues T12 and L13 of SEQ ID NO: 143. In some aspects, a polypeptide described herein consists essentially of the amino acid sequence set forth in SEQ ID NO: 143 (GYTYFTTVTVETLETQPGEEA) with one or more amino acid modifications (e.g., substitutions). In some aspects, a polypeptide described herein consists essentially of the amino acid sequence set forth in SEQ ID NO: 143 (GYTYFTTVTVETLETQPGEEA) with two amino acid modifications (e.g., substitutions). In some aspects, the amino acid modifications are at residues T12 and L13 of SEQ ID NO: 143.

In some aspects, a polypeptide described herein (e.g., comprising the FAM19A5 binding domain of LRRC4B) comprises the amino acid sequence GYTYFTTVTVEPYETQPGEE (SEQ ID NO: 123). In some aspects, a polypeptide described herein (e.g., comprising the FAM19A5 binding domain of LRRC4B) consists of the amino acid sequence GYTYFTTVTVEPYETQPGEE (SEQ ID NO: 123). In some aspects, a polypeptide described herein (e.g., comprising the FAM19A5 binding domain of LRRC4B) consists essentially of the amino acid sequence GYTYFTTVTVEPYETQPGEE (SEQ ID NO: 123).

In some aspects, a polypeptide described herein (e.g., comprising the FAM19A5 binding domain of LRRC4B) comprises the amino acid sequence GYTYFTTVTVEMRETQPGEE (SEQ ID NO: 124). In some aspects, a polypeptide described herein (e.g., comprising the FAM19A5 binding domain of LRRC4B) consists of the amino acid sequence GYTYFTTVTVEMRETQPGEE (SEQ ID NO: 124). In some aspects, a polypeptide described herein (e.g., comprising the FAM19A5 binding domain of LRRC4B) consists essentially of the amino acid sequence GYTYFTTVTVEMRETQPGEE (SEQ ID NO: 124).

In some aspects, a polypeptide described herein (e.g., comprising the FAM19A5 binding domain of LRRC4B) comprises the amino acid sequence GYTYFTTVTVEIFETQPGEE (SEQ ID NO: 125). In some aspects, a polypeptide described herein (e.g., comprising the FAM19A5 binding domain of LRRC4B) consists of the amino acid sequence GYTYFTTVTVEIFETQPGEE (SEQ ID NO: 125). In some aspects, a polypeptide described herein (e.g., comprising the FAM19A5 binding domain of LRRC4B) consists essentially of the amino acid sequence GYTYFTTVTVEIFETQPGEE (SEQ ID NO: 125).

In some aspects, a polypeptide described herein (e.g., comprising the FAM19A5 binding domain of LRRC4B) comprises the amino acid sequence GYTYFTTVTVEHFETQPGEE (SEQ ID NO: 126). In some aspects, a polypeptide described herein (e.g., comprising the FAM19A5 binding domain of LRRC4B) consists of the amino acid sequence GYTYFTTVTVEHFETQPGEE (SEQ ID NO: 126). In some aspects, a polypeptide described herein (e.g., comprising the FAM19A5 binding domain of LRRC4B) consists essentially of the amino acid sequence GYTYFTTVTVEHFETQPGEE (SEQ ID NO: 126).

In some aspects, a polypeptide described herein (e.g., comprising the FAM19A5 binding domain of LRRC4B) comprises the amino acid sequence GYTYFTTVTVEWYETQPGEE (SEQ ID NO: 127). In some aspects, a polypeptide described herein (e.g., comprising the FAM19A5 binding domain of LRRC4B) consists of the amino acid sequence GYTYFTTVTVEWYETQPGEE (SEQ ID NO: 127). In some aspects, a polypeptide described herein (e.g., comprising the FAM19A5 binding domain of LRRC4B) consists essentially of the amino acid sequence GYTYFTTVTVEWYETQPGEE (SEQ ID NO: 127).

In some aspects, a polypeptide described herein (e.g., comprising the FAM19A5 binding domain of LRRC4B) comprises the amino acid sequence GYTYFTTVTVEQRETQPGEE (SEQ ID NO: 128). In some aspects, a polypeptide described herein (e.g., comprising the FAM19A5 binding domain of LRRC4B) consists of the amino acid sequence GYTYFTTVTVEQRETQPGEE (SEQ ID NO: 128). In some aspects, a polypeptide described herein (e.g., comprising the FAM19A5 binding domain of LRRC4B) consists essentially of the amino acid sequence GYTYFTTVTVEQRETQPGEE (SEQ ID NO: 128).

In some aspects, a polypeptide described herein (e.g., comprising the FAM19A5 binding domain of LRRC4B) comprises the amino acid sequence GYTYFTTVTVEWFETQPGEE (SEQ ID NO: 129). In some aspects, a polypeptide described herein (e.g., comprising the FAM19A5 binding domain of LRRC4B) consists of the amino acid sequence GYTYFTTVTVEWFETQPGEE (SEQ ID NO: 129). In some aspects, a polypeptide described herein (e.g., comprising the FAM19A5 binding domain of LRRC4B) consists essentially of the amino acid sequence GYTYFTTVTVEWFETQPGEE (SEQ ID NO: 129).

In some aspects, a polypeptide described herein (e.g., comprising the FAM19A5 binding domain of LRRC4B) comprises the amino acid sequence GYTYFTTVTVEERETQPGEE (SEQ ID NO: 130). In some aspects, a polypeptide described herein (e.g., comprising the FAM19A5 binding domain of LRRC4B) consists of the amino acid sequence GYTYFTTVTVEERETQPGEE (SEQ ID NO: 130). In some aspects, a polypeptide described herein (e.g., comprising the FAM19A5 binding domain of LRRC4B) consists essentially of the amino acid sequence GYTYFTTVTVEERETQPGEE (SEQ ID NO: 130).

In some aspects, a polypeptide described herein (e.g., comprising the FAM19A5 binding domain of LRRC4B) comprises the amino acid sequence GYTYFTTVTVEDYETQPGEE (SEQ ID NO: 131). In some aspects, a polypeptide described herein (e.g., comprising the FAM19A5 binding domain of LRRC4B) consists of the amino acid sequence GYTYFTTVTVEDYETQPGEE (SEQ ID NO: 131). In some aspects, a polypeptide described herein (e.g., comprising the FAM19A5 binding domain of LRRC4B) consists essentially of the amino acid sequence GYTYFTTVTVEDYETQPGEE (SEQ ID NO: 131).

In some aspects, a polypeptide described herein (e.g., comprising the FAM19A5 binding domain of LRRC4B) comprises the amino acid sequence GYTYFTTVTVEFFETQPGEE (SEQ ID NO: 132). In some aspects, a polypeptide described herein (e.g., comprising the FAM19A5 binding domain of LRRC4B) consists of the amino acid sequence GYTYFTTVTVEFFETQPGEE (SEQ ID NO: 132). In some aspects, a polypeptide described herein (e.g., comprising the FAM19A5 binding domain of LRRC4B) consists essentially of the amino acid sequence GYTYFTTVTVEFFETQPGEE (SEQ ID NO: 132).

In some aspects, a polypeptide described herein (e.g., comprising the FAM19A5 binding domain of LRRC4B) comprises the amino acid sequence GYTYFTTVTVEHYETQPGEE (SEQ ID NO: 133). In some aspects, a polypeptide described herein (e.g., comprising the FAM19A5 binding domain of LRRC4B) consists of the amino acid sequence GYTYFTTVTVEHYETQPGEE (SEQ ID NO: 133). In some aspects, a polypeptide described herein (e.g., comprising the FAM19A5 binding domain of LRRC4B) consists essentially of the amino acid sequence GYTYFTTVTVEHYETQPGEE (SEQ ID NO: 133).

In some aspects, a polypeptide described herein (e.g., comprising the FAM19A5 binding domain of LRRC4B) comprises the amino acid sequence GYTYFTTVTVEMMETQPGEE (SEQ ID NO: 134). In some aspects, a polypeptide described herein (e.g., comprising the FAM19A5 binding domain of LRRC4B) consists of the amino acid sequence GYTYFTTVTVEMMETQPGEE (SEQ ID NO: 134). In some aspects, a polypeptide described herein (e.g., comprising the FAM19A5 binding domain of LRRC4B) consists essentially of the amino acid sequence GYTYFTTVTVEMMETQPGEE (SEQ ID NO: 134).

In some aspects, a polypeptide described herein (e.g., comprising the FAM19A5 binding domain of LRRC4B) comprises the amino acid sequence GYTYFTTVTVEDFETQPGEE (SEQ ID NO: 135). In some aspects, a polypeptide described herein (e.g., comprising the FAM19A5 binding domain of LRRC4B) consists of the amino acid sequence GYTYFTTVTVEDFETQPGEE (SEQ ID NO: 135). In some aspects, a polypeptide described herein (e.g., comprising the FAM19A5 binding domain of LRRC4B) consists essentially of the amino acid sequence GYTYFTTVTVEDFETQPGEE (SEQ ID NO: 135).

In some aspects, a polypeptide described herein (e.g., comprising the FAM19A5 binding domain of LRRC4B) comprises the amino acid sequence GYTYFTTVTVEDIETQPGEE (SEQ ID NO: 136). In some aspects, a polypeptide described herein (e.g., comprising the FAM19A5 binding domain of LRRC4B) consists of the amino acid sequence GYTYFTTVTVEDIETQPGEE (SEQ ID NO: 136). In some aspects, a polypeptide described herein (e.g., comprising the FAM19A5 binding domain of LRRC4B) consists essentially of the amino acid sequence GYTYFTTVTVEDIETQPGEE (SEQ ID NO: 136).

In some aspects, a polypeptide described herein (e.g., comprising the FAM19A5 binding domain of LRRC4B) comprises the amino acid sequence GYTYFTTVTVELIETQPGEE (SEQ ID NO: 137). In some aspects, a polypeptide described herein (e.g., comprising the FAM19A5 binding domain of LRRC4B) consists of the amino acid sequence GYTYFTTVTVELIETQPGEE (SEQ ID NO: 137). In some aspects, a polypeptide described herein (e.g., comprising the FAM19A5 binding domain of LRRC4B) consists essentially of the amino acid sequence GYTYFTTVTVELIETQPGEE (SEQ ID NO: 137).

In some aspects, a polypeptide described herein (e.g., comprising the FAM19A5 binding domain of LRRC4B) comprises the amino acid sequence GYTYFTTVTVEEIETQPGEE (SEQ ID NO: 138). In some aspects, a polypeptide described herein (e.g., comprising the FAM19A5 binding domain of LRRC4B) consists of the amino acid sequence GYTYFTTVTVEEIETQPGEE (SEQ ID NO: 138). In some aspects, a polypeptide described herein (e.g., comprising the FAM19A5 binding domain of LRRC4B) consists essentially of the amino acid sequence GYTYFTTVTVEEIETQPGEE (SEQ ID NO: 138).

In some aspects, a polypeptide described herein (e.g., comprising the FAM19A5 binding domain of LRRC4B) comprises the amino acid sequence GYTYFTTVTVEAFETQPGEE (SEQ ID NO: 139). In some aspects, a polypeptide described herein (e.g., comprising the FAM19A5 binding domain of LRRC4B) consists of the amino acid sequence GYTYFTTVTVEAFETQPGEE (SEQ ID NO: 139). In some aspects, a polypeptide described herein (e.g., comprising the FAM19A5 binding domain of LRRC4B) consists essentially of the amino acid sequence GYTYFTTVTVEAFETQPGEE (SEQ ID NO: 139).

In some aspects, a polypeptide described herein (e.g., comprising the FAM19A5 binding domain of LRRC4B) comprises the amino acid sequence GYTYFTTVTVEHHETQPGEE (SEQ ID NO: 140). In some aspects, a polypeptide described herein (e.g., comprising the FAM19A5 binding domain of LRRC4B) consists of the amino acid sequence GYTYFTTVTVEHHETQPGEE (SEQ ID NO: 140). In some aspects, a polypeptide described herein (e.g., comprising the FAM19A5 binding domain of LRRC4B) consists essentially of the amino acid sequence GYTYFTTVTVEHHETQPGEE (SEQ ID NO: 140).

In some aspects, a polypeptide described herein (e.g., comprising the FAM19A5 binding domain of LRRC4B) comprises the amino acid sequence GYTYFTTVTVEPFETQPGEE (SEQ ID NO: 141). In some aspects, a polypeptide described herein (e.g., comprising the FAM19A5 binding domain of LRRC4B) consists of the amino acid sequence GYTYFTTVTVEPFETQPGEE (SEQ ID NO: 141). In some aspects, a polypeptide described herein (e.g., comprising the FAM19A5 binding domain of LRRC4B) consists essentially of the amino acid sequence GYTYFTTVTVEPFETQPGEE (SEQ ID NO: 141).

In some aspects, a polypeptide described herein (e.g., comprising the FAM19A5 binding domain of LRRC4B) comprises the amino acid sequence GYTYFTTVTVEDWETQPGEE (SEQ ID NO: 142). In some aspects, a polypeptide described herein (e.g., comprising the FAM19A5 binding domain of LRRC4B) consists of the amino acid sequence GYTYFTTVTVEDWETQPGEE (SEQ ID NO: 142). In some aspects, a polypeptide described herein (e.g., comprising the FAM19A5 binding domain of LRRC4B) consists essentially of the amino acid sequence GYTYFTTVTVEDWETQPGEE (SEQ ID NO: 142).

In some aspects, a polypeptide described herein (e.g., comprising the FAM19A5 binding domain of LRRC4B) comprises the amino acid sequence GYTYFTTVTVEPYETQPGEEA (SEQ ID NO: 144). In some aspects, a polypeptide described herein (e.g., comprising the FAM19A5 binding domain of LRRC4B) consists of the amino acid sequence GYTYFTTVTVEPYETQPGEEA (SEQ ID NO: 144). In some aspects, a polypeptide described herein (e.g., comprising the FAM19A5 binding domain of LRRC4B) consists essentially of the amino acid sequence GYTYFTTVTVEPYETQPGEEA (SEQ ID NO: 144).

In some aspects, a polypeptide described herein (e.g., comprising the FAM19A5 binding domain of LRRC4B) comprises the amino acid sequence GYTYFTTVTVEHFETQPGEEA (SEQ ID NO: 145). In some aspects, a polypeptide described herein (e.g., comprising the FAM19A5 binding domain of LRRC4B) consists of the amino acid sequence GYTYFTTVTVEHFETQPGEEA (SEQ ID NO: 145). In some aspects, a polypeptide described herein (e.g., comprising the FAM19A5 binding domain of LRRC4B) consists essentially of the amino acid sequence GYTYFTTVTVEHFETQPGEEA (SEQ ID NO: 145).

In some aspects, a polypeptide described herein (e.g., comprising the FAM19A5 binding domain of LRRC4B) comprises the amino acid sequence GYTYFTTVTVEQRETQPGEEA (SEQ ID NO: 146). In some aspects, a polypeptide described herein (e.g., comprising the FAM19A5 binding domain of LRRC4B) consists of the amino acid sequence GYTYFTTVTVEQRETQPGEEA (SEQ ID NO: 146). In some aspects, a polypeptide described herein (e.g., comprising the FAM19A5 binding domain of LRRC4B) consists essentially of the amino acid sequence GYTYFTTVTVEQRETQPGEEA (SEQ ID NO: 146).

In some aspects, a polypeptide described herein (e.g., comprising the FAM19A5 binding domain the amino of LRRC4B) comprises acid sequence GYTYFTTVTVEWYETQPGEEA (SEQ ID NO: 147). In some aspects, a polypeptide described herein (e.g., comprising the FAM19A5 binding domain of LRRC4B) consists of the amino acid sequence GYTYFTTVTVEWYETQPGEEA (SEQ ID NO: 147). In some aspects, a polypeptide described herein (e.g., comprising the FAM19A5 binding domain of LRRC4B) consists essentially of the amino acid sequence GYTYFTTVTVEWYETQPGEEA (SEQ ID NO: 147).

In some aspects, a polypeptide described herein (e.g., comprising the FAM19A5 binding domain of LRRC4B) comprises the amino acid sequence GYTYFTTVTVEMRETQPGEEA (SEQ ID NO: 148). In some aspects, a polypeptide described herein (e.g., comprising the FAM19A5 binding domain of LRRC4B) consists of the amino acid sequence GYTYFTTVTVEMRETQPGEEA (SEQ ID NO: 148). In some aspects, a polypeptide described herein (e.g., comprising the FAM19A5 binding domain of LRRC4B) consists essentially of the amino acid sequence GYTYFTTVTVEMRETQPGEEA (SEQ ID NO: 148).

In some aspects, a polypeptide described herein (e.g., comprising the FAM19A5 binding domain of LRRC4B) comprises the amino acid sequence GYTYFTTVTVEIFETQPGEEA (SEQ ID NO: 149). In some aspects, a polypeptide described herein (e.g., comprising the FAM19A5 binding domain of LRRC4B) consists of the amino acid sequence GYTYFTTVTVEIFETQPGEEA (SEQ ID NO: 149). In some aspects, a polypeptide described herein (e.g., comprising the FAM19A5 binding domain of LRRC4B) consists essentially of the amino acid sequence GYTYFTTVTVEIFETQPGEEA (SEQ ID NO: 149).

In some aspects, a LRRC4 family mimic molecule described herein comprises one or more components that can improve the ability of the polypeptide to inhibit the interaction between a FAM19A5 protein and members of the LRRC4 protein family. For instance, in some aspects, a molecule comprises (i) any of the polypeptides described herein and (ii) one or more additional amino acids at the N-terminus of the polypeptide, the C-terminus of the polypeptide, or both the N-terminus and the C-terminus of the polypeptide. In some aspects, a molecule useful for the present disclosure comprises at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, or at least about 20 additional amino acids at the N-terminus of the polypeptide. In some aspects, a molecule comprises at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, or at least about additional 20 amino acids at the C-terminus of the polypeptide. In some aspects, a molecule comprises: (i) at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, or at least about 20 additional amino acids at the N-terminus of the polypeptide; and (ii) at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, or at least about 20 additional amino acids at the C-terminus of the polypeptide.

In some aspects, a LRRC4 family mimic molecule comprises: (i) a polypeptide having the amino acid sequence set forth in SEQ ID NO: 29 (YTYFTTVTVE) and (ii) at least 1 additional amino acid at the N-terminus of the polypeptide. In some aspects, a LRRC4 family mimic molecule comprises: (i) a polypeptide having the amino acid sequence set forth in SEQ ID NO: 29 (YTYFTTVTVE) and (ii) at least 1 additional amino acid at the C-terminus of the polypeptide. In some aspects, a LRRC4 family mimic molecule comprises: (i) a polypeptide having the amino acid sequence set forth in SEQ ID NO: 29 (YTYFTTVTVE) and (ii) at least one additional amino acid at both the N-terminus and the C-terminus. In some aspects, a LRRC4 family mimic molecule useful for the present disclosure comprises the amino acid sequence set forth in SEQ ID NO: 18 (GYTYFTTVTVETLETQPGEE). In some aspects, a LRRC4 family mimic molecule consists of the amino acid sequence set forth in SEQ ID NO: 18 (GYTYFTTVTVETLETQPGEE). In some aspects, a LRRC4 family mimic molecule consists essentially of the amino acid sequence set forth in SEQ ID NO: 18 (GYTYFTTVTVETLETQPGEE). In some aspects, a LRRC4 family mimic molecule useful for the present disclosure comprises the amino acid sequence set forth in SEQ ID NO: 17 (GYTYFTTVTVETLETQ). In some aspects, a LRRC4 family mimic molecule consists of the amino acid sequence set forth in SEQ ID NO: 17 (GYTYFTTVTVETLETQ). In some aspects, a LRRC4 family mimic molecule consists essentially of the amino acid sequence set forth in SEQ ID NO: 17 (GYTYFTTVTVETLETQ). In some aspects, a LRRC4 family mimic molecule useful for the present disclosure comprises the amino acid sequence set forth in SEQ ID NO: 19 (GYTYFTTVTVETLETQPGEKEPPGPTTD). In some aspects, a LRRC4 family mimic molecule consists of the amino acid sequence set forth in SEQ ID NO: 19 (GYTYFTTVTVETLETQPGEKEPPGPTTD). In some aspects, a LRRC4 family mimic molecule consists essentially of the amino acid sequence set forth in SEQ ID NO: 19 (GYTYFTTVTVETLETQPGEKEPPGPTTD).

In some aspects, a LRRC4 family mimic molecule useful for the present disclosure comprises: (i) a polypeptide having the amino acid sequence set forth in SEQ ID NO: 30 (YSFFTTVTVE) and (ii) at least 1 additional amino acid at the N-terminus of the polypeptide. In some aspects, a LRRC4 family mimic molecule comprises: (i) a polypeptide having the amino acid sequence set forth in SEQ ID NO: 30 (YSFFTTVTVE) and (ii) at least 1 additional amino acid at the C-terminus of the polypeptide. In some aspects, a LRRC4 family mimic molecule comprises: (i) a polypeptide having the amino acid sequence set forth in SEQ ID NO: 30 (YSFFTTVTVE) and (ii) at least one additional amino acid at both the N-terminus and C-terminus. In some aspects, a LRRC4 family mimic molecule useful for the present disclosure comprises the amino acid sequence set forth in SEQ ID NO: 20 (NYSFFTTVTVETTEISPEDTTRK). In some aspects, a LRRC4 family mimic molecule consists of the amino acid sequence set forth in SEQ ID NO: 20 (NYSFFTTVTVETTEISPEDTTRK). In some aspects, a LRRC4 family mimic molecule consists essentially of the amino acid sequence set forth in SEQ ID NO: 20 (NYSFFTTVTVETTEISPEDTTRK).

In some aspects, a LRRC4 family mimic molecule useful for the present disclosure comprises: (i) a polypeptide having the amino acid sequence set forth in SEQ ID NO: 31 (FSYFSTVTVE) and (ii) at least 1 additional amino acid at the N-terminus of the polypeptide. In some aspects, a LRRC4 family mimic molecule comprises: (i) a polypeptide having the amino acid sequence set forth in SEQ ID NO: 31 (FSYFSTVTVE) and (ii) at least 1 additional amino acid at the C-terminus of the polypeptide. In some aspects, a LRRC4 family mimic molecule comprises: (i) a polypeptide having the amino acid sequence set forth in SEQ ID NO: 31 (FSYFSTVTVE) and (ii) at least one additional amino acid at both the N-terminus and the C-terminus. In some aspects, a LRRC4 family mimic molecule useful for the present disclosure comprises the amino acid sequence set forth in SEQ ID NO: 21 (NFSYFSTVTVETMEPSQDERTTR). In some aspects, a LRRC4 family mimic molecule consists of the amino acid sequence set forth in SEQ ID NO: 21 (NFSYFSTVTVETMEPSQDERTTR). In some aspects, a LRRC4 family mimic molecule consists essentially of the amino acid sequence set forth in SEQ ID NO: 21 (NFSYFSTVTVETMEPSQDERTTR).

In some aspects, a polypeptide of a LRRC4 family mimic molecule comprises an amino acid sequence that is at least about 40%, at least about 50%, at least about 60%, at least about 70%, at least about 80%, or at least about 90% identical to the amino acid sequence set forth in SEQ ID NO: 29 (YTYFTTVTVE), wherein the polypeptide is capable of binding to a FAM19A5 protein and wherein the amino acid sequence further comprises one or more hydrophobic amino acids at the N-terminus. In some aspects, the hydrophobic amino acids comprise at least two amino acids, at least three amino acids, at least four amino acids, at least five amino acids, at least six amino acids, at least seven amino acids, at least eight amino acids, at least nine amino acids, at least 10 amino acids, at least 15 amino acids, at least 20 amino acids, at least 25 amino acids, at least 30 amino acids, at least 35 amino acids, at least 40 amino acids, at least 45 amino acids, or at least 50 amino acids at the N terminus.

In some aspects, a polypeptide of a LRRC4 family mimic molecule comprises an amino acid sequence that is at least about 40%, at least about 50%, at least about 60%, at least about 70%, at least about 80%, or at least about 90% identical to the amino acid sequence set forth in SEQ ID NO: 29 (YTYFTTVTVE), wherein the polypeptide is capable of binding to a FAM19A5 protein and wherein the amino acid sequence further comprises at the N-terminus and/or at the C-terminus one or more amino acids. In some aspects, the one or more amino acids linked to the N-terminus and/or C-terminus comprise one or more amino acid sequences derived from a LRRC4B protein. In some aspects, the one or more amino acids linked to the N-terminus comprises at least two amino acids, at least three amino acids, at least four amino acids, at least five amino acids, at least six amino acids, at least seven amino acids, at least eight amino acids, at least 10 amino acids, at least 15 amino acids, at least 20 amino acids, at least 25 amino acids, at least 30 amino acids, at least 35 amino acids, at least 40 amino acids, at least 45 amino acids, or at least 50 amino acids at the N-terminus. In some aspects, the one or more amino acids linked to the C-terminus comprise at least two amino acids, at least three amino acids, at least four amino acids, at least five amino acids, at least six amino acids, at least seven amino acids, at least eight amino acids, at least 10 amino acids, at least 15 amino acids, at least 20 amino acids, at least 25 amino acids, at least 30 amino acids, at least 35 amino acids, at least 40 amino acids, at least 45 amino acids, or at least 50 amino acids at the C-terminus. In some aspects, the one or more amino acids linked to the N-terminus and/or C-terminus are linked via a linker. In some aspects, the linker is a peptide linker.

In some aspects, the one or more additional amino acids that are added at the N-terminus and/or the C-terminus can comprise any suitable amino acids known in the art. In some aspects, the one or more additional amino acids are hydrophilic amino acids. In some aspects, the one or more additional amino acids can comprise D-amino acids. Not to be bound by any one theory, in some aspects, the addition of one or more D-amino acids at the N-terminus and/or the C-terminus of the polypeptide can enhance the persistence of the LRRC4 family mimic molecule, e.g., when administered to a subject. For instance, the inclusion of the D-amino acids can protect the polypeptide from protease and peptidase degradation within the blood of the subject. Accordingly, as demonstrated herein (see, e.g., Example 10), in some aspects, a polypeptide useful for the present disclosure can comprise both D-amino acids and L-amino acids. For instance, in some aspects, a polypeptide described herein comprises a D-amino acid at the N-terminus and L-amino acid at all other amino acid residues. In some aspects, a polypeptide described herein comprises a D-amino acid at the C-terminus and L-amino acid at all other amino acid residues. In some aspects, a polypeptide described herein comprises a D-amino acid at both the N-terminus and the C-terminus, and L-amino acid at all other amino acid residues.

As described herein, in some aspects, a LRRC4 family mimic molecule described above comprises a polypeptide having an amino acid sequence set forth in any one of SEQ ID NOs: 29 (YTYFTTVTVE), 30 (YSFFTTVTVE), and 31 (FSYFSTVTVE), with one, two, three, four, five, or six amino acids different from the amino acid sequence (e.g., substitutions).

In some aspects, a LRRC4 family mimic molecule useful for the present disclosure comprises additional modifications at the N-terminus, C-terminus, or both the N-terminus and the C-terminus of the polypeptide, wherein the additional modifications can increase the stability of the polypeptide. For instance, in some aspects, the N-terminal end of the polypeptide has been methylated. Non-limiting examples of additional modifications that can be performed at the N-terminus and/or C-terminus include: Fmoc, PEGylation, acetylation, or combinations thereof. In some aspects, to increase stability, the polypeptide can be cyclized. Any suitable methods known in the art can be used to make such modifications.

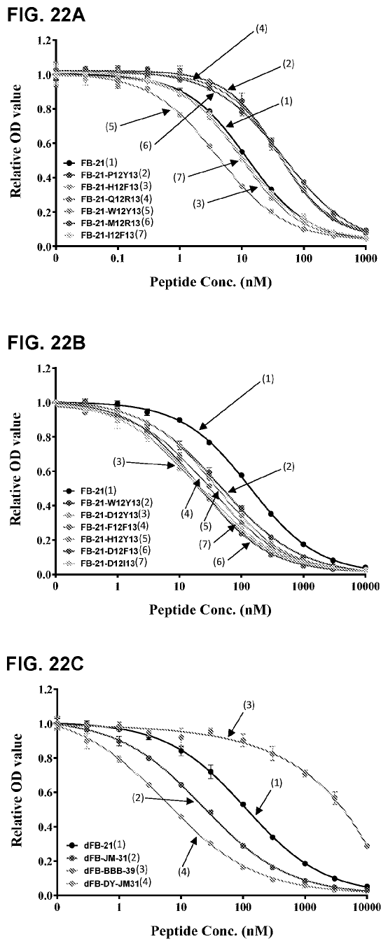

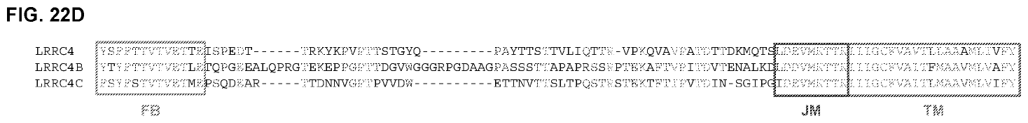

As further described elsewhere in the present disclosure, in some aspects, a molecule useful for the present disclosure comprises a FAM19A5 binding domain of members of the LRRC4 family protein and an additional moiety, which is capable of improving one or more properties of the molecules (e.g., binding affinity of the molecules to the FAM19A5 protein). As demonstrated herein (see, e.g., Example 10), Applicant has identified that the addition of the juxta-membrane sequence of members of the LRRC4 protein family can greatly improve the binding affinity of the molecules to FAM19A5 protein. The juxta-membrane sequence is highly conserved among members of the LRRC4 family and set forth in SEQ ID NO: 151 (LDEVMKTTK) (LRRC4 and LRRC4B) and SEQ ID NO: 152 (IDEVMKTTK) (LRRC4C) (see also FIG. 22D).

Accordingly, in some aspects, a molecule described herein comprises the FAM19A5 binding domain of the LRRC4 protein (i.e., YSFFTTVTVE; SEQ ID NO: 30) and the juxta-membrane sequence set forth in SEQ ID NO: 151 (LDEVMKTTK). In some aspects, a molecule described herein comprises the FAM19A5 binding domain of the LRRC4 protein (i.e., YSFFTTVTVE; SEQ ID NO: 30) and the juxta-membrane sequence set forth in SEQ ID NO: 152 (IDEVMKTTK). In some aspects, a molecule described herein comprises the FAM19A5 binding domain of the LRRC4B protein (i.e., YTYFTTVTVE; SEQ ID NO: 29) and the juxta-membrane sequence set forth in SEQ ID NO: 151 (LDEVMKTTK). In some aspects, a molecule described herein comprises the FAM19A5 binding domain of the LRRC4 protein (i.e., YTYFTTVTVE; SEQ ID NO: 29) and the juxta-membrane sequence set forth in SEQ ID NO: 152 (IDEVMKTTK). In some aspects, a molecule described herein comprises the FAM19A5 binding domain of the LRRC4B protein (i.e., FSYFSTVTVE; SEQ ID NO: 31) and the juxta-membrane sequence set forth in SEQ ID NO: 151 (LDEVMKTTK). In some aspects, a molecule described herein comprises the FAM19A5 binding domain of the LRRC4 protein (i.e., FSYFSTVTVE; SEQ ID NO: 31) and the juxta-membrane sequence set forth in SEQ ID NO: 152 (IDEVMKTTK). In some aspects, the juxta-membrane is added to the C-terminus of the molecule.

As is apparent from the present disclosure, any of the modifications described herein to improve one or more properties of a molecule (e.g., amino acid substitutions, addition of a juxta-membrane sequence, D-amino acids) can be used in combination. For instance, in some aspects, a molecule useful for the present disclosure (e.g., polypeptide) comprises: (i) the amino acid sequence set forth in SEQ ID NO: 18 (GYTYFTTVTVETLETQPGEE) with amino acid modifications at residues T12 and L13; and (ii) a juxta-membrane sequence (e.g., SEQ ID NO: 151 or SEQ ID NO: 152) at the C-terminus of the molecule. In some aspects, a molecule useful for the present disclosure (e.g., polypeptide) comprises: (i) the amino acid sequence set forth in SEQ ID NO: 18 (GYTYFTTVTVETLETQPGEE) with amino acid modifications at residues T12 and L13; (ii) D-amino acids at the N-terminus and/or C-terminus; and (iii) a juxta-membrane sequence (e.g., SEQ ID NO: 151 or SEQ ID NO: 152) at the C-terminus of the molecule. In some aspects, a molecule useful for the present disclosure (e.g., polypeptide) comprises: (i) the amino acid sequence set forth in SEQ ID NO: 17 (GYTYFTTVTVETLETQ) with amino acid modifications at residues T12 and L13; and (ii) a juxta-membrane sequence (e.g., SEQ ID NO: 151 or SEQ ID NO: 152) at the C-terminus of the molecule. In some aspects, a molecule useful for the present disclosure (e.g., polypeptide) comprises: (i) the amino acid sequence set forth in SEQ ID NO: 17 (GYTYFTTVTVETLETQ) with amino acid modifications at residues T12 and L13; (ii) D-amino acids at the N-terminus and/or C-terminus; and (iii) a juxta-membrane sequence (e.g., SEQ ID NO: 151 or SEQ ID NO: 152) at the C-terminus of the molecule. In some aspects, a molecule useful for the present disclosure (e.g., polypeptide) comprises: (i) the amino acid sequence set forth in SEQ ID NO: 19 (GYTYFTTVTVETLETQPGEKEPPGPTTD) with amino acid modifications at residues T12 and L13; and (ii) a juxta-membrane sequence (e.g., SEQ ID NO: 151 or SEQ ID NO: 152) at the C-terminus of the molecule. In some aspects, a molecule useful for the present disclosure (e.g., polypeptide) comprises: (i) the amino acid sequence set forth in SEQ ID NO: 19 (GYTYFTTVTVETLETQPGEKEPPGPTTD) with amino acid modifications at residues T12 and L13; (ii) D-amino acids at the N-terminus and/or C-terminus; and (iii) a juxta-membrane sequence (e.g., SEQ ID NO: 151 or SEQ ID NO: 152) at the C-terminus of the molecule. In some aspects, a molecule useful for the present disclosure (e.g., polypeptide) comprises: (i) the amino acid sequence set forth in SEQ ID NO: 143 (GYTYFTTVTVETLETQPGEEA) with amino acid modifications at residues T12 and L13; and (ii) a juxta-membrane sequence (e.g., SEQ ID NO: 151 or SEQ ID NO: 152) at the C-terminus of the molecule. In some aspects, a molecule useful for the present disclosure (e.g., polypeptide) comprises: (i) the amino acid sequence set forth in SEQ ID NO: 143 (GYTYFTTVTVETLETQPGEEA) with amino acid modifications at residues T12 and L13; (ii) D-amino acids at the N-terminus and/or C-terminus; and (iii) a juxta-membrane sequence (e.g., SEQ ID NO: 151 or SEQ ID NO: 152) at the C-terminus of the molecule.

In some aspects, a LRRC4 family mimic molecule described herein can comprise one or more additional peptides that allow the molecule to be specifically targeted to different tissues, e.g., when administered to a subject. For instance, in some aspects, a LRRC4 family mimic molecule comprises a peptide that allows the molecule to penetrate across the blood-brain barrier (also referred to herein as “BBB shuttles”). Examples of such BBB shuttles are known in the art. Non-limiting examples are provided in Table 5 (below). See, e.g., Oller-Salvia et al., Chem Soc Rev 45:4690 (2016).

TABLE 5
BBB Shuttles

| SEQ ID | | |
|---|---|---|
| NO | Peptide | Sequence |
| 32 | Angiopep-2 | TFFYGGSRGKRNNFKTEEY-OH |
| 33 | ApoB (3371-3409) | SSVIDALQYKLEGTTRLTRKR |
| | | GLKLATALSLSNKFVEGS |
| 34 | ApoE (159-167)₂ | (LRKLRKRLL)₂ |
| 35 | Peptide-22 | Ac-C(&)MPRLRGC(&)-NH₂ |
| 36 | THR | THRPPMWSPVWP-NH₂ |
| 37 | THR retro-enantio | pwvpswmpprht-NH₂ |
| 38 | CRT | C(&)RTIGPSVC(&) |
| 39 | Leptin30 | YQQILTSMPSRNVIQIS |
| | | NDLENLRDLLHVL |
| 40 | RVG29 | YTIWMPENPRPGTPCDI |
| | | FTNSRGKRASNG-OH |
| 41 | CDX | GreirtGraerwsekf-OH |
| 42 | Apamin | C(&₁)NC(&₂)KAPETALC(&₁)- |
| | | ARRC(&₂)QQH-NH₂ |
| 43 | MiniAp-4 | [Dap](&)KAPETALD(&) |
| 44 | GSH | γ-L-glutamyl-CG-OH |
| 45 | G23 | HLNILSTLWKYRC |
| 46 | g7 | GFtGFLS(O-b-Glc)-NH₂ |
| 47 | TGN | TGNYKALHPHNG |
| 48 | TAT (47-57) | YGRKKRRQRRR-NH2 |
| 49 | SynB1 | RGGRLSYSRRRFSTSTGR |
| 50 | Diketopiperazines | &(N-MePhe)-(N-MePhe) |
| | | Diketopiperazines |
| 51 | PhPro | (Phenylproline)4-NH₂ |

Nomenclature for cyclic peptides (&) is adapted to the 3-letter amino acid code from the one described in Spengler et al., J Pept Res 65: 550-555 (2005); [Dap] stands for diaminopropionic acid.

In some aspects, a LRRC4 family mimic molecule useful for the present disclosure comprises a fusion protein. For instance, in some aspects, a LRRC4 family mimic molecule described herein can comprise: (i) any of the polypeptides of the present disclosure, and (ii) a half-life extending moiety. Any suitable half-life extending moieties known in the art can be used to generate the fusion proteins of the present disclosure. Non-limiting examples of such half-life extending moieties include: a Fc, albumin, an albumin-binding polypeptide, Pro/Ala/Ser (PAS), a C-terminal peptide (CTP) of the ß subunit of human chorionic gonadotropin, polyethylene glycol (PEG), long unstructured hydrophilic sequences of amino acids (XTEN), hydroxyethyl starch (HES), an albumin-binding small molecule, or a combination thereof.

In some aspects, a LRRC4 family mimic molecule comprises a protein-drug conjugate. For instance, in some aspects, the polypeptide of the LRRC4 family mimic can be conjugated to a therapeutic agent, such as those that are useful for treating a disease or disorder.

The protein-drug conjugates described herein can be prepared by methods known in the art. In some aspects, conjugation methods result in linkages which are substantially (or nearly) non-immunogenic, e.g., peptide- (i.e., amide-), sulfide-, (sterically hindered), disulfide-, hydrazone-, and ether linkages. These linkages are nearly non-immunogenic and show reasonable stability within serum (see, e.g., Senter, P. D., Curr. Opin. Chem. Biol. 13 (2009) 235-244; WO 2009/059278; WO 95/17886, each of which is incorporated herein by reference in its entirety).

Depending on the biochemical nature of the moiety and the polypeptides, different conjugation strategies can be employed (see, e.g., Hackenberger, C. P. R., and Schwarzer, D., Angew. Chem. Int. Ed. Engl. 47 (2008) 10030-10074). In some aspects, site specific reaction and covalent coupling is based on transforming a natural amino acid into an amino acid with a reactivity which is orthogonal to the reactivity of the other functional groups present. For example, a specific cysteine within a rare sequence context can be enzymatically converted in an aldehyde (see Frese, M. A., and Dierks, T., ChemBioChem. 10 (2009) 425-427). It is also possible to obtain a desired amino acid modification by utilizing the specific enzymatic reactivity of certain enzymes with a natural amino acid in a given sequence context (see, e.g., Taki, M. et al., Prot. Eng. Des. Sel. 17 (2004) 119-126; Gautier, A. et al., Chem. Biol. 15 (2008) 128-136; and Protease-catalyzed formation of C—N bonds is used by Bordusa, F., Highlights in Bioorganic Chemistry (2004) 389-403).

Site specific reaction and covalent coupling can also be achieved by the selective reaction of terminal amino acids with appropriate modifying reagents. The reactivity of an N-terminal cysteine with benzonitrils (see Ren, H. et al., Angew. Chem. Int. Ed. Engl. 48 (2009) 9658-9662) can be used to achieve a site-specific covalent coupling. Native chemical ligation can also rely on C-terminal cysteine residues (Taylor, E. Vogel; Imperiali, B, Nucleic Acids and Molecular Biology (2009), 22 (Protein Engineering), 65-96).

The moiety can also be a synthetic peptide or peptide mimic. In such cases, a polypeptide can be chemically synthesized, amino acids with orthogonal chemical reactivity can be incorporated during such synthesis (see, e.g., de Graaf, A. J. et al., Bioconjug. Chem. 20 (2009) 1281-1295). To obtain a mono-labeled polypeptide, the conjugate with 1:1 stoichiometry can be separated by chromatography from other conjugation side-products. This procedure can be facilitated using a dye labeled binding pair member and a charged linker. With this kind of labeled and highly negatively charged binding pair member, mono conjugated polypeptides are easily separated from non-labeled polypeptides and polypeptides which carry more than one linker, since the difference in charge and molecular weight can be used for separation. The fluorescent dye can be useful for purifying the complex from un-bound components, like a labeled monovalent binder.

### IV. Pharmaceutical Compositions

Provided herein are compositions comprising a LRRC4 family mimic molecule, nucleic acids, vectors, cells, or protein conjugates described herein having the desired degree of purity in a physiologically acceptable carrier, excipient or stabilizer (Remington's Pharmaceutical Sciences (1990) Mack Publishing Co., Easton, PA). Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS® or polyethylene glycol (PEG).

In some aspects, a pharmaceutical composition useful for the present disclosure comprises any of the LRRC4 family mimic molecules, nucleic acids, vectors, cells, protein conjugates described herein, and optionally one or more additional prophylactic or therapeutic agents, in a pharmaceutically acceptable carrier. In some aspects, pharmaceutical compositions comprise any of the LRRC4 family mimic molecules, nucleic acids, vectors, cells, protein conjugates described herein, and optionally one or more additional prophylactic of therapeutic agents, in a pharmaceutically acceptable carrier. In some aspects, the LRRC4 family mimic molecules, nucleic acids, vectors, cells, protein conjugates described herein are the only active ingredient included in the pharmaceutical composition. Pharmaceutical compositions described herein can be useful in inhibiting, reducing, and/or dissociating the interaction of a FAM19A5 protein and members of the LRRC4 protein family. As described elsewhere in the present disclosure, inhibiting, reducing, and/or dissociating the interaction of a FAM19A5 protein and members of the LRRC4 protein family can improve neural circuit formation (e.g., by promoting neurite outgrowth and synaptic formation).

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcellulose, hydroxypropyl methylcellulose and polyvinylpyrrolidone. Emulsifying agents include Polysorbate 80 (TWEEN® 80). A sequestering or chelating agent of metal ions includes EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

A pharmaceutical composition can be formulated for any route of administration to a subject. Specific examples of routes of administration include intranasal, oral, parenterally, intrathecally, intra-cerebroventricularly, pulmonarily, subcutaneously, or intraventricularly. Parenteral administration, characterized by either subcutaneous, intramuscular or intravenous injection, is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Preparations for parenteral administration of a LRRC4 family mimic molecule described herein include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions can be aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Topical mixtures comprising a LRRC4 family mimic molecule are prepared as described for the local and systemic administration. The resulting mixture can be a solution, suspension, emulsions or the like and can be formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

A pharmaceutical composition (e.g., comprising any of the LRRC4 family mimic molecules, nucleic acids, vectors, cells, or protein conjugates described herein) can be formulated as an aerosol for topical application, such as by inhalation (see, e.g., U.S. Pat. Nos. 4,044,126, 4,414,209 and 4,364,923). These formulations for administration to the respiratory tract can be in the form of an aerosol or solution for a nebulizer, or as a microfine powder for insufflations, alone or in combination with an inert carrier such as lactose. In such a case, the particles of the formulation can, in some aspects, have diameters of less than about 50 microns, e.g., less than about 10 microns.

A pharmaceutical composition (e.g., comprising any of the LRRC4 family mimic molecules, nucleic acids, vectors, cells, or protein conjugates described herein) can be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the antibody alone or in combination with other pharmaceutically acceptable excipients can also be administered.

Transdermal patches, including iontophoretic and electrophoretic devices, are well known to those of skill in the art, and can be used to administer any of the LRRC4 family mimic molecules, nucleic acids, vectors, cells, or protein-conjugates described herein). For example, such patches are disclosed in U.S. Pat. Nos. 6,267,983, 6,261,595, 6,256,533, 6,167,301, 6,024,975, 6,010,715, 5,985,317, 5,983,134, 5,948,433, and 5,860,957.

In some aspects, a pharmaceutical composition described herein is a lyophilized powder, which can be reconstituted for administration as solutions, emulsions and other mixtures. It can also be reconstituted and formulated as solids or gels. The lyophilized powder is prepared by dissolving any of the LRRC4 family mimic molecules, nucleic acids, vectors, cells, or protein-conjugates described herein described herein, or a pharmaceutically acceptable derivative thereof, in a suitable solvent. In some aspects, the lyophilized powder is sterile. The solvent can contain an excipient which improves the stability or other pharmacological component of the powder or reconstituted solution, prepared from the powder. Excipients that can be used include, but are not limited to, dextrose, sorbitol, fructose, corn syrup, xylitol, glycerin, glucose, sucrose or other suitable agent. The solvent can also contain a buffer, such as citrate, sodium, or potassium phosphate or other such buffer known to those of skill in the art. In some aspects, the buffer is at about neutral pH. Subsequent sterile filtration of the solution followed by lyophilization under standard conditions known to those of skill in the art provides the desired formulation. In some aspects, the resulting solution can be apportioned into vials for lyophilization. Each vial can contain a single dosage or multiple dosages of the compound (e.g., any of the LRRC4 family mimic molecules, nucleic acids, vectors, cells, or protein-conjugates). The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

In some aspects, a pharmaceutical composition comprising any of the LRRC4 family mimic molecules, nucleic acids, vectors, cells, or protein-conjugates described herein can also be formulated to be targeted to a particular tissue, receptor, or other area of the body of the subject to be treated. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542, and 5,709,874.

The compositions to be used for in vivo administration can be sterile. In some aspects, this can be accomplished by filtration through, e.g., sterile filtration membranes.

### V. Nucleic Acids, Vectors, Host Cells

Further aspect described herein pertains to one or more nucleic acid molecules (also referred to herein as “nucleic acids” or derivatives thereof) that encode a molecule (e.g., a LRRC4 family mimic molecule described herein). The nucleic acids can be present in whole cells, in a cell lysate, or in a partially purified or substantially pure form. In some aspects, the nucleic acid is a DNA sequence and/or an RNA sequence (e.g., mRNA). In some aspects, the nucleic acids comprise a modified nucleotide analog. A nucleic acid is “isolated” or “rendered substantially pure” when purified away from other cellular components or other contaminants, e.g., other cellular nucleic acids (e.g., other chromosomal DNA, e.g., the chromosomal DNA that is linked to the isolated DNA in nature) or proteins, by standard techniques, including alkaline/SDS treatment, CsCl banding, column chromatography, restriction enzymes, agarose gel electrophoresis and others well known in the art. See, F. Ausubel, et al., ed. (1987) Current Protocols in Molecular Biology, Greene Publishing and Wiley Interscience, New York. In some aspects, a nucleic acid molecule can or cannot contain intronic sequences. In some aspects, the nucleic acid is a cDNA molecule. Nucleic acids described herein can be obtained using standard molecular biology techniques known in the art.

In some aspects, the present disclosure provides a vector comprising an isolated nucleic acid molecule encoding a therapeutic agent disclosed herein (e.g., a LRRC4 family mimic molecule described herein). Suitable vectors for the disclosure include, but are not limited to, expression vectors, viral vectors, and plasmid vectors. In some aspects, the vector is a viral vector.

As used herein, an “expression vector” refers to any nucleic acid construct which contains the necessary elements for the transcription and translation of an inserted coding sequence, or in the case of a RNA viral vector, the necessary elements for replication and translation, when introduced into an appropriate host cell. Expression vectors can include plasmids, phagemids, viruses, and derivatives thereof.

As used herein, “viral vectors” include, but are not limited to, nucleic acid sequences from the following viruses: retrovirus, such as Moloney murine leukemia virus, Harvey murine sarcoma virus, murine mammary tumor virus, and Rous sarcoma virus; lentivirus; adenovirus; adeno-associated virus; SV40-type viruses; polyomaviruses; Epstein-Barr viruses; papilloma viruses; herpes virus; vaccinia virus; polio virus; and RNA virus such as a retrovirus. Certain viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA.

In some aspects, a vector is derived from an adeno-associated virus. In some aspects, a vector is derived from a lentivirus. Examples of the lentiviral vectors are disclosed in WO9931251, W09712622, W09817815, W09817816, and WO9818934, each which is incorporated herein by reference in its entirety.

Other vectors include plasmid vectors. See, e.g., Sambrook et al., Molecular Cloning: A Laboratory Manual, Second Edition, Cold Spring Harbor Laboratory Press, 1989. In the last few years, plasmid vectors have been found to be particularly advantageous for delivering genes to cells in vivo because of their inability to replicate within and integrate into a host genome. These plasmids, however, having a promoter compatible with the host cell, can express a peptide from a gene operably encoded within the plasmid. Some commonly used plasmids available from commercial suppliers include pBR322, pUC18, pUC19, various pcDNA plasmids, pRC/CMV, various pCMV plasmids, pSV40, and pBlueScript. Additional examples of specific plasmids include pcDNA3.1, catalog number V79020; pcDNA3.1/hygro, catalog number V87020; pcDNA4/myc-His, catalog number V86320; and pBudCE4.1, catalog number V53220, all from Invitrogen (Carlsbad, CA.). Additionally, plasmids can be custom designed using standard molecular biology techniques to remove and/or add specific fragments of DNA.

Also encompassed herein is a method for making a molecule disclosed herein (e.g., a LRRC4 family mimic molecule described herein). In some aspects, such a method can comprise expressing the molecule (e.g., a LRRC4 family mimic molecule described herein) in a cell comprising a nucleic acid molecule encoding the molecule. Host cells comprising these nucleotide sequences are encompassed herein. Non-limiting examples of host cell that can be used include immortal hybridoma cell, NS/0 myeloma cell, 293 cell, Chinese hamster ovary (CHO) cell, HeLa cell, human amniotic fluid-derived cell (CapT cell), COS cell, or combinations thereof.

### VI. Kits

Also provided herein are kits comprising one or more of the LRRC4 family mimic molecules, nucleic acids, vectors, cells, protein conjugates, or compositions described herein. In some aspects, provided herein is a pharmaceutical pack or kit comprising one or more containers filled with one or more of the ingredients of the pharmaceutical compositions described herein, such as one or more LRRC4 family mimic molecule provided herein, optional an instruction for use. In some aspects, the kits contain a pharmaceutical composition described herein and any prophylactic or therapeutic agent, such as those described herein.

### VII. Methods of the Disclosure

As demonstrated herein, the LRRC4 family mimic molecules, nucleic acids, vectors, cells, protein conjugates, or compositions are useful in inhibiting, reducing, and/or dissociating the interaction between a FAM19A5 protein and LRRC4 protein family member. Accordingly, in some aspects, provided herein is a method of inhibiting, reducing, and/or dissociating a formation of a complex between a FAM19A5 protein and LRRC4 protein family member in a subject in need thereof, comprising administering to the subject an effective amount of any of the LRRC4 family mimic molecules, nucleic acids, vectors, cells, protein conjugates, or compositions described herein. In some aspects, after the administration, the formation of the complex is decreased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%, compared to a reference (e.g., corresponding value in the subject prior to the administration or the value in a corresponding subject who did not receive the administration).

As described elsewhere in the present disclosure, the binding of a FAM19A5 protein to members of the LRRC4 protein family can inhibit the activity of the LRRC4 protein family members. For instance, in some aspects, the formation of the FAM19A5-LRRC4 family protein complexes can lead to impaired neural circuit formation, resulting in an imbalance in the dynamic gain and loss of synapses, which is fundamental to the healthy function of neurons in the central and peripheral nervous systems.

Accordingly, in some aspects, the decrease in the formation of the complex between a FAM19A5 protein and members of the LRRC4 protein family can increase the activity of the members of the LRRC4 protein family. In some aspects, after the administration, the activity of the members of the LRRC4 protein family is increased by at least about 0.5-fold, at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 35-fold, at least about 40-fold, at least about 45-fold, or at least about 50-fold, compared to a reference (e.g., corresponding value in the subject prior to the administration or value in a corresponding subject that did not receive the administration). Non-limiting examples of such activity can include neurite outgrowth, neuronal migration, and the formation and functional assembly of synaptic contacts.

As is apparent from the present disclosure, in some aspects, the present disclosure is directed to a method of increasing a neurite outgrowth and/or synapse formation in neurons, comprising contacting the neurons with any of the LRRC4 family mimic molecules, nucleic acids, vectors, cells, protein conjugates, or compositions described herein. In some aspects, the contacting occurs in vivo (e.g., in a subject in need thereof). In such aspects, the method can further comprise administering any of the LRRC4 family mimic molecules, nucleic acids, vectors, cells, protein conjugates, or compositions to the subject prior to the contacting. In some aspects, the contacting occurs ex vivo. In some aspects, the contacting increases neurite outgrowth in the neurons by at least about 0.5-fold, at least about 1-fold, at least about 2-fold, at least about 3-fold, at least about 4-fold, at least about 5-fold, at least about 10-fold, at least about 20-fold, at least about 30-fold, at least about 40-fold, or at least about 50-fold, compared to a reference (e.g., neurite outgrowth in a corresponding neuron that was not contacted with any of the polypeptides, molecules, nucleic acids, vectors, cells, protein conjugates, or compositions described herein). In some aspects, the contacting increases synapse formation in the neuron by at least about 0.5-fold, at least about 1-fold, at least about 2-fold, at least about 3-fold, at least about 4-fold, at least about 5-fold, at least about 10-fold, at least about 20-fold, at least about 30-fold, at least about 40-fold, or at least about 50-fold, compared to a reference (e.g., synapse formation in a corresponding neuron that was not contacted with any of the LRRC4 family mimic molecules, nucleic acids, vectors, cells, protein conjugates, or compositions described herein).

In some aspects, the therapeutic effects of the LRRC4 family mimic molecules, nucleic acids, vectors, cells, protein conjugates, or combinations (e.g., decreased formation of the complex between a FAM19A5 protein and members of the LRRC4 protein family; increased neurite outgrowth; and/or increased synapse formation) can reduce one or more symptoms of a disease or condition, such as those associated with impaired neural circuit formation.

Accordingly, in some aspects, the present disclosure is directed to a method of treating a disease or condition in a subject in need thereof, comprising administering to the subject a therapeutically effective amount of any of the LRRC4 family mimic molecules, nucleic acids, vectors, cells, protein conjugates, or compositions described herein, wherein the disease or condition is selected from an amyotrophic lateral sclerosis (ALS), Alzheimer's disease, glaucoma, diabetic retinopathy, neuropathic pain, spinal cord injury, traumatic brain injury, stroke, Parkinson's disease, or combinations thereof. As described further below, in some aspects, provided herein is a method of treating an amyotrophic lateral sclerosis (ALS), comprising administering to the subject any of the LRRC4 family mimic molecules, nucleic acids, vectors, cells, protein conjugates, or compositions described herein. In some aspects, present disclosure provides a method of treating an Alzheimer's disease, comprising administering to the subject any of the LRRC4 family mimic molecules, nucleic acids, vectors, cells, protein conjugates, or compositions described herein. In some aspects, present disclosure provides a method of treating a glaucoma, comprising administering to the subject any of the LRRC4 family mimic molecules, nucleic acids, vectors, cells, protein conjugates, or compositions described herein. In some aspects, present disclosure provides a method of treating a diabetic retinopathy, comprising administering to the subject any of the LRRC4 family mimic molecules, nucleic acids, vectors, cells, protein conjugates, or compositions described herein. In some aspects, present disclosure provides a method of treating a neuropathic pain, comprising administering to the subject any of the LRRC4 family mimic molecules, nucleic acids, vectors, cells, protein conjugates, or compositions described herein. In some aspects, present disclosure provides a method of treating a spinal cord injury, comprising administering to the subject any of the LRRC4 family mimic molecules, nucleic acids, vectors, cells, protein conjugates, or compositions described herein. In some aspects, present disclosure provides a method of treating a traumatic brain injury, comprising administering to the subject any of the LRRC4 family mimic molecules, nucleic acids, vectors, cells, protein conjugates, or compositions described herein. In some aspects, present disclosure provides a method of treating a stroke, comprising administering to the subject any of the LRRC4 family mimic molecules, nucleic acids, vectors, cells, protein conjugates, or compositions described herein. In some aspects, present disclosure provides a method of treating a Parkinson's disease, comprising administering to the subject any of the LRRC4 family mimic molecules, nucleic acids, vectors, cells, protein conjugates, or compositions described herein.

Accordingly, some aspects of the present disclosure is directed to a method of treating ALS in a subject in need thereof, comprising administering to the subject any of the LRRC4 family mimic molecules, nucleic acids, vectors, cells, protein conjugates, or compositions described herein. In some aspects, ALS that can be treated with present disclosure comprises a sporadic ALS, familial ALS, or both. As used herein, the term “sporadic” ALS refers to ALS that is not associated with any family history of ALS occurrence. Approximately about 90% or more of the ALS diagnosis are for sporadic ALS. As used herein, the term “familial” ALS refers to ALS that occurs more than once within a family, suggesting a genetic component to the disease. In some aspects, ALS that can be treated with the present disclosure comprises primary lateral sclerosis (PLS). PLS can affect upper motor neurons in the arms and legs. More than 75% of people with apparent PLS, however, develop lower motor neuron signs within four years of symptom onset, meaning that a definite diagnosis of PLS cannot be made until then. PLS has a better prognosis than classic ALS, as it progresses slower, results in less functional decline, does not affect the ability to breathe, and causes less severe weight loss. In some aspects, ALS comprises progressive muscular atrophy (PMA). PMA can affect lower motor neurons in the arms and legs. While PMA is associated with. longer survival on average than classic ALS, it still progresses to other spinal cord regions over time, eventually leading to respiratory failure and death. Upper motor neuron signs can develop late in the course of PMA, in which case the diagnosis might be changed to classic ALS.

In some aspects, administering any of the therapeutic agents described herein (e.g., any of the LRRC4 family mimic molecules, nucleic acids, vectors, cells, protein conjugates, or compositions described herein) can improve one or more symptoms associated with ALS. Non-limiting examples of symptoms include: difficulty walking or doing normal daily activities; tripping and falling; weakness of the limbs; slurred speech; trouble swallowing; muscle cramps and twitching; inappropriate crying, laughing, or yawning; dementia; cognitive and behavioral changes; and combinations thereof.

As demonstrated herein (see, e.g., Example 11), in some aspects, the present disclosure provides a method of treating an Alzheimer's disease in a subject in need thereof, comprising administering any of the LRRC4 family mimic molecules, nucleic acids, vectors, cells, protein conjugates, or compositions described herein. Not to be bound by any one theory, in some aspects, treating an Alzheimer's disease comprises reducing an amyloid beta (Aβ) plaque load in the subject (e.g., suffering from Alzheimer's disease). As used herein, “amyloid beta plaque” refers to all forms of aberrant deposition of amyloid beta including large aggregates and small associations of a few amyloid beta peptides and can contain any variation of the amyloid beta peptides. Amyloid beta (Aβ) plaque is known to cause neuronal changes, e.g., aberrations in synapse composition, synapse shape, synapse density, loss of synaptic conductivity, changes in dendrite diameter, changes in dendrite length, changes in spine density, changes in spine area, changes in spine length, or changes in spine head diameter. In some aspects, an increase in Aβ plaque load can result in synapse loss in neurons. Accordingly, in some aspects, provided herein is a method of reducing synapse loss in neurons, comprising contacting a neuron with any of the LRRC4 family mimic molecules, nucleic acids, vectors, cells, protein conjugates, or compositions described herein. In some aspects, the contacting can occur in vivo. In some aspects, the contacting can occur ex vivo.

As also demonstrated herein (see, e.g., Example 13), in some aspects, the present disclosure provides a method of treating Parkinson's disease in a subject in need thereof, comprising administering any of the LRRC4 family mimic molecules, nucleic acids, vectors, cells, protein conjugates, or compositions described herein. As used herein, the term “Parkinson's disease” (PD) refers to neurodegenerative disorder leading to motor and non-motor manifestations (i.e., symptoms) and characterized by extensive degeneration of dopaminergic neurons in the nigrostriatal system. Non-limiting examples of motor and non-motor manifestations of PD are provided elsewhere in the present disclosure. Proteinopathy (α-synuclein abnormal aggregation) is a hallmark of PD. Other exemplary features of PD include dopaminergic neuron damage, mitochondrial dysfunction, neuroinflammation, protein homeostasis (e.g., autophagic clearance of damaged proteins and organelles glial cell dysfunction), and combinations thereof.

As demonstrated herein (see, e.g., Example 14), in some aspects, the therapeutic agents provided herein (e.g., any of the LRRC4 family mimic molecules, nucleic acids, vectors, cells, protein conjugates, or compositions described herein) can be useful in increasing a threshold or latency to an external stimulus (e.g., mechanical and/or thermal stimulus) in a subject in need thereof. Accordingly, in some aspects, after the administration, the subject has a higher threshold to an external stimuli compared to a reference control (e.g., corresponding subject who did not receive a LRRC4 family mimic molecule described herein). As used herein, the term “threshold to an external stimuli” refers to the amount of pressure (from the external stimuli) before a subject responds to the stimuli (e.g., by pulling away).

As will be apparent to those skilled in the arts, such a therapeutic effect can be useful in treating one or more symptoms associated with a neuropathic pain. Accordingly, in some aspects, provided herein is a method of treating, preventing, or ameliorating a neuropathic pain in a subject in need thereof, comprising administering to the subject any of the LRRC4 family mimic molecules, nucleic acids, vectors, cells, protein conjugates, or compositions described herein.

In some aspects, a neuropathic pain is a central neuropathic pain, i.e., a pain due to injury or damage affecting any level of the CNS (e.g., brain injury and spinal cord injury), including the central somatosensory nervous system, or associated with or as a result of a disease or disorder such as stoke, multiple sclerosis, or lateral medullary infarction. In some aspects, a neuropathic pain is a peripheral neuropathic pain, a pain due to injury or damage affecting any level of the peripheral nerves system (e.g., injury of a motor nerve, a sensory nerve, an autonomic nerve, or a combination thereof), or resulting from or associated with a disease or disorder.

In some aspects, the therapeutic agents provided herein (e.g., any of the LRRC4 family mimic molecules, nucleic acids, vectors, cells, protein conjugates, or compositions described herein) can be useful in treating retinopathies. In some aspects, a retinopathy that can be treated with the present disclosure comprises a diabetic retinopathy. The term “diabetic retinopathy” comprises all types of diabetic retinopathy including, but not limited to, non-proliferative diabetic retinopathy (NPDR), proliferative diabetic retinopathy (PDR), diabetic maculopathy, and diabetic macular edema. Not to be bound by any one theory, in some aspects, treating a retinopathy (e.g., diabetic retinopathy) comprises improving a retinal potential in a subject in need thereof. In some aspects, an improved retinal potential comprises an increase in the values for A-wave, B-wave, and/or oscillatory potential, compared to a reference (e.g., corresponding subject that was not treated with a LRRC4 family mimic molecule described herein). It will be apparent to those skilled in the arts that treating retinopathies can be useful in treating other types of eye disorders, including but not limited to, macular degeneration and glaucoma.

In some aspects, the methods described herein (e.g., increasing neurite outgrowth, increasing synapse formation, and/or decreasing formation of a complex between FAM19A5 and members of the LRRC4 protein family) can comprise administering an additional therapeutic agent to the subject. The additional therapeutic agent can include any known agents to treat and/or alleviate one or more symptoms associated with any of the above-described indications. In some aspects, the additional therapeutic agent comprises an acetylcholinesterase inhibitor. In some aspects, the additional therapeutic agent comprises a dopamine agonist. In some aspects, the additional therapeutic agent comprises a dopamine receptor antagonist. In some aspects, the additional therapeutic agent comprises an antipsychotic. In some aspects, the additional therapeutic agent comprises a monoamine oxidase (MAO) inhibitor. In some aspects, the additional therapeutic agent comprises a catechol O-methyltransferase (COMT) inhibitor. In some aspects, the additional therapeutic agent comprises a N-methyl-D-aspartate (NMDA) receptor antagonist. In some aspects, the additional therapeutic agent comprises an immunomodulatory. In some aspects, the additional therapeutic agent comprises an immunosuppressant.

Non-limiting examples of such agents include: Tetrabenazine (XENAZINE®), antipsychotic drugs, such as haloperidol (HALDOL®), chlorpromazine, risperidone (rRIDPERDAL®), quetiapine (SEROQUEL®), levodopa (with or without Carbidopa) (LODOSYN®), dopamine agonists such as pramipexole (MIRAPEX®), ropinirole (REQUIP®), and rotigotine (NEUPRO®), and apomorphine (APOKYN®), selegiline (ELDEPRYL®, ZELAPAR®), rasagiline (AZILECT®), Entacapone (COMTAN®), benztropine (COGENTIN®), trihexyphenidyl, amantadine, Donepezil (ARICEPT®), Galantamine (RAZADYNE®), Rivastigmine (EXELON®), Glatiramer acetate (COPAXONE®), Dimethyl fumarate (TECFIDERA®), Fingolimod (GILENYA®), Teriflunomide (AUBAGIO®), Natalizumab (TYSABRI®), Alemtuzumab (LEMTRADA®), Mitoxantrone (NOVANTRONE®), riluzole (RILUTEK®), physostigmine salicylate (ANTILIRIUM®), physostigmine sulfate (ESERINE®), metrifonate, neostigmine, ganstigmine, pyridostigmine (MESTINON®), ambenonium (MYTELASE®), demarcarium, Debio 9902 (also known as ZT-1®; Debiopharmladostigil, NP-0361,), tacrine (COGNEX®), tolserine, velnacrine maleate, memoquin, huperzine A (HUP-A®; NeuroHitech), phenserine, edrophonium (ENLON®, TENSILON®), INM-176, apomorphine (APOKYN®), bromocriptine (PARLODEL®), cabergoline (DOSTINEX®), dihydrexidine, dihydroergocryptine, fenoldopam (CORLOPAM®), lisuride (DOPERGIN®), terguride spergolide (PERMAX®), piribedil (TRIVASTAL®, TRASTAL®), quinpirole, SKF-82958 (GlaxoSmithKline), cariprazine, pardoprunox, sarizotan, chlorpromazine, fluphenazine loxzpine, resperidone, thioridazine, thiothixene, trifluoperazine, 7-hydroxyamoxapine, droperidol (INAPSINE®, DRIDOL®, DROPLETAN®), domperidone (MOTILIUM®), L-741742, L-745870, raclopride, SB-277011A, SCH-23390, ecopipam, SKF-83566, metoclopramide (REGLAN®), lurasidone (LATUDA®, also known as SM-13496; Dainippon Sumitomo), aripiprazole (ABILIFY®), chlorpromazine (THORAZINE®), iloperidone (FANAPTA®), flupentixol decanoate (DEPIXOL®, FLUANXOL®), reserpine (SERPLAN®), pimozide (ORAP®), fluphenazine decanoate, fluphenazine hydrochloride, prochlorperazine (COMPRO®), asenapine (SAPHRIS®), loxapine (LOXITANE®), molindone (MOBAN®), perphenazine, thioridazine, thiothixine, trifluoperazine (STELAZINE®), ramelteon, clozapine (CLOZARIL®), norclozapine (ACP-104), paliperidone (INVEGA®), melperone, olanzapine (ZYPREXA®), talnetant, amisulpride, ziprasidone (GEODON®), blonanserin (LONASEN®), ACP-103 (Acadia Pharmaceuticals), selegiline hydrochloride (I-deprenyl, ELDEPRYL®, ZELAPAR®), dimethylselegilene, brofaromine, phenelzine (NARDIL®), tranylcypromine (PARNATE®), moclobemide (AURORIX®, MANERIX®), befloxatone, safinamide, isocarboxazid (MARPLAN®), nialamide (NIAMID®), iproniazide (MARSILID®, IPROZID®, IPRONID®), CHF-3381 (Chiesi Farmaceutici), iproclozide, toloxatone (HUMORYL®, PERENUM®), bifemelane, desoxypeganine, harmine (also known as telepathine or banasterine), harmaline, linezolid (ZYVOX®, ZYVOXID®), pargyline (EUDATIN®, SUPIRDYL®), nitecapone, tolcapone (TASMAR®), tropolone, memantine (NAMENDA®, AXURA®, EBIXA®), amantadine (SYMMETREL®), acamprosate (CAMPRAL®), besonprodil, ketamine (KETALAR®), delucemine, dexanabinol, dexefaroxan, dextromethorphan, dextrorphan, traxoprodil, CP-283097, himantane, idantadol, ipenoxazone, L-701252 (Merck), lancicemine, levorphanol (DROMORAN®), LY-233536 and LY-235959 (both Lilly), methadone, (DOLOPHINE®), neramexane, perzinfotel, phencyclidine, tianeptine (STABLON®), dizocilpine (also known as MK-801), EAB-318 (Wyeth), ibogaine, voacangine, tiletamine, aptiganel (CERESOTAT®), gavestinel, remacimide, MBP-8298 (synthetic myelin basic protein peptide), roquinimex (LINOMIDE®), laquinimod (also known as ABR-215062 and SAIK-MS), ABT-874 (human anti-IL-12 antibody; Abbott), rituximab (RITUXAN®), leflunomide, ciclesonide, daclizumab (ZENAPAX®), methotrexate (TREXALL®, RHEUMATREX®), suplatast tosilate, mycophenolate mofetil (CELLCEPT®), mycophenolate sodium (MYFORTIC®), azathioprine (AZASAN®, IMURAN®), mercaptopurine (PURI-NETHOL®), cyclophosphamide (NEOSAR®, CYTOXAN®), voclosporin, PUR-118, AMG 357, AMG 811, BCT197, chlorambucil (LEUKERAN®), cladribine (LEUSTATIN®, MYLINAX®), alpha-fetoprotein, etanercept (ENBREL®), leflunomide, ciclesonide chloroquine, hydroxychloroquine, d-penicillamine, auranofin, sulfasalazine, sodium aurothiomalate, cyclosporine, cromolyn, infliximab, adalimumab, certolizumab pegol, golimumab, rituximab, ocrelizumab, ofatumumab, 4-benzyloxy-5-((5-undecyl-2H-pyrrol-2-ylidene)methyl)-2,2′-bi-1H-pyrrole (also known as PNU-156804), and combinations thereof.

In some aspects, any of the LRRC4 family mimic molecule, nucleic acids, vectors, cells, protein conjugates, or compositions described herein is administered intravenously, orally, parenterally, transthecally, intrathecally, intra-cerebroventricularly, intravitreally, pulmonarily, subcutaneously, intradermally, intramuscularly, or intraventricularly.

The following examples are offered by way of illustration and not by way of limitation.

### EXAMPLES

### Example 1: Analysis of the Interaction Between FAM19A5 and Members of the LRRC4 Protein Family

To understand the interaction between FAM19A5 and members of the LRRC4 protein family, HEK293 cells were modified to express a FLAG-tagged member of the LRRC4 protein family, i.e., LRRC4C protein, LRRC4 protein, or LRRC4B protein. Then, the HEK293 cells or primary cortical neurons were treated with recombinant FAM19A5 protein (1 μM) for 30 minutes, and the binding between FAM19A5 protein and the different members of the LRRC4 protein family was assessed using both co-immunoprecipitation and immunofluorescence assays.

For the co-immunoprecipitation assay, cell lysates from the different FAM19A5-treated HEK293 cells were collected and immunoprecipitated with an anti-FLAG antibody, anti-FAM19A5 (1-65) antibody, or a human IgG antibody (control). Immunoprecipitated proteins were immunoblotted with anti-FLAG and anti-FAM19A5 (3-2) antibodies. For the immunofluorescence assay, the HEK293 cells treated with the recombinant FAM19A5 protein were immunostained with anti-FAM19A5 (3-2) (to detect FAM19A5 protein expression) and anti-FLAG antibodies (to detect member of the LRRC4 protein family). The primary cortical neurons treated with the recombinant FAM19A5 protein were immunostained with anti-FAM19A5 (3-2) and anti-LRRC4B antibodies. The nuclei were stained with Hoechst33342 (blue).

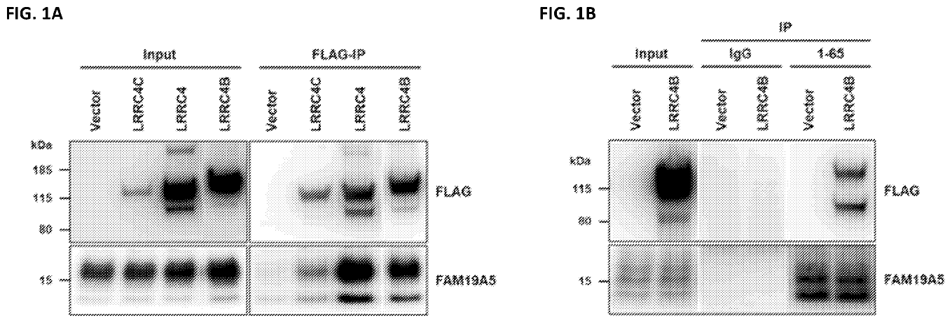

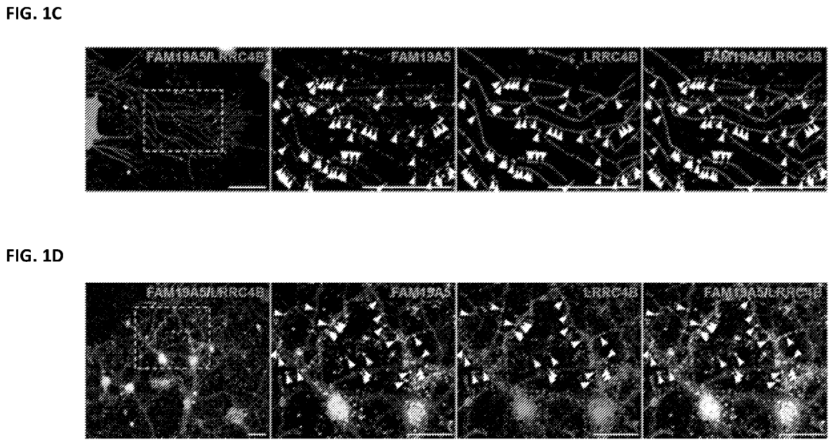

As shown in FIG. 1A, anti-FLAG antibody was able to co-immunoprecipitate the FAM19A5 protein. Similarly, as shown in FIG. 1B, the anti-FAM19A5 (1-65) antibody was able to specifically co-immunoprecipitate the LRRC4B protein. Similar results were observed using the immunofluorescence assay. In both the LRRC4B-expressing HEK293 cells and primary cortical neurons, the FAM19A5 protein was largely associated with dendrite-like processes or neurites where LRRC4B protein was highly expressed (see FIGS. 1C and 1D), suggesting the interaction between members of the LRRC4 protein family (e.g., LRRC4B) and FAM19A5 protein.

Next, to assess whether the above results were specific to certain isoforms of the FAM19A5 protein, HEK293 cells were co-transfected with cDNA encoding the FLAG-tagged LRRC4B protein and cDNA encoding either isoform 1 or isoform 2 of the FAM19A5 protein. Then, binding was assessed using both immunofluorescence and co-immunoprecipitation assays.

For the immunofluorescence assay, co-transfected HEK293 cells were immunostained with anti-FAM19A5 (1-65) and anti-FLAG antibodies to determine the subcellular localization of FAM19A5 and LRRC4B proteins, respectively. Nuclei were stained with Hoechst33342 (blue). For the co-immunoprecipitation assay, cell lysates from the co-transfected HEK293 cells were immunoprecipitated with an anti-FLAG antibody, anti-FAM19A5 (1-65) antibody, anti-FAM19A5 (3-2) antibody, or a human IgG antibody (control). Immunoprecipitated proteins were immunoblotted with anti-FLAG and anti-FAM19A5 (3-2) antibodies.

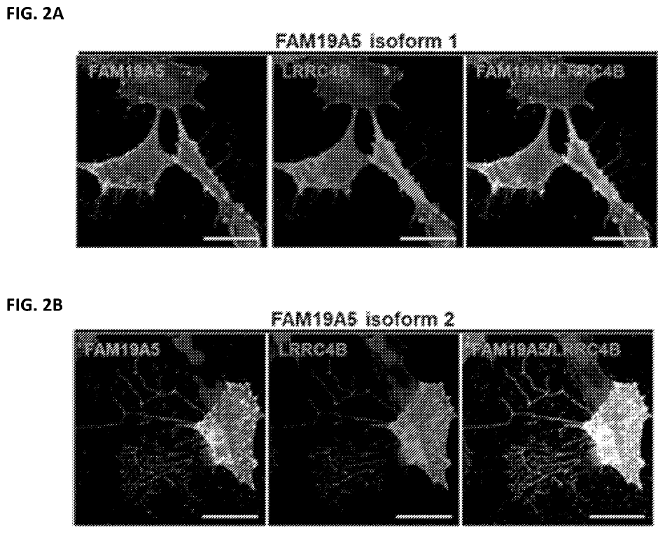

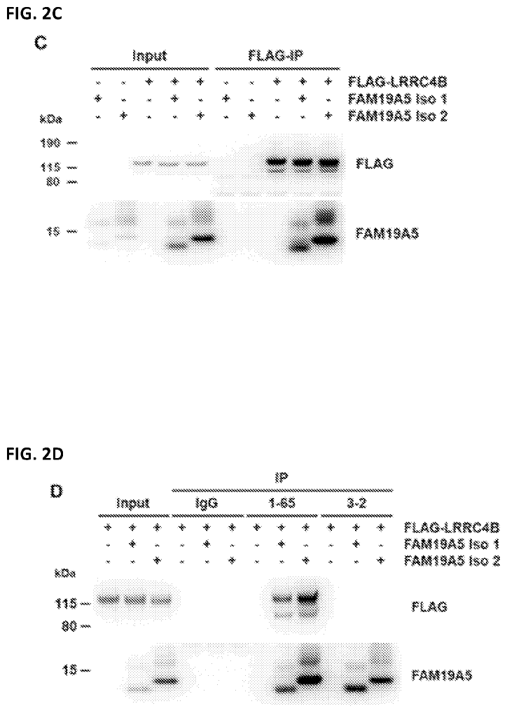

Similar to the earlier results, in the co-transfected HEK293 cells, both isoforms of the FAM19A5 protein appeared highly co-localized with the LRRC4B protein, particularly in vesicle-like puncta near the plasma membrane and dendrite-like processes (see FIGS. 2A and 2B). Likewise, immunoprecipitation with the anti-FLAG and anti-FAM19A5 (1-65) antibodies confirmed the interaction between LRRC4B protein and both isoforms of the FAM19A5 protein. For instance, the anti-FLAG antibody was able to co-immunoprecipitate both isoform 1 and 2 of the FAM19A5 protein (see FIG. 2C). Similarly, the anti-FAM19A5 (1-65) antibody was able to co-immunoprecipitate the LRRC4B protein (see FIG. 2D). Interestingly, the anti-FAM19A5 (3-2) antibody did not co-immunoprecipitate the LRRC4B protein. Not to be bound by any one theory, the differences observed with the 1-65 and 3-2 anti-FAM19A5 antibodies could be due to their binding epitopes, as these antibodies are known to bind to different epitopes within a FAM19A5 protein. See U.S. Publ. No. 20200299373, which is incorporated herein by reference in its entirety.

The above results demonstrate the interaction between the FAM19A5 protein and different members of the LRRC4 protein family (e.g., LRRC4B). By inhibiting, reducing, and/or dissociating this interaction, in some aspects, polypeptides of the present disclosure could be useful in regulating biological activities associated with such an interaction.

### Example 2: Identification of the FAM19A5 Protein Binding Domain of LRRC4B Protein

To determine the specific motif or domain of the LRRC4B protein that is responsible for the binding to a FAM19A5 protein, various FLAG-tagged LRRC4B deletion constructs were generated (see Table 6 below). HEK293 cells were transfected with the different deletion constructs and then treated with recombinant FAM19A5 protein as described in Example 1. Then, the cell lysates from the different HEK293 cells were immunoprecipitated with the anti-FLAG antibody. The immunoprecipitated proteins were immunoblotted with anti-FLAG and anti-FAM19A5 (3-2) antibodies.

TABLE 6
FLAG-tagged LRRC4B deletion constructs

| Construct # | Description | Amino Acid Sequence |
|---|---|---|
| 1 | Amino acids 36-713 | AGGGGVAVTSAAGGGSPPATSCPVACSCSNQASRVICTRRDLAEVP |
| “LRRC4B | of SEQ ID NO: 2 | ASIPVNTRYLNLQENGIQVIRTDTFKHLRHLEILQLSKNLVRKIEV |
| (36-713)” | | GAFNGLPSLNTLELFDNRLTTVPTQAFEYLSKLRELWLRNNPIESI |
| | | PSYAFNRVPSLRRLDLGELKRLEYISEAAFEGLVNLRYLNLGMCNL |
| | | KDIPNLTALVRLEELELSGNRLDLIRPGSFQGLTSLRKLWLMHAQV |
| | | ATIERNAFDDLKSLEELNLSHNNLMSLPHDLFTPLHRLERVHLNHN |
| | | PWHCNCDVLWLSWWLKETVPSNTTCCARCHAPAGLKGRYIGELDQS |
| | | HFTCYAPVIVEPPTDLNVTEGMAAELKCRTGTSMTSVNWLTPNGTL |
| | | MTHGSYRVRISVLHDGTLNFTNVTVQDTGQYTCMVTNSAGNTTASA |
| | | TLNVSAVDPVAAGGTGSGGGGPGGSGGVGGGSGGYTYFTTVTVETL |
| | | ETQPGEEALQPRGTEKEPPGPTTDGVWGGGRPGDAAGPASSSTTAP |
| | | APRSSRPTEKAFTVPITDVTENALKDLDDVMKTTKIIIGCFVAITF |
| | | MAAVMLVAFYKLRKQHQLHKHHGPTRTVEIINVEDELPAASAVSVA |
| | | AAAAVASGGGVGGDSHLALPALERDHLNHHHYVAAAFKAHYSSNPS |
| | | GGGCGGKGPPGLNSIHEPLLFKSGSKENVQETQI (SEQ ID NO: |
| | | 52) |
| 2 | Amino acids 157- | LSKLRELWLRNNPIESIPSYAFNRVPSLRRLDLGELKRLEYISEAA |
| “LRRC4B | 713 of SEQ ID NO: 2 | FEGLVNLRYLNLGMCNLKDIPNLTALVRLEELELSGNRLDLIRPGS |
| (157-713)” | | FQGLTSLRKLWLMHAQVATIERNAFDDLKSLEELNLSHNNLMSLPH |
| | | DLFTPLHRLERVHLNHNPWHCNCDVLWLSWWLKETVPSNTTCCARC |
| | | HAPAGLKGRYIGELDQSHFTCYAPVIVEPPTDLNVTEGMAAELKCR |
| | | TGTSMTSVNWLTPNGTLMTHGSYRVRISVLHDGTLNFTNVTVQDTG |
| | | QYTCMVTNSAGNTTASATLNVSAVDPVAAGGTGSGGGGPGGSGGVG |
| | | GGSGGYTYFTTVTVETLETQPGEEALQPRGTEKEPPGPTTDGVWGG |
| | | GRPGDAAGPASSSTTAPAPRSSRPTEKAFTVPITDVTENALKDLDD |
| | | VMKTTKIIIGCFVAITFMAAVMLVAFYKLRKQHQLHKHHGPTRTVE |
| | | IINVEDELPAASAVSVAAAAAVASGGGVGGDSHLALPALERDHLNH |
| | | HHYVAAAFKAHYSSNPSGGGCGGKGPPGLNSIHEPLLFKSGSKENV |
| | | QETQI (SEQ ID NO: 53) |
| 3 | Amino acids 230- | RLEELELSGNRLDLIRPGSFQGLTSLRKLWLMHAQVATIERNAFDD |
| “LRRC4B | 713 of SEQ ID NO: 2 | LKSLEELNLSHNNLMSLPHDLFTPLHRLERVHLNHNPWHCNCDVLW |
| (230-713)” | | LSWWLKETVPSNTTCCARCHAPAGLKGRYIGELDQSHFTCYAPVIV |
| | | EPPTDLNVTEGMAAELKCRTGTSMTSVNWLTPNGTLMTHGSYRVRI |
| | | SVLHDGTLNFTNVTVQDTGQYTCMVTNSAGNTTASATLNVSAVDPV |
| | | AAGGTGSGGGGPGGSGGVGGGSGGYTYFTTVTVETLETQPGEEALQ |
| | | PRGTEKEPPGPTTDGVWGGGRPGDAAGPASSSTTAPAPRSSRPTEK |
| | | AFTVPITDVTENALKDLDDVMKTTKIIIGCFVAITFMAAVMLVAFY |
| | | KLRKQHQLHKHHGPTRTVEIINVEDELPAASAVSVAAAAAVASGGG |
| | | VGGDSHLALPALERDHLNHHHYVAAAFKAHYSSNPSGGGCGGKGPP |
| | | GLNSIHEPLLFKSGSKENVQETQI (SEQ ID NO: 54) |
| 4 | Amino acids 364- | PVIVEPPTDLNVTEGMAAELKCRTGTSMTSVNWLTPNGTLMTHGSY |
| “LRRC4B | 713 of SEQ ID NO: 2 | RVRISVLHDGTLNFTNVTVQDTGQYTCMVTNSAGNTTASATLNVSA |
| (364-713)” | | VDPVAAGGTGSGGGGPGGSGGVGGGSGGYTYFTTVTVETLETQPGE |
| | | EALQPRGTEKEPPGPTTDGVWGGGRPGDAAGPASSSTTAPAPRSSR |
| | | PTEKAFTVPITDVTENALKDLDDVMKTTKIIIGCFVAITFMAAVML |
| | | VAFYKLRKQHQLHKHHGPTRTVEIINVEDELPAASAVSVAAAAAVA |
| | | SGGGVGGDSHLALPALERDHLNHHHYVAAAFKAHYSSNPSGGGCGG |
| | | KGPPGLNSIHEPLLFKSGSKENVQETQI (SEQ ID NO: 55) |
| 5 | Amino acids 453- | VSAVDPVAAGGTGSGGGGPGGSGGVGGGSGGYTYFTTVTVETLETQ |
| “LRRC4B | 713 of SEQ ID NO: 2 | PGEEALQPRGTEKEPPGPTTDGVWGGGRPGDAAGPASSSTTAPAPR |
| (453-713)” | | SSRPTEKAFTVPITDVTENALKDLDDVMKTTKIIIGCFVAITFMAA |
| | | VMLVAFYKLRKQHQLHKHHGPTRTVEIINVEDELPAASAVSVAAAA |
| | | AVASGGGVGGDSHLALPALERDHLNHHHYVAAAFKAHYSSNPSGGG |
| | | CGGKGPPGLNSIHEPLLFKSGSKENVOETQI (SEQ ID NO: 56) |
| 6 | Amino acids 577- | IIIGCFVAITFMAAVMLVAFYKLRKQHQLHKHHGPTRTVEIINVED |
| “LRRC4B | 713 of SEQ ID NO: 2 | ELPAASAVSVAAAAAVASGGGVGGDSHLALPALERDHLNHHHYVAA |
| (577-713)” | | AFKAHYSSNPSGGGCGGKGPPGLNSIHEPLLFKSGSKENVQETQI |
| | | (SEQ ID NO: 57) |
| 7 | Amino acids 36-576 | AGGGGVAVTSAAGGGSPPATSCPVACSCSNQASRVICTRRDLAEVP |
| “LRRC4B | of SEQ ID NO: 2 | ASIPVNTRYLNLQENGIQVIRTDTFKHLRHLEILQLSKNLVRKIEV |
| (36-576)” | | GAFNGLPSLNTLELFDNRLTTVPTQAFEYLSKLRELWLRNNPIESI |
| | | PSYAFNRVPSLRRLDLGELKRLEYISEAAFEGLVNLRYLNLGMCNL |
| | | KDIPNLTALVRLEELELSGNRLDLIRPGSFQGLTSLRKLWLMHAQV |
| | | ATIERNAFDDLKSLEELNLSHNNLMSLPHDLFTPLHRLERVHLNHN |
| | | PWHCNCDVLWLSWWLKETVPSNTTCCARCHAPAGLKGRYIGELDQS |
| | | HFTCYAPVIVEPPTDLNVTEGMAAELKCRTGTSMTSVNWLTPNGTL |
| | | MTHGSYRVRISVLHDGTLNFTNVTVQDTGQYTCMVTNSAGNTTASA |
| | | TLNVSAVDPVAAGGTGSGGGGPGGSGGVGGGSGGYTYFTTVTVETL |
| | | ETQPGEEALQPRGTEKEPPGPTTDGVWGGGRPGDAAGPASSSTTAP |
| | | APRSSRPTEKAFTVPITDVTENALKDLDDVMKTTK (SEQ ID NO: |
| | | 58) |
| 8 | Amino acids 36-363 | AGGGGVAVTSAAGGGSPPATSCPVACSCSNQASRVICTRRDLAEVP |
| “LRRC4B | of SEQ ID NO: 2 | ASIPVNTRYLNLQENGIQVIRTDTFKHLRHLEILQLSKNLVRKIEV |
| (36-363)” | | GAFNGLPSLNTLELFDNRLTTVPTQAFEYLSKLRELWLRNNPIESI |
| | | PSYAFNRVPSLRRLDLGELKRLEYISEAAFEGLVNLRYLNLGMCNL |
| | | KDIPNLTALVRLEELELSGNRLDLIRPGSFQGLTSLRKLWLMHAQV |
| | | ATIERNAFDDLKSLEELNLSHNNLMSLPHDLFTPLHRLERVHLNHN |
| | | PWHCNCDVLWLSWWLKETVPSNTTCCARCHAPAGLKGRYIGELDQS |
| | | HFTCYA (SEQ ID NO: 59) |
| 9 | Amino acids A364- | AGGGGVAVTSAAGGGSPPATSCPVACSCSNQASRVICTRRDLAEVP |
| “LRRC4B | 576 of SEQ ID NO: 2 | ASIPVNTRYLNLQENGIQVIRTDTFKHLRHLEILQLSKNLVRKIEV |
| (A364-576) | | GAFNGLPSLNTLELFDNRLTTVPTQAFEYLSKLRELWLRNNPIESI |
| | | PSYAFNRVPSLRRLDLGELKRLEYISEAAFEGLVNLRYLNLGMCNL |
| | | KDIPNLTALVRLEELELSGNRLDLIRPGSFQGLTSLRKLWLMHAQV |
| | | ATIERNAFDDLKSLEELNLSHNNLMSLPHDLFTPLHRLERVHLNHN |
| | | PWHCNCDVLWLSWWLKETVPSNTTCCARCHAPAGLKGRYIGELDQS |
| | | HFTCYAIIIGCFVAITFMAAVMLVAFYKLRKQHQLHKHHGPTRTVE |
| | | IINVEDELPAASAVSVAAAAAVASGGGVGGDSHLALPALERDHLNH |
| | | HHYVAAAFKAHYSSNPSGGGCGGKGPPGLNSIHEPLLFKSGSKENV |
| | | QETQI (SEQ ID NO: 60) |
| 10 | Amino acids 364- | PVIVEPPTDLNVTEGMAAELKCRTGTSMTSVNWLTPNGTLMTHGSY |
| “LRRC4B | 576 of SEQ ID NO: 2 | RVRISVLHDGTLNFTNVTVQDTGQYTCMVTNSAGNTTASATLNVSA |
| (364-576)” | | VDPVAAGGTGSGGGGPGGSGGVGGGSGGYTYFTTVTVETLETQPGE |
| | | EALQPRGTEKEPPGPTTDGVWGGGRPGDAAGPASSSTTAPAPRSSR |
| | | PTEKAFTVPITDVTENALKDLDDVMKTTK (SEQ ID NO: 61) |
| 11 | Amino acids 453- | VSAVDPVAAGGTGSGGGGPGGSGGVGGGSGGYTYFTTVTVETLETQ |
| “LRRC4B | 576 of SEQ ID NO: 2 | PGEEALQPRGTEKEPPGPTTDGVWGGGRPGDAAGPASSSTTAPAPR |
| (453-576)” | | SSRPTEKAFTVPITDVTENALKDLDDVMKTTK (SEQ ID NO: |
| | | 62) |
| 12 | Amino acids 484- | YTYFTTVTVETLETQPGEEALQPRGTEKEPPGPTTDGVWGGGRPGD |
| “LRRC4B | 576 of SEQ ID NO: 2 | AAGPASSSTTAPAPRSSRPTEKAFTVPITDVTENALKDLDDVMKTT |
| (484-576)” | | K (SEQ ID NO: 63) |
| 13 | Amino acids 498- | QPGEEALQPRGTEKEPPGPTTDGVWGGGRPGDAAGPASSSTTAPAP |
| “LRRC4B | 576 of SEQ ID NO: 2 | RSSRPTEKAFTVPITDVTENALKDLDDVMKTTK (SEQ ID NO: |
| (498-576)” | | 64) |

As shown in FIGS. 3A and 3B, all deletion constructs comprising the threonine-rich domain of the LRRC4B protein (“Thr” in FIG. 3A) were able to bind to the FAM19A5 protein with varying degree. These constructs included: LRRC4B (36-713) (i.e., construct #1); LRRC4B (157-713) (i.e., construct #2); LRRC4B (230-713) (i.e., construct #3); LRRC4B (364-713) (i.e., construct #4); LRRC4B (453-713) (i.e., construct #5); LRRC4B (36-576) (i.e., construct #7); LRRC4B (364-576) (i.e., construct #10); LRRC4B (453-576) (i.e., construct #11); and LRRC4B (484-576) (i.e., construct #12). In particular, the amino acid sequence at positions 484-497 of the LRRC4B protein appeared to have an important role in binding, as the deletion construct containing amino acids 484-576 (i.e., construct #12) was able to bind to FAM19A5 protein, whereas the deletion construct containing amino acids 498-576 (i.e., construct #13) was not able to do so (see FIGS. 3A and 3B).

Next, to confirm the above co-immunoprecipitation results, an ELISA assay was used to measure the binding of the FAM19A5 protein to either the full-length ectodomain of members of LRRC4 protein family or various LRRC4B ectodomain protein fragments. Specifically, an ELISA plate was coated with one of the following LRRC4B ectodomain protein, which was conjugated to human Fc (100 nM/well): (1) full-length ectodomain of LRRC4 protein (amino acid residues 39-527 of SEQ ID NO: 1) (SEQ ID NO: 4); (2) full-length ectodomain of LRRC4B protein (amino acids 36-576 of SEQ ID NO: 2; i.e., construct #7 in Table 6) (SEQ ID NO: 5); (3) full-length ectodomain of LRRC4C protein (amino acids 45-527 of SEQ ID NO: 3) (SEQ ID NO: 6); (4) LRRC4B ectodomain fragment (amino acids 453-576 of SEQ ID NO: 2; i.e., construct #11 in Table 6) (SEQ ID NO: 7); (5) LRRC4B ectodomain fragment (amino acids 484-576 of SEQ ID NO: 2; i.e., construct #12 in Table 6) (SEQ ID NO: 8); (6) LRRC4B ectodomain fragment (amino acids 482-576 of SEQ ID NO: 2) (SEQ ID NO: 9); (7) LRRC4B ectodomain fragment (amino acids 482-497 of SEQ ID NO: 2) (SEQ ID NO: 10); and (8) LRRC4B ectodomain fragment (amino acids 498-576 of SEQ ID NO: 2; i.e., construct #13 in Table 6) (SEQ ID NO: 11). Then, recombinant FAM19A5 proteins (0.005, 0.01, 0.025, 0.05, 0.1, 0.25, 0.5, 1, 2.5, 5, and 10 nM) were added to the relevant wells, and the plates were incubated at 37° C. for 1 hour. Afterwards, the amount of LRRC4B-bound FAM19A5 protein was detected using HRP-conjugated anti-FAM19A5 (1-65) antibody.

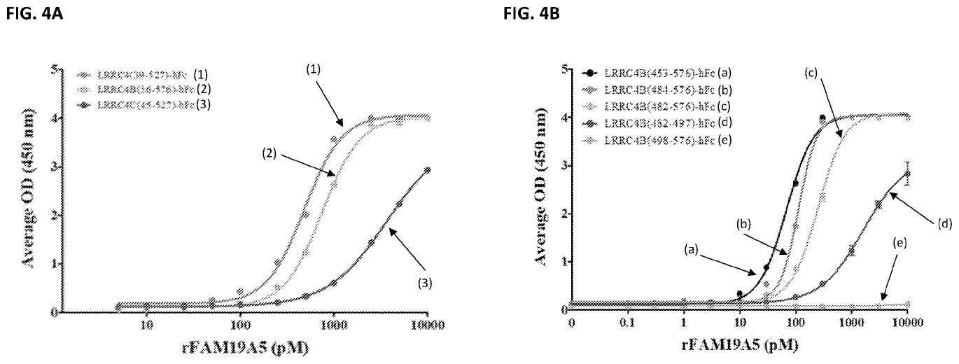

As shown in FIG. 4A, the full-length ectodomain of all members of the LRRC4 protein family (i.e., LRRC4, LRRC4B, and LRRC4C proteins) was able to find to FAM19A5 protein to varying degrees. The full-length ectodomains of the LRRC4 and LRRC4B proteins bound to the FAM19A5 protein with an EC50 of 0.48 nM and 0.64 nM, respectively. No saturation in binding was observed for the full-length LRRC4C protein at 10 nM concentration of FAM19A5. And, in agreement with the co-immunoprecipitation assay, LRRC4B ectodomain protein fragments containing the sequence at positions 484-497 of SEQ ID NO: 2 had substantial affinity to FAM19A5, whereas ectodomain protein fragment that lacked this sequence (i.e., LRRC4B(498-576)) failed to bind to FAM19A5 (see FIG. 4B).

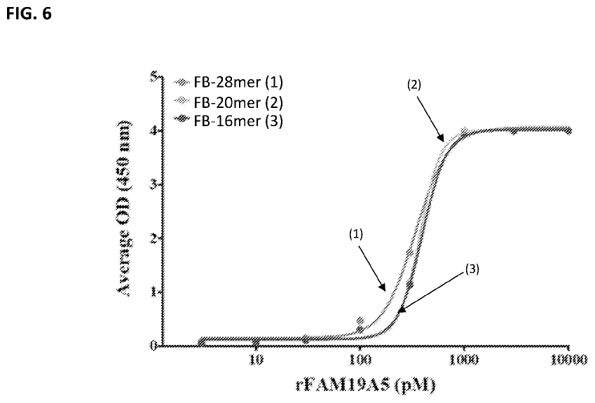

As further confirmation, three synthetic polypeptides comprising amino acids 484-497 of SEQ ID NO: 2 (i.e., YTYFTTVTVETLET; SEQ ID NO: 65) were constructed: (1) FB-16 (GYTYFTTVTVETLETQ; SEQ ID NO: 17), (2) FB-20 (GYTYFTTVTVETLETQPGEE; SEQ ID NO: 18), and (3) FB-28 (GYTYFTTVTVETLETQPGEKEPPGPTTD; SEQ ID NO: 19). The peptides differed in their total length. The ability of these polypeptides to bind to recombinant FAM19A5 protein was assessed using an ELISA assay as described above. As seen in FIG. 6B, each of FB-16, FB-20, and FB-28 was able to bind to recombinant FAM19A5 protein with high affinity, similar to the LRRC4B ectodomain protein fragments that contained amino acids 484-497 of SEQ ID NO: 2 (i.e., SEQ ID NO: 65).

Collectively, the above results suggest the importance of an amino acid sequence within positions 484-497, and in particular positions 484-493 (i.e., YTYFTTVTVE; SEQ ID NO: 29), of the LRRC4B protein (i.e., SEQ ID NO: 65) in binding to FAM19A5 protein (also referred to herein as “FAM19A5 binding domain”). As shown in FIG. 20, this sequence is generally evolutionarily conserved in the LRRC4 protein family among different vertebrates.

### Example 3: Identification of the FAM19A5 Protein Binding Domain of Other Members of the LRRC4 Protein Family

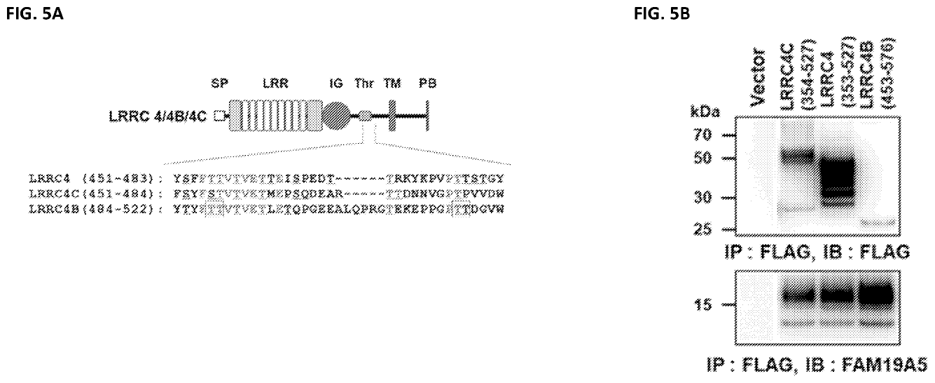

As described in Example 2, all members of the LRRC4 protein family were capable of binding to FAM19A5 protein to varying degrees. Therefore, to compare the bindings domains, the amino acid sequences of the LRRC4B, LRRC4, and LRRC4C proteins were aligned. As shown in FIG. 5A, comparison of the amino acid sequence at positions 484-522 of LRRC4B with those at corresponding positions of LRRC4 and LRRC4C proteins showed much similarity. Therefore, to assess whether the corresponding positions of LRRC4 and LRRC4C were important in the binding of these proteins to FAM19A5 protein, cDNAs encoding either (i) LRRC4C protein fragment (amino acids 354-527 of SEQ ID NO: 3; SEQ ID NO: 66) or (ii) LRRC4 protein fragment (amino acids 353-527 of SEQ ID NO: 1; SEQ ID NO: 67) were constructed (see Table 7). HEK293 cells were transfected to express either of the protein fragments and then treated with recombinant FAM19A5 protein as described in Example 1. Then, the cell lysates from the different HEK293 cells were immunoprecipitated with the anti-FLAG antibody. The immunoprecipitated proteins were immunoblotted with anti-FLAG and anti-FAM19A5 (3-2) antibodies.

TABLE 7
LRRC4C and LRRC4 protein fragments

| Construct | Description | Amino Acid Sequence |
|---|---|---|
| “LRRC4C | Amino acids 354- | VIVEPPADLNVTEGMAAELKCRASTSLTSVSWITPNGTVMTHGAYK |
| (354-527)” | 527 of SEQ ID NO: 3 | VRIAVLSDGTLNFTNVTVQDTGMYTCMVSNSVGNTTASATLNVTAA |
| | | TTTPFSYFSTVTVETMEPSQDEARTTDNNVGPTPVVDWETTNVTTS |
| | | LTPQSTRSTEKTFTIPVTDINSGIPGIDEVMKTTK (SEQ ID NO: |
| | | 66) |
| “LRRC4 | Amino acids 353- | PFIMDAPRDLNISEGRMAELKCRTPPMSSVKWLLPNGTVLSHASRH |
| (353-527)” | 527 of SEQ ID NO: 1 | PRISVLNDGTLNFSHVLLSDTGVYTCMVTNVAGNSNASAYLNVSTA |
| | | ELNTSNYSFFTTVTVETTEISPEDTTRKYKPVPTTSTGYQPAYTTS |
| | | TTVLIQTTRVPKQVAVPATDTTDKMQTSLDEVMKTTK (SEQ ID |
| | | NO: 67) |

As shown in FIG. 5B, both the LRRC4C and LRRC4 peptide fragments were able to bind to the FAM19A5 protein. This result highlights the similarity in the binding domains of the different members of the LRRC4 family.

### Example 4: Analysis of the Role of FAM19A5 Binding Domain in Inhibiting the Interaction of FAM19A5 and LRRC4B Protein

Since LRRC4B protein fragments containing the binding domain described in Example 2 (e.g., LRRC4B (453-576); i.e., construct #11 in Table 6) was able to bind to FAM19A5 protein with high affinity, it was next assessed whether such protein fragments could compete with naturally existing LRRC4B protein for binding to FAM19A5 protein, and thereby, dissociate the FAM19A5-LRRC4 protein family complex. Briefly, HEK293 cells expressing both FAM19A5 isoform 2 and LRRC4B protein were treated in vitro for 30 minutes with either LRRC4B (453-576)-hFc or mutant LRRC4B (453-576)-hFc (includes alanine substitutions at positions 488 and 489 of SEQ ID NO: 2; SEQ ID NO: 16) protein fragments. Then, cells were immunostained with anti-FAM19A5 (1-65) and anti-LRRC4B antibodies to determine the expression of FAM19A5 and full-length LRRC4B proteins, respectively. The hFc-fused LRRC4B protein fragments were determined using an anti-hIgG antibody. The nuclei were stained with Hoechst 33342.

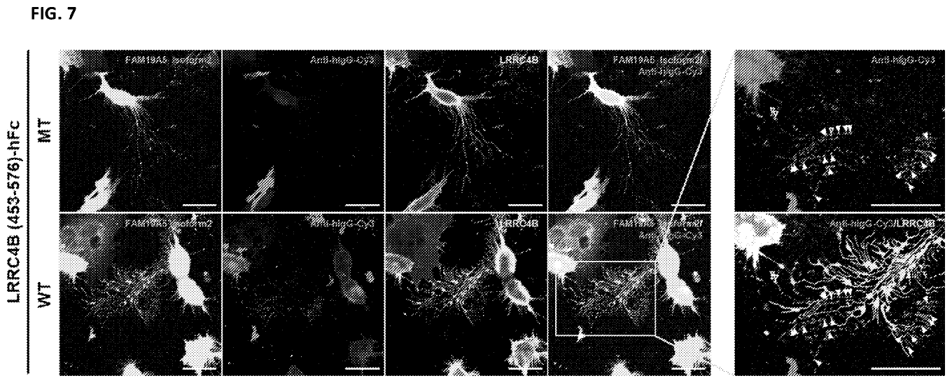

As observed earlier (see FIGS. 2A and 2B), when FAM19A5 protein was bound to the full-length LRRC4B protein, the complex was highly colocalized particularly in the plasma membrane and dendrite-like processes. In cells that were treated with LRRC4B (453-576)-hFc, FAM19A5 was largely dissociated from the full-length LRRC4B protein (see FIG. 7, bottom row). In contrast, in HEK293 cells treated with the mutant LRRC4B (453-576)-hFc, FAM19A5 protein remained largely bound to the full-length LRRC4B protein, suggesting the importance of the FAM19A5 family binding domain identified in Example 2.

Next, to further assess the role that the binding domain of the LRRC4B protein has on the interaction between FAM19A5 protein and members of the LRRC4 protein family, a competitive inhibition assay was used to determine whether different LRRC4B deletion constructs from Example 2 could inhibit the binding of FAM19A5 to the full-length ectodomain of the LRRC4B protein (i.e., amino acids 36-576 of SEQ ID NO: 2; SEQ ID NO: 5). Briefly, 100 nM of the full-length ectodomain of the LRRC4B protein was used to coat a plate, and then recombinant FAM19A5 protein (5 ng/mL) was added to the plate in combination with the following LRRC4B deletion constructs (increasing concentrations): (1) LRRC4B (453-576) (i.e., construct #11 in Table 6); (2) LRRC4B (453-576) mutant (comprises alanine substitutions at positions 488 and 489 of SEQ ID NO: 2; SEQ ID NO: 16); (3) LRRC4B (484-576) (i.e., construct #12 in Table 6); (4) LRRC4B (482-576); (5) LRRC4B (482-497); and (6) LRRC4B (498-576). The plates were incubated at 37° C., and then, the amount of FAM19A5 bound to the coated LRRC4B ectodomain protein was measured using HRP-conjugated anti-FAM19A5 (1-65) antibody.

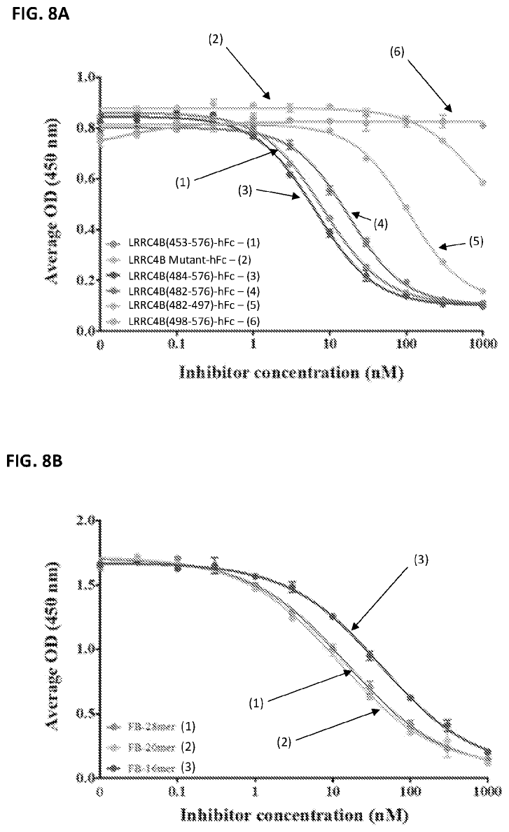

As shown in FIG. 8A, LRRC4B (453-576) was able to inhibit FAM19A5 protein from binding to the coated full-length LRRC4B ectodomain protein. Other LRRC4B protein fragments containing amino acid residues 484-497 of SEQ ID NO: 2 (i.e., binding domain of the LRRC4B protein; SEQ ID NO: 65) were also able to inhibit the interaction between FAM19A5 and the full-length LRRC4B ectodomain protein—see LRRC4B (484-576), LRRC4B (482-576), and LRRC4B (482-497). Similar results were observed with the synthetic peptides FB-16, FB-20, and FB-28 (see FIG. 8B). In contrast, LRRC4B protein fragments lacking amino acid residues 484-497 of SEQ ID NO: 2 were much inferior in their ability to inhibit the interaction—see LRRC4B mutant and LRRC4B (498-576) (see FIG. 8A).

Collectively, the above results demonstrate that peptide fragments (e.g., synthetic) containing the binding domain of the LRRC4B protein could be useful in suppressing the formation of FAM19A5-LRRC4B protein complex.

### Example 5: Analysis of the Role of the Binding Domain of LRRC4 Protein Family in Inhibiting FAM19A5-LRRC4 Protein Family Complex Formation

As described in Example 3, there appeared to be much similarity in the binding domains of the different members of the LRRC4 family. Therefore, to assess whether polypeptides comprising the LRRC4B binding domain can also inhibit other members of the LRRC4 protein family from binding to FAM19A5 protein, ELISA plates were coated with one of the following proteins (100 nM/well): (1) full-length ectodomain of LRRC4 protein (amino acid residues 39-527 of SEQ ID NO: 1; SEQ ID NO: 4); (2) full-length ectodomain of LRRC4B protein (amino acids 36-576 of SEQ ID NO: 2; i.e., construct #7 in Table 6; SEQ ID NO: 5); and (3) full-length ectodomain of LRRC4C protein (amino acids 45-527 of SEQ ID NO: 3; SEQ ID NO: 6). Then, recombinant FAM19A5 protein (5 ng/mL) was added in combination with one of the following: (i) LRRC4B (484-576) protein fragment, (ii) LRRC4B (453-576, AA) protein fragment, and (iii) synthetic FB-20 peptide. The plates were incubated at 37° C., and then, the amount of FAM19A5 protein bound to the coated LRRC4, LRRC4B, or LRRC4C proteins was measured using anti-FAM19A5 (1-65) antibody.

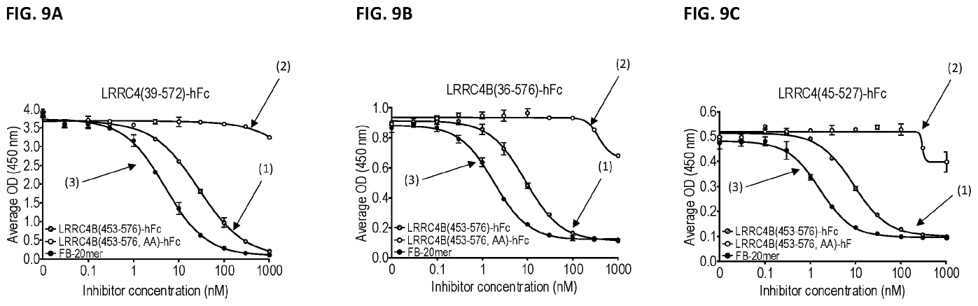

As shown in FIGS. 9A, 9B, and 9C, both the LRRC4B (484-576) protein fragment and the synthetic FB-20 peptide were able to inhibit FAM19A5 protein from binding to the coated LRRC4 and LRRC4C proteins. And, in agreement with the earlier data, the LRRC4B fragment with the alanine substitutions at positions 488 and 489 of SEQ ID NO: 2, i.e., LRRC4B mutant (SEQ ID NO: 16), had minimal effect.

Next, to assess whether polypeptides comprising the binding domains of LRRC4 or LRRC4C proteins can have similar inhibitory effects on LRRC4B protein binding, the following synthetic peptides were constructed: (1) FBC4-23 (contains the binding domain of the LRRC4 protein, i.e., YSFFTTVTVETTE); and (2) FBC4C-23 (contains the binding domain of the LRRC4C protein, i.e., FSYFSTVTVETME). Then, the ability of the peptides to inhibit the binding of LRRC4 protein family members to FAM19A5 protein was assessed using a competitive inhibition assay. Briefly, plates were coated with 100 nM of either LRRC4B protein fragment #1 (amino acids 36-576 of SEQ ID NO: 2; SEQ ID NO: 5) or LRRC4B protein fragment #2 (amino acids 453-576 of SEQ ID NO: 2; SEQ ID NO: 7). Then, recombinant FAM19A5 protein (5 ng/ml for plates coated with LRRC4B fragment #1; and 1 ng/mL for plates coated with LRRC4B fragment #2) was added to the plates in combination with 20 nM of the FBC4-23, FBC4C-23, and FB-20 peptides. The plates were incubated at 37° C. and then, the amount of FAM19A5 protein bound to the coated LRRC4B protein fragments was assessed using HRP-conjugated anti-FAM19A5 (1-65) antibody.

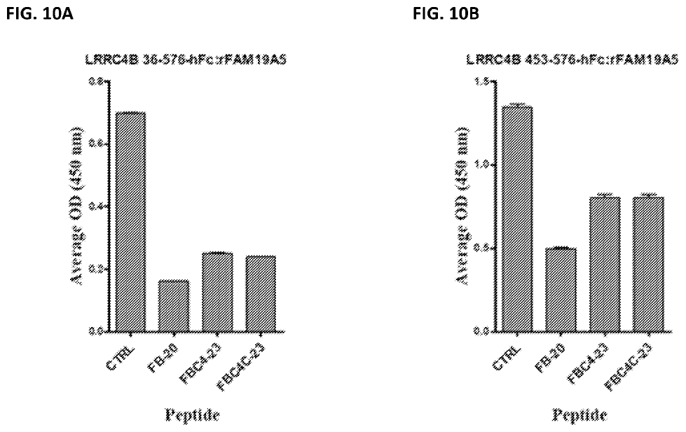

As shown in FIGS. 10A and 10B; and Table 8 (below), all three peptides (i.e., FB-20, FBC4-23, and FBC4C-23) significantly decreased the interaction between FAM19A5 protein and the coated LRRC4B protein fragments.

TABLE 8

Inhibition rate (%)

| | LRRC4B (amino acids | LRRC4B (amino acids |
|---|---|---|
| Peptide | 36-576)-hFc | 453-576)-hFc |
| FB-20 | 77.0 | 63.2 |
| FBC4-23 | 64.1 | 40.4 |
| FBC4C-23 | 65.7 | 40.4 |

Collectively, the above results demonstrate that peptides comprising the FAM19A5 binding domain of any member of the LRRC4 protein family can inhibit the interaction between FAM19A5 and LRRC4B proteins, and thus, further highlighting the conserved nature of the FAM19A5 binding domain of the LRRC4 protein family members.

### Example 6: Identification of the Minimal FAM19A5 Binding Domain Sequence Required for Inhibiting the Interaction Between FAM19A5 Protein and Members of the LRRC4 Protein Family

Next, to determine the minimal sequence required to inhibit the interaction of FAM19A5 and members of the LRRC4 protein family, ten FB-20 peptide variants were constructed by serially deleting one or more amino acids from the N-terminus or C-terminus of the FB-20 peptide. See Table 9. Then, a competitive inhibition assay was used to assess the ability of the different FB-20 peptide variants to inhibit the interaction between FAM19A5 and LRRC4B protein. Again, plates were coated with 100 nM of either LRRC4B protein fragment #1 (amino acids 36-576 of SEQ ID NO: 2; SEQ ID NO: 5) or LRRC4B protein fragment #2 (amino acids 453-576 of SEQ ID NO: 2; SEQ ID NO: 7). Then, recombinant FAM19A5 protein (5 ng/ml for plates coated with LRRC4B fragment #1; and 1 ng/mL for plates coated with LRRC4B fragment #2) was added to the plates in combination with 20 nM of the different peptides described above. After incubating the plates at 37° C., the amount of FAM19A5 protein bound to the coated LRRC4B protein fragments was assessed using HRP-conjugated anti-FAM19A5 (1-65) antibody.

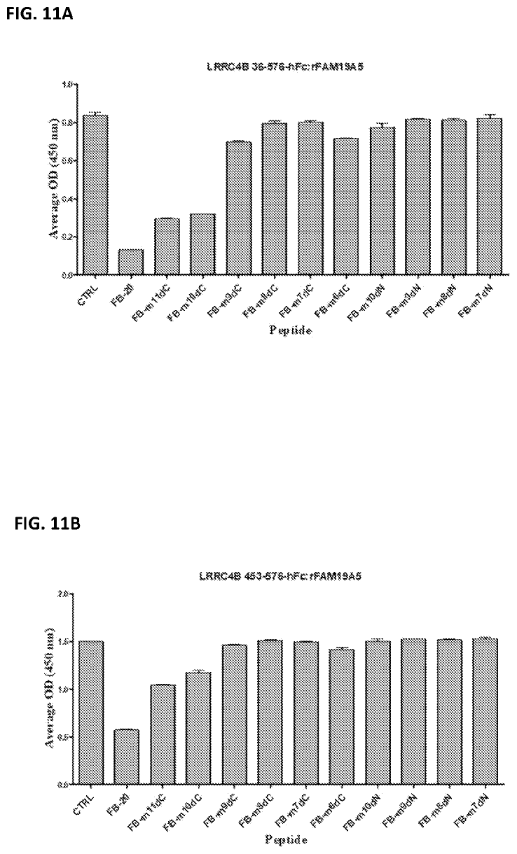

As shown in FIGS. 11A and 11B; and Table 9, peptide fragments comprising the first ten amino acids of the LRRC4B protein binding domain (i.e., YTYFTTVTVE; SEQ ID NO: 29) substantially inhibited the interaction between FAM19A5 and the coated LRRC4B protein fragments (see “FB-m11dC” and “FB-m10dC”). In contrast, peptide fragments lacking one or more amino acids at positions 1-10 of the LRRC4B protein binding domain failed to significantly inhibit the FAM19A5-LRRC4B protein interaction (see “FB-m10dC,” “FB-m9dC,” “FB-m8dC,” “FB-m7dC,” “FB-m6dC,” “FB-m10dN,” “FB-m9dN,” “FB-m8dN,” and “FB-m7dN”).

TABLE 9

| | | Inhibition rate (%) | |
|---|---|---|---|
| | | LRRC4B (amino | LRRC4B (amino |
| | | acids 36-576)- | acids 453-576)- |
| Peptide | Amino Acid Sequence | hFc | hFc |
| FB-20 | GYTYFTTVTVETLETQPGEE | 84.3 | 62.0 |
| | (SEQ ID NO: 18) | | |
| FB-m11dC | KNYTYFTTVTVETNKETQ | 65.0 | 30.4 |
| | (SEQ ID NO: 68) | | |
| FB-m10dC | KNYTYFTTVTVENKETQ (SEQ | 62.0 | 21.9 |
| | ID NO: 69) | | |
| FB-m9dC | KNYTYFTTVTVNKETQ (SEQ | 16.8 | 2.7 |
| | ID NO: 70) | | |
| FB-m8dC | KNYTYFTTVTNKETQ (SEQ | 5.1 | −0.9 |
| | ID NO: 71) | | |
| FB-m7dC | KNYTYFTTVNKETQ (SEQ ID | 4.4 | 0.4 |
| | NO: 72) | | |
| FB-m6dC | KNYTYFTTNKETQ (SEQ ID | 14.9 | 5.5 |
| | NO: 73) | | |
| FB-m10dN | KNTYFTTVTVETNKETQ (SEQ | 7.7 | 0.0 |
| | ID NO: 74) | | |
| FB-m9dN | KNYFTTVTVETNKETQ (SEQ | 2.5 | −1.6 |
| | ID NO: 75) | | |
| FB-m8dN | KNFTTVTVETNKETQ (SEQ | 3.3 | −1.1 |
| | ID NO: 76) | | |
| FB-m7dN | KNTTVTVETNKETQ (SEQ ID | 2.0 | −1.8 |
| | NO: 77) | | |

The above results suggest the importance of at least the first ten amino acids of the FAM19A5 binding domain of LRRC4 protein family members in inhibiting, reducing, and/or dissociating the interaction between LRRC4 protein family members and FAM19A5 protein.

### Example 7: Identification of Important Binding Domain Residues for Inhibiting Interaction Between LRRC4 Protein Family Members and FAM19A5 Protein

To identify the critical amino acid residues, multiple FB-20 peptide mutants were constructed, in which the individual residues of the core binding domain (i.e., YTYFTTVTVETLE; SEQ ID NO: 15) were substituted with alanine (A) or asparagine (N). See Table 10. Then, the ability of these FB-20 peptide mutants to inhibit the interaction between LRRC4B and FAM19A5 proteins was assessed, as described in Examples 3 and 4.

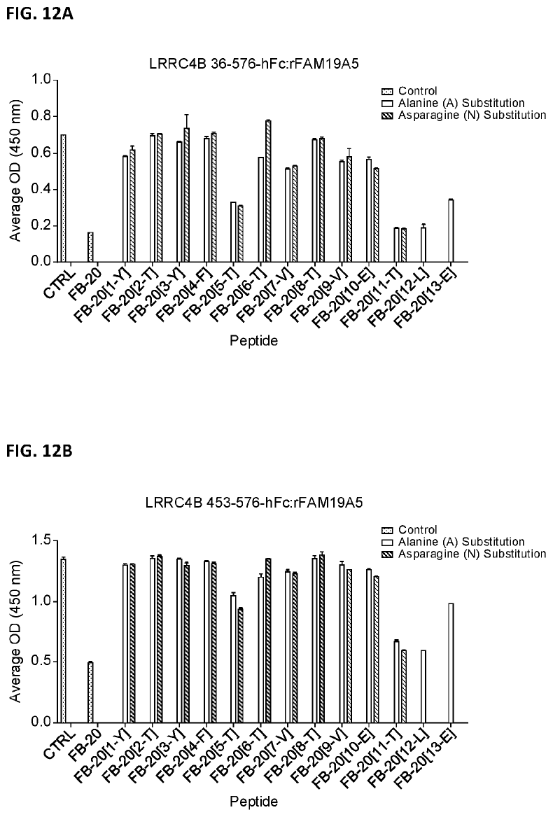

As shown in FIGS. 12A and 12B; and Table 10, FB-20 peptide mutants with either alanine or asparagine substitutions at positions 5, 11, 12, and 13 of the core binding domain were still able to significantly inhibit the interaction between FAM19A5 and LRRC4B proteins. In contrast, alanine or asparagine substitutions at positions 1, 2, 3, 4, 6, 7, 8, 9, and 10 resulted in marked reduction in the ability of the peptide to inhibit the binding of LRRC4B protein to FAM19A5, suggesting the importance of these amino acid positions within the core binding domain in inhibiting, reducing, and/or dissociating the interaction between FAM19A5 and members of the LRRC4 protein family.

TABLE 10

| | | Inhibition rate (%) | |
|---|---|---|---|
| | | LRRC4B (amino | LRRC4B (amino |
| | | acids 36-576)- | acids 453-576)- |
| Peptide | Amino Acid Sequence | hFc | hFc |
| FB-20 | GYTYFTTVTVETLETQPGEE | 77.0 | 63.2 |
| | (SEQ ID NO: 18) | | |
| FB-20[A1] | GATYFTTVTVETLETQPGEE | 17.2 | 3.6 |
| | (SEQ ID NO: 78) | | |
| FB-20[A2] | GYAYFTTVTVETLETQPGEE | 1.0 | −0.4 |
| | (SEQ ID NO: 79) | | |
| FB-20[A3] | GYTAFTTVTVETLETQPGEE | 5.9 | 0.0 |
| | (SEQ ID NO: 80) | | |
| FB-20[A4] | GYTYATTVTVETLETQPGEE | 2.7 | 1.5 |
| | (SEQ ID NO: 81) | | |
| FB-20[A5] | GYTYFATVTVETLETQPGEE | 53.4 | 22.2 |
| | (SEQ ID NO: 82) | | |
| FB-20[A6] | GYTYFTAVTVETLETQPGEE | 17.9 | 11.0 |
| | (SEQ ID NO: 83) | | |
| FB-20[A7] | GYTYFTTATVETLETQPGEE | 26.8 | 7.9 |
| | (SEQ ID NO: 84) | | |
| FB-20[A8] | GYTYFTTVAVETLETQPGEE | 3.9 | −0.4 |
| | (SEQ ID NO: 85) | | |
| FB-20[A9] | GYTYFTTVTAETLETQPGEE | 21.5 | 3.8 |
| | (SEQ ID NO: 86) | | |
| FB-20[A10] | GYTYFTTVTVATLETQPGEE | 19.6 | 6.4 |
| | (SEQ ID NO: 87) | | |
| FB-20[A11] | GYTYFTTVTVEALETQPGEE | 73.4 | 50.3 |
| | (SEQ ID NO: 88) | | |
| FB-20[A12] | GYTYFTTVTVETAETQPGEE | 73.0 | 56.2 |
| | (SEQ ID NO: 89) | | |
| FB-20[A13] | GYTYFTTVTVETLATQPGEE | 51.4 | 27.2 |
| | (SEQ ID NO: 90) | | |
| FB-20[N1] | GNTYFTTVTVETLETQPGEE | 11.9 | 3.1 |
| | (SEQ ID NO: 91) | | |
| FB-20[N2] | GYNYFTTVTVETLETQPGEE | −0.7 | −2.0 |
| | (SEQ ID NO: 92) | | |
| FB-20[N3] | GYTNFTTVTVETLETQPGEE | −5.0 | 4.0 |
| | (SEQ ID NO: 93) | | |
| FB-20[N4] | GYTYNTTVTVETLETQPGEE | −1.3 | 2.5 |
| | (SEQ ID NO: 94) | | |
| FB-20[N5] | GYTYFNTVTVETLETQPGEE | 56.1 | 30.5 |
| | (SEQ ID NO: 95) | | |
| FB-20[N6] | GYTYFTNVTVETLETQPGEE | −11.4 | −0.4 |
| | (SEQ ID NO: 96) | | |
| FB-20[N7] | GYTYFTTNTVETLETQPGEE | 24.5 | 8.3 |
| | (SEQ ID NO: 97) | | |
| FB-20[N8] | GYTYFTTVNVETLETQPGEE | 2.9 | −2.4 |
| | (SEQ ID NO: 98) | | |
| FB-20[N9] | GYTYFTTVTNETLETQPGEE | 17.5 | 6.5 |
| | (SEQ ID NO: 99) | | |
| FB-20[N10] | GYTYFTTVTVNTLETQPGEE | 27.0 | 10.8 |
| | (SEQ ID NO: 100) | | |
| FB-20[N11] | GYTYFTTVTVENLETQPGEE | 74.1 | 55.6 |
| | (SEQ ID NO: 101) | | |

### Example 8: Analysis of the Therapeutic Effects of Polypeptides Comprising the Binding Domain of LRRC4 Protein Family Members

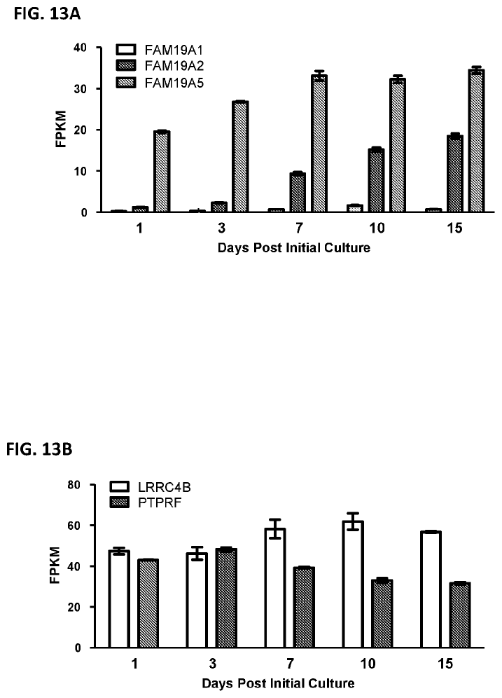

To assess the therapeutic potential of the polypeptides described herein, the transcript levels of FAM19A5, LRRC4B, and PTPRF (postsynaptic partner of LRRC4B) was assessed in primary hippocampal neurons (derived from mouse brain at day 1 postnatal) at various time points post-culture using RNA-sequencing. As shown in FIG. 13A, even as early as day 1 post-culture, FAM19A5 transcript level was significantly higher compared to other members of the FAM19 family, and remained high as far out as day 15 post-culture. As shown in FIG. 13B, the primary hippocampal neurons also expressed high transcript levels of both LRRC4B and PTPRF, which were again maintained until at least day 15 post-culture. The high expression level of these genes in primary neurons suggests their importance in various aspects of neurogenesis.

Next, primary cortical neurons (day 1 postnatal) were cultured in vitro with varying concentrations (0.006 to 60 nM) of the LRRC4B (453-576) protein fragment (i.e., amino acid residues 453-576 of SEQ ID NO: 2; SEQ ID NO: 7), and then at day 3 post-initial culture, the effect on neurite growth was assessed by immunostaining the cells with anti-beta-tubulin III antibody. Cells cultured with DMSO (“Veh”) were used as control.

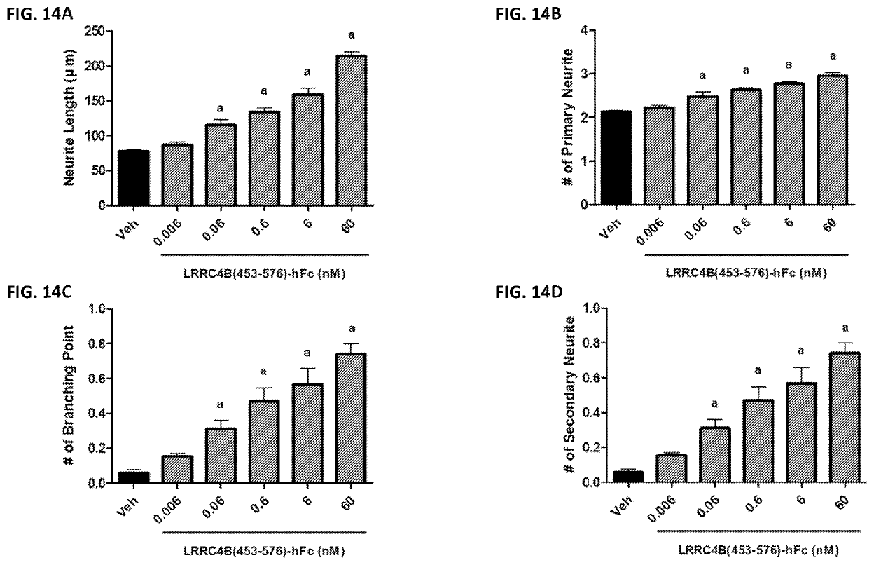

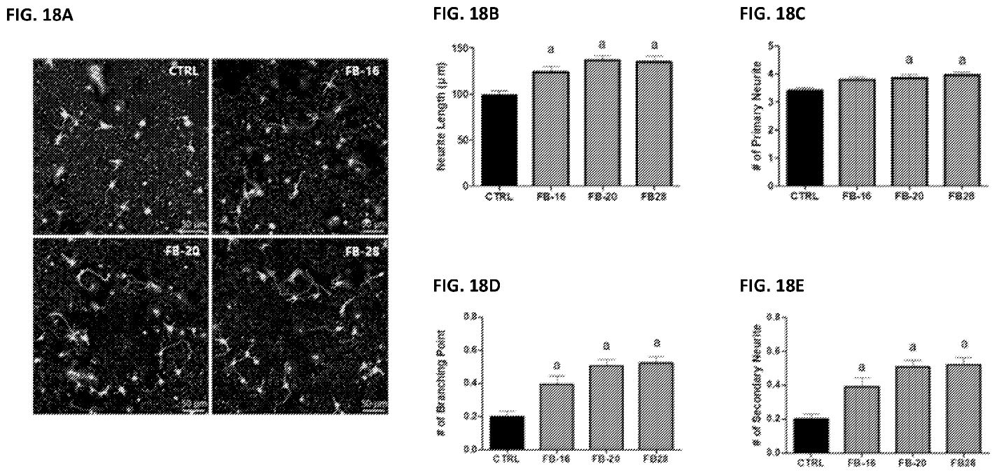

As shown in FIGS. 14A, 14B, 14C, and 14D, primary cortical neurons treated with the LRRC4B (453-576) protein fragment exhibited increased neurite growth in a dose-dependent manner. Compared to the control group, LRRC4B protein fragment treated neurons had increased neurite length (FIG. 14A), increased number of primary and secondary neurites (FIGS. 14B and 14D, respectively), and increased number of branching points (FIG. 14C). Increased neurite outgrowth was also observed when the FB-16, FB-20, and FB-28 peptides were used instead of the LRRC4B (453-576) protein fragment (see FIGS. 18A, 18B, 18C, 18D, and 18E). Among FB-16, FB-20, and FB-28 peptides, they all seemed to have similar positive effects on neurite outgrowth.

Next, it is generally known that neurites that outgrow from other neurites are capable of differentiating into axons, forming presynapses. Other neurites remain minor neurites and differentiate into dendrites, forming postsynapses. Therefore, whether the LRRC4B (453-576) protein fragment can also affect pre- and post-synaptic formation was also assessed. Briefly, mouse primary hippocampal neurons were cultured in vitro with the LRRC4B (453-576) protein fragment (6 or 60 nM). Control cells were treated with either DMSO (“Veh”) or the mutant LRRC4B (453-576) protein fragment which is not capable of binding to FAM19A5 protein (comprises alanine substitutions at positions 488 and 489). Then, at days 3 and 6 post initial culture, the expression level of synaptophysin (SYP; a presynaptic marker) was assessed. At day 7 post initial culture, the expression level of postsynaptic density 95 (PSD95; a postsynaptic marker) was assessed.

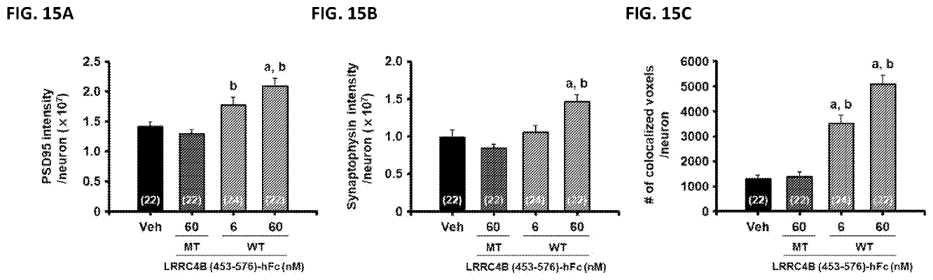

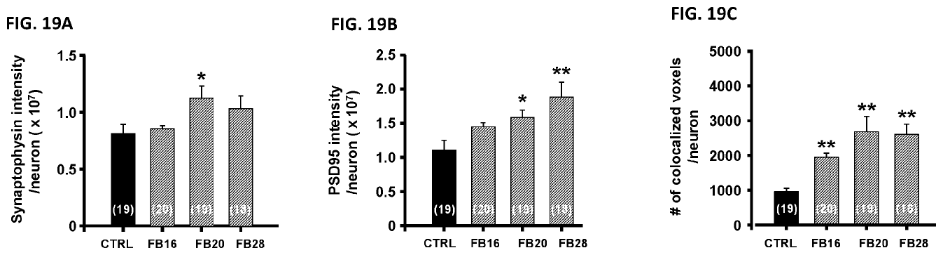

As shown in FIGS. 15A and 15B, the LRRC4B (453-576) protein fragment (at both concentrations) increased both SYP and PSD95 expression in the neurons, confirming that the increased neurite outgrowth observed can lead to increased synaptic formation. In support, as shown in FIG. 15C, in the peptide-treated mouse primary hippocampal neurons, there was increased number of puncta that were colabeled with SYP and PSD95, indicating merging between the presynapses and postsynapses. As shown in FIGS. 19A, 19B, and 19C, similar results were observed with the FB-16, FB-20, and FB-28 peptides (60 nM for each peptide).

Additionally, to confirm the above effect on neurite outgrowth in vivo, APP/PS1 mice (Alzheimer mouse model) were used. APP/PS1 mice exhibit synapse loss in CA1 of the hippocampus at 4 months after birth as revealed by 50% reductions in puncta co-labeled for pre- and postsynaptic markers such as SYP and PSD95, respectively. Hong et al., Science 352(6286): 712-716 (May 2016). These synaptic loss and neuron loss are likely associated with impairments in spatial learning and memory ability. Yoshiyama et al., Neuron 53: 337-351 (2007). CA1 in the hippocampus is the main destination for the inputs from the EC to the hippocampus. Information from the EC reaches CA1 via two main pathways. One is the direct perforant pathway from the EC to CA1 and the other is indirect pathway using the trisynaptic circuit from the EC to dentate gyrus (1^{st} synapse) to CA3 (2^{nd} synapse) to CA1 (3^{rd} synapse). Therefore, whether the administration of the LRRC4B (453-576) protein fragment (contains the FAM19A5 binding domain of the LRRC4B protein) has any effect on synaptic connections in the hippocampus, particularly in CA1 and CA3 areas, were investigated. Briefly, the APP/PS1 mice were treated with (i) the wild-type LRRC4B (amino acid residues 453-576 of SEQ ID NO: 2) protein fragment (SEQ ID NO: 7) or (ii) the mutant LRRC4B protein fragment (i.e., comprising alanine substitutions at positions 488 and 489 of SEQ ID NO: 2) (SEQ ID NO: 16).

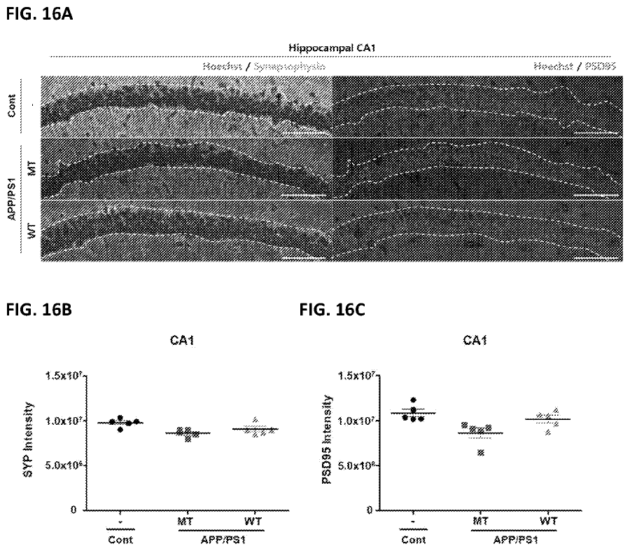

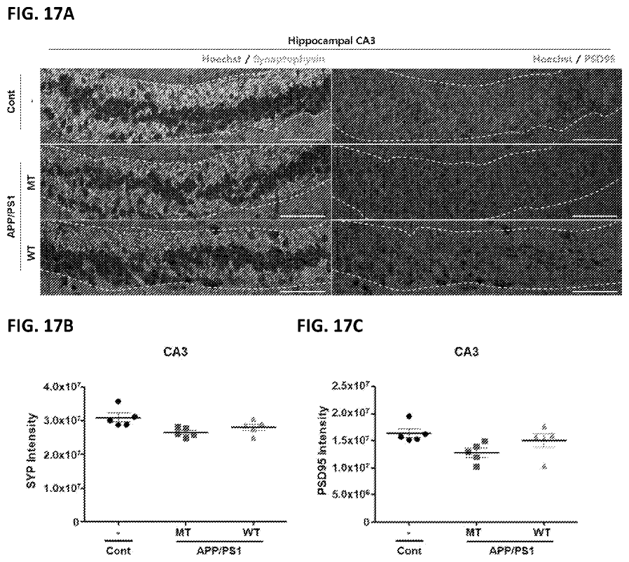

As shown in FIGS. 16A-16C and 17A-17C, compared to APP/PS1 mice treated with the mutant LRRC4B protein fragment, APP/PS1 mice treated with the wild-type LRRC4B protein fragment exhibited increased SYP and PSD95 immunoreactivities in CA1 and CA3 of the mice. The levels were similar to that observed in the untreated normal animals (“Cont”).

Collectively, the above results demonstrate that any peptides comprising the core binding domain of members of the LRRC4 protein family could serve as a decoy receptor for FAM19A5, and thereby, prevent the inhibitory effect of FAM19A5 protein on the activity (e.g., promoting neurite outgrowth and synapse formation) of members of LRRC4 protein family.

### Example 9: In Silico Residue Scanning of FAM19A5-LRRC4 Family Complex Using Schrodinger Platform

To further characterize the residues that play a role in the interaction between FAM19A5 protein and members of the LRRC4 family, SCHRODINGER BIOLUMINATE® was used to conduct an in silico alanine scanning at every single non-alanine residue of the FAM19A5-LRRC4 family member complex, and the change in Gibbs free energy was determined to represent the binding affinity for each of the amino acid residues. Specifically, all the non-alanine residues of FB-20 (i.e., GYTYFTTVTVETLETQPGEE; SEQ ID NO: 18), which is a fragment of the LRRC4B protein and comprising the FAM19A5 binding domain, were mutated to alanine. Sequences for the different FB-20 peptide variants are provided in Table 11 (below).

TABLE 11
FB-20 Peptide Variant Protein Sequences

| Peptide | Amino Acid Sequence |
|---|---|
| FB-20 | AYTYFTTVTVETLETQPGEE |
| (G1A) | |
| (SEQ ID NO: 103) | |
| FB-20 | GATYFTTVTVETLETQPGEE |
| (Y2A) | |
| (SEQ ID NO: 78) | |
| FB-20 | GYAYFTTVTVETLETQPGEE |
| (T3A) | |
| (SEQ ID NO: 79) | |
| FB-20 | GYTAFTTVTVETLETQPGEE |
| (Y4A) | |
| (SEQ ID NO: 80) | |
| FB-20 | GYTYATTVTVETLETQPGEE |
| (F5A) | |
| (SEQ ID NO: 81) | |
| FB-20 | GYTYFATVTVETLETQPGEE |
| (T6A) | |
| (SEQ ID NO: 82) | |
| FB-20 | GYTYFTAVTVETLETQPGEE |
| (T7A) | |
| (SEQ ID NO: 83) | |
| FB-20 | GYTYFTTATVETLETQPGEE |
| (V8A) | |
| (SEQ ID NO: 84) | |
| FB-20 | GYTYFTTVAVETLETQPGEE |
| (T9A) | |
| (SEQ ID NO: 85) | |
| FB-20 | GYTYFTTVTAETLETQPGEE |
| (V10A) | |
| (SEQ ID NO: 86) | |
| FB-20 | GYTYFTTVTVATLETQPGEE |
| (E11A) | |
| (SEQ ID NO: 87) | |
| FB-20 | GYTYFTTVTVEALETQPGEE |
| (T12A) | |
| (SEQ ID NO: 88) | |
| FB-20 | GYTYFTTVTVETAETQPGEE |
| (L13A) | |
| (SEQ ID NO: 89) | |
| FB-20 | GYTYFTTVTVETLATQPGEE |
| (E14A) | |
| (SEQ ID NO: 90) | |
| FB-20 | GYTYFTTVTVETLEAQPGEE |
| (T15A) | |
| (SEQ ID NO: 117) | |
| FB-20 | GYTYFTTVTVETLETAPGEE |
| (Q16A) | |
| (SEQ ID NO: 118) | |
| FB-20 | GYTYFTTVTVETLETQAGEE |
| (P17A) | |
| (SEQ ID NO: 119) | |
| FB-20 | GYTYFTTVTVETLETQPAEE |
| (G18A) | |
| (SEQ ID NO: 120) | |
| FB-20 | GYTYFTTVTVETLETQPGAE |
| (E19A) | |
| (SEQ ID NO: 121) | |
| FB-20 | GYTYFTTVTVETLETQPGEA |
| (E20A) | |
| (SEQ ID NO: 122) | |

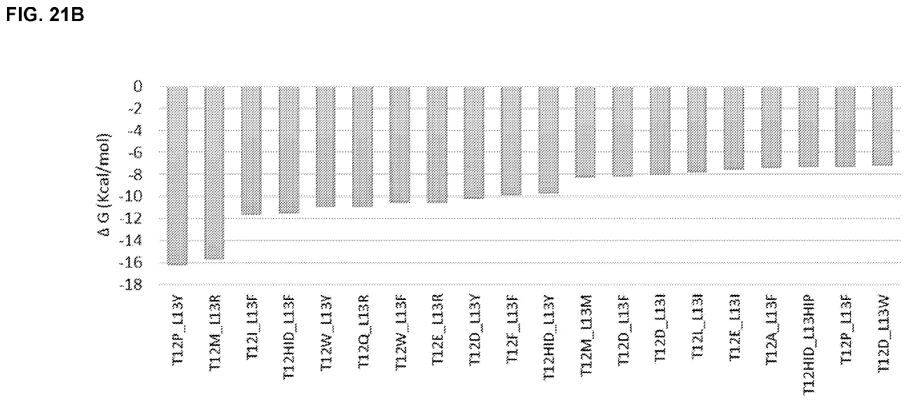

As shown in FIG. 21A (and in agreement with the earlier data—see, e.g., Example 7), certain specific residues of the FB-20 peptide fragment (e.g., from Y2 to E11 residues) seemed to be important in the interaction between FAM19A5 protein and LRRC4B, since there was a great increase in the free energy change upon the introduction of the alanine mutation at these residues. Similarly, certain residues (e.g., T12 and L13 residues) appeared to have minimal role, since the alanine substitution on those residues did not greatly alter the protein-peptide binding affinity.

Next, to assess whether the binding affinity of the FB-20 peptide fragments can be improved, the T12 and L13 residues (which appeared to have minimal role in the interaction between FAM19A5 protein and LRRC4B) were substituted with all other possible amino acid substitutions, and then, binding affinity was determined using SCHRODINGER BIOLUMINATE®. Since histidine could have three different molecular structures upon its protonation state (abbreviated as HIP, HID, and HIE; HIP: +1 charged, both δ- and ε-nitrogens protonated; HID: neutral, δ-nitrogen protonated; HIE: neutral, ε-nitrogen protonated), each residue was able to be substituted with 21 different other amino acids. Therefore, the double mutation on both T12 and L13 generated the 441 mutants. The sequences for the top twenty FB-20 peptide double mutants (at T12 and L13) predicted to enhance binding affinity between FAM19A5 and LRRC4B are provided in Table 12 (below).

TABLE 12
Sequences for Exemplary FB-20 Peptide
Fragments with T12 and/or L13 Substitutions

| Peptide | Amino Acid Sequence |
|---|---|
| FB-20 (T12P & L13Y) (i.e., P12Y13) (SEQ ID NO: 123) | GYTYFTTVTVEPYETQPGEE |
| FB-20 (T12M & L13R) (i.e., M12R13) (SEQ ID NO: 124) | GYTYFTTVTVEMRETQPGEE |
| FB-20 (T12I & L13F) (i.e., I12F13) (SEQ ID NO: 125) | GYTYFTTVTVEIFETQPGEE |
| FB-20 (T12HID & L13F) (i.e., H12F13) (SEQ ID NO: 126) | GYTYFTTVTVEHFETQPGEE |
| FB-20 (T12W & L13Y) (i.e., W12Y13) (SEQ ID NO: 127) | GYTYFTTVTVEWYETQPGEE |
| FB-20 (T12Q & L13R) (i.e., Q12R13) (SEQ ID NO: 128) | GYTYFTTVTVEQRETQPGEE |
| FB-20 (T12W & L13F) (i.e., W12F13) (SEQ ID NO: 129) | GYTYFTTVTVEWFETQPGEE |
| FB-20 (T12E & L13R) (i.e., E12R13) (SEQ ID NO: 130) | GYTYFTTVTVEERETQPGEE |
| FB-20 (T12D & L13Y) (i.e., D12Y13) (SEQ ID NO: 131) | GYTYFTTVTVEDYETQPGEE |
| FB-20 (T12F & L13F) (i.e., F12F13) (SEQ ID NO: 132) | GYTYFTTVTVEFFETQPGEE |
| FB-20 (T12HID & L13Y) (i.e., H12Y13) (SEQ ID NO: 133) | GYTYFTTVTVEHYETQPGEE |
| FB-20 (T12M & L13M) (i.e., M12M13) (SEQ ID NO: 134) | GYTYFTTVTVEMMETQPGEE |
| FB-20 (T12D & L13F) (i.e., D12F13) (SEQ ID NO: 135) | GYTYFTTVTVEDFETQPGEE |
| FB-20 (T12D & L13I) (i.e., D12I13) (SEQ ID NO: 136) | GYTYFTTVTVEDIETQPGEE |
| FB-20 (T12L & L13I) (i.e., L12113) (SEQ ID NO: 137) | GYTYFTTVTVELIETQPGEE |
| FB-20 (T12E & L131) (i.e., E12I13) (SEQ ID NO: 138) | GYTYFTTVTVEEIETQPGEE |
| FB-20 (T12A & L13F) (i.e., A12F13) (SEQ ID NO: 139) | GYTYFTTVTVEAFETQPGEE |
| FB-20 (T12HID & L13HIP) (i.e., H12H13) (SEQ ID NO: 140) | GYTYFTTVTVEHHETQPGEE |
| FB-20 (T12P & L13F) (i.e., P12F13) (SEQ ID NO: 141) | GYTYFTTVTVEPFETQPGEE |
| FB-20 (T12D & L13W) (i.e., D12W13) (SEQ ID NO: 142) | GYTYFTTVTVEDWETQPGEE |

As shown in FIG. 21B, LRRC4B peptide fragments comprising certain T12/L13 double mutants (e.g., T12P-L13Y and T12I-L13F) exhibited increased binding affinity to FAM19A5 protein.

The above results further confirm that certain amino acid residues (e.g., Y2 to E11) of the LRRC4B peptide fragment are important in binding to FAM19A5 protein. The above results further demonstrate that the binding affinity of the LRRC4B peptide fragments can be improved, e.g., by mutating amino acid residues that do not naturally plan an important role in binding, and thereby, help stabilize the interaction between the polypeptides described herein (which comprise the FAM19A5 binding domain of members of the LRRC4 protein family) and FAM19A5 protein.

### Example 10: Binding Affinity Analysis of Various FB-21 Peptide Mutants

The in silico analysis provided in Example 9 highlighted that certain T12 and L13 double mutants might be important in improving the binding affinity of the polypeptides of the present disclosure to the FAM19A5 protein. Therefore, the ability of both the wild-type FB-21 peptide (which is the same as the FB-20 peptide described herein except that the FB-21 peptide additionally contains an alanine at the C-terminus) and several FB-21 mutants were tested for their inhibitor effect on hFc-fused hLRRC4B and FAM19A5 complex formation. Specifically, the sequences for the different FB-21 peptide fragments tested are provided in Table 13 (below). Briefly, plate was coated with 100 nM of LRRC4B(453-576, TT/TT)-hFc and then 1 ng/ml of rFAM19A5 was incubated at 37° C. in the presence of increasing concentrations of the different FB-21 peptide fragments (0.03, 0.1, 0.3, 1, 3, 10, 30, 100, 300 and 1000 nM). The LRRC4B-bound FAM19A5 levels were measured using HRP-conjugated 1-65 antibody.

TABLE 13
Sequences for Exemplary
FB-21 Peptide Fragments

| Peptide | Amino Acid Sequence |
|---|---|
| FB-21 (wild-type) | GYTYFTTVTVETLETQPGEEA |
| (SEQ ID NO: 143) | |
| FB-21 (P12Y13) | GYTYFTTVTVEPYETQPGEEA |
| (SEQ ID NO: 144) | |
| FB-21 (H12F13) | GYTYFTTVTVEHFETQPGEEA |
| (SEQ ID NO: 145) | |
| FB-21 (Q12R13) | GYTYFTTVTVEQRETQPGEEA |
| (SEQ ID NO: 146) | |
| FB-21 (W12Y13) | GYTYFTTVTVEWYETQPGEEA |
| (SEQ ID NO: 147) | |
| FB-21 (M12R13) | GYTYFTTVTVEMRETQPGEEA |
| (SEQ ID NO: 148) | |
| FB-21 (I12F13) | GYTYFTTVTVEIFETQPGEEA |
| (SEQ ID NO: 149) | |

As shown in FIG. 22A (and in agreement with the data provided in Example 9), several of the FB-21 peptide mutants tested were able to inhibit the interaction between hFc-fused hLRRC4B protein and FAM19A5 protein (see Table 14 for IC50 of inhibition). For instance, compared with the wild-type FB-21, the ability of the FB-21 (W12Y13) mutant to dissociate the LRRC4B-FAM19A5 complex was increased by 2.9-folds.

TABLE 14
Inhibition (IC50) on Complex Formulation
between human LRRC4B protein and
recombinant FAM19A5 protein (1 ng/mL)

| Peptide | IC50 (nM) |
|---|---|
| FB-21 (wild-type) | 11.9 |
| (SEQ ID NO: 143) | |
| FB-21 (P12Y13) | 39 |
| (SEQ ID NO: 144) | |
| FB-21 (H12F13) | 9.8 |
| (SEQ ID NO: 145) | |
| FB-21 (Q12R13) | 39.9 |
| (SEQ ID NO: 146) | |
| FB-21 (W12Y13) | 4.1 |
| (SEQ ID NO: 147) | |
| FB-21 (M12R13) | 47.9 |
| (SEQ ID NO: 148) | |
| FB-21 (I12F13) | 9.9 |
| (SEQ ID NO: 149) | |

To further assess the inhibitory effect of the FB-21 mutants, the ability of additional FB-21 mutants described in Example 9 were tested for their ability to inhibit the interaction between FAM19A5 protein and LRRC4B protein. Briefly, plates were coated with 100 nM of His-TEV LRRC4B and then 1 ng/mL of rFAM19A5 was incubated at 37° C. in the presence of increasing concentrations of the FB-21 peptide fragments (0.3, 1, 3, 10, 30, 100, 300, 1000, 3000 and 10000 nM). The LRRC4B-bound FAM19A5 levels were measured using HRP-conjugated 1-65 SS01 antibody. As shown in FIG. 22B (see Table 15 for IC50), most (if not all) of the FB-21 mutants had improved ability in inhibiting the interaction between LRRC4B and recombinant FAM19A5 protein. For instance, the FB-21 (D12Y13) mutant was 2.4-fold and 7-fold more effective in dissociating LRRC4B-FAM19A5 complex formation, as compared to FB-21 and FB-21 (W12Y13), respectively.

TABLE 15
Inhibition (IC50) on Complex Formulation
between anti-FAM19A5 antibody and
recombinant FAM19A5 protein (1 ng/ml)

| Peptide | IC50 (nM) |
|---|---|
| FB-21 (wild-type) | 138.1 |
| (SEQ ID NO: 143) | |
| FB-21 (W12Y13) | 46.7 |
| (SEQ ID NO: 147) | |
| FB-21 (D12Y13) | 19.2 |
| (SEQ ID NO: 131) | |
| FB-21 (F12F13) | 31.5 |
| (SEQ ID NO: 132) | |
| FB-21 (H12Y13) | 39.6 |
| (SEQ ID NO: 133) | |
| FB-21 (D12F13) | 21.2 |
| (SEQ ID NO: 135) | |
| FB-21 (D12I13) | 21.8 |
| (SEQ ID NO: 136) | |

Next, to assess whether other properties of the polypeptides described herein can be improved (e.g., solubility, prevention of degradation from protease and peptidase, and in vivo administration), the following additional FB-21 peptide mutants were constructed and tested for their ability to inhibit the interaction between FAM19A5 protein and LRRC4B: (1) d-form FB-21 peptide (“dFB-21”), (2) d-form FB-21 peptide with juxta-membrane (JM) sequence (“dFB-JM-31”), (3) d-form FB-21 peptide with BBB penetrating sequence at each end of the sequence (“dFB-BBB-39”), and (4) d-form FB-21 mutant peptide with DY replacement and additional JM sequence (“dFB-DY-JM31”). The sequence for d-form FB-21 peptide is set forth in SEQ ID NO: 153 (nYTYFTTVTVETLETQPGEEa; wherein the lowercase amino acids represent D-form of the amino acid, and the uppercase amino acids represent L-form of the amino acid). The sequence for dFB-BBB-39 is set forth in SEQ ID NO: 154 (nYTYFTTVTVETLETQPGEEALRKLRKRLLLRKLRKRLl; wherein the lowercase amino acids represent D-form of the amino acid, and the uppercase amino acids represent L-form of the amino acid). The sequence for dFB-JM-31 is set forth in SEQ ID NO: 155 (nYTYFTTVTVETLETQPGEEALDEVMKTTKa; wherein the lowercase amino acids represent D-form of the amino acid, and the uppercase amino acids represent L-form of the amino acid). The sequence for dFB-DY-JM31 is set forth in SEQ ID NO: 156 (nYTYFTTVTVEDYETQPGEEALDEVMKTTKa; wherein the lowercase amino acids represent D-form of the amino acid, and the uppercase amino acids represent L-form of the amino acid). The overall experimental methods were the same as those described immediately above. As shown in FIG. 22D, the JM sequence is a conserved motif in the juxta-membrane region of the LRRC4 family gene. As shown in FIG. 22C, the FB-21 containing JM-motif including mutant peptide, which was substituted from T12L13 to D12Y13, was more effective in inhibiting complex formation by 4-fold compared to the wild-type FB-21.

The above results confirm that certain modifications described herein (e.g., amino acid substitutions at T12 and L13 residues of the FAM19A5 binding domain of members of the LRRC4 family; and addition of the juxta-membrane motif of the LRRC4 family members) can improve the ability of the polypeptides of the present disclosure on inhibiting the interaction between FAM19A5 protein and members of the LRRC4 protein family.

### Example 11: Effect of Polypeptides Comprising the FAM19A5 Binding Domain of LRRC4 Protein Family Members on Amyloid Beta-Induced Synapse Loss

Alzheimer's disease (AD) is tightly associated with the dysmetabolism of amyloid-β (Aβ). To begin assessing whether the polypeptides provided herein (i.e., comprising the FAM19A5 binding domain of LRRC4 protein family members) could have any therapeutic effects on AD, the effect of various FB-21 peptide fragments described herein on the deformation of synapse induced by toxic Aβ oligomer and following structural recovery was assessed. Specifically, the following FB-21 peptide fragments were tested: FB-21, FB-JM-31 and FB-BBB-39 (see Example 10).

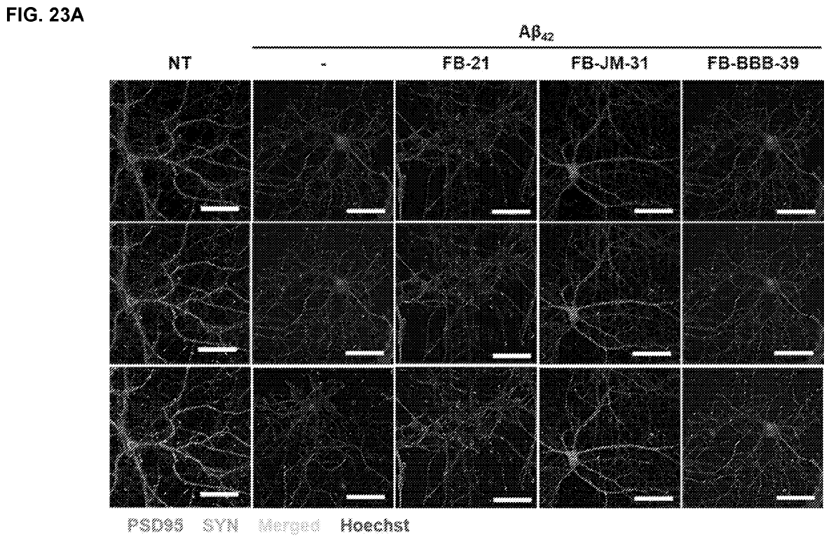

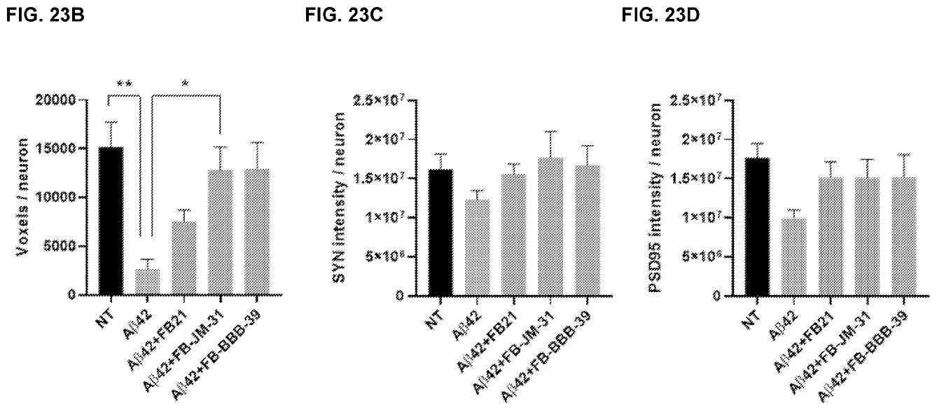

As shown in FIG. 23B, only FB peptide with JM sequence with significant increase in colocalized voxels for PSD95 and synaptophysin when co-treated with toxic Aβ oligomer. Additionally, no significant changes in intensity were observed for PSD95 and synaptophysin (see FIGS. 23C and 23D). These results highlight the structural preservation and neuroprotective property of the polypeptides provided herein (i.e., comprising the FAM19A5 binding domain of LRRC4 protein family members) against toxic Aβ oligomers.

### Example 12: Effect of Polypeptides Comprising the FAM19A5 Binding Domain of LRRC4 Protein Family Members on Neurite Outgrowth

A spinal cord injury (SCI) is damage to the spinal cord which causes temporary or permanent changes in its motor and/or sensation and/or autonomic function in the parts of the body served by the spinal cord below the level of the injury. In most cases the damage results from physical trauma such as falls, car accidents, or sports injuries, but it can also result from nontraumatic causes such as infection or tumors. To evaluate the regenerative capacity of the motor neurons after injury such as SCI, the therapeutic effect of the polypeptides described herein on spinal motor neurons was assessed. Briefly, mouse spinal motor neurons sampled at postnatal day 1 were treated with 10 nM of the FB-21 peptide fragments (dFB-dWY-JM31 and dFB-DY-JM31) at 1 and 2 DIV and immunostained with Tau-5 antibody at 3 DIV. Non-treated cells (“NT”) were used as control.

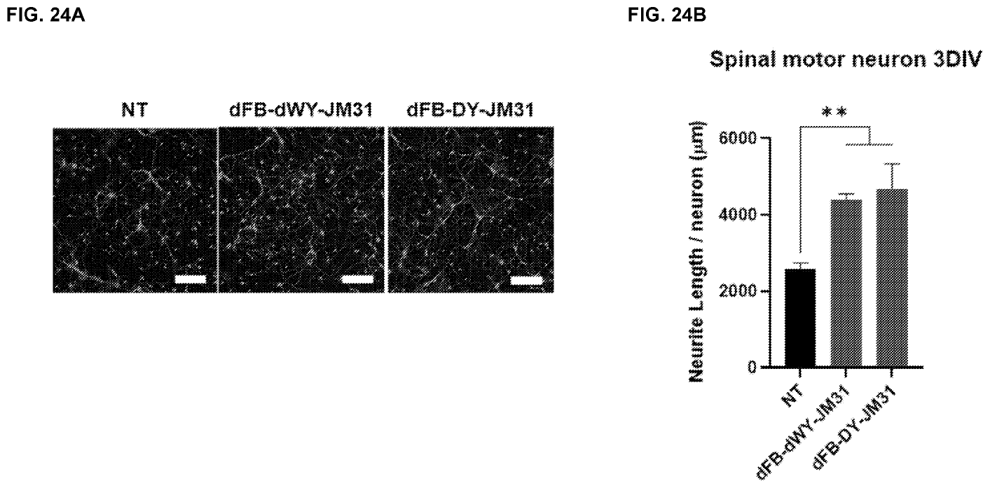

As shown in FIGS. 24A and 24B, in SCI-induced mice treated with a LRRC4B peptide fragment described herein, an increase in total neurite length of spinal motor neurons was observed. Such results further highlight the therapeutic potential of the polypeptides described herein (i.e., comprising the FAM19A5 binding domain of LRRC4 protein family members), including for the treatment of SCI.

### Example 13: Effect of Polypeptides Comprising the FAM19A5 Binding Domain of LRRC4 Protein Family Members on 6-OHDA Induced Cell Death

Parkinson's disease (PD) is a long-term degenerative disorder of the central nervous system which mainly affects the motor system via degeneration of dopaminergic neurons. To evaluate the possible neuro-protective capability on PD, the effect of the polypeptides described herein on neurodegeneration and cell death of dopaminergic neurons (often seen in PD) was assessed. Briefly, Lund human mesencephalic (LUHMES) cells were differentiated into dopaminergic neurons and treated with 6-OHDA (a known neurotoxin which induces PD-like degeneration of dopaminergic neurons) alone or in combination with varying doses (10, 30, and 100 nM) of a FB-21 peptide fragment (dFB-dWY-JM31) for 12 hours. Some of the LUHMES cells were treated with the FB-21 peptide fragment alone (i.e., no 6-OHDA treatment). Then, luminescence expression was measured using CellTiter-Glo assay.

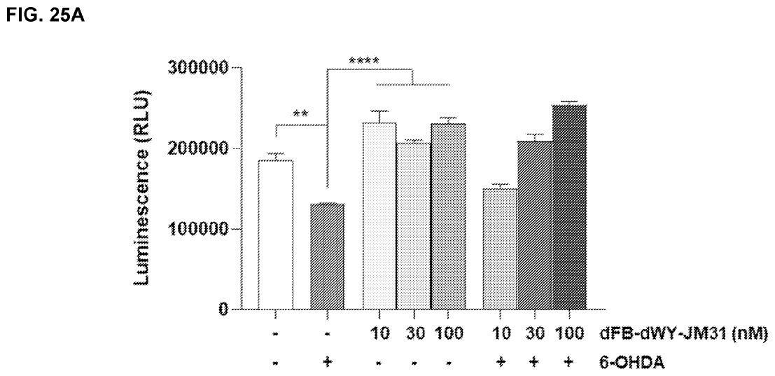

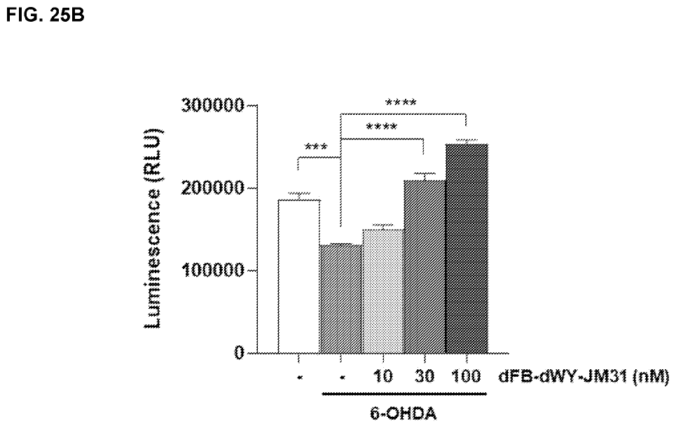

As shown in FIG. 25, treatment of 6-OHDA with the FB-21 peptide (dFB-dWY-JM31) showed a dose-dependent reversal of LUHMES cell viability, highlighting the potential use of the polypeptides described herein (i.e., comprising the FAM19A5 binding domain of a LRRC4 protein family member) as a novel therapeutic for the treatment of PD.

### Example 14: Effect of Polypeptides Comprising the FAM19A5 Binding Domain of LRRC4 Protein Family Members on Neuropathic Pain

Neuropathic pain is an intractable disease caused by damage or injury to the nerves of peripheral and central nervous system. The pain is usually described as a burning sensation and affected areas are often sensitive to the touch. In order to evaluate the analgesic effect of the polypeptides described herein under neuropathic condition, a chronic constriction injury (CCI) animal model was used. Briefly, after CCI induction, the animals were intrathecally injected with vehicle or 50 μg of FB-21 peptide variant (dFB-dDY-JM31) twice a week for five times. Then, mechanical allodynia was measured by Von Frey test at days 8, 11, 15, and 18 post CCI induction.

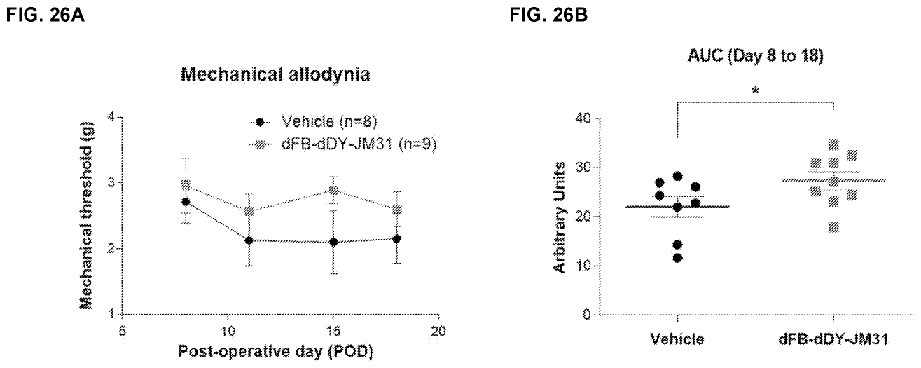

As shown in FIG. 26A, in CCI-induced mice treated with the FB-21 peptide fragment (dFB-dDY-JM31), there was an increased paw withdrawal threshold (PWT) at all time points assessed as compared to the vehicle group. The significant difference between the treated and control animals was even more apparent when the overall result was converted to Area Under Curve (AUC) (see FIG. 26B). These results demonstrate that administration of the polypeptides provided herein (e.g., dFB-dDY-JM31) could be useful in reverting mechanical allodynia induced by CCI, highlighting the potential use as an analgesic.

### Example 15: Effect of Polypeptides Comprising the FAM19A5 Binding Domain of LRRC4 Protein Family Members on Retinal Dysfunction and Modulation of Neural Oscillation

To evaluate the therapeutic effects of the polypeptides provided herein (i.e., comprising the FAM19A5 binding domain of LRRC4 protein family members) on retinal dysfunction (e.g., induced by diabetic retinopathy (DR)), the amplitude of bipolar cell and Muller cell responses-related b-wave was measured in transgenic diabetic model mice (db/db) via electroretinogram (ERG) test. Vehicle or 10 μg of dFB-dDY-JM31 was treated weekly via intravitreal (ivt) injection from 12 to 18 weeks.

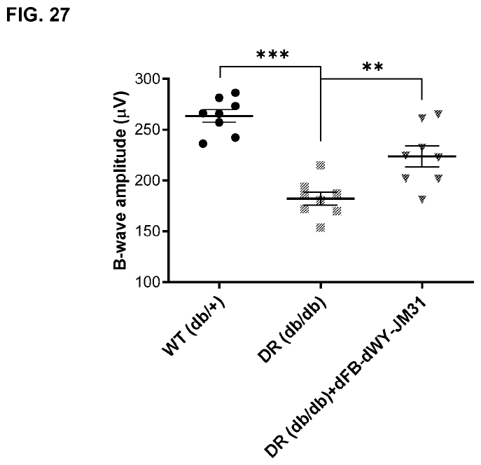

As shown in FIG. 27, db/db control animals exhibited significantly reduced B-wave amplitude as compared to the wild-type littermates (db/+). However, when the db/db control animals were treated with the FB-21 peptide fragment (dFB-dWY-JM31), there was a significant increase in the amplitude of B-wave. Such results highlight the therapeutic potential of the polypeptides described herein (i.e., comprising the FAM19A5 binding domain of LRRC4 protein family members) on retinal dysfunction, including that induced by DR.

### Example 16: Effect of Polypeptides Comprising the FAM19A5 Binding Domain of LRRC4 Protein Family Members on Brain Lesion Caused by Traumatic Brain Injury

To further evaluate the therapeutic effects of the polypeptides described herein, a mouse model of traumatic brain injury (i.e., cold-induced TBI) was used. Approximately 24 hours after TBI-induction, the animals were treated (via intranasal administration) with either a vehicle control or dFB-dWY-JM31 peptide (100 μg). Then, about 24 hours after treatment, brain tissues were obtained and stained with Hoechst.

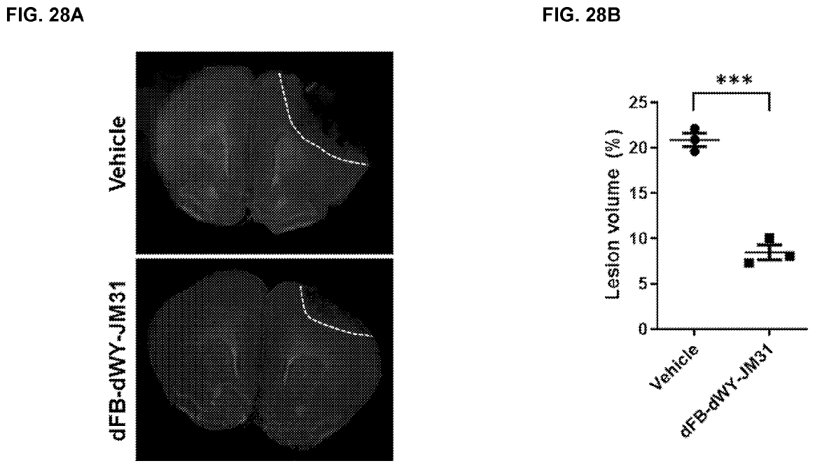

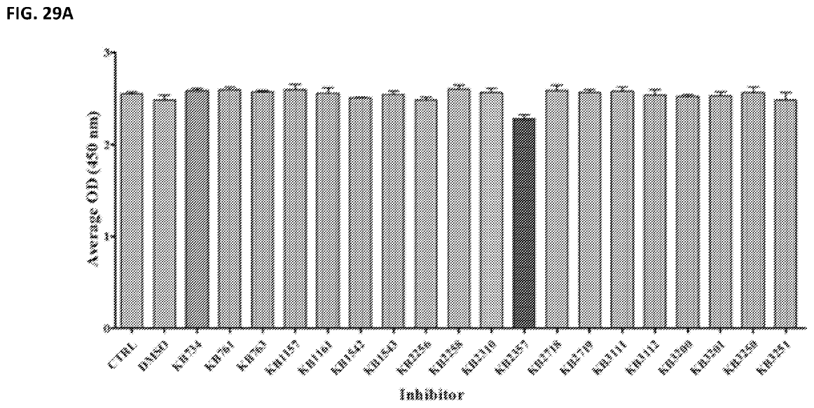

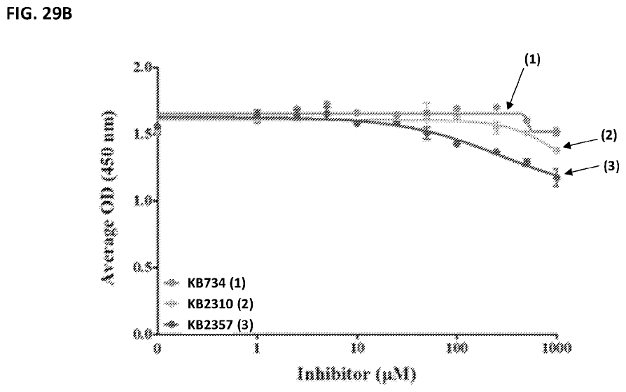

As shown in FIG. 28, compared to the TBI-control animals (i.e., treated with the vehicle control after TBI induction), the TBI animals treated with the FB-21 peptide fragment exhibited significantly decreased lesion volume. These results demonstrate that the polypeptides described herein (e.g., dFB-dWY-JM31) can reduce brain lesion size induced by TBI, showing potential as a therapeutic agent for TBI.

### Example 17: Development of Chemical Compounds that can Inhibit Interaction Between LRRC4 Protein Family Members and FAM19A5

To develop additional approaches to inhibiting, reducing, and/or dissociating the interaction between members of the LRRC4 protein family and FAM19A5, a series of scaffold chemical compounds were developed as described below.

Reagents and conditions: (i) tert-Butyldimethylsilyl chloride, imidazole, CH₂Cl₂, rt, 18 h; (ii) PPh₃, CBr₄, CH₂Cl₂, rt, 3 h; (iii) n-BuLi 1.6 M in hexanes (3.0 eq.), THF, rt, 2 h; (iv) n-BuLi 1.6 M in hexanes (1.1 eq.), N-methoxy-N-methylhexanamide, THF, −78° C. to rt, 16 h; (v) NaBH₄, MeOH, rt, 1 h; (vi) RuCl[(R,R)-TsDPEN(mesitylene)], KOH, 2-propanol, 4 h; (vii) RuCl[(S,S)-TsDPEN(mesitylene)], KOH, 2-propanol, 4 h; (viii) Lindlar cat., H₂ gas, 1,4-benzoquinone, MeOH, 0° C., 1 h; (ix) n-Bu₄NF, 0.1 M in THF, rt, 1 h.

Synthesis of 4-((tert-Butyldimethylsilyl)oxy)-3-methoxybenzaldehyde (compound 1): To a stirred solution of vanillin (1.00 g, 6.57 mmol) was dissolved in dry CH₂Cl₂ (50 mL) was added imidazole (1.29 g, 18.96 mmol) and tert-butyldimethylsilyl chloride (1.42 g, 9.39 mmol) at 0° C. The reaction mixture was stirred under argon for 16 h at room temperature, quenched with water, and extracted with CH₂Cl₂ (3×25 mL). The organic layer was washed with water, dried over MgSO₄, filtered, and concentrated under reduced pressure. The crude residue was purified by column chromatography on silica gel (Hexane/EtOAc=4:1, v/v) to furnish compound 17 in 94% yield as a colorless oil. Rf=0.89 (Hexane/EtOAc=1:1, v/v). ¹H NMR (600 MHZ, CDCl₃) δ 9.85 (s, 1H), 7.40 (d, J=1.9 Hz, 1H), 7.37 (dd, J=4.0 and 8.0 Hz, 1H), 6.97 (d, J=8.0 Hz, 1H), 3.87 (s, 3H), 1.00 (s, 9H), 0.20 (s, 6H); ¹³C NMR (150 MHz, CDCl₃) δ 191.19, 151.80, 151.51, 131.09, 126.40, 120.87, 110.25, 55.61, 25.74, 18.66, −4.41.

Synthesis of tert-Butyl(4-(2,2-dibromovinyl)-2-methoxyphenoxy)dimethylsilane (compound 2): To a stirred solution of CBr₄ (3.37 g, 10.14 mmol) was dissolved in dry CH₂Cl₂ (30 mL) was slowly added PPh₃ (5.32 g, 20.28 mmol) at 0° C. After stirring under argon for 1 h at the same temperature, a solution of compound 1 (1.35 g, 5.07 mmol) in dry CH₂Cl₂ (10 mL) was slowly added over 10 min. The reaction mixture was stirred under argon for 2 h and extracted with CH₂Cl₂ (3×25 mL). The organic layer was washed with water, dried over MgSO₄, filtered, and concentrated under reduced pressure. The crude residue was purified by column chromatography on silica gel (Hexane/EtOAc=15:1, v/v) to furnish compound 2 in 98% yield as a colorless oil. R_{f}=0.87 (Hexane/EtOAc=8:1, v/v). ¹H NMR (600 MHZ, CDCl₃) δ 7.42 (s, 1H), 7.19 (d, J=2.0 Hz, 1H), 7.03 (dd, J=4.1 and 8.3 Hz, 1H), 6.84 (d, J=8.2 Hz, 1H), 3.83 (s, 3H), 1.02 (s, 9H), 0.19 (s, 6H); ¹³C NMR (150 MHz, CDCl₃) δ 150.62, 145.64, 136.59, 128.77, 121.96, 120.68, 112.03, 87.10, 55.51, 25.69, 25.66, 18.47, −4.58.

Synthesis of tert-Butyl(4-ethynyl-2-methoxyphenoxy)dimethylsilane (compound 3): To a stirred solution of compound 2 (1.41 g, 3.34 mmol) in dry THF (20 mL) was added n-BuLi (1.6 M in hexanes, 5.30 mL, 8.35 mmol) at −78° C. The reaction mixture was stirred under argon for 2 h at the same temperature, quenched with aqueous NH₄Cl (10 mL) and extracted with EtOAc (3×25 mL). The organic layer was washed with water, dried over MgSO₄, filtered, and concentrated under reduced pressure. The crude residue was purified by column chromatography on silica gel (Hexane/EtOAc=16:1 to 10:1, v/v) to furnish compound 3 in 96% yield as a colorless oil. R_{f}=0.80 (Hexane/EtOAc=8:1, v/v). ¹H NMR (600 MHz, CDCl₃) δ 7.00 (dd, J=4.0 and 8.1 Hz, 1H), 6.97 (d, J=1.9 Hz, 1H), 3.80 (s, 3H), 2.99 (s, 1H), 0.99 (s, 9H), 0.15 (s, 6H); ¹³C NMR (150 MHz, CDCl₃) δ 150.66, 146.23, 125.46, 120.90, 115.66, 115.04, 83.96, 75.57, 55.45, 25.67, 18.47, −4.64.

Synthesis of 1-(4-((tert-Butyldimethylsilyl)oxy)-3-methoxyphenyl)oct-1-yn-3-one (compound 4): To a stirred solution of compound 3 (400 mg) in THF (40 mL) was added n-BuLi (1.1 eq.) at −78° C. The solution was stirred under argon for 1 h at the same temperature then N-methoxy-N-methylhexanamide (364 mg, 2.28 mmol) was added dropwise. The reaction mixture was stirred under argon at the same temperature until TLC analysis indicated complete conversion (12 h), quenched with aqueous NH₄Cl (10 mL), and extracted with EtOAc (3×25 mL). The organic layer was washed with brine, dried over MgSO₄, filtered, and concentrated under reduced pressure. The crude residue was purified by column chromatography on silica gel (Hexane/ether=120:1 to 80:1, v/v) to furnish compound 4. R_{f}=0.75 (Hexane/EtOAc=8:1, v/v). ¹H NMR (600 MHZ, CDCl₃) δ 7.12 (d, J=8.1 Hz, 1H), 7.07 (s, 1H), 6.84 (d, J=8.2 Hz, 1H), 3.82 (s, 3H), 2.65 (t, J=7.4 Hz, 2H), 1.77-1.75 (m, 2H), 1.38-1.37 (m, 4H), 1.00 (s, 9H), 0.93 (t, J=6.5 Hz, 3H), 0.18 (s, 6H); ¹³C NMR (150 MHz, CDCl₃) δ 188.36, 150.90, 148.19, 127.18, 121.14, 116.48, 112.74, 91.88, 87.48, 55.51, 45.41, 31.21, 25.62, 23.99, 22.43, 18.48, 13.92, −4.59.

Synthesis of 1-(4-((tert-Butyldimethylsilyl)oxy)-3-methoxyphenyl)oct-1-yn-3-ol (compound 5): To a stirred solution of 4 (210 mg, 0.582 mmol) in MeOH 15 mL was added NaBH₄ (33.0 mg, 0.874 mmol) at 0° C. The reaction mixture was stirred under argon for 1 h at the room temperature. The reaction mixture was concentrated, then extracted with EtOAc (3×25 mL). The organic layer was washed with water, dried over MgSO₄, filtered, and concentrated under reduced pressure. The crude residue was purified by column chromatography on silica gel (Hexane/EtOAc=8:1, v/v) to furnish compound 5 in 77% yield as colorless oil. R_{f}=0.59 (Hexane/EtOAc=4:1, v/v). ¹H NMR (600 MHZ, CDCl₃) δ 6.94-6.92 (m, 2H), 6.77 (d, J=7.9 Hz, 1H), 4.58 (q, J=6.4 Hz, 1H), 3.79 (s, 3H), 1.92 (d, J=5.2 Hz, 1H), 1.81-1.76 (m, 2H), 1.53-1.49 (m, 2H), 1.35-1.34 (m, 4H), 0.98 (s, 9H), 0.91 (t, J=7.0 Hz, 3H), 0.15 (s, 6H); ¹³C NMR (150 MHz, CDCl₃) δ 150.66, 145.84, 124.96, 120.90, 115.69, 115.31, 88.72, 85.01, 63.08, 55.44, 37.97, 31.51, 25.68, 24.95, 22.60, 18.47, 14.04, −4.65.

Synthesis of ((Z)-1-(4-((tert-butyldimethylsilyl)oxy)-3-methoxyphenyl)oct-1-en-3-ol (compound 6): To a solution of 5 (70.0 mg, 0.19 mmol) in MeOH (3 mL) was added quinoline (7 mg, 0.06 mmol) and Lindlar catalyst (7 mg, 0.02 mmol) and the mixture was stirred at 0° C. for 1 h under an atmosphere of hydrogen (balloon). After the complete conversion of compound 5 (TLC, toluene/EtOAc=10:1, v/v), the reaction mixture was filtered through a pad of Celite, and washed with MeOH (3×5 mL). The combined filtrates were concentrated under reduced pressure to give the crude product, which was purified by silica gel column chromatography (toluene/EtOAc=10:1, v/v) to furnish compound 6 as colorless oil. R_{f}=0.36 (toluene/EtOAc=10:1, v/v). ¹H NMR (600 MHz, CDCl₃) δ 6.83 (d, J=1.8 Hz, 1H), 6.81 (d, J=8.4 Hz, 1H), 6.77 (dd, J=1.8 and 8.1 Hz, 1H), 6.46 (d, J=11.4 Hz, 1H), 5.57 (dd, J=9.0 and 12.0 Hz, 1H), 4.60 (dd, J=7.2 and 15.3 Hz, 1H), 3.80 (s, 3H), 1.69-1.62 (m, 1H), 1.61 (brs, 1H), 1.59-1.52 (m, 1H), 1.46-1.21 (m, 6H), 1.00 (s, 9H), 0.88 (t, J=6.6 Hz, 3H), 0.16 (s, 6H).

Synthesis of (Z)-4-(3-Hydroxyoct-1-en-1-yl)-2-methoxyphenol (compound 7, “KB2356”): To a stirred solution of compound 6 in THF (5 mL) was added tetrabutylammonium fluoride solution (1M in THF, 2.0 eq.) at 0° C. The reaction mixture was stirred under argon at the same temperature until TLC analysis indicated complete conversion (typically 1 h), quenched with aqueous NH₄Cl (10 mL), and extracted with EtOAc (3×25 mL). The organic layer was washed with brine, dried over MgSO₄, filtered, and concentrated under reduced pressure. The crude residue was purified by column chromatography on silica gel (Hexane/EtOAc) to furnish compound 7 in 70% yield (for two steps from compound 5) as a colorless oil. R_{f}=0.21 (toluene/EtOAc=10:1, v/v). ¹H NMR (600 MHZ, CDCl₃) δ 6.89 (d, J=8.4 Hz, 1H), 6.88-6.86 (m, 1H), 6.85-6.81 (m, 1H), 6.48 (d, J=11.4 Hz, 1H), 5.64 (s, 1H), 5.57 (dd, J=9.0 and 11.7 Hz, 1H), 4.56-4.51 (m, 1H), 3.90 (s, 3H), 1.71-1.63 (m, 1H), 1.61-1.54 (m, 1H), 1.52 (brs, 1H), 1.46-1.23 (m, 6H), 0.88 (t, J=6.6 Hz, 3H); ¹³C NMR (150 MHz, CDCl₃) δ 146.36, 145.14, 133.27, 131.25, 129.15, 122.29, 114.34, 111.50, 68.20, 56.01, 37.94, 31.94, 25.31, 22.74, 14.13. HRMS m/z calculated for C₁₅H₂₂O₃ [M-H]−: 249.1496. found: 249.1523. >95% purity.

General procedure for compounds 8 and 9: A 0.1 M solution of compound 4 in 2-propanol was added KOH and catalyst with the ratio of compound 4:catalyst:KOH=200:1:1.2. The reaction mixture was stirred under argon at room temperature until TLC analysis indicated complete conversion (typically 4 h) and concentrated under reduced pressure. The crude residue was purified by column chromatography on silica gel to furnish stereoselective compounds. The ee values were determined by the chiral HPLC analyses on a chiral column (CHIRALPAK IG, 10% ethanol in hexane).

Synthesis of (R)-1-(4-((tert-Butyldimethylsilyl)oxy)-3-methoxyphenyl)oct-1-yn-3-ol (compound 8): Compound 8 was prepared in 88% yield as colorless oil following the same procedure as described in the above general procedure with compound 4 (150 mg, 0.416 mmol) in 2-propanol (4.16 mL), RuCl[(R,R)-TsDPEN(mesitylene)] (1.29 mg, 2.08 μmol) and KOH (0.138 mg, 2.50 μmol). The crude residue was purified by column chromatography on silica gel (Hexane/EtOAc=12:1, v/v) to furnish compound 8. R_{f}=0.59 (Hexane/EtOAc=4:1, v/v). ¹H NMR (600 MHz, CDCl₃) δ 6.94-6.92 (m, 2H), 6.77 (d, J=7.9 Hz, 1H), 4.58 (q, J=6.4 Hz, 1H), 3.79 (s, 3H), 1.90 (d, J=5.5 Hz, 1H), 1.81-1.76 (m, 2H), 1.53-1.50 (m, 2H), 1.36-1.33 (m, 4H), 0.98 (s, 9H), 0.91 (t, J=7.0 Hz, 3H), 0.15 (s, 6H); ¹³C NMR (150 MHz, CDCl₃) δ 150.66, 145.84, 124.95, 120.90, 115.68, 115.30, 88.71, 85.02, 63.09, 55.44, 37.97, 31.51, 25.68, 24.95, 22.60, 18.47, 14.04, −4.65.

Synthesis of (S)-1-(4-((tert-Butyldimethylsilyl)oxy)-3-methoxyphenyl)oct-1-yn-3-ol (compound 9): Compound 9 was prepared in 68% yield as colorless oil following the same procedure as described in compound 8 with starting materials as follows; compound 4 (150 mg, 0.416 mmol) in 2-propanol (4.16 mL), RuCl[(S,S)-TsDPEN(mesitylene)] (1.29 mg, 2.08 μmol) and KOH (0.138 mg, 2.50 μmol). The crude residue was purified by column chromatography on silica gel (Hexane/EtOAc=12:1, v/v) to furnish compound 9. R_{f}=0.59 (Hexane/EtOAc=4:1, v/v). ¹H NMR (600 MHZ, CDCl₃) δ 6.94-6.92 (m, 2H), 6.77 (d, J=7.9 Hz, 1H), 4.58 (t, J=6.5 Hz, 1H), 3.79 (s, 3H), 1.91 (s, 1H), 1.81-1.76 (m, 2H), 1.53-1.49 (m, 2H), 1.35-1.34 (m, 4H), 0.98 (s, 9H), 0.91 (t, J=7.0 Hz, 3H), 0.15 (s, 6H); ¹³C NMR (150 MHz, CDCl₃) δ 150.66, 145.84, 124.95, 120.90, 115.68, 115.31, 88.71, 85.01, 63.08, 55.44, 37.97, 31.51, 25.68, 24.95, 22.60, 18.47, 14.04, −4.65.

General procedure for compounds 10 and 11: To a stirred solution of the silyl protected compound in THF (5 mL) was added tetrabutylammonium fluoride solution (1M in THF, 2.0 eq.) at 0° C. The reaction mixture was stirred under argon at the same temperature until TLC analysis indicated complete conversion (typically 1 h), quenched with aqueous NH₄Cl (10 mL), and extracted with EtOAc (3×25 mL). The organic layer was washed with brine, dried over MgSO₄, filtered, and concentrated under reduced pressure. The crude residue was purified by column chromatography on silica gel (Hexane/EtOAc) to furnish compound.

Synthesis of (R)-4-(3-Hydroxyoct-1-yn-1-yl)-2-methoxyphenol (compound 10, “KB2357”): Compound 10 (28.0 mg, 0.077 mmol) was prepared in 94% yield as a white oil, by following the general procedure. R_{f}=0.21 (Hexane/EtOAc=5:1, v/v). ¹H NMR (600 MHz, CDCl₃) δ 6.98 (dd, J=1.7 and 8.2 Hz, 1H), 6.92 (d, J=1.7 Hz, 1H), 6.84 (d, J=8.2 Hz, 1H), 5.72 (brs, 1H), 4.58 (q, J=6.4 Hz, 1H), 3.89 (s, 3H), 1.87-1.73 (m, 3H), 1.58-1.47 (m, 2H), 1.39-1.30 (m, 4H), 0.91 (t, J=7.2 Hz, 3H); ¹³C NMR (150 MHz, CDCl₃) δ 146.29, 146.12, 125.67, 114.46, 114.23, 113.93, 88.32, 84.97, 63.10, 55.98, 37.98, 31.50, 24.94, 22.59, 14.03. HRMS m/z calculated for C₁₅H₂₀O₃ [M-H]⁻: 247.1339. found: 247.1397. >95% purity.

Synthesis of (S)-4-(3-Hydroxyoct-1-yn-1-yl)-2-methoxyphenol (compound 23c, “KB2358”): Compound 23c (20.0 mg, 0.055 mmol) was prepared in 93% yield as a yellow oil, by following the general procedure. R_{f}=0.21 (Hexane/EtOAc=5:1, v/v). ¹H NMR (600 MHZ, CDCl₃) δ 6.97 (t, J=6.6 Hz, 1H), 6.92 (d, J=1.8 Hz, 1H), 6.84 (d, J=7.8 Hz, 1H), 4.58 (t, J=6.6 Hz, 1H), 3.88 (s, 3H), 1.89-1.76 (m, 2H), 1.63-1.55 (m, 2H), 1.43-1.31 (m, 4H), 0.90 (t, J=7.2 Hz, 3H); ¹³C NMR (150 MHz, CDCl₃) δ 146.30, 146.15, 125.65, 114.50, 114.21, 113.96, 88.34, 84.97, 63.09, 55.97, 37.97, 31.50, 24.95, 22.59, 14.03. HRMS m/z calculated for C₁₅H₂₀O₃ [M-H]⁻: 247.1339. found: 247.1415. >95% purity.

In particular, the following compounds were produced: (1) KB734, (2) KB761, (3) KB763, (4) KB1157, (5) KB1161, (6) KB1542, (7) KB1543, (8) KB2256, (9) KB2258, (10) KB2310, (11) KB2357, (12) KB2718, (13) KB2719, (14) KB3111, (15) KB3112, (16) KB3220, (17) KB3201, (18) KB3250, and (19) KB3251. Then, the ability of the above chemical compounds to inhibit the binding of LRRC4 protein family members to FAM19A5 protein was assessed using a competitive inhibition assay. Briefly, ELISA plates were coated with 100 nM of a Fc conjugated LRRC4B fragment (amino acids 453-576 of SEQ ID NO: 2; SEQ ID NO: 7). Then, recombinant FAM19A5 protein (1 ng/mL) was added to the plates in combination with one of the above chemical compounds (100 μM). Recombinant FAM19A5 protein alone (“CTRL”) or in combination with DMSO was used as control. The plates were incubated at 37° C. and then, the amount of FAM19A5 protein bound to the coated LRRC4B protein fragment was assessed using HRP-conjugated anti-FAM19A5 (1-65) antibody.

As shown in FIG. 29A, of the 19 different compounds tested, KB2357 had the greatest effect, as it was able to reduce the interaction between FAM19A5 and the coated LRRC4B protein fragment by about 10% compared to the controls. The other 18 compounds tested did not have any substantial effect compared to the controls.

Next, to assess whether increasing the concentration of the chemical compounds could improve the inhibitory effect, increasing concentrations of KB734, KB2310, and KB2357 compounds were subjected to the same competitive inhibition assay described above. KB734 and KB2310 were used as negative controls, as they did not inhibit the interaction between FAM19A5 and LRRC4B protein. As shown in FIG. 29B, increasing the concentration of the KB2357 compound resulted in greater reduction in the interaction between FAM19A5 and the LRRC4B protein fragment. Increasing the concentration of KB734 and KB2310 did not have a clear effect.

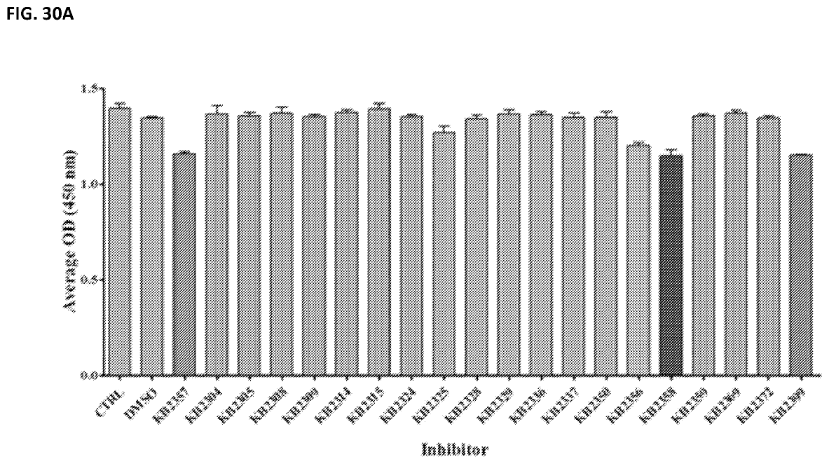

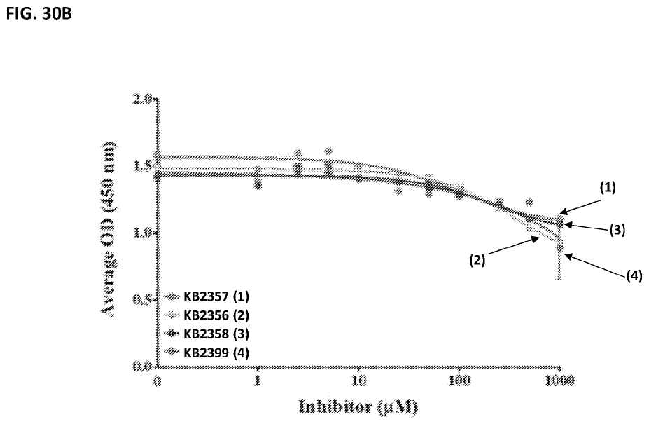

To further assess the inhibitory effect of the KB2357 chemical compound on FAM19A5 protein and members of the LRRC4 protein family, the following derivatives of the KB2357 compound were synthesized as described earlier: (1) KB2304, (2) KB2305, (3) KB2308, (4) KB2309, (5) KB2314, (6) KB2315, (7) KB2324, (8) KB2325, (9) KB2328, (10) KB2329, (11) KB2336, (12) KB2337, (13) KB2350, (14) KB2356, (15) KB2358, (16) KB2359, (17) KB2369, (18) KB2372, and (19) KB2399. The derivative compounds were also tested in the competitive inhibition assay as described above. As seen in FIGS. 30A and 30B, KB2356, KB2358, and KB2399 behaved similarly to KB2357, and were able to reduce the interaction in a dose-dependent manner between LRRC4 protein family members and FAM19A5.

The above results confirm that at least the KB2357 chemical compound and several of its derivatives (i.e., KB2356, KB2358, and KB2399) can be useful in inhibiting, reducing, and/or dissociating the interaction between members of the LRRC4 protein family and FAM19A5, particularly at higher concentrations (e.g., greater than or equal to about 100 μM).

### Example 18: Methods and Material

The examples provided herein (see above) use one or more of the following methods:

### Aβ42 Preparation

Aβ42 (#20276) peptide was purchased from AnaSpec (Fremont, USA). A lyophilized aliquot (1 mg) of Aβ42 peptide was dissolved in 80 μl of 1% NH₄OH and then in 920 μl of sterile phosphate-buffered saline (PBS) to get stock solution with concentration 1 mg/ml (stored as 100 μl aliquots at −20° C.). Working Aβ solutions were made one day prior to the treatment by diluting stock concentration to 100 nM final Aβ peptide concentrations in Neurobasal medium (Gibco, Life technologies, USA). Working solutions were incubated at 4° C. for 24 h to obtain the oligomeric conditions as described by Zheng et al., Amyloid 20(1): 13-20 (2013), which is incorporated herein by reference in its entirety. At the day of the usage working solutions were centrifuged at 14000 g, 4° C., 10 min to purify oligomeric Aβ fraction from fibrils.

### Primary Hippocampal Neuron Culture

Primary hippocampal neurons were prepared from postnatal pups (postnatal day 1) of C57BL/6 (Nara Biotech, Seoul, Korea) as previously described by Beaudoin et al., Nature protocols 7(9): 1741-1754 (2012), which is incorporated herein by reference in its entirety. Briefly, cortices were dissected in Hank's buffered salt solution (HBSS) (Invitrogen, Carlsbad, CA, USA) and digested with 2.5% trypsin for 15 min at 37° C. The supernatant was removed, and the tissues were washed with HBSS. The tissues were gently triturated, and the dissociated cells were seeded at 8×10⁵ cells per dish on poly-D-lysine-coated glass coverslips in a 60 mm culture dishes in minimum Eagle's medium (MEM) supplemented with 0.5% glucose, 1 mM pyruvate, 1.2 mM L-glutamine and 12% fetal bovine serum. Six hours after plating, the medium was replaced with Neurobasal media (Invitrogen, Carlsbad, CA, USA) supplemented with 2% B-27 and 0.5 mM L-glutamine. Cells were maintained at 37° C. in a 5% CO₂-humified incubator. Neurobasal media was half-changed every 3-4 days.

### Primary Spinal Motor Neuron Culture

Primary spinal motor neurons were prepared from postnatal pups (postnatal day 1) of C57BL/6 (Nara Biotech, Seoul, Korea) as previously described by Eldeiry et al., JoVE (Journal of Visualized Experiments) 125: 255856 (2017), which is incorporated herein by reference in its entirety. Briefly, spinal cord was dissected in Dulbecco's phosphate-buffered saline (DPBS) (Gibco, Life technologies, USA). and digested with papain (2.5 mg/ml) for 30 min at 30° ° C. The supernatant was centrifuged and removed, and the tissues were washed with Hibernate A (Gibco, Life technologies, USA) supplemented with 2% B-27 and 0.5 mM L-glutamine. The tissues were gently triturated, and the dissociated cells were seeded at 3×10⁵ cells per well on poly-D-lysine and laminin (Thermofisher scientific, USA) coated glass coverslips in a 12-well plate in Neurobasal media (Invitrogen, Carlsbad, CA, USA) supplemented with 2% B-27 and 0.5 mM L-glutamine. Cells were maintained at 37° C. in a 5% CO₂-humified incubator.

### Immunostaining

Primary neurons were fixed with 4% paraformaldehyde (PFA) at appropriate DIV. The cells were blocked with 3% bovine serum albumin (BSA) and 0.1% Triton X-100 in phosphate-buffered saline (PBS) for 1 h at room temperature. Primary antibodies were then applied to the cells overnight at 4° C. Primary antibodies used in this study were mouse anti-Tau5 (Invitrogen, California, United States), rabbit anti-PSD95 (Invitrogen), and mouse anti-synaptophysin (Sigma). After several washes with PBS, appropriate fluorescent conjugated secondary antibodies were applied with Hoechst 33342 (Invitrogen) for 30 min at room temperature. Subsequently, cell images were obtained using a confocal microscope (Leica, Wetzlar, Germany).

### Quantitative Analysis of Synaptogenesis

Hippocampal neurons were treated with 6.6 nM FB-21, 6.6 nM of FB-13-JM and 6.6 nM of FB-13-BBBX2 at 14, 17 and 20 DIV to determine the level of synaptogenesis at 21 DIV by immunostaining SYP, a presynapse marker protein and PSD95, a postsynapse marker protein. To quantify fluorescence intensity for SYN and PSD95 and number of colocalized voxels between SYN and PSD95 signals, z-stack confocal images of 3 μm in depth were converted to 3D images using IMARIS software (IMARIS9.0, Bitplane AG, Zurich, Switzerland). The “Surface tool” of the IMARIS software was used to exclude all the signals detected in neuronal cell bodies, and number of colocalized voxels between SYN and PSD95 signals in neurites were calculated using the “Coloc tool”. Then, total fluorescence intensity for SYN and PSD95 in neurites were acquired.

### Quantitative Analysis of Neurite Outgrowth

Mouse hippocampal neurons were treated with LRRC4B peptides at 1 and 2 DIV to determine neurite growth. It was measured by using 3 different parameters, which are total neurites length, number of primary and secondary neurites. After the neurons were stained with beta-tubulin III at 3 DIV, neurites length and branch points were measured using Fiji (Image J, NIH, Bethesda). Individual neurons were selected by hand and these parameters were counted using the Simple neurite tracer plugin. Mouse spinal motor neurons sampled at postnatal day 1 were treated with 10 nM of NS101 and LRRC4B-peptides (dFB-dWY-JM31 and dFB-DY-JM31) at 1 and 2 DIV and immunostained with Tau-5 antibody at 3 DIV. Total neurite length and number of somas in images were measured by Neurphology Image J plugin.

### LUHMES Cell Culture and Differentiation

LUHMES human neuronal precursor cells were obtained from ATCC (CRL 2927). As previously described by Harischandra et al., Biochimica et Biophysica Acta (BBA)-Molecular Basis of Disease 1866(4): 165533 (2020), which is incorporated herein by reference in its entirety. Briefly, plastic culture plates were pre-coated with 50 μg/mL poly-l-ornithine (Sigma Aldrich) overnight, washed twice with cell culture grade water (Invitrogen) at the end of the incubation, and then incubated with 1 μg/mL fibronectin (Sigma) overnight. Cells were proliferated inside an CO₂ incubator maintained at 37° C. using proliferation medium composed of Advanced Dulbecco's modified Eagle's medium (DMEM)/F12, N-2 supplement (1×), 2 mM L-glutamine and 40 ng/ml recombinant basic fibroblast growth factor (bFGF) (Sigma). During proliferation, half of the media was changed every other day and cells were enzymatically dissociated with 0.025% trypsin and subcultured when cultures reached 80% confluency. Briefly, 3.5×106 cells were seeded in a pre-coated T75 flask in proliferation medium and incubated for 24 h. Differentiation was induced the next day by replacing the medium with freshly prepared differentiation medium and incubating for 48 h. The differentiation medium was composed of Advanced DMEM/F12, N-2 supplement (1×), 2 mM L-glutamine, 1 mM dibutyryl cAMP, 1 μg/mL tetracycline and 2 ng/mL recombinant human GDNF (R&D Systems). At the end of the 48-h incubation, cells were dissociated with 0.025% trypsin/EDTA and replated on pre-coated plates at a cell density of 1.5×10⁵ cells/cm² in differentiation medium. Once cells were replated, differentiation medium was changed every other day, and unless mentioned otherwise, all experiments were done on the fifth day of differentiation.

### Quantitative Analysis of Cell Viability

Differentiated LUHMES cell were treated with 10, 30, 100 nM dFB-dWY-JM31 to determine the level of cell viability against 5 μM 6-hydroxydopamine hydrobromide (Tocris) by CellTiter-Glo Luminescent Cell Viability Assay (Promega). To determine the level of cell viability, CellTiter-Glo reagent were added 1:1 ratio volume at cell culture medium present in each well and mix contents on an orbital shaker to induce cell lysis. Allow the plate to incubate at room temperature for 10 minutes to stabilize luminescent signal and luminescence signal was read in a microplate reader (Synergy H1, Biotek). Each experiment is performed three replicates.

### Chronic Constriction Injury (CCI)

CCI was performed on healthy subjects that are normal during the habituation period. The first surgery date was set at Day 0. SD rats were removed from the anesthesia chamber and fixed. After sterilizing the surgical site with Povidone (Betadine) and 70% alcohol, the skin of the left lower extremity was incised (0.5 cm behind the skin and about 3˜4 cm parallel to the femoral process). A small hole was made with forceps, and a curved needle holder was inserted into the hole to separate the sciatic nerve from the dullness. While observing with a microscope, the membrane (fascia) on both sides of the sciatic nerve was held with forceps and incised by a microscissor. The nerve was tied three times 1 mm apart with 4-0 suture.

### Measurement of Paw Withdrawal Threshold (PWT)

Each rat was habituated in a test environment for at least 30 minutes. To measure 50% paw withdrawal threshold (PWT), 0.4, 0.6, 1.0, 2.0, 4.0, 6.0, 8.0, and 15.0 g of von-frey filaments were used. 2.0 g of von-frey filament was applied to the hindlimb of CCI rats for 4-5 seconds. When the rat showed symptoms (raising one's feet or twitching), the scale-down of 2.0 g was applied to the rat. When the rat did not show any symptom, the scale-up of 2.0 g was applied to the rat. In this way, 0.4 g to 15.0 g of von-frey filaments were applied consequently. When the point that the changed response occurred (starting from 2.0 g of von-frey filament, the time when it started to react or when it started not reacting from 2.0 g of von-frey filament), 5 times more stimulations were applied to get a PWT.

Measurement of Electroretinogram (ERG) in a Mouse Model of Diabetic Retinopathy (db/db)

For evaluation of diabetic retinal neurodegeneration, db/db and db/+ mice were used. See, e.g., Bogdanov et al., PLOS One 9(5): e97302 (2014), which is incorporated herein by reference in its entirety. ERG was recorded to measure electrical signals emitted by the retina in response to flashes of light. Each mouse underwent ophthalmologic examination to test ERG according to the International Society for Clinical Electrophysiology of Vision standard, and each mouse was habituated for 12 hours in a dark chamber. After the injection (at 6 weeks and at 10 weeks), dark adaptation ERG was performed. ERG amplitudes of b-wave were measured and when the b-wave elicited by light intensity of −0.9 log cd·sm⁻² was compared between the groups. The analysis of ERG was carried out using the LabScribeERG (iWorx DataAcquisition Software) program.

### Cold-Induced Traumatic Brain Injury (TBI)

After anesthetizing the mice by isoflurane inhalant exposure, each mouse was steadily placed in a stereotaxic device. A 3.0 mm incision was made on the midline scalp. Cold-induced TBI was executed by applying the tip (2.5 mm) of liquid nitrogen-cooled (−80° C.) copper cylinder rod on the right frontal skull for 45 seconds to produce a cryogenic lesion. See, e.g., Keskin et al., Neural regeneration research 12(5): 761-764 (2017), which is incorporated herein by reference in its entirety. Forty-eight hours after trauma and twenty-four hours after vehicle or dFB-dWY-JM31 peptide injection via intranasal administration, all animals were sacrificed by cardiac perfusion.

### Quantitative Analysis of TBI Brain Lesion

The brains from traumatized mice were removed and brain sections were obtained as a total of 8-9 consecutive coronal sections (20 μm thick) throughout the brain, and they were stained with Hoechst (ThermoFisher, Waltham, MA, USA). The boundary for the injured and non-injured areas was distinguished by Image J software program (NIH, Bethesda, MD, USA). The area of injury was assessed by subtracting the area of the non-lesioned ipsilateral hemisphere from that on the contralateral side. The volume of injury was calculated by integrating these lesioned areas. All 8-9 cross sections were individually measured, and corresponding volumes were calculated.

### Statistical Analysis

All statistical analysis was performed using GraphPad Prism 5 (GraphPad Software Inc., California, United States), and the data are shown as the mean±standard error of the mean (SEM). Statistical significance was evaluated using Student's t-tests and/or one-way analysis of variance (ANOVA) with Bonferroni post hoc tests. A p-value of less than 0.05 was considered statistically significant.

It is to be appreciated that the Detailed Description section, and not the Summary and Abstract sections, is intended to be used to interpret the claims. The Summary and Abstract sections can set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, internet sites, and accession numbers/database sequences (including both polynucleotide and polypeptide sequences) cited herein are hereby incorporated by reference in their entirety for all purposes to the same extent as if each individual publication, patent, patent application, internet site, or accession number/database sequence were specifically and individually indicated to be so incorporated by reference.

What is claimed is:

1. A Leucine Rich Repeat Containing 4 (“LRRC4”) family mimic molecule that is capable of inhibiting, reducing, and/or dissociating an interaction between a Family with Sequence Similarity 19, Member A5 (“FAM19A5”) protein and a member of a LRRC4 protein family.

2. The LRRC4 family mimic molecule of claim 1, which is not an antibody or an antigen-binding portion thereof.

3. The LRRC4 family mimic molecule of claim 1 or 2, which comprises a peptide.

4. The LRRC4 family mimic molecule of claim 1 or 2, which is a small molecule.

5. The LRRC4 family mimic molecule of claim 4, which is a small molecule of formula (I): 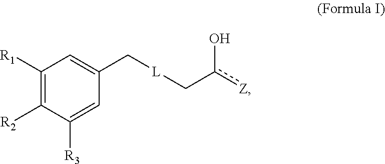
or a pharmaceutically acceptable salt thereof, wherein:
(i) R₁, R₂ and R₃ are independently selected from hydrogen, fluoro, chloro, bromo, methyl, ethyl, n-propyl, iso-propyl, n-butyl, iso-butyl, t-butyl, n-pentyl, iso-pentyl, fluoromethyl, difluoromethyl, trifluoromethyl, 2-fluoroethyl, 1-fluoroethyl, 2,2-difluoroethyl, 1,2-difluoroethyl, 1,1-difluoroethyl, 2,2,2-trifluoroethyl, methoxy, ethoxy, n-propyloxy, iso-propyloxy, n-butoxy, trifluoromethoxy, difluoromethoxy, fluoromethoxy, acetyl, propionyl, n-butanoyl, iso-butanoyl, n-pentanoyl, nitro, amino, N-methylamino, N-ethylamino, N-n-propylamino, N,N-dimethylamino, N-acetylamino, N-propionylamino, N-(trifluoroacetyl)amino, formyl, hydroxy, methylthio, ethylthio, n-propylthio, methylsulfonyl, ethylsulfonyl, n-propylsulfonyl, phenyl, hydroxymethyl, 1-hydroxyethyl and 2-hydroxyethyl;
(ii) [symbol] is a single or double bond;
(iii) Z is selected from a straight chain or branched (C₁-C₈)alkyl, a straight chain or branched (C₂-C₈)alkenyl, a straight chain or branched (C₂-C₈)alkynyl, (C₃-C₈)cycloalkyl, (C₅-C₈)cycloalkenyl, (3-8 membered) heterocycloalkyl, (C₇-C₁₄)bicycloalkyl, (C₇-C₁₄) bicycloalkenyl, (7-14 membered) heterobicycloalkyl, (C₆-C₁₀) aryl, (5-10-membered) heteroaryl, and —CH—C(O)—CH═CH-Q, wherein Q is (C₃-C₈)cycloalkyl, (C₅-C₈)cycloalkenyl, (3-8 membered)heterocycloalkyl, (C₆-C₁₀)aryl, and (5-6-membered)heteroaryl; wherein each cycloalkyl, cycloalkenyl, heterocyclylalkyl, aryl, and heteroaryl are optionally substituted with one, two, three, four, or five substituents independently selected from (C₁-C₆)alkoxy, (C₁-C₆)alkyl, halo, (C₁-C₆)haloalkoxy, nitro, amino, N-methylamino, N-ethylamino, N—N-propylamino, N,N-dimethylamino, formyl, and hydroxy, and
(iv) L is single, double or triple bond, and
wherein the LRRC4 family mimic molecule is not a small molecule selected from: 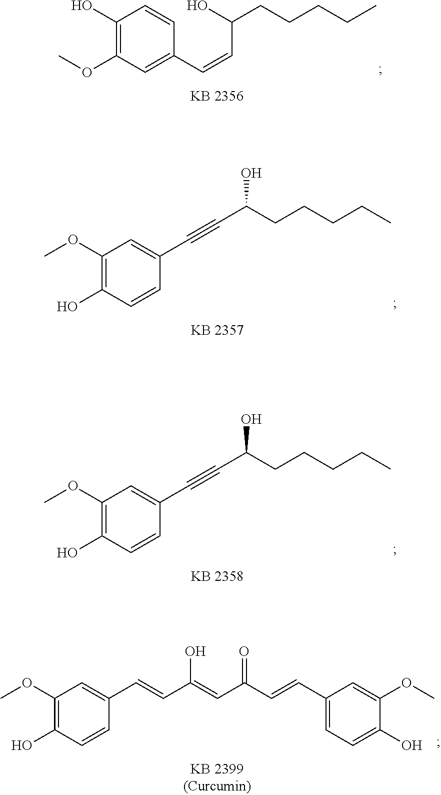
or a pharmaceutically acceptable salt thereof.

6. The LRRC4 family mimic molecule of claim 4, which is a small molecule of formula (II): 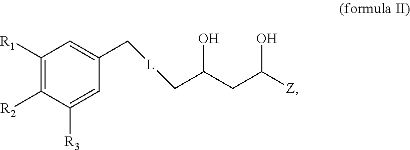
or a pharmaceutically acceptable salt thereof, wherein:
(i) R1, R2 and R3 are independently selected from hydrogen, fluoro, chloro, bromo, methyl, ethyl, n-propyl, iso-propyl, n-butyl, iso-butyl, t-butyl, n-pentyl, iso-pentyl, fluoromethyl, difluoromethyl, trifluoromethyl, 2-fluoroethyl, 1-fluoroethyl, 2,2-difluoroethyl, 1,2-difluoroethyl, 1,1-difluoroethyl, 2,2,2-trifluoroethyl, methoxy, ethoxy, n-propyloxy, iso-propyloxy, n-butoxy, trifluoromethoxy, difluoromethoxy, fluoromethoxy, acetyl, propionyl, n-butanoyl, iso-butanoyl, n-pentanoyl, nitro, amino, N-methylamino, N-ethylamino, N-n-propylamino, N,N-dimethylamino, N-acetylamino, N-propionylamino, N-(trifluoroacetyl)amino, formyl, hydroxy, methylthio, ethylthio, n-propylthio, methylsulfonyl, ethylsulfonyl, n-propylsulfonyl, phenyl, hydroxymethyl, 1-hydroxyethyl and 2-hydroxyethyl,
(ii) Z is selected from a straight chain or branched (C₁-C₈)alkyl, a straight chain or branched (C₂-C₈)alkenyl, a straight chain or branched (C₂-C₈)alkynyl, (C₃-C₈)cycloalkyl, (C₅-C₈)cycloalkenyl, (3-8 membered) heterocycloalkyl, (C₇-C₁₄)bicycloalkyl, (C₇-C₁₄) bicycloalkenyl, (7-14 membered) heterobicycloalkyl, (C₆-C₁₀) aryl, (5-10-membered) heteroaryl, and —CH═CH-Q, wherein Q is (C₃-C₈)cycloalkyl, (C₅-C₈)cycloalkenyl, (3-8 membered)heterocycloalkyl, (C₆-C₁₀)aryl, and (5-6-membered)heteroaryl; wherein each cycloalkyl, cycloalkenyl, heterocyclylalkyl, aryl, and heteroaryl are optionally substituted with one, two, three, four, or five substituents independently selected from (C₁-C₆)alkoxy, (C₁-C₆)alkyl, halo, (C₁-C₆)haloalkoxy, nitro, amino, N-methylamino, N-ethylamino, N—N-propylamino, N,N-dimethylamino, formyl, and hydroxy, and
(iii) L is single, double or triple bond.

7. The LRRC4 family mimic molecule of claim 6, which is selected from: 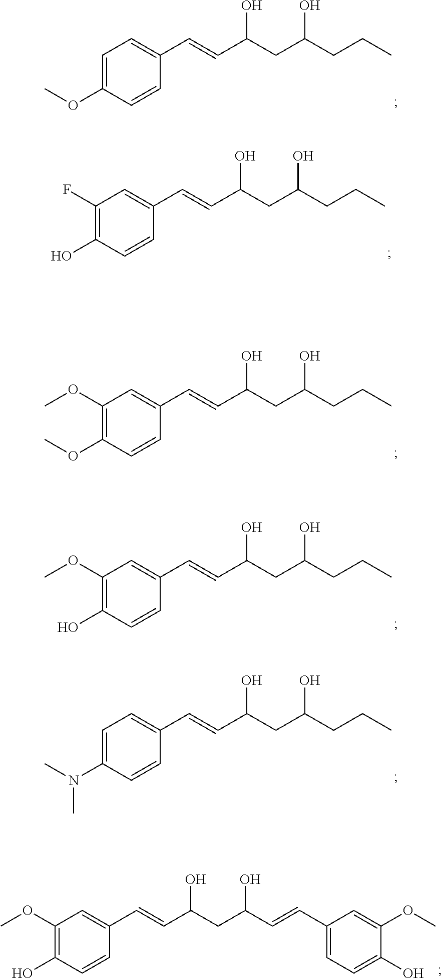
or a pharmaceutically acceptable salt thereof.

8. The LRRC4 family mimic molecule of claim 4, which is a small molecule of formula (III): 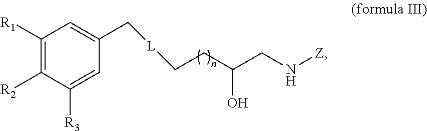
or a pharmaceutically acceptable salt thereof, wherein:
(i) R1, R2 and R3 are independently selected from hydrogen, fluoro, chloro, bromo, methyl, ethyl, n-propyl, iso-propyl, n-butyl, iso-butyl, t-butyl, n-pentyl, iso-pentyl, fluoromethyl, difluoromethyl, trifluoromethyl, 2-fluoroethyl, 1-fluoroethyl, 2,2-difluoroethyl, 1,2-difluoroethyl, 1,1-difluoroethyl, 2,2,2-trifluoroethyl, methoxy, ethoxy, n-propyloxy, iso-propyloxy, n-butoxy, trifluoromethoxy, difluoromethoxy, fluoromethoxy, acetyl, propionyl, n-butanoyl, iso-butanoyl, n-pentanoyl, nitro, amino, N-methylamino, N-ethylamino, N-n-propylamino, N,N-dimethylamino, N-acetylamino, N-propionylamino, N-(trifluoroacetyl)amino, formyl, hydroxy, methylthio, ethylthio, n-propylthio, methylsulfonyl, ethylsulfonyl, n-propylsulfonyl, phenyl, hydroxymethyl, 1-hydroxyethyl and 2-hydroxyethyl,
(ii) Z is selected from a straight chain or branched (C₁-C₈)alkyl, a straight chain or branched (C₂-C₈)alkenyl, a straight chain or branched (C₂-C₈)alkynyl, —Y—(C₃-C₈)cycloalkyl, —Y—(C₅-C₈)cycloalkenyl, —Y-(3-8 membered) heterocycloalkyl, —Y—(C₇-C₁₄)bicycloalkyl, —Y—(C₇-C₁₄) bicycloalkenyl, —Y-(7-14 membered) heterobicycloalkyl, —Y—(C₆-C₁₀)aryl, and —Y-(5-10-membered) heteroaryl, wherein Y is a bond or a C₁-C₃ straight or branched alkylene, and wherein the cycloalkyl, the cycloalkenyl, the heterocyclylalkyl, the aryl, and the heteroaryl are optionally substituted with one, two, three, four, or five substituents independently selected from C₁-C₆alkoxy, C₁-C₆alkyl, halo, C₁-C₆haloalkoxy, nitro, amino, N-methylamino, N-ethylamino, N-N-propylamino, N,N-dimethylamino, formyl, and hydroxy,
(iii) L is single, double or triple bond, and
(iv) n is 0 or 1.

9. The LRRC4 family mimic molecule of claim 8, which is selected from: 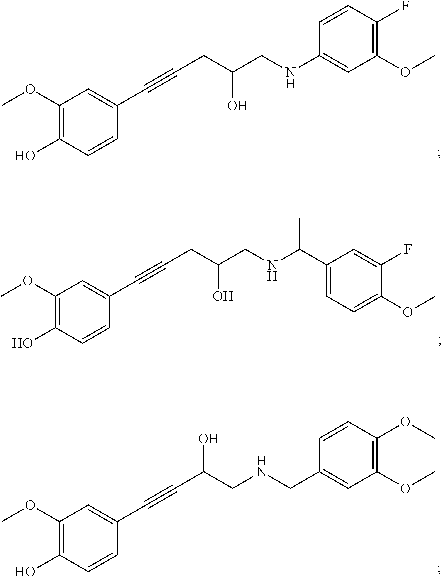
or a pharmaceutically acceptable salt thereof.

10. The LRRC4 family mimic molecule of any one of claims 1 to 3, which comprises a polypeptide comprising, consisting of, or consisting essentially of a domain of a LRRC4 protein family member, wherein the domain is capable of binding to a FAM19A5 protein (“FAM19A5 binding domain”), and wherein the polypeptide is shorter than the corresponding full-length LRRC4 protein family member (SEQ ID NO: 4; SEQ ID NO: 5; or SEQ ID NO: 6).

11. The LRRC4 family mimic molecule of claim 10, wherein the FAM19A5 binding domain is about 10 to about 23 amino acids in length.

12. The LRRC4 family mimic molecule of claim 11, wherein the FAM19A5 binding domain is about 10, about 11, about 12, about 13, about 14, about 15, about 16, about 17, about 18, about 19, about 20, about 21, about 22, or about 23 amino acids in length.

13. The LRRC4 family mimic molecule of claim 12, wherein the FAM19A5 binding domain is about 10 amino acids in length.

14. The LRRC4 family mimic molecule of any one of claims 10 to 13, wherein the polypeptide comprises an amino acid sequence having the following formula (from N-terminus to C-terminus): 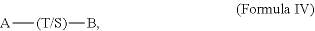
wherein:
(i) A comprises X1-(T/S)-(Y/F)-F-X5; wherein:
X1 is tyrosine (Y), phenylalanine (F), valine (V), leucine (L), or isoleucine (I);
(T/S) is threonine (T) or serine (S);
(Y/F) is tyrosine (Y) or Phenylalanine (F); and
X5 is any amino acid; and
(ii) B comprises (V/I)-T-V-(E/V); wherein:
(V/I) is valine (V) or isoleucine (I); and
(E/V) is glutamic acid (E) or valine (V).

15. The LRRC4 family mimic molecule of any one of claims 10 to 13, wherein the polypeptide comprises an amino acid sequence having the following formula (from N-terminus to C-terminus): 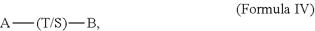
wherein:
(i) A comprises (Y/W/M)-(T/Y)-(Y/W)-(F/Y/W)-(T/Y); wherein:
(Y/W/M) is tyrosine (Y), tryptophan (W), or methionine (M);
(T/Y) is threonine (T) or tyrosine (Y);
(Y/W) is tyrosine (Y) or tryptophan (W); and
(F/Y/W) is phenylalanine (F), tyrosine (Y), or tryptophan (W); and
(ii) B comprises X7-(T/S/Y)-X9-X10; wherein:
X7 is valine (V), tyrosine (Y), phenylalanine (F), leucine (L), tryptophan (W), or methionine (M);
(T/S/Y) is threonine (T), serine (S), or tyrosine (Y);
X9 is valine (V), isoleucine (I), tyrosine (Y), phenylalanine (F), leucine (L), tryptophan (W), or methionine (M); and
X10 is glutamic acid (E), aspartic acid (D), isoleucine (I), tyrosine (Y), phenylalanine (F), methionine (M), or tryptophan (W).

16. A LRRC4 family mimic molecule comprising a polypeptide, which comprises an amino sequence having the following formula (from N-terminus to C-terminus): 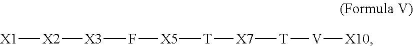
wherein:
X1 is Y, F, V, L, or I;
X2 is T or S;
X3 is Y or F;
X5 is any amino acid;
X7 is V or I; and/or
X10 is E or V, and
wherein the polypeptide is capable of binding to a FAM19A5 protein, and thereby, inhibiting, reducing, and/or dissociating an interaction between the FAM19A5 protein and a member of the LRRC4 protein family.

17. A LRRC4 family mimic molecule comprising a polypeptide, which comprises an amino acid sequence having the following formula: (from N-terminus to C-terminus): 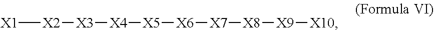
wherein:
X1 is Y, F, V, L, I, W, or M;
X2 is T, S, or Y;
X3 is Y, F, or W;
X4 is F, Y, or W;
X5 is any amino acids, e.g., T, S, or Y;
X6 is T, S, or Y;
X7 is V, I, Y, F, L, W, or M;
X8 is T, S, or Y;
X9 is V, I, Y, F, L, W, or M; and/or
X10 is E, D, V, I, Y, F, M, or W, and
wherein the polypeptide is capable of binding to a FAM19A5 protein, and thereby, inhibiting, reducing, and/or dissociating an interaction between the FAM19A5 protein and a member of the LRRC4 protein family.

18. The LRRC4 family mimic molecule of claim 17, wherein X1 is Y, F, V, L, or I.

19. The LRRC4 family mimic molecule of claim 17 or 18, wherein X2 is T or S.

20. The LRRC4 family mimic molecule of any one of claims 17 to 19, wherein X3 is Y or F.

21. The LRRC4 family mimic molecule of any one of claims 17 to 20, wherein X4 is F.

22. The LRRC4 family mimic molecule of any one of claims 17 to 21, wherein X5 is T or S.

23. The LRRC4 family mimic molecule of any one of claims 17 to 22, wherein X6 is T.

24. The LRRC4 family mimic molecule of any one of claims 17 to 23, wherein X7 is V or I.

25. The LRRC4 family mimic molecule of any one of claims 17 to 24, wherein X8 is T.

26. The LRRC4 family mimic molecule of any one of claims 17 to 25, wherein X9 is V.

27. The LRRC4 family mimic molecule of any one of claims 17 to 26, wherein X10 is E or V.

28. The LRRC4 family mimic molecule of any one of claims 10 to 27, wherein the polypeptide comprises the amino acid sequence set forth in SEQ ID NO: 29 (YTYFTTVTVE).

29. The LRRC4 family mimic molecule of any one of claims 10 to 27, wherein the polypeptide consists of the amino acid sequence set forth in SEQ ID NO: 29 (YTYFTTVTVE).

30. The LRRC4 family mimic molecule of any one of claims 10 to 27, wherein the polypeptide comprises the amino acid sequence set forth in SEQ ID NO: 20 (NYSFFTTVTVETTEISPEDTTRK).

31. The LRRC4 family mimic molecule of any one of claims 10 to 27, wherein the polypeptide consists of the amino acid sequence set forth in SEQ ID NO: 20 (NYSFFTTVTVETTEISPEDTTRK).

32. The LRRC4 family mimic molecule of any one of claims 1 to 19, wherein the polypeptide comprises the amino acid sequence set forth in SEQ ID NO: 30 (YSFFTTVTVE).

33. The LRRC4 family mimic molecule of any one of claims 1 to 19, wherein the polypeptide consists of the amino acid sequence set forth in SEQ ID NO: 30 (YSFFTTVTVE).

34. The LRRC4 family mimic molecule of any one of claims 10 to 27, wherein the polypeptide comprises the forth in SEQ ID NO: 21 amino acid sequence set (NFSYFSTVTVETMEPSQDERTTR).

35. The LRRC4 family mimic molecule of any one of claims 10 to 27, wherein the polypeptide consists of the amino acid sequence set forth in SEQ ID NO: 21 (NFSYFSTVTVETMEPSQDERTTR).

36. The LRRC4 family mimic molecule of any one of claims 1 to 19, wherein the polypeptide comprises the amino acid sequence set forth in SEQ ID NO: 31 (FSYFSTVTVE).

37. The LRRC4 family mimic molecule of any one of claims 1 to 19, wherein the polypeptide consists of the amino acid sequence set forth in SEQ ID NO: 31 (FSYFSTVTVE).

38. The LRRC4 family mimic molecule of any one of claims 10 to 27, wherein the polypeptide comprises the amino acid sequence set forth in SEQ ID NO: 18 (GYTYFTTVTVETLETQPGEE).

39. The LRRC4 family mimic molecule of any one of claims 10 to 27, wherein the polypeptide consists of the amino acid sequence set forth in SEQ ID NO: 18 (GYTYFTTVTVETLETQPGEE).

40. The LRRC4 family mimic molecule of claim 30 or 31, wherein amino acid residues T12 and L13 are modified (e.g., substituted) relative to the corresponding residues of SEQ ID NO: 18.

41. The LRRC4 family mimic molecule of claim 32, wherein the polypeptide comprises the amino acid sequence set forth in any one of SEQ ID NOs: 123-142.

42. The LRRC4 family mimic molecule of claim 32, wherein the polypeptide consists of the amino acid sequence set forth in any one of SEQ ID NOs: 123-142.

43. The LRRC4 family mimic molecule of any one of claims 40 to 42, wherein one or more of the amino acid residues are in the form of a D-amino acid.

44. The LRRC4 family mimic molecule of any one of claims 1 to 19, wherein the polypeptide comprises the amino acid sequence set forth in SEQ ID NO: 17 (GYTYFTTVTVETLETQ).

45. The LRRC4 family mimic molecule of any one of claims 1 to 19, wherein the polypeptide consists of the amino acid sequence set forth in SEQ ID NO: 17 (GYTYFTTVTVETLETQ).

46. The LRRC4 family mimic molecule of any one of claims 1 to 19, wherein the polypeptide comprises the amino acid sequence set forth in SEQ ID NO: 19 (GYTYFTTVTVETLETQPGEKEPPGPTTD).

47. The LRRC4 family mimic molecule of any one of claims 1 to 19, wherein the polypeptide consists of the amino acid sequence set forth in SEQ ID NO: 19 (GYTYFTTVTVETLETQPGEKEPPGPTTD).

48. The LRRC4 family mimic molecule of any one of claims 1 to 19, wherein the polypeptide comprises the amino acid sequence set forth in SEQ ID NO: 143 (GYTYFTTVTVETLETQPGEEA).

49. The LRRC4 family mimic molecule of any one of claims 1 to 19, wherein the polypeptide consists of the amino acid sequence set forth in SEQ ID NO: 143 (GYTYFTTVTVETLETQPGEEA).

50. The LRRC4 family mimic molecule of claim 47 or 48, wherein amino acid residues T12 and L13 are modified (e.g., substituted) relative to the corresponding residues of SEQ ID NO: 143.

51. The LRRC4 family mimic molecule of claim 50, wherein the polypeptide comprises the amino acid sequence set forth in any one of SEQ ID NOs: 123-149.

52. The LRRC4 family mimic molecule of claim 50, wherein the polypeptide consists of the amino acid sequence set forth in any one of SEQ ID NOs: 123-149.

53. The LRRC4 family mimic molecule of any one of claims 16 to 52, wherein the amino acid at X2 is phosphorylated or O-glycosylated.

54. The LRRC4 family mimic molecule of any one of claims 1 to 53, wherein the polypeptide is conjugated to a moiety.

55. The LRRC4 family mimic molecule of claim 54, wherein the moiety is capable of increasing one or more of the following properties of the polypeptide: (1) binding affinity to a FAM19A5 protein, (2) solubility, (3) resistance to degradation from protease and/or peptidase, (4) suitability for in vivo administration, (5) ability to inhibit FAM19A5-LRRC4 protein family member interaction, or (6) any combination of (1) to (5).

56. The LRRC4 family mimic molecule of claim 53 or 54, wherein the moiety comprises a juxta-membrane sequence of the LRRC4 protein family members.

57. The LRRC4 family mimic molecule of claim 56, wherein the juxta-membrane comprises the sequence set forth in SEQ ID NO: 151 (LDEVMKTTK) or SEQ ID NO: 152 (IDEVMKTTK).

58. The LRRC4 family mimic molecule of claim 56, wherein the juxta-membrane consists of the sequence set forth in SEQ ID NO: 151 (LDEVMKTTK) or SEQ ID NO: 152 (IDEVMKTTK).

59. A LRRC4 family mimic molecule comprising a polypeptide, which comprises an amino acid sequence having at least about 60%, at least about 70%, at least about 80%, at least about 90%, at least about 95%, or at least about 99% sequence identity to the amino acid sequence set forth in SEQ ID NO: 29 (YTYFTTVTVE), wherein the polypeptide is capable of binding to a FAM19A5 protein, and thereby, inhibiting, reducing, and/or dissociating an interaction between the FAM19A5 protein and a member of the LRRC4 protein family.

60. A LRRC4 family mimic molecule comprising a polypeptide, which comprises an amino acid sequence that is at least about 70%, at least about 75%, at least about 80%, at least about 85%, at least about 90%, at least about 95%, at least about 96%, at least about 97%, at least about 98%, or about 99% identical to the amino acid sequence set forth in SEQ ID NO: 5, SEQ ID NO: 4, or SEQ ID NO: 6 and contains at least one amino acid modification relative to the amino acid sequence set forth in SEQ ID NO: 5, SEQ ID NO: 4, or SEQ ID NO: 6, respectively, and wherein the polypeptide is capable of binding to a FAM19A5 protein, and thereby, inhibiting, reducing, and/or dissociating an interaction between the FAM19A5 protein and a member of the LRRC4 protein family.

61. The LRRC4 family mimic molecule of claim 60, wherein the at least one amino acid modification increases the binding of the polypeptide to the FAM19A5 protein.

62. The LRRC4 family mimic molecule of claim 60 or 61, wherein the at least one amino acid modification increases the stability of the polypeptide.

63. The LRRC4 family mimic molecule of claim 61 or 62, wherein the increase in the binding and/or stability improves the ability of the polypeptide to inhibit, reduce, and/or dissociate the interaction between the FAM19A5 protein and the member of the LRRC4 protein family.

64. The LRRC4 family mimic molecule of claim 63, wherein the ability of the polypeptide to inhibit, reduce, and/or dissociate the interaction between a FAM19A5 protein and a member of the LRRC4 protein family is increased by at least about 0.5-fold, at least about 1-fold, at least about 2-fold, at least about 3-fold, at least about 4-fold, at least about 5-fold, at least about 10-fold, at least about 20-fold, at least about 30-fold, at least about 40-fold, or at least about 50-fold, compared to a corresponding polypeptide without the at least one amino acid modification.

65. The LRRC4 family mimic molecule of any one of claims 60 to 64, wherein the amino acid residue at position 453 of SEQ ID NO: 5 is T or modified to S or Y.

66. The LRRC4 family mimic molecule of any one of claims 60 to 65, wherein the amino acid residue at position 454 of SEQ ID NO: 5 is T or modified to S or Y.

67. The LRRC4 family mimic molecule of any one of claims 60 to 66, wherein the amino acid residue at position 449 of SEQ ID NO: 5 is Y or modified to F, V, L, I, W, or M.

68. The LRRC4 family mimic molecule of any one of claims 60 to 67, wherein the amino acid residue at position 450 of SEQ ID NO: 5 is T or modified to S or Y.

69. The LRRC4 family mimic molecule of any one of claims 60 to 68, wherein the amino acid residue at position 451 of SEQ ID NO: 5 is Y or modified to F or W.

70. The LRRC4 family mimic molecule of any one of claims 60 to 69, wherein the amino acid residue at position 452 of SEQ ID NO: 5 is F or modified to Y or W.

71. The LRRC4 family mimic molecule of any one of claims 60 to 70, wherein the amino acid residue at position 455 of SEQ ID NO: 5 is V or modified to I, Y, F, L, W, or M.

72. The LRRC4 family mimic molecule of any one of claims 60 to 71, wherein the amino acid residue at position 456 of SEQ ID NO: 5 is T or modified to S or Y.

73. The LRRC4 family mimic molecule of any one of claims 60 to 72, wherein the amino acid residue at position 457 of SEQ ID NO: 5 is V or modified to I, Y, F, L, W, or M.

74. The LRRC4 family mimic molecule of any one of claims 60 to 73, wherein the amino acid residue at position 458 of SEQ ID NO: 5 is E or modified to D, V, I, Y, F, M, or W.

75. The LRRC4 family mimic molecule of any one of claims 60 to 74, wherein one or more of the amino acid residues are in a D-form.

76. The LRRC4 family mimic molecule of claim 75, wherein the D-form amino acid is at the N-terminus, C-terminus, or both.

77. The LRRC4 family mimic molecule of any one of claims 60 to 76, which is conjugated to a moiety.

78. The LRRC4 family mimic molecule of claim 77, wherein the moiety is capable of increasing one or more of the following properties of the polypeptide: (1) binding affinity to a FAM19A5 protein, (2) solubility, (3) resistance to degradation from protease and/or peptidase, (4) suitability for in vivo administration, (5) ability to inhibit FAM19A5-LRRC4 protein family member interaction, or (6) any combination of (1) to (5).

79. The LRRC4 family mimic molecule of claim 77 or 78, wherein the moiety comprises a juxta-membrane sequence of the LRRC4 protein family members.

80. The LRRC4 family mimic molecule of claim 79, wherein the juxta-membrane comprises the sequence set forth in SEQ ID NO: 151 (LDEVMKTTK) or SEQ ID NO: 152 (IDEVMKTTK).

81. The LRRC4 family mimic molecule of claim 79, wherein the juxta-membrane consists of the sequence set forth in SEQ ID NO: 151 (LDEVMKTTK) or SEQ ID NO: 152 (IDEVMKTTK).

82. The LRRC4 family mimic molecule of any one of claims 1 to 81, which does not comprise the transmembrane domain and/or the intracellular domain of a members of the LRRC4 protein family.

83. The LRRC4 family mimic molecule of any one of claims 1 to 82, which is capable of competing with the member of the LRRC4 protein family for binding to the FAM19A5 protein.

84. The LRRC4 family mimic molecule of any one of claims 1 to 83, wherein the member of the LRRC4 protein family comprises a LRRC4 protein, LRRC4B protein, LRRC4C protein, or combinations thereof.

85. The LRRC4 family mimic molecule of any one of claims 10 to 84, which further comprises one or more additional amino acids at the N-terminus of the polypeptide, the C-terminus of the polypeptide, or both the N-terminus and the C-terminus of the polypeptide.

86. The LRRC4 family mimic molecule of claim 85, wherein the one or more additional amino acids are hydrophilic amino acids.

87. The LRRC4 family mimic molecule of claim 85 or 86, wherein the one or more additional amino acids are D-amino acids.

88. The LRRC4 family mimic molecule of any one of claims 10 to 87, wherein the N-terminus, C-terminus, or both the N-terminus and the C-terminus of the polypeptide comprise a modification which increases the stability of the polypeptide.

89. The LRRC4 family mimic molecule of claim 88, wherein the modification comprises a Fmoc, PEGylation, acetylation, methylation, cyclization, or combinations thereof.

90. The LRRC4 family mimic molecule of any one of claims 10 to 89, which is a fusion protein.

91. The LRRC4 family mimic molecule of any one of claims 10 to 90, further comprises a half-life extending moiety.

92. The LRRC4 family mimic molecule of claim 91, wherein the half-life extending moiety comprises a Fc, albumin, an albumin-binding polypeptide, Pro/Ala/Ser (PAS), a C-terminal peptide (CTP) of the β subunit of human chorionic gonadotropin, polyethylene glycol (PEG), long unstructured hydrophilic sequences of amino acids (XTEN), hydroxyethyl starch (HES), an albumin-binding small molecule, or a combination thereof.

93. A nucleic acid encoding the LRRC4 family mimic molecule of any one of claims 10 to 92.

94. The nucleic acid of claim 93, which is a DNA or a RNA.

95. The nucleic acid of claim 94, which is a mRNA.

96. The nucleic acid of any one of claims 93 to 95, comprising a nucleic acid analog.

97. A vector comprising the nucleic acid of any one of claims 93 to 96.

98. A cell comprising the vector of claim 97.

99. A protein conjugate comprising the LRRC4 family mimic molecule of any one of claims 10 to 92, linked to an agent.

100. A composition comprising the LRRC4 family mimic molecule of any one of claims 1 to 92, the nucleic acid of any one of claims 93 to 96, the vector of claim 97, the cell of claim 98, or the protein conjugate of claim 99.

101. The composition of claim 100, which further comprises a pharmaceutically acceptable carrier.

102. A kit comprising the LRRC4 family mimic molecule of any one of claims 1 to 92, the nucleic acid of any one of claims 93 to 96, the vector of claim 97, the cell of claim 98, the protein conjugate of claim 99, or the composition of claim 100 or 101, and instructions for use.

103. A method of producing a molecule that is capable of inhibiting, reducing, and/or dissociating an interaction between a FAM19A5 protein and a member of a LRRC4 protein family, comprising synthesizing the molecule of any one of claims 1 to 9 or culturing the cell of claim 98 under suitable conditions such that the molecule is produced.

104. The method of claim 103, further comprising isolating the molecule which has been produced.

105. A method of increasing a neurite outgrowth and/or synapse formation in neurons, comprising contacting a neuron with the LRRC4 family mimic molecule of any one of claims 1 to 92, the nucleic acid of any one of claims 93 to 96, the vector of claim 97, the cell of claim 98, the protein conjugate of claim 99, or the composition of claim 100 or 101.

106. The method of claim 105, wherein the contacting occurs in vivo in a subject in need thereof.

107. The method of claim 106, which comprises administering the LRRC4 family mimic molecule, the nucleic acid, the vector, the cell, the protein conjugate, or the composition to the subject prior to the contacting.

108. The method of claim 107, wherein the contacting occurs ex vivo.

109. The method of any one of claims 105 to 108, wherein the contacting increases neurite outgrowth in the neuron by at least about 0.5-fold, at least about 1-fold, at least about 2-fold, at least about 3-fold, at least about 4-fold, at least about 5-fold, at least about 10-fold, at least about 20-fold, at least about 30-fold, at least about 40-fold, or at least about 50-fold, compared to a reference (e.g., neurite outgrowth in a corresponding neuron that was not contacted).

110. The method of any one of claims 105 to 109, wherein the contacting increases synapse formation in the neuron by at least about 0.5-fold, at least about 1-fold, at least about 2-fold, at least about 3-fold, at least about 4-fold, at least about 5-fold, at least about 10-fold, at least about 20-fold, at least about 30-fold, at least about 40-fold, or at least about 50-fold, compared to a reference (e.g., synapse formation in a corresponding neuron that was not contacted).

111. The method of any one of claims 105 to 110, wherein the increase in neurite outgrowth and/or synapse formation reduces one or more symptoms associated with a disease or condition selected from an amyotrophic lateral sclerosis (ALS), Alzheimer's disease, glaucoma, diabetic retinopathy, neuropathic pain, spinal cord injury, traumatic brain injury, stroke, Parkinson's disease, or combinations thereof.

112. A method of inhibiting or decreasing a formation of a complex between a FAM19A5 protein and a member of the LRRC4 protein family in a subject in need thereof, comprising administering to the subject the LRRC4 family mimic molecule of any one of claims 1 to 92, the nucleic acid of any one of claims 93 to 96, the vector of claim 97, the cell of claim 98, the protein conjugate of claim 99, or the composition of claim 100 or 101.

113. The method of claim 112, wherein the formation of a complex between a FAM19A5 protein and a member of the LRRC4 protein family is decreased by at least about 5%, at least about 10%, at least about 20%, at least about 30%, at least about 40%, at least about 50%, at least about 60%, at least about 70%, at least about 80%, at least about 90%, or about 100% after the administration.

114. The method of claim 112 or 113, wherein the decrease in the formation of a complex between a FAM19A5 protein and a member of the LRRC4 protein family increases an activity of the member of the LRRC4 protein family in the subject.

115. A method of treating a disease or condition in a subject in need thereof, comprising administering to the subject the LRRC4 family mimic molecule of any one of claims 1 to 92, the nucleic acid of any one of claims 93 to 96, the vector of claim 97, the cell of claim 98, the protein conjugate of claim 99, or the composition of claim 100 or 101, wherein the disease or condition is selected from an amyotrophic lateral sclerosis (ALS), Alzheimer's disease, glaucoma, diabetic retinopathy, neuropathic pain, spinal cord injury, traumatic brain injury, stroke, Parkinson's disease, or combinations thereof.

116. A method of increasing a neurite outgrowth and/or synapse formation in neurons, comprising contacting a neuron with a Leucine Rich Repeat Containing 4 (“LRRC4”) family mimic molecule, wherein the LRRC4 family mimic molecule is capable of inhibiting, reducing, and/or dissociating an interaction between a Family with Sequence Similarity 19, Member A5 (“FAM19A5”) protein and a member of a LRRC4 protein family, and wherein the LRRC4 family mimic molecule is a small molecule selected from: 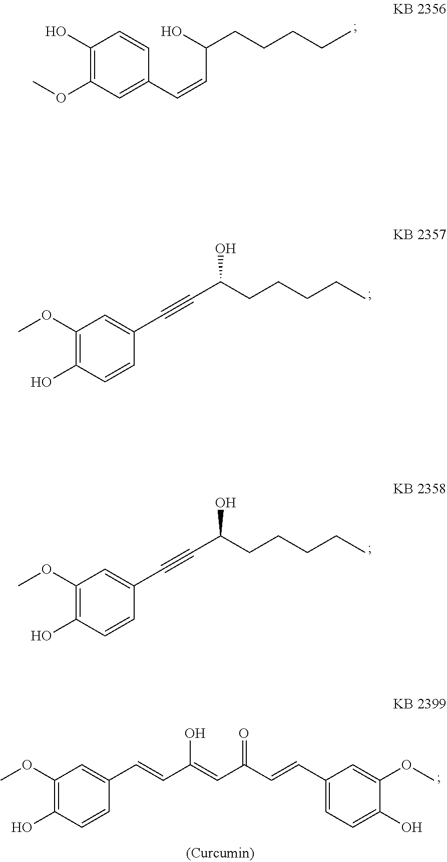
or a pharmaceutically acceptable salt thereof.

117. The method of claim 116, wherein the contacting occurs in vivo in a subject in need thereof.

118. The method of claim 117, which comprises administering the LRRC4 family mimic molecule to the subject prior to the contacting.

119. The method of claim 118, wherein the contacting occurs ex vivo.

120. The method of any one of claims 116 to 119, wherein the contacting increases neurite outgrowth in the neuron by at least about 0.5-fold, at least about 1-fold, at least about 2-fold, at least about 3-fold, at least about 4-fold, at least about 5-fold, at least about 10-fold, at least about 20-fold, at least about 30-fold, at least about 40-fold, or at least about 50-fold, compared to a reference (e.g., neurite outgrowth in a corresponding neuron that was not contacted).

121. The method of any one of claims 116 to 120, wherein the contacting increases synapse formation in the neuron by at least about 0.5-fold, at least about 1-fold, at least about 2-fold, at least about 3-fold, at least about 4-fold, at least about 5-fold, at least about 10-fold, at least about 20-fold, at least about 30-fold, at least about 40-fold, or at least about 50-fold, compared to a reference (e.g., synapse formation in a corresponding neuron that was not contacted).

122. The method of any one of claims 116 to 121, wherein the increase in neurite outgrowth and/or synapse formation reduces one or more symptoms associated with a disease or condition selected from an amyotrophic lateral sclerosis (ALS), Alzheimer's disease, glaucoma, diabetic retinopathy, neuropathic pain, spinal cord injury, traumatic brain injury, stroke, Parkinson's disease, or combinations thereof.

123. A method of inhibiting or decreasing a formation of a complex between a FAM19A5 protein and a member of the LRRC4 protein family in a subject in need thereof, comprising administering to the subject a Leucine Rich Repeat Containing 4 (“LRRC4”) family mimic molecule, wherein the LRRC4 family mimic molecule is capable of inhibiting, reducing, and/or dissociating an interaction between a Family with Sequence Similarity 19, Member A5 (“FAM19A5”) protein and a member of a LRRC4 protein family, and wherein the LRRC4 family mimic molecule is a small molecule selected from: 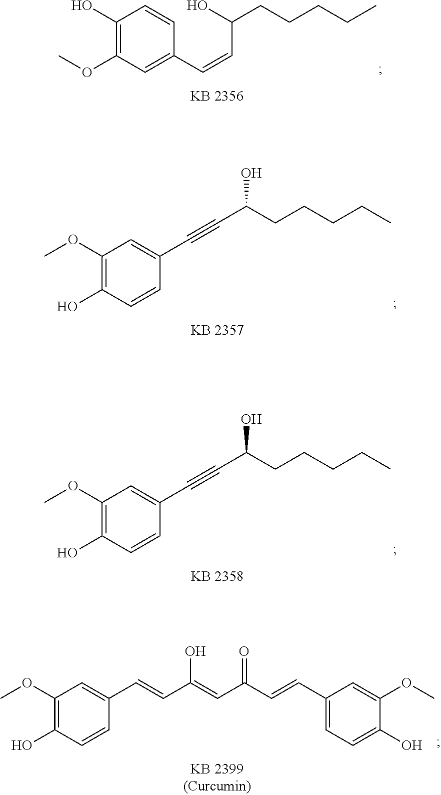
or a pharmaceutically acceptable salt thereof.

124. The method of claim 123, wherein the formation of a complex between a FAM19A5 protein and a member of the LRRC4 protein family is decreased by at least about 5%, at least about 10%, at least about 20%, at least about 30%, at least about 40%, at least about 50%, at least about 60%, at least about 70%, at least about 80%, at least about 90%, or about 100% after the administration.

125. The method of claim 123 or 124, wherein the decrease in the formation of a complex between a FAM19A5 protein and a member of the LRRC4 protein family increases an activity of the member of the LRRC4 protein family in the subject.

126. A method of treating a disease or condition in a subject in need thereof, comprising administering to the subject a Leucine Rich Repeat Containing 4 (“LRRC4”) family mimic molecule, wherein the LRRC4 family mimic molecule is capable of inhibiting, reducing, and/or dissociating an interaction between a Family with Sequence Similarity 19, Member A5 (“FAM19A5”) protein and a member of a LRRC4 protein family, wherein the LRRC4 family mimic molecule is a small molecule selected from: 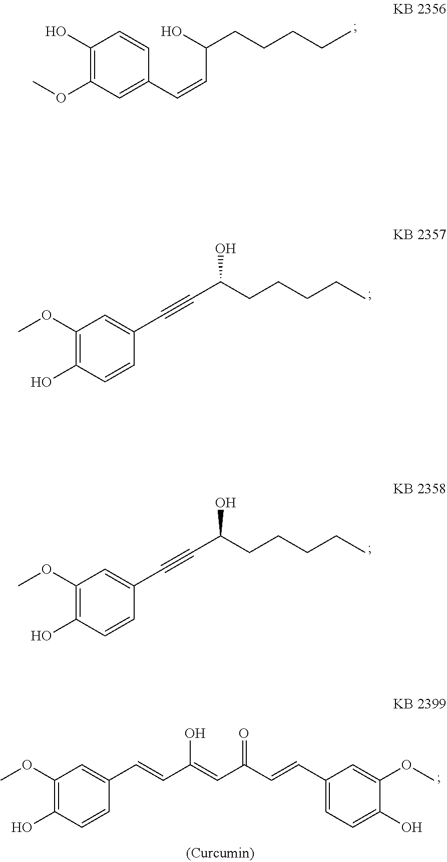
or a pharmaceutically acceptable salt thereof, and wherein the disease or condition is selected from an amyotrophic lateral sclerosis (ALS), Alzheimer's disease, glaucoma, diabetic retinopathy, neuropathic pain, spinal cord injury, traumatic brain injury, stroke, Parkinson's disease, or combinations thereof.


SEQUENCE LISTING (156 sequences)

SEQ ID NO: 1 - protein, 653 aa, synthetic construct
MKLLWQVTVHHHTWNAILLPFVYLTAQVWILCAAIAAAASAGPQNCPSVCSCSNQFSKVVCTRRGLSEVPQGIPSNTRYLNLMENNIQMIQADTFRHLHHLEVLQLGRNSIRQIEVGAFNGLASLNTLELFDNWLTVIPSGAFEYLSKLRELWLRNNPIESIPSYAFNRVPSLMRLDLGELKKLEYISEGAFEGLFNLKYLNLGMCNIKDMPNLTPLVGLEELEMSGNHFPEIRPGSFHGLSSLKKLWVMNSQVSLIERNAFDGLASLVELNLAHNNLSSLPHDLFTPLRYLVELHLHHNPWNCDCDILWLAWWLREYIPTNSTCCGRCHAPMHMRGRYLVEVDQASFQCSAPFIMDAPRDLNISEGRMAELKCRTPPMSSVKWLLPNGTVLSHASRHPRISVLNDGTLNFSHVLLSDTGVYTCMVTNVAGNSNASAYLNVSTAELNTSNYSFFTTVTVETTEISPEDTTRKYKPVPTTSTGYQPAYTTSTTVLIQTTRVPKQVAVPATDTTDKMQTSLDEVMKTTKIIIGCFVAVTLLAAAMLIVFYKLRKRHQQRSTVTAARTVEIIQVDEDIPAATSAAATAAPSGVSGEGAVVLPTIHDHINYNTYKPAHGAHWTENSLGNSLHPTVTTISEPYIIQTHTKDKVQETQI

SEQ ID NO: 2 - protein, 713 aa, synthetic construct
MARARGSPCPPLPPGRMSWPHGALLFLWLFSPPLGAGGGGVAVTSAAGGGSPPATSCPVACSCSNQASRVICTRRDLAEVPASIPVNTRYLNLQENGIQVIRTDTFKHLRHLEILQLSKNLVRKIEVGAFNGLPSLNTLELFDNRLTTVPTQAFEYLSKLRELWLRNNPIESIPSYAFNRVPSLRRLDLGELKRLEYISEAAFEGLVNLRYLNLGMCNLKDIPNLTALVRLEELELSGNRLDLIRPGSFQGLTSLRKLWLMHAQVATIERNAFDDLKSLEELNLSHNNLMSLPHDLFTPLHRLERVHLNHNPWHCNCDVLWLSWWLKETVPSNTTCCARCHAPAGLKGRYIGELDQSHFTCYAPVIVEPPTDLNVTEGMAAELKCRTGTSMTSVNWLTPNGTLMTHGSYRVRISVLHDGTLNFTNVTVQDTGQYTCMVTNSAGNTTASATLNVSAVDPVAAGGTGSGGGGPGGSGGVGGGSGGYTYFTTVTVETLETQPGEEALQPRGTEKEPPGPTTDGVWGGGRPGDAAGPASSSTTAPAPRSSRPTEKAFTVPITDVTENALKDLDDVMKTTKIIIGCFVAITFMAAVMLVAFYKLRKQHQLHKHHGPTRTVEIINVEDELPAASAVSVAAAAAVASGGGVGGDSHLALPALERDHLNHHHYVAAAFKAHYSSNPSGGGCGGKGPPGLNSIHEPLLFKSGSKENVQETQI

SEQ ID NO: 3 - protein, 640 aa, synthetic construct
MLNKMTLHPQQIMIGPRFNRALFDPLLVVLLALQLLVVAGLVRAQTCPSVCSCSNQFSKVICVRKNLREVPDGISTNTRLLNLHENQIQIIKVNSFKHLRHLEILQLSRNHIRTIEIGAFNGLANLNTLELFDNRLTTIPNGAFVYLSKLKELWLRNNPIESIPSYAFNRIPSLRRLDLGELKRLSYISEGAFEGLSNLRYLNLAMCNLREIPNLTPLIKLDELDLSGNHLSAIRPGSFQGLMHLQKLWMIQSQIQVIERNAFDNLQSLVEINLAHNNLTLLPHDLFTPLHHLERIHLHHNPWNCNCDILWLSWWIKDMAPSNTACCARCNTPPNLKGRYIGELDQNYFTCYAPVIVEPPADLNVTEGMAAELKCRASTSLTSVSWITPNGTVMTHGAYKVRIAVLSDGTLNFTNVTVQDTGMYTCMVSNSVGNTTASATLNVTAATTTPFSYFSTVTVETMEPSQDEARTTDNNVGPTPVVDWETTNVTTSLTPQSTRSTEKTFTIPVTDINSGIPGIDEVMKTTKIIIGCFVAITLMAAVMLVIFYKMRKQHHRQNHHAPTRTVEIINVDDEITGDTPMESHLPMPAIEHEHLNHYNSYKSPFNHTTTVNTINSIHSSVHEPLLIRMNSKDNVQETQI

SEQ ID NO: 4 - protein, 489 aa, synthetic construct
ASAGPQNCPSVCSCSNQFSKVVCTRRGLSEVPQGIPSNTRYLNLMENNIQMIQADTFRHLHHLEVLQLGRNSIRQIEVGAFNGLASLNTLELFDNWLTVIPSGAFEYLSKLRELWLRNNPIESIPSYAFNRVPSLMRLDLGELKKLEYISEGAFEGLFNLKYLNLGMCNIKDMPNLTPLVGLEELEMSGNHFPEIRPGSFHGLSSLKKLWVMNSQVSLIERNAFDGLASLVELNLAHNNLSSLPHDLFTPLRYLVELHLHHNPWNCDCDILWLAWWLREYIPTNSTCCGRCHAPMHMRGRYLVEVDQASFQCSAPFIMDAPRDLNISEGRMAELKCRTPPMSSVKWLLPNGTVLSHASRHPRISVLNDGTLNFSHVLLSDTGVYTCMVTNVAGNSNASAYLNVSTAELNTSNYSFFTTVTVETTEISPEDTTRKYKPVPTTSTGYQPAYTTSTTVLIQTTRVPKQVAVPATDTTDKMQTSLDEVMKTTK

SEQ ID NO: 5 - protein, 541 aa, synthetic construct
AGGGGVAVTSAAGGGSPPATSCPVACSCSNQASRVICTRRDLAEVPASIPVNTRYLNLQENGIQVIRTDTFKHLRHLEILQLSKNLVRKIEVGAFNGLPSLNTLELFDNRLTTVPTQAFEYLSKLRELWLRNNPIESIPSYAFNRVPSLRRLDLGELKRLEYISEAAFEGLVNLRYLNLGMCNLKDIPNLTALVRLEELELSGNRLDLIRPGSFQGLTSLRKLWLMHAQVATIERNAFDDLKSLEELNLSHNNLMSLPHDLFTPLHRLERVHLNHNPWHCNCDVLWLSWWLKETVPSNTTCCARCHAPAGLKGRYIGELDQSHFTCYAPVIVEPPTDLNVTEGMAAELKCRTGTSMTSVNWLTPNGTLMTHGSYRVRISVLHDGTLNFTNVTVQDTGQYTCMVTNSAGNTTASATLNVSAVDPVAAGGTGSGGGGPGGSGGVGGGSGGYTYFTTVTVETLETQPGEEALQPRGTEKEPPGPTTDGVWGGGRPGDAAGPASSSTTAPAPRSSRPTEKAFTVPITDVTENALKDLDDVMKTTK

SEQ ID NO: 6 - protein, 483 aa, synthetic construct
QTCPSVCSCSNQFSKVICVRKNLREVPDGISTNTRLLNLHENQIQIIKVNSFKHLRHLEILQLSRNHIRTIEIGAFNGLANLNTLELFDNRLTTIPNGAFVYLSKLKELWLRNNPIESIPSYAFNRIPSLRRLDLGELKRLSYISEGAFEGLSNLRYLNLAMCNLREIPNLTPLIKLDELDLSGNHLSAIRPGSFQGLMHLQKLWMIQSQIQVIERNAFDNLQSLVEINLAHNNLTLLPHDLFTPLHHLERIHLHHNPWNCNCDILWLSWWIKDMAPSNTACCARCNTPPNLKGRYIGELDQNYFTCYAPVIVEPPADLNVTEGMAAELKCRASTSLTSVSWITPNGTVMTHGAYKVRIAVLSDGTLNFTNVTVQDTGMYTCMVSNSVGNTTASATLNVTAATTTPFSYFSTVTVETMEPSQDEARTTDNNVGPTPVVDWETTNVTTSLTPQSTRSTEKTFTIPVTDINSGIPGIDEVMKTTK

SEQ ID NO: 7 - protein, 124 aa, synthetic construct
VSAVDPVAAGGTGSGGGGPGGSGGVGGGSGGYTYFTTVTVETLETQPGEEALQPRGTEKEPPGPTTDGVWGGGRPGDAAGPASSSTTAPAPRSSRPTEKAFTVPITDVTENALKDLDDVMKTTK

SEQ ID NO: 8 - protein, 93 aa, synthetic construct
YTYFTTVTVETLETQPGEEALQPRGTEKEPPGPTTDGVWGGGRPGDAAGPASSSTTAPAPRSSRPTEKAFTVPITDVTENALKDLDDVMKTTK

SEQ ID NO: 9 - protein, 95 aa, synthetic construct
GGYTYFTTVTVETLETQPGEEALQPRGTEKEPPGPTTDGVWGGGRPGDAAGPASSSTTAPAPRSSRPTEKAFTVPITDVTENALKDLDDVMKTTK

SEQ ID NO: 10 - protein, 16 aa, synthetic construct
GGYTYFTTVTVETLET

SEQ ID NO: 11 - protein, 79 aa, synthetic construct
QPGEEALQPRGTEKEPPGPTTDGVWGGGRPGDAAGPASSSTTAPAPRSSRPTEKAFTVPITDVTENALKDLDDVMKTTK

SEQ ID NO: 12 - protein, 33 aa, synthetic construct
LNVSAVDPVAAGGTGSGGGGPGGSGGVGGGSGG

SEQ ID NO: 13 - protein, 34 aa, synthetic construct
LNVSAVDPVAAGGTGSGGGGPGGSGGVGGGSGGY

SEQ ID NO: 14 - protein, 39 aa, synthetic construct
YTYFTTVTVETLETQPGEEALQPRGTEKEPPGPTTDGVW

SEQ ID NO: 15 - protein, 13 aa, synthetic construct
YTYFTTVTVETLE

SEQ ID NO: 16 - protein, 124 aa, synthetic construct
VSAVDPVAAGGTGSGGGGPGGSGGVGGGSGGYTYFAAVTVETLETQPGEEALQPRGTEKEPPGPTTDGVWGGGRPGDAAGPASSSTTAPAPRSSRPTEKAFTVPITDVTENALKDLDDVMKTTK

SEQ ID NO: 17 - protein, 16 aa, synthetic construct
GYTYFTTVTVETLETQ

SEQ ID NO: 18 - protein, 20 aa, synthetic construct
GYTYFTTVTVETLETQPGEE

SEQ ID NO: 19 - protein, 28 aa, synthetic construct
GYTYFTTVTVETLETQPGEKEPPGPTTD

SEQ ID NO: 20 - protein, 23 aa, synthetic construct
NYSFFTTVTVETTEISPEDTTRK

SEQ ID NO: 21 - protein, 23 aa, synthetic construct
NFSYFSTVTVETMEPSQDERTTR

SEQ ID NO: 22 - protein, 132 aa, synthetic construct
MAPSPRTGSRQDATALPSMSSTFWAFMILASLLIAYCSQLAAGTCEIVTLDRDSSQPRRTIARQTARCACRKGQIAGTTRARPACVDARIIKTKQWCDMLPCLEGEGCDLLINRSGWTCTQPGGRIKTTTVS

SEQ ID NO: 23 - protein, 125 aa, synthetic construct
MQLLKALWALAGAALCCFLVLVIHAQFLKEGQLAAGTCEIVTLDRDSSQPRRTIARQTARCACRKGQIAGTTRARPACVDARIIKTKQWCDMLPCLEGEGCDLLINRSGWTCTQPGGRIKTTTVS

SEQ ID NO: 24 - protein, 53 aa, synthetic construct
MYHHREWPARIIKTKQWCDMLPCLEGEGCDLLINRSGWTCTQPGGRIKTTTVS

SEQ ID NO: 25-26 - skipped (2 entries)

SEQ ID NO: 27 - protein, 10 aa, synthetic construct
XXXFXTXTVX

SEQ ID NO: 28 - skipped

SEQ ID NO: 29 - protein, 10 aa, synthetic construct
YTYFTTVTVE

SEQ ID NO: 30 - protein, 10 aa, synthetic construct
YSFFTTVTVE

SEQ ID NO: 31 - protein, 10 aa, synthetic construct
FSYFSTVTVE

SEQ ID NO: 32 - protein, 19 aa, synthetic construct
TFFYGGSRGKRNNFKTEEY

SEQ ID NO: 33 - protein, 39 aa, synthetic construct
SSVIDALQYKLEGTTRLTRKRGLKLATALSLSNKFVEGS

SEQ ID NO: 34 - protein, 18 aa, synthetic construct
LRKLRKRLLLRKLRKRLL

SEQ ID NO: 35 - protein, 8 aa, synthetic construct
CMPRLRGC

SEQ ID NO: 36 - protein, 12 aa, synthetic construct
THRPPMWSPVWP

SEQ ID NO: 37 - protein, 12 aa, synthetic construct
PWVPSWMPPRHT

SEQ ID NO: 38 - protein, 9 aa, synthetic construct
CRTIGPSVC

SEQ ID NO: 39 - protein, 30 aa, synthetic construct
YQQILTSMPSRNVIQISNDLENLRDLLHVL

SEQ ID NO: 40 - protein, 29 aa, synthetic construct
YTIWMPENPRPGTPCDIFTNSRGKRASNG

SEQ ID NO: 41 - protein, 16 aa, synthetic construct
GREIRTGRAERWSEKF

SEQ ID NO: 42 - protein, 18 aa, synthetic construct
CNCKAPETALCARRCQQH

SEQ ID NO: 43 - protein, 8 aa, synthetic construct
KAPETALD

SEQ ID NO: 44 - skipped

SEQ ID NO: 45 - protein, 13 aa, synthetic construct
HLNILSTLWKYRC

SEQ ID NO: 46 - protein, 7 aa, synthetic construct
GFTGFLS

SEQ ID NO: 47 - protein, 12 aa, synthetic construct
TGNYKALHPHNG

SEQ ID NO: 48 - protein, 11 aa, synthetic construct
YGRKKRRQRRR

SEQ ID NO: 49 - protein, 18 aa, synthetic construct
RGGRLSYSRRRFSTSTGR

SEQ ID NO: 50 - skipped

SEQ ID NO: 51 - protein, 4 aa, synthetic construct
PPPP

SEQ ID NO: 52 - protein, 678 aa, synthetic construct
AGGGGVAVTSAAGGGSPPATSCPVACSCSNQASRVICTRRDLAEVPASIPVNTRYLNLQENGIQVIRTDTFKHLRHLEILQLSKNLVRKIEVGAFNGLPSLNTLELFDNRLTTVPTQAFEYLSKLRELWLRNNPIESIPSYAFNRVPSLRRLDLGELKRLEYISEAAFEGLVNLRYLNLGMCNLKDIPNLTALVRLEELELSGNRLDLIRPGSFQGLTSLRKLWLMHAQVATIERNAFDDLKSLEELNLSHNNLMSLPHDLFTPLHRLERVHLNHNPWHCNCDVLWLSWWLKETVPSNTTCCARCHAPAGLKGRYIGELDQSHFTCYAPVIVEPPTDLNVTEGMAAELKCRTGTSMTSVNWLTPNGTLMTHGSYRVRISVLHDGTLNFTNVTVQDTGQYTCMVTNSAGNTTASATLNVSAVDPVAAGGTGSGGGGPGGSGGVGGGSGGYTYFTTVTVETLETQPGEEALQPRGTEKEPPGPTTDGVWGGGRPGDAAGPASSSTTAPAPRSSRPTEKAFTVPITDVTENALKDLDDVMKTTKIIIGCFVAITFMAAVMLVAFYKLRKQHQLHKHHGPTRTVEIINVEDELPAASAVSVAAAAAVASGGGVGGDSHLALPALERDHLNHHHYVAAAFKAHYSSNPSGGGCGGKGPPGLNSIHEPLLFKSGSKENVQETQI

SEQ ID NO: 53 - protein, 557 aa, synthetic construct
LSKLRELWLRNNPIESIPSYAFNRVPSLRRLDLGELKRLEYISEAAFEGLVNLRYLNLGMCNLKDIPNLTALVRLEELELSGNRLDLIRPGSFQGLTSLRKLWLMHAQVATIERNAFDDLKSLEELNLSHNNLMSLPHDLFTPLHRLERVHLNHNPWHCNCDVLWLSWWLKETVPSNTTCCARCHAPAGLKGRYIGELDQSHFTCYAPVIVEPPTDLNVTEGMAAELKCRTGTSMTSVNWLTPNGTLMTHGSYRVRISVLHDGTLNFTNVTVQDTGQYTCMVTNSAGNTTASATLNVSAVDPVAAGGTGSGGGGPGGSGGVGGGSGGYTYFTTVTVETLETQPGEEALQPRGTEKEPPGPTTDGVWGGGRPGDAAGPASSSTTAPAPRSSRPTEKAFTVPITDVTENALKDLDDVMKTTKIIIGCFVAITFMAAVMLVAFYKLRKQHQLHKHHGPTRTVEIINVEDELPAASAVSVAAAAAVASGGGVGGDSHLALPALERDHLNHHHYVAAAFKAHYSSNPSGGGCGGKGPPGLNSIHEPLLFKSGSKENVQETQI

SEQ ID NO: 54 - protein, 484 aa, synthetic construct
RLEELELSGNRLDLIRPGSFQGLTSLRKLWLMHAQVATIERNAFDDLKSLEELNLSHNNLMSLPHDLFTPLHRLERVHLNHNPWHCNCDVLWLSWWLKETVPSNTTCCARCHAPAGLKGRYIGELDQSHFTCYAPVIVEPPTDLNVTEGMAAELKCRTGTSMTSVNWLTPNGTLMTHGSYRVRISVLHDGTLNFTNVTVQDTGQYTCMVTNSAGNTTASATLNVSAVDPVAAGGTGSGGGGPGGSGGVGGGSGGYTYFTTVTVETLETQPGEEALQPRGTEKEPPGPTTDGVWGGGRPGDAAGPASSSTTAPAPRSSRPTEKAFTVPITDVTENALKDLDDVMKTTKIIIGCFVAITFMAAVMLVAFYKLRKQHQLHKHHGPTRTVEIINVEDELPAASAVSVAAAAAVASGGGVGGDSHLALPALERDHLNHHHYVAAAFKAHYSSNPSGGGCGGKGPPGLNSIHEPLLFKSGSKENVQETQI

SEQ ID NO: 55 - protein, 350 aa, synthetic construct
PVIVEPPTDLNVTEGMAAELKCRTGTSMTSVNWLTPNGTLMTHGSYRVRISVLHDGTLNFTNVTVQDTGQYTCMVTNSAGNTTASATLNVSAVDPVAAGGTGSGGGGPGGSGGVGGGSGGYTYFTTVTVETLETQPGEEALQPRGTEKEPPGPTTDGVWGGGRPGDAAGPASSSTTAPAPRSSRPTEKAFTVPITDVTENALKDLDDVMKTTKIIIGCFVAITFMAAVMLVAFYKLRKQHQLHKHHGPTRTVEIINVEDELPAASAVSVAAAAAVASGGGVGGDSHLALPALERDHLNHHHYVAAAFKAHYSSNPSGGGCGGKGPPGLNSIHEPLLFKSGSKENVQETQI

SEQ ID NO: 56 - protein, 261 aa, synthetic construct
VSAVDPVAAGGTGSGGGGPGGSGGVGGGSGGYTYFTTVTVETLETQPGEEALQPRGTEKEPPGPTTDGVWGGGRPGDAAGPASSSTTAPAPRSSRPTEKAFTVPITDVTENALKDLDDVMKTTKIIIGCFVAITFMAAVMLVAFYKLRKQHQLHKHHGPTRTVEIINVEDELPAASAVSVAAAAAVASGGGVGGDSHLALPALERDHLNHHHYVAAAFKAHYSSNPSGGGCGGKGPPGLNSIHEPLLFKSGSKENVQETQI

SEQ ID NO: 57 - protein, 137 aa, synthetic construct
IIIGCFVAITFMAAVMLVAFYKLRKQHQLHKHHGPTRTVEIINVEDELPAASAVSVAAAAAVASGGGVGGDSHLALPALERDHLNHHHYVAAAFKAHYSSNPSGGGCGGKGPPGLNSIHEPLLFKSGSKENVQETQI

SEQ ID NO: 58 - protein, 541 aa, synthetic construct; identical to SEQ ID NO: 5

SEQ ID NO: 59 - protein, 328 aa, synthetic construct
AGGGGVAVTSAAGGGSPPATSCPVACSCSNQASRVICTRRDLAEVPASIPVNTRYLNLQENGIQVIRTDTFKHLRHLEILQLSKNLVRKIEVGAFNGLPSLNTLELFDNRLTTVPTQAFEYLSKLRELWLRNNPIESIPSYAFNRVPSLRRLDLGELKRLEYISEAAFEGLVNLRYLNLGMCNLKDIPNLTALVRLEELELSGNRLDLIRPGSFQGLTSLRKLWLMHAQVATIERNAFDDLKSLEELNLSHNNLMSLPHDLFTPLHRLERVHLNHNPWHCNCDVLWLSWWLKETVPSNTTCCARCHAPAGLKGRYIGELDQSHFTCYA

SEQ ID NO: 60 - protein, 465 aa, synthetic construct
AGGGGVAVTSAAGGGSPPATSCPVACSCSNQASRVICTRRDLAEVPASIPVNTRYLNLQENGIQVIRTDTFKHLRHLEILQLSKNLVRKIEVGAFNGLPSLNTLELFDNRLTTVPTQAFEYLSKLRELWLRNNPIESIPSYAFNRVPSLRRLDLGELKRLEYISEAAFEGLVNLRYLNLGMCNLKDIPNLTALVRLEELELSGNRLDLIRPGSFQGLTSLRKLWLMHAQVATIERNAFDDLKSLEELNLSHNNLMSLPHDLFTPLHRLERVHLNHNPWHCNCDVLWLSWWLKETVPSNTTCCARCHAPAGLKGRYIGELDQSHFTCYAIIIGCFVAITFMAAVMLVAFYKLRKQHQLHKHHGPTRTVEIINVEDELPAASAVSVAAAAAVASGGGVGGDSHLALPALERDHLNHHHYVAAAFKAHYSSNPSGGGCGGKGPPGLNSIHEPLLFKSGSKENVQETQI

SEQ ID NO: 61 - protein, 213 aa, synthetic construct
PVIVEPPTDLNVTEGMAAELKCRTGTSMTSVNWLTPNGTLMTHGSYRVRISVLHDGTLNFTNVTVQDTGQYTCMVTNSAGNTTASATLNVSAVDPVAAGGTGSGGGGPGGSGGVGGGSGGYTYFTTVTVETLETQPGEEALQPRGTEKEPPGPTTDGVWGGGRPGDAAGPASSSTTAPAPRSSRPTEKAFTVPITDVTENALKDLDDVMKTTK

SEQ ID NO: 62-64 - skipped (3 entries)

SEQ ID NO: 65 - protein, 14 aa, synthetic construct
YTYFTTVTVETLET

SEQ ID NO: 66 - protein, 173 aa, synthetic construct
VIVEPPADLNVTEGMAAELKCRASTSLTSVSWITPNGTVMTHGAYKVRIAVLSDGTLNFTNVTVQDTGMYTCMVSNSVGNTTASATLNVTAATTTPFSYFSTVTVETMEPSQDEARTTDNNVGPTPVVDWETTNVTTSLTPQSTRSTEKTFTIPVTDINSGIPGIDEVMKTTK

SEQ ID NO: 67 - protein, 175 aa, synthetic construct
PFIMDAPRDLNISEGRMAELKCRTPPMSSVKWLLPNGTVLSHASRHPRISVLNDGTLNFSHVLLSDTGVYTCMVTNVAGNSNASAYLNVSTAELNTSNYSFFTTVTVETTEISPEDTTRKYKPVPTTSTGYQPAYTTSTTVLIQTTRVPKQVAVPATDTTDKMQTSLDEVMKTTK

SEQ ID NO: 68 - protein, 18 aa, synthetic construct
KNYTYFTTVTVETNKETQ

SEQ ID NO: 69 - protein, 17 aa, synthetic construct
KNYTYFTTVTVENKETQ

SEQ ID NO: 70 - protein, 16 aa, synthetic construct
KNYTYFTTVTVNKETQ

SEQ ID NO: 71 - protein, 15 aa, synthetic construct
KNYTYFTTVTNKETQ

SEQ ID NO: 72 - protein, 14 aa, synthetic construct
KNYTYFTTVNKETQ

SEQ ID NO: 73 - protein, 13 aa, synthetic construct
KNYTYFTTNKETQ

SEQ ID NO: 74 - protein, 17 aa, synthetic construct
KNTYFTTVTVETNKETQ

SEQ ID NO: 75 - protein, 16 aa, synthetic construct
KNYFTTVTVETNKETQ

SEQ ID NO: 76 - protein, 15 aa, synthetic construct
KNFTTVTVETNKETQ

SEQ ID NO: 77 - protein, 14 aa, synthetic construct
KNTTVTVETNKETQ

SEQ ID NO: 78 - protein, 20 aa, synthetic construct
GATYFTTVTVETLETQPGEE

SEQ ID NO: 79 - protein, 20 aa, synthetic construct
GYAYFTTVTVETLETQPGEE

SEQ ID NO: 80 - protein, 20 aa, synthetic construct
GYTAFTTVTVETLETQPGEE

SEQ ID NO: 81 - protein, 20 aa, synthetic construct
GYTYATTVTVETLETQPGEE

SEQ ID NO: 82 - protein, 20 aa, synthetic construct
GYTYFATVTVETLETQPGEE

SEQ ID NO: 83 - protein, 20 aa, synthetic construct
GYTYFTAVTVETLETQPGEE

SEQ ID NO: 84 - protein, 20 aa, synthetic construct
GYTYFTTATVETLETQPGEE

SEQ ID NO: 85 - protein, 20 aa, synthetic construct
GYTYFTTVAVETLETQPGEE

SEQ ID NO: 86 - protein, 20 aa, synthetic construct
GYTYFTTVTAETLETQPGEE

SEQ ID NO: 87 - protein, 20 aa, synthetic construct
GYTYFTTVTVATLETQPGEE

SEQ ID NO: 88 - protein, 20 aa, synthetic construct
GYTYFTTVTVEALETQPGEE

SEQ ID NO: 89 - protein, 20 aa, synthetic construct
GYTYFTTVTVETAETQPGEE

SEQ ID NO: 90 - protein, 20 aa, synthetic construct
GYTYFTTVTVETLATQPGEE

SEQ ID NO: 91 - protein, 20 aa, synthetic construct
GNTYFTTVTVETLETQPGEE

SEQ ID NO: 92 - protein, 20 aa, synthetic construct
GYNYFTTVTVETLETQPGEE

SEQ ID NO: 93 - protein, 20 aa, synthetic construct
GYTNFTTVTVETLETQPGEE

SEQ ID NO: 94 - protein, 20 aa, synthetic construct
GYTYNTTVTVETLETQPGEE

SEQ ID NO: 95 - protein, 20 aa, synthetic construct
GYTYFNTVTVETLETQPGEE

SEQ ID NO: 96 - protein, 20 aa, synthetic construct
GYTYFTNVTVETLETQPGEE

SEQ ID NO: 97 - protein, 20 aa, synthetic construct
GYTYFTTNTVETLETQPGEE

SEQ ID NO: 98 - protein, 20 aa, synthetic construct
GYTYFTTVNVETLETQPGEE

SEQ ID NO: 99 - protein, 20 aa, synthetic construct
GYTYFTTVTNETLETQPGEE

SEQ ID NO: 100 - protein, 20 aa, synthetic construct
GYTYFTTVTVNTLETQPGEE

SEQ ID NO: 101 - protein, 20 aa, synthetic construct
GYTYFTTVTVENLETQPGEE

SEQ ID NO: 102 - skipped

SEQ ID NO: 103 - protein, 20 aa, synthetic construct
AYTYFTTVTVETLETQPGEE

SEQ ID NO: 104-116 - skipped (13 entries)

SEQ ID NO: 117 - protein, 20 aa, synthetic construct
GYTYFTTVTVETLEAQPGEE

SEQ ID NO: 118 - protein, 20 aa, synthetic construct
GYTYFTTVTVETLETAPGEE

SEQ ID NO: 119 - protein, 20 aa, synthetic construct
GYTYFTTVTVETLETQAGEE

SEQ ID NO: 120 - protein, 20 aa, synthetic construct
GYTYFTTVTVETLETQPAEE

SEQ ID NO: 121 - protein, 20 aa, synthetic construct
GYTYFTTVTVETLETQPGAE

SEQ ID NO: 122 - protein, 20 aa, synthetic construct
GYTYFTTVTVETLETQPGEA

SEQ ID NO: 123 - protein, 20 aa, synthetic construct
GYTYFTTVTVEPYETQPGEE

SEQ ID NO: 124 - protein, 20 aa, synthetic construct
GYTYFTTVTVEMRETQPGEE

SEQ ID NO: 125 - protein, 20 aa, synthetic construct
GYTYFTTVTVEIFETQPGEE

SEQ ID NO: 126 - protein, 20 aa, synthetic construct
GYTYFTTVTVEHFETQPGEE

SEQ ID NO: 127 - protein, 20 aa, synthetic construct
GYTYFTTVTVEWYETQPGEE

SEQ ID NO: 128 - protein, 20 aa, synthetic construct
GYTYFTTVTVEQRETQPGEE

SEQ ID NO: 129 - protein, 20 aa, synthetic construct
GYTYFTTVTVEWFETQPGEE

SEQ ID NO: 130 - protein, 20 aa, synthetic construct
GYTYFTTVTVEERETQPGEE

SEQ ID NO: 131 - protein, 20 aa, synthetic construct
GYTYFTTVTVEDYETQPGEE

SEQ ID NO: 132 - protein, 20 aa, synthetic construct
GYTYFTTVTVEFFETQPGEE

SEQ ID NO: 133 - protein, 20 aa, synthetic construct
GYTYFTTVTVEHYETQPGEE

SEQ ID NO: 134 - protein, 20 aa, synthetic construct
GYTYFTTVTVEMMETQPGEE

SEQ ID NO: 135 - protein, 20 aa, synthetic construct
GYTYFTTVTVEDFETQPGEE

SEQ ID NO: 136 - protein, 20 aa, synthetic construct
GYTYFTTVTVEDIETQPGEE

SEQ ID NO: 137 - protein, 20 aa, synthetic construct
GYTYFTTVTVELIETQPGEE

SEQ ID NO: 138 - protein, 20 aa, synthetic construct
GYTYFTTVTVEEIETQPGEE

SEQ ID NO: 139 - protein, 20 aa, synthetic construct
GYTYFTTVTVEAFETQPGEE

SEQ ID NO: 140 - protein, 20 aa, synthetic construct
GYTYFTTVTVEHHETQPGEE

SEQ ID NO: 141 - protein, 20 aa, synthetic construct
GYTYFTTVTVEPFETQPGEE

SEQ ID NO: 142 - protein, 20 aa, synthetic construct
GYTYFTTVTVEDWETQPGEE

SEQ ID NO: 143 - protein, 21 aa, synthetic construct
GYTYFTTVTVETLETQPGEEA

SEQ ID NO: 144 - protein, 21 aa, synthetic construct
GYTYFTTVTVEPYETQPGEEA

SEQ ID NO: 145 - protein, 21 aa, synthetic construct
GYTYFTTVTVEHFETQPGEEA

SEQ ID NO: 146 - protein, 21 aa, synthetic construct
GYTYFTTVTVEQRETQPGEEA

SEQ ID NO: 147 - protein, 21 aa, synthetic construct
GYTYFTTVTVEWYETQPGEEA

SEQ ID NO: 148 - protein, 21 aa, synthetic construct
GYTYFTTVTVEMRETQPGEEA

SEQ ID NO: 149 - protein, 21 aa, synthetic construct
GYTYFTTVTVEIFETQPGEEA

SEQ ID NO: 150 - skipped

SEQ ID NO: 151 - protein, 9 aa, synthetic construct
LDEVMKTTK

SEQ ID NO: 152 - protein, 9 aa, synthetic construct
IDEVMKTTK

SEQ ID NO: 153 - protein, 21 aa, synthetic construct
NYTYFTTVTVETLETQPGEEA

SEQ ID NO: 154 - protein, 39 aa, synthetic construct
NYTYFTTVTVETLETQPGEEALRKLRKRLLLRKLRKRLL

SEQ ID NO: 155 - protein, 31 aa, synthetic construct
NYTYFTTVTVETLETQPGEEALDEVMKTTKA

SEQ ID NO: 156 - protein, 31 aa, synthetic construct
NYTYFTTVTVEDYETQPGEEALDEVMKTTKA